SEQUENCE LISTING (300 sequences)

SEQ ID NO: 1 - protein, 58 aa, Artificial Sequence
  Description of Artificial Sequence: Synthetic polypeptide
  MOD_RES (1)..(4): Any non-cysteine amino acid or not present
  MOD_RES (6)..(9): Any amino acid
  MOD_RES (10)..(10): Asp or Glu
  MOD_RES (11)..(11): Asp, Gly, Ser, Val, Asn, Ile, Ala or Thr
  MOD_RES (13)..(13): Pro, Arg, His, Asn, Ser, Thr, Ala, Gly, Lys or Gln
  MOD_RES (15)..(15): Arg, Lys, Ala, Ser, Gly, Met, Asn or Gln
  MOD_RES (16)..(16): Ala, Gly, Ser, Asp or Asn
  MOD_RES (17)..(17): Ala, Asn, Ser, Ile, Gly, Val, Gln or Thr
  MOD_RES (18)..(18): His, Leu, Gln or Ala
  MOD_RES (19)..(19): Pro, Gln, Leu, Asn or Ile
  MOD_RES (20)..(20): Any amino acid
  MOD_RES (21)..(21): Trp, Phe, Tyr, His or Ile
  MOD_RES (22)..(29): Any amino acid
  MOD_RES (31)..(31): Glu, Asp, Gln, Asn, Ser, Ala, Val, Leu, Ile or Thr
  MOD_RES (32)..(32): Glu, Gln, Asp, Asn, Pro, Thr, Leu, Ser, Ala, Gly or Val
  MOD_RES (34)..(34): Ile, Thr, Ser, Val, Ala, Asn, Gly or Leu
  MOD_RES (35)..(35): Tyr, Trp or Phe
  MOD_RES (39)..(39): Glu, Gly, Ala, Ser or Asp
  MOD_RES (40)..(50): Any amino acid
  MOD_RES (52)..(54): Any amino acid
  MOD_RES (56)..(58): Any non-cysteine amino acid or not present
XXXXCXXXXXXGXCXXXXXXXXXXXXXXXCXXFXXGGCXXXXXXXXXXXXCXXXCXXX

SEQ ID NO: 2 - protein, 60 aa, Artificial Sequence
  Description of Artificial Sequence: Synthetic polypeptide
EAMHSFCAFKADDGPCRAAHPRWFFNIFTRQCEEFIYGGCEGNQNRFESLEECKKMCTRD

SEQ ID NO: 3 - protein, 124 aa, Artificial Sequence
  Description of Artificial Sequence: Synthetic polypeptide
FYSHSAQSELTQPPSASAAPGQRVTISCSGSSSNIGSNTVTWYQKLPGTAPKLLIYNNYERPSGVPARFSGSKSGTSASLAISGLQSEDEADYYCATWDDSLIANYVFGSGTKVTVLGQPKANP

SEQ ID NO: 4 - protein, 58 aa, Artificial Sequence
  Description of Artificial Sequence: Synthetic polypeptide
MHSFCAFKADDGPCKANHLRFFFNIFTRQCEEFSYGGCGGNQNRFESLEECKKMCTRD

SEQ ID NO: 5 - protein, 58 aa, Artificial Sequence
  Description of Artificial Sequence: Synthetic polypeptide
MHSFCAFKADDGHCKANHQRFFFNIFTRQCEEFTYGGCGGNQNRFESLEECKKMCTRD

SEQ ID NO: 6 - protein, 58 aa, Artificial Sequence
  Description of Artificial Sequence: Synthetic polypeptide
MHSFCAFKADDGHCKANHQRFFFNIFTRQCEQFTYGGCAGNQNRFESLEECKKMCTRD

SEQ ID NO: 7 - protein, 58 aa, Artificial Sequence
  Description of Artificial Sequence: Synthetic polypeptide
MHSFCAFKADDGHCKASLPRFFFNIFTRQCEEFIYGGCGGNQNRFESLEECKKMCTRD

SEQ ID NO: 8 - protein, 58 aa, Artificial Sequence
  Description of Artificial Sequence: Synthetic polypeptide
MHSFCAFKADDGHCKANHQRFFFNIFTRQCEEFSYGGCGGNQNRFESLEECKKMCTRD

SEQ ID NO: 9 - protein, 58 aa, Artificial Sequence
  Description of Artificial Sequence: Synthetic polypeptide
MHSFCAFKADDGHCKGAHLRFFFNIFTRQCEEFIYGGCEGNQNRFESLEECKKMCTRD

SEQ ID NO: 10 - protein, 58 aa, Artificial Sequence
  Description of Artificial Sequence: Synthetic polypeptide
MHSFCAFKADDGRCKGAHLRFFFNIFTRQCEEFIYGGCEGNQNRFESLEECKKMCTRD

SEQ ID NO: 11 - protein, 58 aa, Artificial Sequence
  Description of Artificial Sequence: Synthetic polypeptide
MHSFCAFKADGGRCRGAHPRWFFNIFTRQCEEFSYGGCGGNQNRFESLEECKKMCTRD

SEQ ID NO: 12 - protein, 58 aa, Artificial Sequence
  Description of Artificial Sequence: Synthetic polypeptide
MHSFCAFKADDGPCRAAHPRWFFNIFTRQCEEFSYGGCGGNQNRFESLEECKKMCTRD

SEQ ID NO: 13 - protein, 58 aa, Artificial Sequence
  Description of Artificial Sequence: Synthetic polypeptide
MHSFCAFKADVGRCRGAHPRWFFNIFTRQCEEFSYGGCGGNQNRFESLEECKKMCTRD

SEQ ID NO: 14 - protein, 58 aa, Artificial Sequence
  Description of Artificial Sequence: Synthetic polypeptide
MHSFCAFKADVGRCRGAQPRFFFNIFTRQCEEFSYGGCGGNQNRFESLEECKKMCTRD

SEQ ID NO: 15 - protein, 58 aa, Artificial Sequence
  Description of Artificial Sequence: Synthetic polypeptide
MHSFCAFKADDGSCRAAHLRWFFNIFTRQCEEFSYGGCGGNQNRFESLEECKKMCTRD

SEQ ID NO: 16 - protein, 58 aa, Artificial Sequence
  Description of Artificial Sequence: Synthetic polypeptide
MHSFCAFKAEGGSCRAAHQRWFFNIFTRQCEEFSYGGCGGNQNRFESLEECKKMCTRD

SEQ ID NO: 17 - protein, 58 aa, Artificial Sequence
  Description of Artificial Sequence: Synthetic polypeptide
MHSFCAFKADDGPCRGAHLRFFFNIFTRQCEEFSYGGCGGNQNRFESLEECKKMCTRD

SEQ ID NO: 18 - protein, 58 aa, Artificial Sequence
  Description of Artificial Sequence: Synthetic polypeptide
MHSFCAFKADDGHCRGALPRWFFNIFTRQCEEFSYGGCGGNQNRFESLEECKKMCTRD

SEQ ID NO: 19 - protein, 58 aa, Artificial Sequence
  Description of Artificial Sequence: Synthetic polypeptide
MHSFCAFKADSGNCRGNLPRFFFNIFTRQCEEFSYGGCGGNQNRFESLEECKKMCTRD

SEQ ID NO: 20 - protein, 58 aa, Artificial Sequence
  Description of Artificial Sequence: Synthetic polypeptide
MHSFCAFKADSGRCRGNHQRFFFNIFTRQCEEFSYGGCGGNQNRFESLEECKKMCTRD

SEQ ID NO: 21 - protein, 58 aa, Artificial Sequence
  Description of Artificial Sequence: Synthetic polypeptide
MHSFCAFKADGGRCRAIQPRWFFNIFTRQCEEFSYGGCGGNQNRFESLEECKKMCTRD

SEQ ID NO: 22 - protein, 58 aa, Artificial Sequence
  Description of Artificial Sequence: Synthetic polypeptide
MHSFCAFKADDGRCRGAHPRWFFNIFTRQCEEFSYGGCGGNQNRFESLEECKKMCTRD

SEQ ID NO: 23 - protein, 108 aa, Artificial Sequence
  Description of Artificial Sequence: Synthetic polypeptide
DIQMTQSPSSLSASVGDRVTITCRASQSVGTYLNWYQQKPGKAPKLLIYATSNLRSGVPSRFSGSGSGTDFTLTISSLQPEDFATYYCQQSYSIPRFTFGPGTKVDIK

SEQ ID NO: 24 - protein, 120 aa, Artificial Sequence
  Description of Artificial Sequence: Synthetic polypeptide
EVQLLESGGGLVQPGGSLRLSCAASGFTFSVYGMVWVRQAPGKGLEWVSVISSSGGSTWYADSVKGRFTISRDNSKNTLYLQMNSLRAEDTAVYYCARPFSRRYGVFDYWGQGTLVTVSS

SEQ ID NO: 25 - protein, 107 aa, Artificial Sequence
  Description of Artificial Sequence: Synthetic polypeptide
DIQMTQSPSSLSASVGDRVTITCRASQGIRNFLAWYQQKPGKVPKLLIYGASALQSGVPSRFSGSGSGTDFTLTISSLQPEDVATYYCQKYNGVPLTFGGGTKVEIK

SEQ ID NO: 26 - protein, 148 aa, Homo sapiens [sequence omitted]

SEQ ID NO: 27 - DNA, 1955 bp, Homo sapiens
acactttgcgttccgcggccccggccccttggtttcctagtcctggctccattcccctctcaggcctagggctgggacccctccccgcccccggtcttggccctgcccccttcaacagacggtccgccccggcccctccccctcgtcccgcccggccctggcaggccccgccccctgcggcctctacctttgacgtcttcccccgggaggtggcgggggtctgcgaccgaatgccggcgggactctgggtcagggcttctggcgggccctgcggggggcagcgaggtgaccgtgaacctgcggctcatggcgcggaaaggagccaggcggccgcggcaaggtccgggatcgcacaagtggctgcaaccaggctctaggagggagaaagagcggatcccccaaccccctccgcccgcccgccccccgcgagacgcggcgccgcgcagggtcctagtgcccgctgtgcgaagggttcctgaatctggccacttcgctgggaggccctgggctccccagtgccacccgaagggcctgaggaggccatctgcagaatctcactctgtcgcccaggccggagtgcagtgtcatgatcttggctcactgcaacctccgcctcccagttcaggagattctcctgcctcagcctcccgggtggctgggattacaagcacagtgcctggcacattatcggcacttgatgactgttgtctaataactgagcttccatacaaaccacctgccgtcctgtactgaaggagaaagagcttccagccggggaggcaggaaatctgggtcctggtcttggttgcatccctgacttcctaaatgacctggagaaggcctctgcctctgctgggatcttgtctgtgctggggcatttgtttccatttccaagggctttttcttcctcgctcagaatttgaccactcactaagaggagcttagtgtggtgtctcacgaagggatcctcctcagccctcacctcggtactggaagacgtcgtgcgtgtccaaaggcaccccggggaacatccggtccacctcgctggcgctccggggatccaccatctgcgccttcacgtcgaacctgcgggcaggcgcggaggagacaggtgctgagccggctagcggacggaccgacggcgcccgggctccccctgccggcggccgcggcggcgctcacctccagaggcgccgcccgctgaacagcagcatcttccccctgccactccggagggccccggtcacctgggccacgtcggcgcccaggcccagcttgtccagacgcctcgggcccagcaccgacgcgcctgtgtacacccacacctggcgccctgcaggggaggagggtcacgtcggtttgggggcgcagagggagcacgtactcctagaacgcgaggagggagattccggcgaggcctttcctagcccgcgtgcccgcagtccctgcaacccaggggcagaggcgctgggtagagcgacgcgagggcgtggagaggagggggcagaaactcagccgcccctacgtttgctaaactgcgtccgccagggggcgtatttttctaaaacgcacaagacgtttcgtgggttatcgatggtctcttgagcctccttgactgatggggattgaccgggcgggggagggaaagtaggtaactaaccagagaagaagaaaagcttcttggagagcggctcctcaaagaccgagtccagcttgcggggcagcgcgggccacttgtcggcgataaggaaggggccctgcggccggctccccctgccctcagagaatcgccagtacttcctgagaaagcgaggagggaaaggacgggctctaagccttggacacagggccagtgggcgggaagggacgggcagcccctccgcaaagccccctcccgcatccacacaaccccgcctcctcacccatccttgaacaaatacagctggttcccaatc

SEQ ID NO: 28 - protein, 730 aa, Mus musculus
MSPWQPLLLALLAFGCSSAAPYQRQPTFVVFPKDLKTSNLTDTQLAEAYLYRYGYTRAAQMMGEKQSLRPALLMLQKQLSLPQTGELDSQTLKAIRTPRCGVPDVGRFQTFKGLKWDHHNITYWIQNYSEDLPRDMIDDAFARAFAVWGEVAPLTFTRVYGPEADIVIQFGVAEHGDGYPFDGKDGLLAHAFPPGAGVQGDAHFDDDELWSLGKGVVIPTYYGNSNGAPCHFPFTFEGRSYSACTTDGRNDGTPWCSTTADYDKDGKFGFCPSERLYTEHGNGEGKPCVFPFIFEGRSYSACTTKGRSDGYRWCATTANYDQDKLYGFCPTRVDATVVGGNSAGELCVFPFVFLGKQYSSCTSDGRRDGRLWCATTSNFDTDKKWGFCPDQGYSLFLVAAHEFGHALGLDHSSVPEALMYPLYSYLEGFPLNKDDIDGIQYLYGRGSKPDPRPPATTTTEPQPTAPPTMCPTIPPTAYPTVGPTVGPTGAPSPGPTSSPSPGPTGAPSPGPTAPPTAGSSEASTESLSPADNPCNVDVFDAIAEIQGALHFFKDGWYWKFLNHRGSPLQGPFLTARTWPALPATLDSAFEDPQTKRVFFFSGRQMWVYTGKTVLGPRSLDKLGLGPEVTHVSGLLPRRLGKALLFSKGRVWRFDLKSQKVDPQSVIRVDKEFSGVPWNSHDIFQYQDKAYFCHGKFFWRVSFQNEVNKVDHEVNQVDDVGYVTYDLLQCP

SEQ ID NO: 29 - DNA, 3185 bp, Mus musculus
ctcaccatgagtccctggcagcccctgctcctggctctcctggctttcggctgcagctctgctgccccttaccagcgccagccgacttttgtggtcttccccaaagacctgaaaacctccaacctcacggacacccagctggcagaggcatacttgtaccgctatggttacacccgggccgcccagatgatgggagagaagcagtctctacggccggctttgctgatgcttcagaagcagctctccctgccccagactggtgagctggacagccagacactaaaggccattcgaacaccacgctgtggtgtcccagacgtgggtcgattccaaaccttcaaaggcctcaagtgggaccatcataacatcacatactggatccaaaactactctgaagacttgccgcgagacatgatcgatgacgccttcgcgcgcgccttcgcggtgtggggcgaggtggcacccctcaccttcacccgcgtgtacggacccgaagcggacattgtcatccagtttggtgtcgcggagcacggagacgggtatcccttcgacggcaaggacggccttctggcacacgcctttccccctggcgccggcgttcagggagatgcccatttcgacgacgacgagttgtggtcgctgggcaaaggcgtcgtgatccccacttactatggaaactcaaatggtgccccatgtcactttcccttcaccttcgagggacgctcctattcggcctgcaccacagacggccgcaacgacggcacgccttggtgtagcacaacagctgactacgataaggacggcaaatttggtttctgccctagtgagagactctacacggagcacggcaacggagaaggcaaaccctgtgtgttcccgttcatctttgagggccgctcctactctgcctgcaccactaaaggccgctcggatggttaccgctggtgcgccaccacagccaactatgaccaggataaactgtatggcttctgccctacccgagtggacgcgaccgtagttgggggcaactcggcaggagagctgtgcgtcttccccttcgtcttcctgggcaagcagtactcttcctgtaccagcgacggccgcagggatgggcgcctctggtgtgcgaccacatcgaacttcgacactgacaagaagtggggtttctgtccagaccaagggtacagcctgttcctggtggcagcgcacgagttcggccatgcactgggcttagatcattccagcgtgccggaagcgctcatgtacccgctgtatagctacctcgagggcttccctctgaataaagacgacatagacggcatccagtatctgtatggtcgtggctctaagcctgacccaaggcctccagccaccaccacaactgaaccacagccgacagcacctcccactatgtgtcccactatacctcccacggcctatcccacagtgggccccacggttggccctacaggcgccccctcacctggccccacaagcagcccgtcacctggccctacaggcgccccctcacctggccctacagcgccccctactgcgggctcttctgaggcctctacagagtctttgagtccggcagacaatccttgcaatgtggatgtttttgatgctattgctgagatccagggcgctctgcatttcttcaaggacggttggtactggaagttcctgaatcatagaggaagcccattacagggccccttccttactgcccgcacgtggccagccctgcctgcaacgctggactccgcctttgaggatccgcagaccaagagggttttcttcttctctggacgtcaaatgtgggtgtacacaggcaagaccgtgctgggccccaggagtctggataagttgggtctaggcccagaggtaacccacgtcagcgggcttctcccgcgtcgtctcgggaaggctctgctgttcagcaaggggcgtgtctggagattcgacttgaagtctcagaaggtggatccccagagcgtcattcgcgtggataaggagttctctggtgtgccctggaactcacacgacatcttccagtaccaagacaaagcctatttctgccatggcaaattcttctggcgtgtgagtttccaaaatgaggtgaacaaggtggaccatgaggtgaaccaggtggacgacgtgggctacgtgacctacgacctcctgcagtgcccttgaactagggctccttctttgcttcaaccgtgcagtgcaagtctctagagaccaccaccaccaccaccacacacaaaccccatccgagggaaaggtgctagctggccaggtacagactggtgatctcttctagagactgggaaggagtggaggcaggcagggctctctctgcccaccgtcctttcttgttggactgtttctaataaacacggatccccaaccttttccagctactttagtcaatcagcttatctgtagttgcagatgcatccgagcaagaagacaactttgtagggtggattctgaccttttatttttgtgtggcgtctgagaattgaatcagctggcttttgtgacaggcacttcaccggctaaaccacctctcccgactccagcccttttatttattatgtatgaggttatgttcacatgcatgtatttaacccacagaatgcttactgtgtgtcgggcgcggctccaaccgctgcataaatattaaggtattcagttgcccctactggaaggtattatgtaactatttctctcttacattggagaacaccaccgagctatccactcatcaaacatttattgagagcatccctagggagccaggctctctactgggcgttagggacagaaatgttggttcttccttcaaggattgctcagagattctccgtgtcctgtaaatctgctgaaaccagaccccagactcctctctctcccgagagtccaactcactcactgtggttgctggcagctgcagcatgcgtatacagcatgtgtgctagagaggtagagggggtctgtgcgttatggttcaggtcagactgtgtcctccaggtgagatgacccctcagctggaactgatccaggaaggataaccaagtgtcttcctggcagtcttttttaaataaatgaataaatgaatatttacttaaaaaaaaaaaaaaaaaaaaaaaaaaaaaaaaaaaaaaaaaaaaaaa

SEQ ID NO: 30 - protein, 730 aa, Mus musculus; identical to SEQ ID NO: 28

SEQ ID NO: 31 - DNA, 21 bp, Artificial Sequence
  Description of Artificial Sequence: Synthetic oligonucleotide
  Description of Combined DNA/RNA Molecule: Synthetic oligonucleotide
gacuugccgcgagacaugatt

SEQ ID NO: 32 - DNA, 21 bp, Artificial Sequence
  Description of Artificial Sequence: Synthetic oligonucleotide
  Description of Combined DNA/RNA Molecule: Synthetic oligonucleotide
ucaugucucgcggcaaguctt

SEQ ID NO: 33 - protein, 58 aa, Artificial Sequence
  Description of Artificial Sequence: Synthetic polypeptide
  MOD_RES (10)..(11): Any amino acid, any non-Cysteine amino acid or any amino acid from the same set of amino acids that are allowed for SEQ ID NO: 1
  MOD_RES (13)..(13): Any amino acid, any non-Cysteine amino acid or any amino acid from the same set of amino acids that are allowed for SEQ ID NO: 1
  MOD_RES (15)..(19): Any amino acid, any non-Cysteine amino acid or any amino acid from the same set of amino acids that are allowed for SEQ ID NO: 1
  MOD_RES (21)..(21): Any amino acid, any non-Cysteine amino acid or any amino acid from the same set of amino acids that are allowed for SEQ ID NO: 1
  MOD_RES (31)..(32): Any amino acid, any non-Cysteine amino acid or any amino acid from the same set of amino acids that are allowed for SEQ ID NO: 1
  MOD_RES (34)..(35): Any amino acid, any non-Cysteine amino acid or any amino acid from the same set of amino acids that are allowed for SEQ ID NO: 1
  MOD_RES (39)..(39): Any amino acid, any non-Cysteine amino acid or any amino acid from the same set of amino acids that are allowed for SEQ ID NO: 1
MHSFCAFKAXXGXCXXXXXRXFFNIFTRQCXXFXXGGCXGNQNRFESLEECKKMCTRD

SEQ ID NO: 34 - DNA, 21 bp, Artificial Sequence
  Description of Artificial Sequence: Synthetic oligonucleotide
  Description of Combined DNA/RNA Molecule: Synthetic oligonucleotide
gacuucgcgggacacaugatt

SEQ ID NO: 35 - DNA, 21 bp, Artificial Sequence
  Description of Artificial Sequence: Synthetic oligonucleotide
  Description of Combined DNA/RNA Molecule: Synthetic oligonucleotide
ucaugugucccgcgaaguctt

SEQ ID NO: 36 - DNA, 1693 bp, Homo sapiens
atgaagtttcttctaatactgctcctgcaggccactgcttctggagctcttcccctgaacagctctacaagcctggaaaaaaataatgtgctatttggtgaaagatacttagaaaaattttatggccttgagataaacaaacttccagtgacaaaaatgaaatatagtggaaacttaatgaaggaaaaaatccaagaaatgcagcacttcttgggtctgaaagtgaccgggcaactggacacatctaccctggagatgatgcacgcacctcgatgtggagtccccgatgtccatcatttcagggaaatgccaggggggcccgtatggaggaaacattatatcacctacagaatcaataattacacacctgacatgaaccgtgaggatgttgactacgcaatccggaaagctttccaagtatggagtaatgttacccccttgaaattcagcaagattaacacaggcatggctgacattttggtggtttttgcccgtggagctcatggagacttccatgcttttgatggcaaaggtggaatcctagcccatgcttttggacctggatctggcattggaggggatgcacatttcgatgaggacgaattctggactacacattcaggaggcacaaacttgttcctcactgctgttcacgagattggccattccttaggtcttggccattctagtgatccaaaggccgtaatgttccccacctacaaatatgttgacatcaacacatttcgcctctctgctgatgacatacgtggcattcagtccctgtatggagacccaaaagagaaccaacgcttgccaaatcctgacaattcagaaccagctctctgtgaccccaatttgagttttgatgctgtcactaccgtgggaaataagatctttttcttcaaagacaggttcttctggctgaaggtttctgagagaccaaagaccagtgttaatttaatttcttccttatggccaaccttgccatctggcattgaagctgcttatgaaattgaagccagaaatcaagtttttctttttaaagatgacaaatactggttaattagcaatttaagaccagagccaaattatcccaagagcatacattcttttggttttcctaactttgtgaaaaaaattgatgcagctgtttttaacccacgtttttataggacctacttctttgtagataaccagtattggaggtatgatgaaaggagacagatgatggaccctggttatcccaaactgattaccaagaacttccaaggaatcgggcctaaaattgatgcagtcttctactctaaaaacaaatactactatttcttccaaggatctaaccaatttgaatatgacttcctactccaacgtatcaccaaaacactgaaaagcaatagctggtttggttgttagaaatggtgtaattaatggtttttgttagttcacttcagcttaataagtatttattgcatatttgctatgtcctcagtgtaccactacttagagatatgtatcataaaaataaaatctgtaaaccataggtaatgattatataaaatacataatatttttcaattttgaaaactctaattgtccattcttgcttgactctactattaagtttgaaaatagttaccttcaaaggccaagagaattctatttgaagcatgctctgtaagttgcttcctaacat

SEQ ID NO: 37 - protein, 108 aa, Artificial Sequence
  Description of Artificial Sequence: Synthetic polypeptide
QDIQMTQSPATLSVSPGERVTLSCKASHSISRNLAWYQQKPGQAPRLLIFGASTRATGIPARFSGSGSGTEFTLTISSLEAEDFAVYYCQQRRNWPVTFGPGTKLDFK

SEQ ID NO: 38 - protein, 124 aa, Artificial Sequence
  Description of Artificial Sequence: Synthetic polypeptide
EVQLLESGGGLVQPGGSLRLSCAASGFTFSQYPMWWVRQAPGKGLEWVSYIVPSGGRTYYADSVKGRFTISRDNSKNTLYLQMNSLRAEDTAVYYCAKDRAYGDYVGWNGFDYWGQGTLVTVSS

SEQ ID NO: 39 - protein, 108 aa, Artificial Sequence
  Description of Artificial Sequence: Synthetic polypeptide
QDIQMTQSPATLSVSPGERATLSCRASQSVSSNLAWYQQKPGQAPRLLIYGASTRATGIPARFSGSGSGTEFTLTISSLQSEDFAVYYCQQRRNWPVTFGQGTRLEII

SEQ ID NO: 40 - protein, 124 aa, Artificial Sequence; identical to SEQ ID NO: 38
  Description of Artificial Sequence: Synthetic polypeptide

SEQ ID NO: 41 - protein, 108 aa, Artificial Sequence
  Description of Artificial Sequence: Synthetic polypeptide
QDIQMTQSPGTLSVSPGERVTLSCRASQSVSSYLAWYQQKPGQAPRLLIYGASTRVTGIPARFSGSGSGTEFTLTISSLQSEDFAVYYCQQRSSWPITFGQGTRLEIK

SEQ ID NO: 42 - protein, 124 aa, Artificial Sequence; identical to SEQ ID NO: 38
  Description of Artificial Sequence: Synthetic polypeptide

SEQ ID NO: 43 - protein, 107 aa, Artificial Sequence
  Description of Artificial Sequence: Synthetic polypeptide
EIVLTQSPATLSLSPGERATLSCRASQSISSFLAWYQQKPGQAPRLLIYDASYRATGIPARFSGSGSGTDFTLTISSLEPEDFAVYYCQQRGNWPITFGQGTRLEIK

SEQ ID NO: 44 - protein, 108 aa, Artificial Sequence
  Description of Artificial Sequence: Synthetic polypeptide
QDIQMTQSPAALSLSPGERATLSCRASQSVSSDLAWYQQKPGQAPRLLIYGASTRATGIPARFSGSRSGTAFTLTISSLEPEDFAVYYCQQRSNWPVTFGQGTKLEIK

SEQ ID NO: 45 - protein, 124 aa, Artificial Sequence; identical to SEQ ID NO: 38
  Description of Artificial Sequence: Synthetic polypeptide

SEQ ID NO: 46 - protein, 124 aa, Artificial Sequence
  Description of Artificial Sequence: Synthetic polypeptide
EVQLLESGGGLVQPGGSLRLSCAASGFTFSWYDMWWVRQAPGKGLEWVSVIYSSGGPTFYADSVKGRFTISRDNSKNTLYLQMNSLRAEDTAVYYCAKDRAYGDYVGWNGFDYWGQGTLVTVSS

SEQ ID NO: 47 - protein, 107 aa, Artificial Sequence; identical to SEQ ID NO: 43
  Description of Artificial Sequence: Synthetic polypeptide

SEQ ID NO: 48 - protein, 124 aa, Artificial Sequence
  Description of Artificial Sequence: Synthetic polypeptide
EVQLLESGGGLVQPGGSLRLSCAASGFTFSVYDMWWVRQAPGKGLEWVSVIYPSGGATYYADSVKGRFTISRDNSKNTLYLQMNSLRAEDTAVYYCAKDRAYGDYVGWNGFDYWGQGTLVTVSS

SEQ ID NO: 49 - protein, 107 aa, Artificial Sequence; identical to SEQ ID NO: 43
  Description of Artificial Sequence: Synthetic polypeptide

SEQ ID NO: 50 - protein, 123 aa, Artificial Sequence
  Description of Artificial Sequence: Synthetic polypeptide
EVQLLESGGGLVQPGGSLRLSCAASGFTFSQYDMWWVRQAPGKGLEWVSVIYPSGTTFYADSVKGRFTISRDNSKNTLYLQMNSLRAEDTAVYYCAKDRAYGDYVGWNGFDYWGQGTLVTVSS

SEQ ID NO: 51 - protein, 107 aa, Artificial Sequence; identical to SEQ ID NO: 43
  Description of Artificial Sequence: Synthetic polypeptide

SEQ ID NO: 52 - protein, 124 aa, Artificial Sequence
  Description of Artificial Sequence: Synthetic polypeptide
EVQLLESGGGLVQPGGSLRLSCAASGFTFSLYDMWWVRQAPGKGLEWVSVIYSSGGYTGYADSVKGRFTISRDNSKNTLYLQMNSLRAEDTAVYYCAKDRAYGDYVGWNGFDYWGQGTLVTVSS

SEQ ID NO: 53 - protein, 470 aa, Homo sapiens
MKFLLILLLQATASGALPLNSSTSLEKNNVLFGERYLEKFYGLEINKLPVTKMKYSGNLMKEKIQEMQHFLGLKVTGQLDTSTLEMMHAPRCGVPDVHHFREMPGGPVWRKHYITYRINNYTPDMNREDVDYAIRKAFQVWSNVTPLKFSKINTGMADILVVFARGAHGDFHAFDGKGGILAHAFGPGSGIGGDAHFDEDEFWTTHSGGTNLFLTAVHEIGHSLGLGHSSDPKAVMFPTYKYVDINTFRLSADDIRGIQSLYGDPKENQRLPNPDNSEPALCDPNLSFDAVTTVGNKIFFFKDRFFWLKVSERPKTSVNLISSLWPTLPSGIEAAYEIEARNQVFLFKDDKYWLISNLRPEPNYPKSIHSFGFPNFVKKIDAAVFNPRFYRTYFFVDNQYWRYDERRQMMDPGYPKLITKNFQGIGPKIDAVFYSKNKYYYFFQGSNQFEYDFLLQRITKTLKSNSWFGC

SEQ ID NO: 54 - protein, 304 aa, Artificial Sequence
  Description of Artificial Sequence: Synthetic polypeptide
MIYTMKKVHALWASVCLLLNLAPAPLNADSEEDEEHTIITDTELPPLKLMHSFCAFKADDGPCKAIMKRFFFNIFTRQCEEFIYGGCEGNQNRFESLEECKKMCTRDNANRIIKTTLQQEKPDFCFLEEDPGICRGYITRYFYNNQTKQCERFKYGGCLGNMNNFETLEECKNICEDGPNGFQVDNYGTQLNAVNNSLTPQSTKVPSLFEFHGPSWCLTPADRGLCRANENRFYYNSVIGKCRPFKYSGCGGNENNFTSKQECLRACKKGFIQRISKGGLIKTKRKRKKQRVKIAYEEIFVKNM

SEQ ID NO: 55 - protein, 58 aa, Artificial Sequence [sequence omitted]

SEQ ID NO: 56 - DNA, 1410 bp, Mus musculus
actctgctgaaaggagtctgcacaatgaaatttctcatgatgattgtgttcttacaggtatctgcctgtggggctgctcccatgaatgacagtgaatttgctgaatggtacttgtcaagattttatgattatggaaaggacagaattccaatgacaaaaacaaaaaccaatagaaacttcctaaaagaaaaactccaggaaatgcagcagttctttgggctagaagcaactgggcaactggacaactcaactctggcaataatgcacatccctcgatgtggagtgcccgatgtacagcatcttagagcagtgccccagaggtcaagatggatgaagcggtacctcacttacaggatctataattacactccggacatgaagcgtgaggatgtagactacatatttcagaaagctttccaagtctggagtgatgtgactcctctaagattcagaaagcttcataaagatgaggctgacattatgatactttttgcatttggagctcacggagacttcaactattttgatggcaaaggtggtacactagcccatgctttttatcctggacctggtattcaaggagatgcacattttgatgaggcagaaacgtggactaaaagttttcaaggcacaaacctcttccttgttgctgttcatgaacttggccattccttggggctgcagcattccaataatccaaagtcaataatgtaccccacctacagataccttaaccccagcacatttcgcctctctgctgatgacatacgtaacattcagtccctctatggagccccagtgaaacccccatccttgacaaaacctagcagtccaccatcaactttctgtcaccaaagcttgagttttgatgctgtcacaacagtgggagagaaaatctttttctttaaagactggttcttctggtggaagcttcctgggagtccagccaccaacattacttctatttcttccatatggccaagcatcccatctggtattcaagctgcttacgaaattgaaagcagaaatcaacttttcctttttaaagatgagaagtactggttaataaacaacttagtaccagagccacactatcccaggagcatatattccctgggcttctctgcatctgtgaagaaggttgatgcagctgtctttgacccacttcgccaaaaggtttatttctttgtggataaacactactggaggtatgatgtgaggcaggagctcatggaccctgcttaccccaagctgatttccacacacttcccaggaatcaagcctaaaattgatgcagtcctctatttcaaaagacactactacatcttccaaggagcctatcaattggaatatgaccccctgttccgtcgtgtcaccaaaacattgaaaagtacaagctggtttggttgt

SEQ ID NO: 57 - protein, 462 aa, Mus musculus
MKFLMMIVFLQVSACGAAPMNDSEFAEWYLSRFYDYGKDRIPMTKTKTNRNFLKEKLQEMQQFFGLEATGQLDNSTLAIMHIPRCGVPDVQHLRAVPQRSRWMKRYLTYRIYNYTPDMKREDVDYIFQKAFQVWSDVTPLRFRKLHKDEADIMILFAFGAHGDFNYFDGKGGTLAHAFYPGPGIQGDAHFDEAETWTKSFQGTNLFLVAVHELGHSLGLQHSNNPKSIMYPTYRYLNPSTFRLSADDIRNIQSLYGAPVKPPSLTKPSSPPSTFCHQSLSFDAVTTVGEKIFFFKDWFFWWKLPGSPATNITSISSIWPSIPSGIQAAYEIESRNQLFLFKDEKYWLINNLVPEPHYPRSIYSLGFSASVKKVDAAVFDPLRQKVYFFVDKHYWRYDVRQELMDPAYPKLISTHFPGIKPKIDAVLYFKRHYYIFQGAYQLEYDPLFRRVTKTLKSTSWFGC

SEQ ID NO: 58 - protein, 4 aa, Artificial Sequence
  Description of Artificial Sequence: Synthetic peptide
GFLG

SEQ ID NO: 59 - protein, 115 aa, Artificial Sequence
  Description of Artificial Sequence: Synthetic polypeptide
EVQLLESGGGLVQPGGSLRLSCAASGFTFSLYSMNWVRQAPGKGLEWVSSIYSSGGSTLYADSVKGRFTISRDNSKNTLYLQMNSLRAEDTAVYYCARGRAFDIWGQGTMVTVSS

SEQ ID NO: 60 - protein, 582 aa, Homo sapiens
MSPAPRPPRCLLLPLLTLGTALASLGSAQSSSFSPEAWLQQYGYLPPGDLRTHTQRSPQSLSAAIAAMQKFYGLQVTGKADADTMKAMRRPRCGVPDKFGAEIKANVRRKRYAIQGLKWQHNEITFCIQNYTPKVGEYATYEAIRKAFRVWESATPLRFREVPYAYIREGHEKQADIMIFFAEGFHGDSTPFDGEGGFLAHAYFPGPNIGGDTHFDSAEPWTVRNEDLNGNDIFLVAVHELGHALGLEHSSDPSAIMAPFYQWMDTENFVLPDDDRRGIQQLYGGESGFPTKMPPQPRTTSRPSVPDKPKNPTYGPNICDGNFDTVAMLRGEMFVFKERWFWRVRNNQVMDGYPMPIGQFWRGLPASINTAYERKDGKFVFFKGDKHWVFDEASLEPGYPKHIKELGRGLPTDKIDAALFWMPNGKTYFFRGNKYYRFNEELRAVDSEYPKNIKVWEGIPESPRGSFMGSDEVFTYFYKGNKYWKFNNQKLKVEPGYPKSALRDWMGCPSGGRPDEGTEEETEVIIIEVDEEGGGAVSAAAVVLPVLLLLLVLAVGLAVFFFRRHGTPRRLLYCQRSLLDKV

SEQ ID NO: 61 - protein, 582 aa, Mus musculus
MSPAPRPSRSLLLPLLTLGTALASLGWAQGSNFSPEAWLQQYGYLPPGDLRTHTQRSPQSLSAAIAAMQKFYGLQVTGKADLATMMAMRRPRCGVPDKFGTEIKANVRRKRYAIQGLKWQHNEITFCIQNYTPKVGEYATFEAIRKAFRVWESATPLRFREVPYAYIREGHEKQADIMILFAEGFHGDSTPFDGEGGFLAHAYFPGPNIGGDTHFDSAEPWTVQNEDLNGNDIFLVAVHELGHALGLEHSNDPSAIMSPFYQWMDTENFVLPDDDRRGIQQLYGSKSGSPTKMPPQPRTTSRPSVPDKPKNPAYGPNICDGNFDTVAMLRGEMFVFKERWFWRVRNNQVMDGYPMPIGQFWRGLPASINTAYERKDGKFVFFKGDKHWVFDEASLEPGYPKHIKELGRGLPTDKIDAALFWMPNGKTYFFRGNKYYRFNEEFRAVDSEYPKNIKVWEGIPESPRGSFMGSDEVFTYFYKGNKYWKFNNQKLKVEPGYPKSALRDWMGCPSGRRPDEGTEEETEVIIIEVDEEGSGAVSAAAVVLPVLLLLLVLAVGLAVFFFRRHGTPKRLLYCQRSLLDKV

SEQ ID NO: 62 - protein, 141 aa, Artificial Sequence
  Description of Artificial Sequence: Synthetic polypeptide
MGWSCIILFLVATATGAHSEVQLLESGGGLVQPGGSLRLSCAASGFTFSWYWMHWVRQAPGKGLEWVSGISPSGGMTMYADSVKGRFTISRDNSKNTLYLQMNSLRAEDTAVYYCARDIVGPYSAGLFDYWGRGTLVTVSS

SEQ ID NO: 63 - DNA, 372 bp, Artificial Sequence
  Description of Artificial Sequence: Synthetic polynucleotide
ttctattctcacagtgcacagagcgaattgactcagccaccgtcagcgtctgcggcccccgggcagagggtcaccatctcttgttctggaagcagctccaacatcggaagtaacactgtaacctggtaccagaagctcccaggaacggcccccaagctcctcatttacaataattatgagcggccctcaggggtccctgcccgattctctggctccaagtctggcacctcagcctccctggccatcagtgggctccagtctgaggatgaggctgattattactgtgcaacatgggatgacagcctgattgccaattacgtcttcggaagtgggaccaaggtcaccgtcctaggtcagcccaaggccaacccc

SEQ ID NO: 64 - DNA, 483 bp, Artificial Sequence
  Description of Artificial Sequence: Synthetic polynucleotide
atgaagaagctcctctttgctatcccgctcgtcgttccttttgtggcccagccggccatggccgaagttcaattgttagagtctggtggcggtcttgttcagcctggtggttctttacgtctttcttgcgctgcttccggattcactttctctccttaccttatgaattgggttcgccaagctcctggtaaaggtttggagtgggtttcttctatctattcttctggtggcggtactggttatgctgactccgttaaaggtcgcttcactatctctagagacaactctaagaatactctctacttgcagatgaacagcttaagggctgaggacacggccgtgtattactgtgcgagaatataccatagcagcagtggacctttctacggtatggacgtctggggccaagggaccacggtcaccgtctcaagcgcctccaccaagggcccatcggtcttcccgctagcaccctcctccaagagc

SEQ ID NO: 65 - protein, 161 aa, Artificial Sequence
  Description of Artificial Sequence: Synthetic polypeptide
MKKLLFAIPLVVPFVAQPAMAEVQLLESGGGLVQPGGSLRLSCAASGFTFSPYLMNWVRQAPGKGLEWVSSIYSSGGGTGYADSVKGRFTISRDNSKNTLYLQMNSLRAEDTAVYYCARIYHSSSGPFYGMDVWGQGTTVTVSSASTKGPSVFPLAPSSKS

SEQ ID NO: 66 - protein, 14 aa, Artificial Sequence
  Description of Artificial Sequence: Synthetic peptide
TGTSSDVGGYNYVS

SEQ ID NO: 67 - protein, 7 aa, Artificial Sequence
  Description of Artificial Sequence: Synthetic peptide
DVSKRPS

SEQ ID NO: 68 - protein, 10 aa, Artificial Sequence
  Description of Artificial Sequence: Synthetic peptide
CSYAGSYTLV

SEQ ID NO: 69 - protein, 5 aa, Artificial Sequence
  Description of Artificial Sequence: Synthetic peptide
TYQMV

SEQ ID NO: 70 - protein, 17 aa, Artificial Sequence
  Description of Artificial Sequence: Synthetic peptide
VIYPSGGPTVYADSVKG

SEQ ID NO: 71 - protein, 15 aa, Artificial Sequence
  Description of Artificial Sequence: Synthetic peptide
GEDYYDSSGPGAFDI

SEQ ID NO: 72 - protein, 110 aa, Artificial Sequence
  Description of Artificial Sequence: Synthetic polypeptide
QSALTQPRSVSGSPGQSVTISCTGTSSDVGGYNYVSWYQQHPGKAPKLMIYDVSKRPSGVPDRFSGSKSGNTASLTISGLQAEDEADYYCCSYAGSYTLVFGGGTKLTVL

SEQ ID NO: 73 - protein, 106 aa, Artificial Sequence
  Description of Artificial Sequence: Synthetic polypeptide
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 74 - protein, 110 aa, Artificial Sequence
  Description of Artificial Sequence: Synthetic polypeptide
QYELTQPRSVSGSPGQSVTISCTGTSSDVGGYNYVSWYQQHPGKAPKLMIYDVSKRPSGVPDRFSGSKSGNTASLTISGLQAEDEADYYCCSYAGSYTLVFGGGTKLTVL

SEQ ID NO: 75 - protein, 124 aa, Artificial Sequence
  Description of Artificial Sequence: Synthetic polypeptide
EVQLLESGGGLVQPGGSLRLSCAASGFTFSTYQMVWVRQAPGKGLEWVSVIYPSGGPTVYADSVKGRFTISRDNSKNTLYLQMNSLRAEDTAVYYCARGEDYYDSSGPGAFDIWGQGTMVTVSS

SEQ ID NO: 76 - DNA, 330 bp, Artificial Sequence
  Description of Artificial Sequence: Synthetic polynucleotide
  CDS (1)..(330)
cagtacgaattgactcagcctcgctcagtgtccgggtctcctggacagtcagtcaccatctcctgcactggaaccagcagtgatgttggtggttataactatgtctcctggtaccaacagcacccaggcaaagcccccaaactcatgatttatgatgtcagtaagcggccctcaggggtccctgatcgcttctctggctccaagtctggcaacacggcctccctgaccatctctgggctccaggctgaggatgaggctgattattactgctgctcatatgcaggcagctacactttggtgttcggcggagggaccaagctgaccgtccta

SEQ ID NO: 77 - DNA, 372 bp, Artificial Sequence
  Description of Artificial Sequence: Synthetic polynucleotide
  CDS (1)..(372)
gaagttcaattgttagagtctggtggcggtcttgttcagcctggtggttctttacgtctttcttgcgctgcttccggattcactttctctacttaccagatggtttgggttcgccaagctcctggtaaaggtttggagtgggtttctgttatctatccttctggtggccctactgtttatgctgactccgttaaaggtcgcttcactatctctagagacaactctaagaatactctctacttgcagatgaacagcttaagggctgaggacacggccgtgtattactgtgcgagaggggaggactactatgatagtagtggcccgggggcttttgatatctggggccaagggacaatggtcaccgtctcaagc

SEQ ID NO: 78 - DNA, 330 bp, Artificial Sequence
  Description of Artificial Sequence: Synthetic polynucleotide
  CDS (1)..(330)
cagagcgccctgacccagcccagaagcgtgtccggcagcccaggccagagcgtgaccatcagctgcaccggcaccagcagcgacgtgggcggctacaactacgtgtcctggtatcagcagcaccccggcaaggcccccaagctgatgatctacgacgtgtccaagaggcccagcggcgtgcccgacaggttcagcggcagcaagagcggcaacaccgccagcctgaccatctccggactgcaggccgaggacgaggccgactactactgctgcagctacgccggcagctacaccctggtgttcggcggagggaccaagctgaccgtgctg

SEQ ID NO: 79 - DNA, 372 bp, Artificial Sequence
  Description of Artificial Sequence: Synthetic polynucleotide
  CDS (1)..(372)
gaggtgcaattgctggaaagcggcggaggactggtgcagccaggcggcagcctgaggctgtcctgcgccgccagcggcttcaccttcagcacctaccagatggtgtgggtgcgccaggccccaggcaagggcctggaatgggtgtccgtgatctaccccagcggcggacccaccgtgtacgccgacagcgtgaagggcaggttcaccatcagcagggacaacagcaagaacaccctgtacctgcagatgaacagcctgagggccgaggacaccgccgtgtactactgcgccaggggcgaggactactacgacagcagcggcccaggcgccttcgacatctggggccagggcacaatggtgaccgtgtccagc

SEQ ID NO: 80 - DNA, 654 bp, Artificial Sequence
  Description of Artificial Sequence: Synthetic polynucleotide
cagagcgccctgacccagcccagaagcgtgtccggcagcccaggccagagcgtgaccatcagctgcaccggcaccagcagcgacgtgggcggctacaactacgtgtcctggtatcagcagcaccccggcaaggcccccaagctgatgatctacgacgtgtccaagaggcccagcggcgtgcccgacaggttcagcggcagcaagagcggcaacaccgccagcctgaccatctccggactgcaggccgaggacgaggccgactactactgctgcagctacgccggcagctacaccctggtgttcggcggagggaccaagctgaccgtgctgggccagcccaaggctgcccccagcgtgaccctgttcccccccagcagcgaggaactgcaggccaacaaggccacactggtgtgcctgatcagcgacttctacccaggcgccgtgaccgtggcctggaaggccgacagcagccccgtgaaggccggcgtggagacaaccacccccagcaagcagagcaacaacaagtacgccgccagcagctacctgagcctgacccccgagcagtggaagtcccacaggtcctacagctgccaggtgacccacgagggcagcaccgtggagaaaaccgtggcccccaccgagtgtagctgatga

SEQ ID NO: 81 - DNA, 1365 bp, Artificial Sequence
  Description of Artificial Sequence: Synthetic polynucleotide
gaggtgcaattgctggaaagcggcggaggactggtgcagccaggcggcagcctgaggctgtcctgcgccgccagcggcttcaccttcagcacctaccagatggtgtgggtgcgccaggccccaggcaagggcctggaatgggtgtccgtgatctaccccagcggcggacccaccgtgtacgccgacagcgtgaagggcaggttcaccatcagcagggacaacagcaagaacaccctgtacctgcagatgaacagcctgagggccgaggacaccgccgtgtactactgcgccaggggcgaggactactacgacagcagcggcccaggcgccttcgacatctggggccagggcacaatggtgaccgtgtccagcgccagcaccaagggccccagcgtgttcccgctagcaccttcctccaagtccacctctggcggcaccgccgctctgggctgcctggtgaaggactacttccctgagcctgtgaccgtgagctggaactctggcgccctgacctccggcgtgcataccttccctgccgtgctgcagtcctccggcctgtactccctgtcctccgtggtgacagtgccttcctcctccctgggcacccagacctacatctgcaacgtgaaccacaagccttccaacaccaaggtggacaagcgggtggagcctaagtcctgcgacaagacccacacctgccctccctgccctgcccctgagctgctgggcggaccctccgtgttcctgttccctcctaagcctaaggacaccctgatgatctcccggacccctgaggtgacctgcgtggtggtggacgtgtcccacgaggacccagaggtgaagtttaattggtatgtggacggcgtggaggtccacaacgccaagaccaagcctcgggaggaacagtacaactccacctaccgggtggtgtccgtgctgaccgtgctgcaccaggactggctgaacggcaaggaatacaagtgcaaagtctccaacaaggccctgcctgcccccatcgagaaaaccatctccaaggccaagggccagcctcgcgagcctcaggtgtacaccctgcctcctagccgggaggaaatgaccaagaaccaggtgtccctgacctgtctggtgaagggcttctacccttccgatatcgccgtggagtgggagtccaacggccagcctgagaacaactacaagaccacccctcctgtgctggactccgacggctccttcttcctgtactccaagctgaccgtggacaagtcccggtggcagcagggcaacgtgttctcctgctccgtgatgcacgaggccctgcacaaccactacacccagaagtccctgtccctgagccctggcaagtga

SEQ ID NO: 82 - protein, 218 aa, Artificial Sequence
  Description of Artificial Sequence: Synthetic polypeptide
QSALTQPRSVSGSPGQSVTISCTGTSSDVGGYNYVSWYQQHPGKAPKLMIYDVSKRPSGVPDRFSGSKSGNTASLTISGLQAEDEADYYCCSYAGSYTLVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSSS

SEQ ID NO: 83 - protein, 455 aa, Artificial Sequence
  Description of Artificial Sequence: Synthetic polypeptide
EVQLLESGGGLVQPGGSLRLSCAASGFTFSTYQMVWVRQAPGKGLEWVSVIYPSGGPTVYADSVKGRFTISRDNSKNTLYLQMNSLRAEDTAVYYCARGEDYYDSSGPGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKS

SEQ ID NO: 84 - protein, 11 aa, Artificial Sequence
  Description of Artificial Sequence: Synthetic peptide
KASHSISRNLA

SEQ ID NO: 85 - protein, 11 aa, Artificial Sequence
  Description of Artificial Sequence: Synthetic peptide
RASQSVSSNLA

SEQ ID NO: 86 - protein, 11 aa, Artificial Sequence
  Description of Artificial Sequence: Synthetic peptide
RASQSVSSYLA

SEQ ID NO: 87 - protein, 11 aa, Artificial Sequence
  Description of Artificial Sequence: Synthetic peptide
RASQSVSSDLA

SEQ ID NO: 88 - protein, 7 aa, Artificial Sequence
  Description of Artificial Sequence: Synthetic peptide
GASTRAT

SEQ ID NO: 89 - protein, 7 aa, Artificial Sequence
  Description of Artificial Sequence: Synthetic peptide
GASTRVT

SEQ ID NO: 90 - protein, 9 aa, Artificial Sequence
  Description of Artificial Sequence: Synthetic peptide
QQRRNWPVT

SEQ ID NO: 91 - protein, 9 aa, Artificial Sequence
  Description of Artificial Sequence: Synthetic peptide
QQRSSWPIT

SEQ ID NO: 92 - protein, 9 aa, Artificial Sequence
  Description of Artificial Sequence: Synthetic peptide
QQRSNWPVT

SEQ ID NO: 93 - protein, 5 aa, Artificial Sequence
  Description of Artificial Sequence: Synthetic peptide
QYPMW

SEQ ID NO: 94 - protein, 17 aa, Artificial Sequence
  Description of Artificial Sequence: Synthetic peptide
YIVPSGGRTYYADSVKG

SEQ ID NO: 95 - protein, 15 aa, Artificial Sequence
  Description of Artificial Sequence: Synthetic peptide
DRAYGDYVGWNGFDY

SEQ ID NO: 96 - protein, 108 aa, Artificial Sequence
  Description of Artificial Sequence: Synthetic polypeptide
QDIQMTQSPSSLSASVGDRVTITCRASQGIRNDLDWYQQKPGTAPKRLIYSASNLQSGVPSRFSGSGSGTEFTLTISNLQPEDLATYFCLQHNSFPLTFGQGTKVEIK

SEQ ID NO: 97 - protein, 116 aa, Artificial Sequence
  Description of Artificial Sequence: Synthetic polypeptide
EVQLLESGGGLVQPGGSLRLSCAASGFTFSLYRMNWVRQAPGKGLEWVSYIGSSGGATAYADSVKGRFTISRDNSKNTLYLQMNSLRAEDTAVYYCARGAWYLDSWGQGTLVTVSS

SEQ ID NO: 98 - protein, 109 aa, Artificial Sequence
  Description of Artificial Sequence: Synthetic polypeptide
QDIQMTQSPGTLSVSPGERATLSCRASQSVSSDLAWYQHKPGQAPRLLIYGVSTKATGVPARFSGSGSGTEFTLTISSLQSEDLAVYYCQQYHNWPPLTFGGGTKVEIK

SEQ ID NO: 99 - protein, 118 aa, Artificial Sequence
  Description of Artificial Sequence: Synthetic polypeptide
EVQLLESGGGLVQPGGSLRLSCAASGFTFSSYTMEWVRQAPGKGLEWVSWISPSGGYTFYADSVKGRFTISRDNSKNTLYLQMNSLRAEDTALYYCARGYSYGSIDLWGRGTLVTVSS

SEQ ID NO: 100 - protein, 58 aa, Artificial Sequence
  Description of Artificial Sequence: Synthetic polypeptide
MHSFCAFKAETGPCRARFDRWFFNIFTRQCEEFIYGGCEGNQNRFESLEECKKMCTRD

SEQ ID NO: 101 - DNA, 363 bp, Artificial Sequence
  Description of Artificial Sequence: Synthetic polynucleotide
ttctattctcacagtgcacaagacatccagatgacccagtctccagccaccctgtctttgtctccaggggaaagagccaccctctcctgcagggccagtcagagtattagcagcttcttagcctggtaccaacagaaacctggccaggctcccaggctcctcatctatgatgcatcgtatagggccactggcatcccagccaggttcagtggcagtgggtctgggacagacttcactctcaccatcagcagcctggagcctgaagattatgcagtttattactgtcagcagcgtggcaactggcctatcaccttcggccaagggacgcggctggagattaaacgaactgtggctgcaccatct

SEQ ID NO: 102 - protein, 121 aa, Artificial Sequence
  Description of Artificial Sequence: Synthetic polypeptide
FYSHSAQDIQMTQSPATLSLSPGERATLSCRASQSISSFLAWYQQKPGQAPRLLIYDASYRATGIPARFSGSGSGTDFTLTISSLEPEDYAVYYCQQRGNWPITFGQGTRLEIKRTVAAPS

SEQ ID NO: 103 - DNA, 486 bp, Artificial Sequence
  Description of Artificial Sequence: Synthetic polynucleotide
atgaagaagctcctctttgctatcccgctcgtcgttccttttgtggcccagccggccatggccgaagttcaattgttagagtctggtggcggtcttgttcagcctggtggttctttacgtctttcttgcgctgcttccggattcactttctctcagtaccctatgtggtgggttcgccaagctcctggtaaaggtttggagtgggtttcttatatcgttccttctggtggccgtacttattatgctgactccgttaaaggtcgcttcactatctctagagacaactctaagaatactctctacttgcagatgaacagcttaagggctgaggacacggccgtgtattactgtgcgaaagatcgggcctacggtgattacgtgggctggaacggttttgactactggggccagggcaccctggtcaccgtctcaagcgcctccaccaagggcccatcggtcttcccgctagcaccctcctccaagagc

SEQ ID NO: 104 - protein, 162 aa, Artificial Sequence
  Description of Artificial Sequence: Synthetic polypeptide
MKKLLFAIPLVVPFVAQPAMAEVQLLESGGGLVQPGGSLRLSCAASGFTFSQYPMWWVRQAPGKGLEWVSYIVPSGGRTYYADSVKGRFTISRDNSKNTLYLQMNSLRAEDTAVYYCAKDRAYGDYVGWNGFDYWGQGTLVTVSSASTKGPSVFPLAPSSKS

SEQ ID NO: 105 - protein, 11 aa, Artificial Sequence
  Description of Artificial Sequence: Synthetic peptide
RASQSISSFLA

SEQ ID NO: 106 - protein, 7 aa, Artificial Sequence
  Description of Artificial Sequence: Synthetic peptide
DASYRAT

SEQ ID NO: 107 - protein, 9 aa, Artificial Sequence
  Description of Artificial Sequence: Synthetic peptide
QQRGNWPIT

SEQ ID NO: 108 - protein, 5 aa, Artificial Sequence
  Description of Artificial Sequence: Synthetic peptide
HYDMW

SEQ ID NO: 109 - protein, 5 aa, Artificial Sequence
  Description of Artificial Sequence: Synthetic peptide
WYDMW

SEQ ID NO: 110 - protein, 5 aa, Artificial Sequence
  Description of Artificial Sequence: Synthetic peptide
VYDMW

SEQ ID NO: 111 - protein, 5 aa, Artificial Sequence
  Description of Artificial Sequence: Synthetic peptide
QYDMW

SEQ ID NO: 112 - protein, 5 aa, Artificial Sequence
  Description of Artificial Sequence: Synthetic peptide
LYDMW

SEQ ID NO: 113 - protein, 17 aa, Artificial Sequence
  Description of Artificial Sequence: Synthetic peptide
VIYSSGGPTFYADSVKG

SEQ ID NO: 114 - protein, 17 aa, Artificial Sequence
  Description of Artificial Sequence: Synthetic peptide
VIYPSGGATYYADSVKG

SEQ ID NO: 115 - protein, 16 aa, Artificial Sequence
  Description of Artificial Sequence: Synthetic peptide
VIYPSGTTFYADSVKG

SEQ ID NO: 116 - protein, 17 aa, Artificial Sequence
  Description of Artificial Sequence: Synthetic peptide
VIYSSGGYTGYADSVKG

SEQ ID NO: 117 - protein, 124 aa, Artificial Sequence
  Description of Artificial Sequence: Synthetic polypeptide
EVQLLESGGGLVQPGGSLRLSCAASGFTFSHYDMWWVRQAPGKGLEWVSVIYSSGGPTFYADSVKGRFTISRDNSKNTLYLQMNSLRAEDTAVYYCAKDRAYGDYVGWNGFDYWGQGTLVTVSS

SEQ ID NO: 118 - protein, 103 aa, Artificial Sequence
  Description of Artificial Sequence: Synthetic polypeptide
DIQMTQSPSSLSASVGDRVTITCRASQSIYTYLNWYQQKPGKAPKLLIYAASSLQSGVPSRFSGSGSGTDFTLTISSLQPEDFATYYCQQAGIFGQGTKLEIK

SEQ ID NO: 119 - protein, 107 aa, Artificial Sequence [sequence omitted]

SEQ ID NO: 120 - protein, 122 aa, Artificial Sequence
  Description of Artificial Sequence: Synthetic polypeptide
EVQLLESGGGLVQPGGSLRLSCAASGFTFSWYWMHWVRQAPGKGLEWVSGISPSGGMTMYADSVKGRFTISRDNSKNTLYLQMNSLRAEDTAVYYCARDIVGPYSAGLFDYWGRGTLVTVSS

SEQ ID NO: 121 - protein, 122 aa, Artificial Sequence
  Description of Artificial Sequence: Synthetic polypeptide
EVQLLESGGGLVQPGGSLRLSCAASGFTFSDYNMHWVRQAPGKGLEWVSYIGPSGGYTHYADSVKGRFTISRDNSKNTLYLQMNSLRAEDTAVYYCAKDIRGAYSSGLFDYWGRGTLVTVSS

SEQ ID NO: 122 - protein, 122 aa, Artificial Sequence
  Description of Artificial Sequence: Synthetic polypeptide
EVQLLESGGGLVQPGGSLRLSCAASGFTFSWYGMHWVRQAPGKGLEWVSGIVSSGGETFYADSVKGRFTISRDNSKNTLYLQMNSLRAEDTAVYYCAKDIRGVFLSGLFDHWGRGTLVTVSS

SEQ ID NO: 123 - protein, 330 aa, Artificial Sequence
  Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 124 - protein, 4 aa, Artificial Sequence
  Description of Artificial Sequence: Synthetic peptide
GVHS

SEQ ID NO: 125 - protein, 23 aa, Artificial Sequence
  Description of Artificial Sequence: Synthetic peptide
DIQMTQSPSSLSASVGDRVTITC

SEQ ID NO: 126 - protein, 11 aa, Artificial Sequence
  Description of Artificial Sequence: Synthetic peptide
RASQSIYTYLN

SEQ ID NO: 127 - protein, 15 aa, Artificial Sequence
  Description of Artificial Sequence: Synthetic peptide
WYQQKPGKAPKLLIY

SEQ ID NO: 128 - protein, 7 aa, Artificial Sequence
  Description of Artificial Sequence: Synthetic peptide
AASSLQS

SEQ ID NO: 129 - protein, 32 aa, Artificial Sequence
  Description of Artificial Sequence: Synthetic polypeptide
GVPSRFSGSGSGTDFTLTISSLQPEDFATYYC

SEQ ID NO: 130 - protein, 5 aa, Artificial Sequence
  Description of Artificial Sequence: Synthetic peptide
QQAGI

SEQ ID NO: 131 - protein, 10 aa, Artificial Sequence
  Description of Artificial Sequence: Synthetic peptide
FGQGTKLEIK

SEQ ID NO: 132 - protein, 19 aa, Artificial Sequence
  Description of Artificial Sequence: Synthetic peptide
MGWSCIILFLVATATGAHS

SEQ ID NO: 133 - protein, 30 aa, Artificial Sequence
  Description of Artificial Sequence: Synthetic polypeptide
EVQLLESGGGLVQPGGSLRLSCAASGFTFS

SEQ ID NO: 134 - protein, 5 aa, Artificial Sequence
  Description of Artificial Sequence: Synthetic peptide
DYWMH

SEQ ID NO: 135 - protein, 5 aa, Artificial Sequence
  Description of Artificial Sequence: Synthetic peptide
DYWLH

SEQ ID NO: 136 - protein, 5 aa, Artificial Sequence
  Description of Artificial Sequence: Synthetic peptide
DYWTH

SEQ ID NO: 137 - protein, 14 aa, Artificial Sequence
  Description of Artificial Sequence: Synthetic peptide
WVRQAPGKGLEWVS

SEQ ID NO: 138 - protein, 17 aa, Artificial Sequence
  Description of Artificial Sequence: Synthetic peptide
GISPSGGMTMYADSVKG

SEQ ID NO: 139 - protein, 17 aa, Artificial Sequence
  Description of Artificial Sequence: Synthetic peptide
GISPSGGSTMYADSVKG

SEQ ID NO: 140 - protein, 17 aa, Artificial Sequence
  Description of Artificial Sequence: Synthetic peptide
GISPSGGMTYYADSVKG

SEQ ID NO: 141 - protein, 17 aa, Artificial Sequence
  Description of Artificial Sequence: Synthetic peptide
GISPSGGSTYYADSVKG

SEQ ID NO: 142 - protein, 17 aa, Artificial Sequence
  Description of Artificial Sequence: Synthetic peptide
GISGSGGMTMYADSVKG

SEQ ID NO: 143 - protein, 17 aa, Artificial Sequence
  Description of Artificial Sequence: Synthetic peptide
GISGSGGSTMYADSVKG

SEQ ID NO: 144 - protein, 17 aa, Artificial Sequence
  Description of Artificial Sequence: Synthetic peptide
GISGSGGMTYYADSVKG

SEQ ID NO: 145 - protein, 17 aa, Artificial Sequence
  Description of Artificial Sequence: Synthetic peptide
GISGSGGSTYYADSVKG

SEQ ID NO: 146 - protein, 32 aa, Artificial Sequence
  Description of Artificial Sequence: Synthetic polypeptide
RFTISRDNSKNTLYLQMNSLRAEDTAVYYCAR

SEQ ID NO: 147 - protein, 13 aa, Artificial Sequence
  Description of Artificial Sequence: Synthetic peptide
DIVGPYSAGLFDY

SEQ ID NO: 148 - protein, 11 aa, Artificial Sequence
  Description of Artificial Sequence: Synthetic peptide
WGRGTLVTVSS

SEQ ID NO: 149 - protein, 17 aa, Artificial Sequence
  Description of Artificial Sequence: Synthetic peptide
ASTKGPSVFPLAPSSKS

SEQ ID NO: 150 - protein, 122 aa, Artificial Sequence
  Description of Artificial Sequence: Synthetic polypeptide
EVQLLESGGGLVQPGGSLRLSCAASGFTFSDYWMHWVRQAPGKGLEWVSGISPSGGMTMYADSVKGRFTISRDNSKNTLYLQMNSLRAEDTAVYYCARDIVGPYSAGLFDYWGRGTLVTVSS

SEQ ID NO: 151 - protein, 229 aa, Artificial Sequence
  Description of Artificial Sequence: Synthetic polypeptide
MGWSCIILFLVATATGVHSDIQMTQSPSSLSASVGDRVTITCRASQSIYTYLNWYQQKPGKAPKLLIYAASSLQSGVPSRFSGSGSGTDFTLTISSLQPEDFATYYCQQAGIFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 152-199 - skipped (48 entries)

SEQ ID NO: 200 - protein, 60 aa, Artificial Sequence
  Description of Artificial Sequence: Synthetic polypeptide
EAMHSFCAFKAETGPCRARFDRWFFNIFTRQCEEFIYGGCEGNQNRFESLEECKKMCTRD

SEQ ID NO: 201-299 - skipped (99 entries)

SEQ ID NO: 300 - protein, 58 aa, Artificial Sequence
  Description of Artificial Sequence: Synthetic polypeptide
  MOD_RES (1)..(4): Any non-Cysteine amino acid or not present
  MOD_RES (6)..(9): Any non-Cysteine amino acid
  MOD_RES (10)..(10): Asp, Glu, Tyr or Gln
  MOD_RES (11)..(11): Thr, Ala, Ser, Val or Asp
  MOD_RES (13)..(13): Pro, Leu or Ala
  MOD_RES (15)..(15): Lys or Arg
  MOD_RES (16)..(16): Ala or Gly
  MOD_RES (17)..(17): Arg, Lys or Ser
  MOD_RES (18)..(18): Phe or Ile
  MOD_RES (19)..(19): Glu, Gln, Asp, Pro, Gly, Ser or Ile
  MOD_RES (21)..(21): Phe, Tyr or Trp
  MOD_RES (22)..(23): Tyr or Phe
  MOD_RES (24)..(29): Any non-Cysteine amino acid
  MOD_RES (31)..(31): Asp, Glu, Thr, Val, Gln or Ala
  MOD_RES (32)..(32): Thr, Ala, Glu, Pro or Gln
  MOD_RES (34)..(34): Val, Ile, Thr, Leu, Phe, Tyr, His, Asp, Ala or Ser
  MOD_RES (35)..(35): Tyr or Trp
  MOD_RES (36)..(36): Gly or Ser
  MOD_RES (39)..(39): Glu, Gly, Asp, Arg, Ala, Gln, Leu, Lys or Met
  MOD_RES (40)..(40): Gly or Ala
  MOD_RES (41)..(42): Any non-Cysteine amino acid
  MOD_RES (43)..(43): Asn or Gly
  MOD_RES (44)..(44): Any non-Cysteine amino acid
  MOD_RES (45)..(45): Phe or Tyr
  MOD_RES (46)..(50): Any non-Cysteine amino acid
  MOD_RES (52)..(54): Any non-Cysteine amino acid
  MOD_RES (56)..(58): Any non-Cysteine amino acid or not present
XXXXCXXXXXXGXCXXXXXRXXXXXXXXXCXXFXXXGCXXXXXXXXXXXXCXXXCXXX

(19) United States
(12) Patent Application Publication
(10) Pub. No.: US 2011/0262396 A1
(43) Pub. Date: Oct. 27, 2011

(54) COMBINATION TREATMENTS COMPRISING PROTEASE BINDING PROTEINS FOR INFLAMMATORY DISORDERS

(75) Inventor: Clive R. Wood, Boston, MA (US)

(73) Assignee: DYAX CORP., Cambridge, MA (US)

(21) Appl. No.: 13/124,046

(22) PCT Filed: Oct. 22, 2009
(86) PCT No.: PCT/US09/61717

§ 371 Date: Jul. 6, 2011

Related U.S. Application Data
(60) Provisional application No. 61/107,384, filed on Oct. 22, 2008.

(51) Int. Cl. A61K 39/395; A61K 38/21; A61P 29/00; A61P 1/00; A61P 25/28; A61P 11/06; A61P 11/00; A61K 38/55; A61P 17/06

(52) U.S. Cl. 424/85.4; 424/158.1; 514/20.3; 424/142.1; 424/133.1; 424/134.1; 514/16.6

(57) ABSTRACT

Provided are methods and compositions for using protease binding proteins in combination with other therapeutic agents to treat inflammatory disorders such as rheumatoid arthritis, psoriasis, multiple sclerosis, systemic sclerosis, asthma, chronic obstructive pulmonary disease, inflammatory bowel disease (e.g., Crohn's and ulcerative colitis). The use of the protease binding protein allows a lower dose of the other therapeutic agents to be used in the methods and compositions, such that side effects of the other therapeutic agents are reduced or removed.

### CROSS REFERENCE TO RELATED APPLICATIONS

This application is a national phase application under 35 U.S.C. §371 of International Application No. PCT/US2009/061717, filed Oct. 22, 2009, which claims priority to U.S. Application Ser. No. 61/107,384, filed on Oct. 22, 2008. The disclosure of the prior applications are considered part of (and are incorporated by reference in) the disclosure of this application.

### SEQUENCE LISTING

The instant application contains a Sequence Listing which has been submitted via EFS-Web and is hereby incorporated by reference in its entirety. Said ASCII copy, created on Nov. 20, 2009, is named D27128WO.txt, and is 160,229 bytes in size.

### BACKGROUND

Immunosuppressive drugs inhibit or prevent activity of the immune system. They are used to: prevent the rejection of transplanted organs and tissues; treat disease (e.g., an autoimmune disease) (e.g., rheumatoid arthritis, multiple sclerosis, myasthenia gravis, systemic lupus erythematosus, Crohn's disease, pemphigus, and ulcerative colitis) and to treat some other non-autoimmune inflammatory diseases (e.g., long term allergic asthma control). Examples are TNFα inhibitors such as REMICADE®, ENBREL®, HUMIRA® and CIMZIA®. Other examples are drugs such as ORENCIA® (CTLA4-Ig (Bristol Myers Squibb)) anti-VLA4 (TYSABRI®), and anti-IL-6 receptor antibody (Chugai/Roche).

These drugs are not without side-effects and risks. Because the majority of them act non-selectively, the immune system is less able to resist infections and the spread of malignant cells. There are also other side-effects, such as hypertension, dyslipidemia, hyperglycemia, peptic ulcers, liver, and kidney injury. The immunosuppressive drugs also interact with other medicines and affect their metabolism and action. Further, the clinical benefit is incomplete with each drug, even if it is selective. For example, there may be a 50% decrease in disease severity (eg ACR50 score), but such benefit is frequently not sufficient to alleviate all symptoms and allow return to normal life. Opportunistic infections are a serious adverse event associated with such drugs. Adhesion molecule blockers such as anti-VLA4 (TYSABRI®), in multiple sclerosis have been associated with the risk of viral infection. This has prohibited the combination of these immunosuppressive drugs.

### SUMMARY

Inhibitors of proteases that contribute to inflammation and its consequences such as cartilage and bone erosion have the potential to arrest disease pathology and not be immunosuppressive. Thus, such protease inhibitors do not lead to infectious disease complications, and may be combined with each other as well as other drugs without undue immunosuppressive effect.

The disclosure provides, inter alia, methods, compositions and kits for combination therapies for treating inflammatory disorders comprising antibody, peptide and/or Kunitz domain-based protease binding proteins and immunosuppressive drugs or other therapeutic agents for inflammatory disorders. Inflammatory disorders to be treated include, but are not limited to, rheumatoid arthritis, psoriasis, multiple sclerosis, systemic sclerosis, asthma, chronic obstructive pulmonary disease, inflammatory bowel disease (e.g., Crohn's and ulcerative colitis). Exemplary protease binding proteins include all members of the metalloprotease family, especially targets such as MMP-14, MMP-9, MMP-12, MMP-7 (“MMP binding proteins”), as well as other proteases such as TACE, ADAM-TS2; serine proteases, including plasmin, hepsin, matriptase, plasma kallikrein and tissue kallikrein 1; and cathepsins, including cathepsins B, S and K. In certain embodiments, the protease binding proteins are protease inhibitors. In certain embodiments, the combination therapies comprise use of an MMP binding protein (e.g., an MMP inhibitor) and a disease-modifying antirheumatic drug (DMARD) such as methotrexate, and/or a biological response modifier (BRM) such as a TNF-α inhibitor (Etanercept (ENBREL®), Adalimumab (HUMIRA®), and Infliximab (REMICADE®)), a CTLA4-Ig (Abatacept (ORENCIA®)) or an anti-CD20 (rituximab (RITUXAN®)). The combination therapies may have additive and/or synergistic benefits, and increase the effect on disease symptoms and progression without increasing the risk of opportunistic infections. In certain embodiments, the drug combination may allow a decrease in dose of the immunosuppressive drug or second-line agent, thereby decreasing the possibility of an adverse event (e.g., side effect).

The antibody, peptide and/or Kunitz domain-based protease binding proteins used in any disclosed method, kit or composition, as well as the immunosuppressive drugs or second-line agents with which they are combined, can have one or more of the characteristics described below in the Detailed Description. Preferred compositions, e.g., used in any method or kit described herein, may further comprise one or more pharmaceutically acceptable buffers, carriers, and excipients, which may provide a desirable feature to the composition including, but not limited to, enhanced administration of the composition to a patient, enhanced circulating half-life of the inhibitor, enhanced compatibility of the composition with patient blood chemistry, enhanced storage of the composition, and/or enhanced efficacy of the composition upon administration to a patient.

In some aspects, the disclosure features a method of treating an inflammatory disorder. The method includes

administering a therapeutically effective amount of a protease binding protein in combination with a therapeutically effective amount of a second agent, wherein the second agent is an agent for the treatment of the inflammatory disorder,

to a subject having or suspected of having the inflammatory disorder.

In some embodiments, the inflammatory disorder is selected from the group consisting of: rheumatoid arthritis, psoriasis, multiple sclerosis, systemic sclerosis, asthma, chronic obstructive pulmonary disease, and inflammatory bowel disease.

In some embodiments, the protease binding protein is a protease inhibitor.

In some embodiments, the protease binding protein binds to a protease selected from the group consisting of: plasma kallikrein, plasmin, MMP-14, MMP-9, MMP-9/-2, and MMP-12.

In some embodiments, the protease binding protein is an inhibitor of plasma kallikrein.

In some embodiments, the inhibitor of plasma kallikrein is selected from the group consisting of: a Kunitz domain containing polypeptide and aplasma kallikrein binding antibody.

In some embodiments, the inhibitor of plasma kallikrein is a Kunitz domain containing polypeptide that comprises the sequence:

Xaa1 Xaa2 Xaa3 Xaa4 Cys Xaa6 Xaa7 Xaa8 Xaa9 Xaa10 Xaa11 Gly Xaa13 Cys Xaa15 Xaa16 Xaa17 Xaa18 Xaa19 Xaa20 Xaa21 Xaa22 Xaa23 Xaa24 Xaa25 Xaa26 Xaa27 Xaa28 Xaa29 Cys Xaa31 Xaa32 Phe Xaa34 Xaa35 Gly Gly Cys Xaa39 Xaa40 Xaa41 Xaa42 Xaa43 Xaa44 Xaa45 Xaa46 Xaa47 Xaa48 Xaa49 Xaa50 Cys Xaa52 Xaa53 Xaa54 Cys Xaa56 Xaa57 Xaa58 (SEQ ID NO:1), wherein Xaa10 is Asp or Glu; Xaa11 is Asp, Gly, Ser, Val, Asn, Ile, Ala or Thr; Xaa13 is Pro, Arg, His, Asn, Ser, Thr, Ala, Gly, Lys or Gln; Xaa15 is Arg, Lys, Ala, Ser, Gly, Met, Asn or Gln; Xaa16 is Ala, Gly, Ser, Asp or Asn; Xaa17 is Ala, Asn, Ser, Ile, Gly, Val, Gln or Thr; Xaa18 is His, Leu, Gln or Ala; Xaa19 is Pro, Gln, Leu, Asn or Ile; Xaa21 is Trp, Phe, Tyr, His or Ile; Xaa31 is Glu, Asp, Gln, Asn, Ser, Ala, Val, Leu, Ile or Thr; Xaa32 is Glu, Gln, Asp Asn, Pro, Thr, Leu, Ser, Ala, Gly or Val; Xaa34 is Ile, Thr, Ser, Val, Ala, Asn, Gly or Leu; Xaa35 is Tyr, Trp or Phe; Xaa39 is Glu, Gly, Ala, Ser or Asp; amino acids Xaa6, Xaa7, Xaa8, Xaa9, Xaa20, Xaa24, Xaa25, Xaa26, Xaa27, Xaa28, Xaa29, Xaa41, Xaa42, Xaa44, Xaa46, Xaa47, Xaa48, Xaa49, Xaa50, Xaa52, Xaa53 and Xaa54 is any amino acid; each of the first four and at last three amino acids of SEQ ID NO:1 can each individually optionally be present or absent and is any non-cysteine amino acid.

In some embodiments, the inhibitor of plasma kallikrein is a Kunitz domain containing polypeptide that comprises the sequence of amino acids 3-60 of SEQ ID NO:2.

In some embodiments, the inhibitor of plasma kallikrein is a Kunitz domain containing polypeptide that consists of the sequence of amino acids 3-60 of SEQ ID NO:2.

In some embodiments, the inhibitor of plasma kallikrein is a Kunitz domain containing polypeptide that comprises the sequence of SEQ ID NO:2.

In some embodiments, the inhibitor of plasma kallikrein is a Kunitz domain containing polypeptide that consists of the sequence of SEQ ID NO:2.

In some embodiments, the protease binding protein is an inhibitor of plasmin.

In some embodiments, the inhibitor of plasmin is selected from the group consisting of: a Kunitz domain containing polypeptide and a plasmin binding antibody.

In some embodiments, the inhibitor of plasmin is a Kunitz domain containing polypeptide that comprises the sequence:

Xaa1-Xaa2-Xaa3-Xaa4-Cys-Xaa6-Xaa7-Xaa8-Xaa9-Xaa10-Xaa11-Gly-Xaa13-Cys-Xaa15-Xaa16Xaa17-Xaa18-Xaa 19-Arg-Xaa21-Xaa22-Xaa23-Xaa24-Xaa25-Xaa26-Xaa27-Xaa28-Xaa29-Cys-Xaa31-Xaa 32-Phe-Xaa34-Xaa35-Xaa36-Gly-Cys-Xaa39-Xaa40-Xaa41-Xaa42-Xaa43-Xaa44-Xaa45-Xaa46-Xaa47-Xaa48-Xaa49-Xaa50-Cys-Xaa52-Xaa53-Xaa54-Cys-Xaa56-Xaa57-Xaa58 (SEQ ID NO:300), wherein

Xaa1, Xaa2, Xaa3, Xaa4, Xaa56, Xaa57 and Xaa58 may each individually be absent; Xaa10 is Asp, Glu, Tyr, or Gln; Xaa11 is Thr, Ala, Ser, Val or Asp; Xaa13 is Pro, Leu or Ala; Xaa15 is Lys or Arg; Xaa16 is Ala or Gly; Xaa17 is Arg, Lys or Ser; Xaa18 is Phe or Ile; Xaa19 is Glu, Gln, Asp, Pro, Gly, Ser or Ile; Xaa21 is Phe, Tyr or Trp; Xaa22 is Tyr or Phe. Xaa23 is Tyr or Phe; Xaa31 is Asp, Glu, Thr, Val, Gln or Ala; Xaa32 is Thr, Ala, Glu, Pro, or Gln; Xaa34 is Val, Ile, Thr, Leu, Phe, Tyr, His, Asp, Ala, or Ser; Xaa35 is Tyr or Trp; Xaa36 is Gly or Ser; Xaa39 is Glu, Gly, Asp, Arg, Ala, Gln, Leu, Lys, or Met; Xaa40 is Gly or Ala; Xaa43 is Asn or Gly; or Xaa45 is Phe or Tyr; and where not specified, Xaa is any non-cysteine amino acid.

In some embodiments, the inhibitor of plasmin is a Kunitz domain containing polypeptide that comprises the sequence of SEQ ID NO:100.

In some embodiments, the inhibitor of plasmin is a Kunitz domain containing polypeptide that consists of the sequence of SEQ ID NO:100.

In some embodiments, the inhibitor of plasmin is a Kunitz domain containing polypeptide that comprises the sequence of SEQ ID NO:200.

In some embodiments, the inhibitor of plasmin is a Kunitz domain containing polypeptide that consists of the sequence of SEQ ID NO:200.

In some embodiments, the protease binding protein is an MMP-14 binding protein.

In some embodiments, the MMP-14 binding protein comprises the 3 heavy chain CDRs and/or the 3 light chain CDRs of DX-2400, DX-2410, M0031-C02, M0031-F01, M0033-H07, M0037-009, M0037-D01, M0038-E06, M0038-F01, M0038-F08, M0039-H08, M0040-A06, M0040-A11, or M0043-G02.

In some embodiments, the MMP-14 binding protein comprises the heavy chain variable region and/or the light chain variable region of DX-2400, DX-2410, M0031-C02, M0031-F01, M0033-H07, M0037-009, M0037-D01, M0038-E06, M0038-F01, M0038-F08, M0039-H08, M0040-A06, M0040-A11, or M0043-G02.

In some embodiments, the MMP-14 binding protein comprises the 3 heavy chain CDRs and/or the 3 light chain CDRs of DX-2400.

In some embodiments, the MMP-14 binding protein comprises the heavy chain variable region and/or the light chain variable region of DX-2400.

In some embodiments, the protease binding protein is an MMP-9 binding protein.

In some embodiments, the MMP-9 binding protein comprises the 3 heavy chain CDRs and/or the 3 light chain CDRs of DX-2802, 539A-M0240-B03, M0078-G07, M0081-D05, M0076-D03, M0072-H07, M0075-D12, M0166-F10, M0279-A03, M0279-B02, M0288-008, or M0281-F06.

In some embodiments, the MMP-9 binding protein comprises the heavy chain variable region and/or the light chain variable region of DX-2802, 539A-M0240-B03, M0078-G07, M0081-D05, M0076-D03, M0072-H07, M0075-D12, M0166-F10, M0279-A03, M0279-B02, M0288-008, or M0281-F06.

In some embodiments, the MMP-9 binding protein comprises the 3 heavy chain CDRs and/or the 3 light chain CDRs of DX-2802.

In some embodiments, the MMP-9 binding protein comprises the heavy chain variable region and/or the light chain variable region of DX-2802.

In some embodiments, the protease binding protein is an MMP-9/-2 binding protein.

In some embodiments, the MMP-9/-2 binding protein comprises the 3 heavy chain CDRs and/or the 3 light chain CDRs of M0237-D02, X0106-A01, X0106-B02, X0106-004, X0106-E4, or X0106-F05.

In some embodiments, the MMP-9/-2 binding protein comprises the heavy chain variable region and/or the light chain variable region of M0237-D02, X0106-A01, X0106-B02, X0106-004, X0106-E4, or X0106-F05.

In some embodiments, the MMP-9/-2 binding protein comprises the 3 heavy chain CDRs and/or the 3 light chain CDRs of M0237-D02.

In some embodiments, the MMP-9/-2 binding protein comprises the heavy chain variable region and/or the light chain variable region of M0237-D02.

In some embodiments, the protease binding protein is an MMP-12 binding protein.

In some embodiments, the MMP-12 binding protein comprises the 3 heavy chain CDRs and/or the 3 light chain CDRs of DX-2712 (also referred to herein as DX-2712HEK), a mutant or variant of DX-2712, 539B-X0041-D02, M0134-A02, M134-A05, M134-A07, M134-A09, M134-A10, M134-A11, M0134-B01, M134-B04, M0134-B08, M0134-B11, M0134-001, M0134-C02, M0134-006, M0134-009, M0134-C10, M0134-C11, M0134-C12, M0134-D02, M0134-D03, M0134-E04, M0134-E07, M0134-E08, M134-E11, M0134-F01, M0134-F05, M0134-G02, M0134-G04, M0134-G07, M0135-A03, M0135-A05, M0135-A06, M0135-A07, M0135-B02, M0135-B08, M0135-001, M0135-C11, M0135-E03, M0135-F03, M0135-F11, M0135-G02, M0135-G03, M0135-G07, M0135-G11, M0135-H03, M0135-H10, M0105-C05, M0105-E11, M0105-F08, M0107-A12, M0108-A02, M0109-G11, M0110-G05, M0129-B11, M0130-A01, M0130-C12, M0130-F06, M0130-H04, M0131-A06, M0131-D03, M0132-A04, M0133-B08, M0133-E05, M0121-E07, M0118-F11, M0125-G07, M0124-E07, M0119-D01, M0119-A02, M0122-006, M0123-G07, M0063-A02, M0063-A04, M0063-B01, M0063-B11, M0063-007, M0063-G01, M0065-E12, M0065-G03, M0065-H05, M0067-A02, M0067-B06, M0067-B09, M0067-C10, M0067-F02, M0067-F06, M0069-A04, M0069-A11, M0069-C02, M0069-D10, M0069-G07, M0071-A01, M0071-B07, M0071-D05, M0071-D09, M0071-H03, M0071-H06, M0087-F09, M0088-F07, M0088-G10, M0088-H10, M0089-001, M0089-F05, M0089-B07, M0089-H11, M0032-E01, M0034-004, M0039-F01, M0041-B05, M0041-G01, M0042-B06, M0006-B10, M0007-H06, M0008-H09, M0009-H08, M0011-H11, M0015-F02, M0016-D01, M0013-D11, M0013-G12, M0013-H06, M0014-009, M0014-G11, M0016-A11, M0016-H05, M0019-C05, M0020-B01, M0022-007, M0025-D04, M0027-E11, 539B-X0041-D02, 539B-X0049-A01, 539B-X0049-B01, 539B-X0049-001, 539B-X0049-D01, 539B-X0049-E01, 539B-X0049-F01, 539B-X0049-G01, 539B-X0049-H01, 539B-X0049-A02, 539B-X0049-B02, 539B-X0049-C02, 539B-X0049-D02, or 539B-X0049-E02.

In some embodiments, the MMP-12 binding protein comprises the heavy chain variable region and/or the light chain variable region of DX-2712, a mutant or variant of DX-2712, 539B-X0041-D02, M0134-A02, M134-A05, M134-A07, M134-A09, M134-A10, M134-A11, M0134-B01, M134-B04, M0134-B08, M0134-B11, M0134-001, M0134-C02, M0134-006, M0134-009, M0134-C10, M0134-C11, M0134-C12, M0134-D02, M0134-D03, M0134-E04, M0134-E07, M0134-E08, M134-E11, M0134-F01, M0134-F05, M0134-G02, M0134-G04, M0134-G07, M0135-A03, M0135-A05, M0135-A06, M0135-A07, M0135-B02, M0135-B08, M0135-001, M0135-C11, M0135-E03, M0135-F03, M0135-F11, M0135-G02, M0135-G03, M0135-G07, M0135-G11, M0135-H03, M0135-H10, M0105-C05, M0105-E11, M0105-F08, M0107-A12, M0108-A02, M0109-G11, M0110-G05, M0129-B11, M0130-A01, M0130-C12, M0130-F06, M0130-H04, M0131-A06, M0131-D03, M0132-A04, M0133-B08, M0133-E05, M0121-E07, M0118-F11, M0125-G07, M0124-E07, M0119-D01, M0119-A02, M0122-006, M0123-G07, M0063-A02, M0063-A04, M0063-B01, M0063-B11, M0063-007, M0063-G01, M0065-E12, M0065-G03, M0065-H05, M0067-A02, M0067-B06, M0067-B09, M0067-C10, M0067-F02, M0067-F06, M0069-A04, M0069-A11, M0069-C02, M0069-D10, M0069-G07, M0071-A01, M0071-B07, M0071-D05, M0071-D09, M0071-H03, M0071-H06, M0087-F09, M0088-F07, M0088-G10, M0088-H10, M0089-001, M0089-F05, M0089-B07, M0089-H11, m0032-E01, M0034-004, M0039-F01, M0041-B05, M0041-G01, M0042-B06, M0006-B10, M0007-H06, M0008-H09, M0009-H08, M0011-H11, M0015-F02, M0016-D01, M0013-D11, M0013-G12, M0013-H06, M0014-009, M0014-G11, M0016-A11, M0016-H05, M0019-C05, M0020-B01, M0022-007, M0025-D04, M0027-E11, 539B-X0041-D02, 539B-X0049-A01, 539B-X0049-B01, 539B-X0049-001, 539B-X0049-D01, 539B-X0049-E01, 539B-X0049-F01, 539B-X0049-G01, 539B-X0049-H01, 539B-X0049-A02, 539B-X0049-B02, 539B-X0049-D02, 539B-X0049-D02, or 539B-X0049-E02.

In some embodiments, the MMP-12 binding protein comprises the 3 heavy chain CDRs and/or the 3 light chain CDRs of DX-2712.

In some embodiments, the MMP-12 binding protein comprises the heavy chain variable region and/or the light chain variable region of DX-2712.

In some embodiments, the second agent is an agent for the treatment of an inflammatory disorder selected from the group of inflammatory disorders consisting of: rheumatoid arthritis, psoriasis, multiple sclerosis, systemic sclerosis, asthma, chronic obstructive pulmonary disease, and inflammatory bowel disease.

In some embodiments, the second agent is an immunosuppressant agent selected from the group consisting of: gancyclovir, etanercept, cyclosporine, tacrolimus, rapamycin, cyclophosphamide, azathioprine, mycophenolate mofetil, methotrexate, cortisol, aldosterone, dexamethasone, a cyclooxygenase inhibitor, a 5-lipoxygenase inhibitor, and leukotriene receptor antagonist.

In some embodiments, the second agent is an agent for the treatment of rheumatoid arthritis and comprises a nonsteroidal anti-inflammatory drug (NSAID), a corticosteroid, a Disease-Modifying Antirheumatic Drug (DMARD), or a biological response modifier (BRM).

In some embodiments, the second agent is an agent for the treatment of rheumatoid arthritis and comprises aspirin, naproxen, ibuprofen, etodolac, gold, salsalte, methotrexate, sulfasalazine, D-penicillamine, azathioprine, cyclophosphamide, chlorambucil, cyclosporine, leflunomide, etanercept, infliximab, anakinra, adalimumab, hydroxychloroquine, chloroquine phosphate, chloroquine sulphate, minocycline, or a CTLA4-Ig.

In some embodiments, the second agent is an agent for the treatment of psoriasis and comprises a topical treatment or a systemic treatment.

In some embodiments, the second agent is an agent for the treatment of psoriasis and comprises a topical treatment selected from the group consisting of coal tar, dithranol, a corticosteroid, a vitamin D3 analogue, and a retinoid.

In some embodiments, the second agent is an agent for the treatment of psoriasis and comprises a systemic treatment selected from the group consisting of methotrexate, cyclosporine, a retinoid, tioguanine, hydroxyurea, sulfasalazine, mycophenolate mofetil, azathioprine, tacrolimus, alefacept, efalizumab, etanercept, and infliximab.

In some embodiments, the second agent is an agent for the treatment of multiple sclerosis and comprises a corticosteroid, an interferon, glatiramer acetate, an immunosuppressant or natalizumab.

In some embodiments, the second agent is an agent for the treatment of systemic sclerosis and comprises an NSAID, a calcium channel blocker, prostacyclin analogue, a dual endothelin-receptor antagonist, methotrexate, cyclosporin, an ACE inhibitor, cyclophosphamide, a steroid, or epoprostenol.

In some embodiments, the second agent is an agent for the treatment of systemic sclerosis and comprises nifedipine.

In some embodiments, the second agent is an agent for the treatment of systemic sclerosis and comprises iloprost.

In some embodiments, the second agent is an agent for the treatment of systemic sclerosis and comprises bosentan.

In some embodiments, the second agent is an agent for the treatment of asthma and comprises a glucocorticoid, a leukotriene modifier, a mast cell stabilizer, an antimuscarinic/anticholinergic, an antihistamines, an IgE blocker, or methotrexate.

In some embodiments, the second agent is an agent for the treatment of asthma and comprises a beta2-adrenoceptor agonist selected from the group consisting of: salbutamol, levalbuterol, terbutaline and bitolterol.

In some embodiments, the second agent is an agent for the treatment of asthma and comprises an adrenergic agonist selected from the group consisting of: inhaled epinephrine and ephedrine tablets.

In some embodiments, the second agent is an agent for the treatment of asthma and comprises an antimuscarinic/anticholinergic selected from the group consisting of: ipratropium, oxitropium, and tiotropium.

In some embodiments, the second agent is an agent for the treatment of asthma and comprises an inhaled glucocorticoid selected from the group consisting of: ciclesonide, beclomethasone, budesonide, flunisolide, fluticasone, mometasone, and triamcinolone.

In some embodiments, the second agent is an agent for the treatment of asthma and comprises a leukotriene modifier selected from the group consisting of: montelukast, zafirlukast, pranlukast, and zileuton.

In some embodiments, the second agent is an agent for the treatment of asthma and comprises a mast cell stabilizer selected from the group consisting of: cromoglicate and nedocromil.

In some embodiments, the second agent is an agent for the treatment of asthma and comprises a methylxanthine selected from the group consisting of: theophylline and aminophylline.

In some embodiments, the second agent is an agent for the treatment of asthma and comprises an antihistamine.

In some embodiments, the second agent is an agent for the treatment of asthma and comprises an IgE blocker selected from the group consisting of: omalizumab and methotrexate.

In some embodiments, the second agent is an agent for the treatment of asthma and comprises a long-acting beta₂-adrenoceptor agonist selected from the group consisting of: salmeterol, formoterol, bambuterol, and albuterol.

In some embodiments, the second agent is an agent for the treatment of asthma and comprises a combination of inhaled steroid and a long-acting bronchodilator selected from the group consisting of: fluticasone/salmeterol and budesonide/formoterol.

In some embodiments, the second agent is an agent for the treatment of chronic obstructive pulmonary disease (COPD) and comprises a bronchodilator.

In some embodiments, the second agent is an agent for the treatment of chronic obstructive pulmonary disease (COPD)and comprises a β₂ agonist, an M₃ antimuscarinic, a leukotriene antagonist, a cromone, a corticosteroid, or a xanthine.

In some embodiments, the second agent is an agent for the treatment of chronic obstructive pulmonary disease (COPD)and comprises a β₂ agonist selected from the group consisting of: Salbutamol, Bambuterol, Clenbuterol, Fenoterol, Formoterol, and Salmeterol.

In some embodiments, the second agent is an agent for the treatment of chronic obstructive pulmonary disease (COPD)and comprises the Ipratropium.

In some embodiments, the second agent is an agent for the treatment of chronic obstructive pulmonary disease (COPD)and comprises a cromone selected from the group consisting of: Cromoglicate and Nedocromil.

In some embodiments, the second agent is an agent for the treatment of chronic obstructive pulmonary disease (COPD)and comprises a leukotriene antagonist selected from the group consisting of: Montelukast, Pranlukast, and Zafirlukast.

In some embodiments, the second agent is an agent for the treatment of chronic obstructive pulmonary disease (COPD)and comprises a corticosteroid antagonist selected from the group consisting of: glucocorticoids, beclomethasone, mometasone, and fluticasone.

In some embodiments, the second agent is an agent for the treatment of chronic obstructive pulmonary disease (COPD)and comprises a xanthine antagonist selected from the group consisting of: theophylline, methylxanthine, and theobromine.

In some embodiments, the second agent is an agent for the treatment of chronic obstructive pulmonary disease (COPD) and comprises Ipratropium or Tiotropium.

In some embodiments, the second agent is an agent for the treatment of inflammatory bowel disease and comprises an immunosuppresant, an anti-TNF binding protein, a cytokine inhibitor, a BRM, or an anti-inflammatory.

In some embodiments, the second agent is an agent for the treatment of inflammatory bowel disease and comprises prednisone, infliximab, azathioprine, methotrexate, 6-mercaptopurine , a mesalamine, a steroid, or CDP571 antibody.

In another aspect, the disclosure features a method of preventing an inflammatory disorder. The method includes:

administering a therapeutically effective amount of a protease binding protein in combination with a therapeutically effective amount of a second agent, wherein the second agent is an agent for the treatment of the inflammatory disorder,

to a subject at risk of having the inflammatory disorder.

In some embodiments, the inflammatory disorder is selected from the group consisting of: rheumatoid arthritis, psoriasis, multiple sclerosis, systemic sclerosis, asthma, chronic obstructive pulmonary disease, and inflammatory bowel disease.

In some embodiments, the protease binding protein is a protease inhibitor.

In some embodiments, the protease binding protein binds to a protease selected from the group consisting of: plasma kallikrein, plasmin, MMP-14, MMP-9, MMP-9/-2, and MMP-12.

In some embodiments, the protease binding protein is an inhibitor of plasma kallikrein.

In some embodiments, the inhibitor of plasma kallikrein is selected from the group consisting of: a Kunitz domain containing polypeptide and aplasma kallikrein binding antibody.

In some embodiments, the inhibitor of plasma kallikrein is a Kunitz domain containing polypeptide that comprises the sequence:

Xaa1 Xaa2 Xaa3 Xaa4 Cys Xaa6 Xaa7 Xaa8 Xaa9 Xaa10 Xaa11 Gly Xaa13 Cys Xaa15 Xaa16 Xaa17 Xaa18 Xaa19 Xaa20 Xaa21 Xaa22 Xaa23 Xaa24 Xaa25 Xaa26 Xaa27 Xaa28 Xaa29 Cys Xaa31 Xaa32 Phe Xaa34 Xaa35 Gly Gly Cys Xaa39 Xaa40 Xaa41 Xaa42 Xaa43 Xaa44 Xaa45 Xaa46 Xaa47 Xaa48 Xaa49 Xaa50 Cys Xaa52 Xaa53 Xaa54 Cys Xaa56 Xaa57 Xaa58 (SEQ ID NO:1), wherein

Xaa10 is Asp or Glu; Xaa11 is Asp, Gly, Ser, Val, Asn, Ile, Ala or Thr; Xaa13 is Pro, Arg, His, Asn, Ser, Thr, Ala, Gly, Lys or Gln; Xaa15 is Arg, Lys, Ala, Ser, Gly, Met, Asn or Gln; Xaa16 is Ala, Gly, Ser, Asp or Asn; Xaa17 is Ala, Asn, Ser, Ile, Gly, Val, Gln or Thr; Xaa18 is His, Leu, Gln or Ala; Xaa19 is Pro, Gln, Leu, Asn or Ile; Xaa21 is Trp, Phe, Tyr, His or Ile; Xaa31 is Glu, Asp, Gln, Asn, Ser, Ala, Val, Leu, Ile or Thr; Xaa32 is Glu, Gln, Asp Asn, Pro, Thr, Leu, Ser, Ala, Gly or Val; Xaa34 is Ile, Thr, Ser, Val, Ala, Asn, Gly or Leu; Xaa35 is Tyr, Trp or Phe; Xaa39 is Glu, Gly, Ala, Ser or Asp; amino acids Xaa6, Xaa7, Xaa8, Xaa9, Xaa20, Xaa24, Xaa25, Xaa26, Xaa27, Xaa28, Xaa29, Xaa41, Xaa42, Xaa44, Xaa46, Xaa47, Xaa48, Xaa49, Xaa50, Xaa52, Xaa53 and Xaa54 is any amino acid; each of the first four and at last three amino acids of SEQ ID NO:1 can each individually optionally be present or absent and is any non-cysteine amino acid.

In some embodiments, the inhibitor of plasma kallikrein is a Kunitz domain containing polypeptide that comprises the sequence of amino acids 3-60 of SEQ ID NO:2.

In some embodiments, the inhibitor of plasma kallikrein is a Kunitz domain containing polypeptide that consists of the sequence of amino acids 3-60 of SEQ ID NO:2.

In some embodiments, the inhibitor of plasma kallikrein is a Kunitz domain containing polypeptide that comprises the sequence of SEQ ID NO:2.

In some embodiments, the inhibitor of plasma kallikrein is a Kunitz domain containing polypeptide that consists of the sequence of SEQ ID NO:2.

In some embodiments, the protease binding protein is an inhibitor of plasmin.

In some embodiments, the inhibitor of plasmin is selected from the group consisting of: a Kunitz domain containing polypeptide and a plasmin binding antibody.

In some embodiments, the inhibitor of plasmin is a Kunitz domain containing polypeptide that comprises the sequence:

Xaa1-Xaa2-Xaa3-Xaa4-Cys-Xaa6-Xaa7-Xaa8-Xaa9-Xaa10-Xaa11-Gly-Xaa13-Cys-Xaa15-Xaa16Xaa17-Xaa18-Xaa 19-Arg-Xaa21-Xaa22-Xaa23-Xaa24-Xaa25-Xaa26-Xaa27-Xaa28-Xaa29-Cys-Xaa31-Xaa 32-Phe-Xaa34-Xaa35-Xaa36-Gly-Cys-Xaa39-Xaa40-Xaa41-Xaa42-Xaa43-Xaa44-Xaa45-Xaa46-Xaa47-Xaa48-Xaa49-Xaa50-Cys-Xaa52-Xaa53-Xaa54-Cys-Xaa56-Xaa57-Xaa58 (SEQ ID NO:300), wherein

Xaa1, Xaa2, Xaa3, Xaa4, Xaa56, Xaa57 and Xaa58 may each individually be absent; Xaa10 is Asp, Glu, Tyr, or Gln; Xaa11 is Thr, Ala, Ser, Val or Asp; Xaa13 is Pro, Leu or Ala; Xaa15 is Lys or Arg; Xaa16 is Ala or Gly; Xaa17 is Arg, Lys or Ser; Xaa18 is Phe or Ile; Xaa19 is Glu, Gln, Asp, Pro, Gly, Ser or Ile; Xaa21 is Phe, Tyr or Trp; Xaa22 is Tyr or Phe. Xaa23 is Tyr or Phe; Xaa31 is Asp, Glu, Thr, Val, Gln or Ala; Xaa32 is Thr, Ala, Glu, Pro, or Gln; Xaa34 is Val, Ile, Thr, Leu, Phe, Tyr, His, Asp, Ala, or Ser; Xaa35 is Tyr or Trp; Xaa36 is Gly or Ser; Xaa39 is Glu, Gly, Asp, Arg, Ala, Gln, Leu, Lys, or Met; Xaa40 is Gly or Ala; Xaa43 is Asn or Gly; or Xaa45 is Phe or Tyr; and where not specified, Xaa is any non-cysteine amino acid.

In some embodiments, the inhibitor of plasmin is a Kunitz domain containing polypeptide that comprises the sequence of SEQ ID NO:100.

In some embodiments, the inhibitor of plasmin is a Kunitz domain containing polypeptide that consists of the sequence of SEQ ID NO:100.

In some embodiments, the inhibitor of plasmin is a Kunitz domain containing polypeptide that comprises the sequence of SEQ ID NO:200.

In some embodiments, the inhibitor of plasmin is a Kunitz domain containing polypeptide that consists of the sequence of SEQ ID NO:200.

In some embodiments, the protease binding protein is an MMP-14 binding protein.

In some embodiments, the MMP-14 binding protein comprises the 3 heavy chain CDRs and/or the 3 light chain CDRs of DX-2400, DX-2410, M0031-C02, M0031-F01, M0033-H07, M0037-009, M0037-D01, M0038-E06, M0038-F01, M0038-F08, M0039-H08, M0040-A06, M0040-A11, or M0043-G02.

In some embodiments, the MMP-14 binding protein comprises the heavy chain variable region and/or the light chain variable region of DX-2400, DX-2410, M0031-C02, M0031-F01, M0033-H07, M0037-009, M0037-D01, M0038-E06, M0038-F01, M0038-F08, M0039-H08, M0040-A06, M0040-A11, or M0043-G02.

In some embodiments, the MMP-14 binding protein comprises the 3 heavy chain CDRs and/or the 3 light chain CDRs of DX-2400.

In some embodiments, the MMP-14 binding protein comprises the heavy chain variable region and/or the light chain variable region of DX-2400.

In some embodiments, the protease binding protein is an MMP-9 binding protein.

In some embodiments, the MMP-9 binding protein comprises the 3 heavy chain CDRs and/or the 3 light chain CDRs of DX-2802, 539A-M0240-B03, M0078-G07, M0081-D05, M0076-D03, M0072-H07, M0075-D12, M0166-F10, M0279-A03, M0279-B02, M0288-008, or M0281-F06.

In some embodiments, the MMP-9 binding protein comprises the heavy chain variable region and/or the light chain variable region of DX-2802, 539A-M0240-B03, M0078-G07, M0081-D05, M0076-D03, M0072-H07, M0075-D12, M0166-F10, M0279-A03, M0279-B02, M0288-008, or M0281-F06.

In some embodiments, the MMP-9 binding protein comprises the 3 heavy chain CDRs and/or the 3 light chain CDRs of DX-2802.

In some embodiments, the MMP-9 binding protein comprises the heavy chain variable region and/or the light chain variable region of DX-2802.

In some embodiments, the protease binding protein is an MMP-9/-2 binding protein.

In some embodiments, the MMP-9/-2 binding protein comprises the 3 heavy chain CDRs and/or the 3 light chain CDRs of M0237-D02, X0106-A01, X0106-B02, X0106-004, X0106-E4, or X0106-F05.

In some embodiments, the MMP-9/-2 binding protein comprises the heavy chain variable region and/or the light chain variable region of M0237-D02, X0106-A01, X0106-B02, X0106-004, X0106-E4, or X0106-F05.

In some embodiments, the MMP-9/-2 binding protein comprises the 3 heavy chain CDRs and/or the 3 light chain CDRs of M0237-D02.

In some embodiments, the MMP-9/-2 binding protein comprises the heavy chain variable region and/or the light chain variable region of M0237-D02.

In some embodiments, the protease binding protein is an MMP-12 binding protein.

In some embodiments, the MMP-12 binding protein comprises the 3 heavy chain CDRs and/or the 3 light chain CDRs of DX-2712, a mutant or variant of DX-2712, 539B-X0041-D02, M0134-A02, M134-A05, M134-A07, M134-A09, M134-A10, M134-A11, M0134-B01, M134-B04, M0134-B08, M0134-B11, M0134-001, M0134-C02, M0134-006, M0134-009, M0134-C10, M0134-C11, M0134-C12, M0134-D02, M0134-D03, M0134-E04, M0134-E07, M0134-E08, M134-E11, M0134-F01, M0134-F05, M0134-G02, M0134-G04, M0134-G07, M0135-A03, M0135-A05, M0135-A06, M0135-A07, M0135-B02, M0135-B08, M0135-001, M0135-C11, M0135-E03, M0135-F03, M0135-F11, M0135-G02, M0135-G03, M0135-G07, M0135-G11, M0135-H03, M0135-H10, M0105-C05, M0105-E11, M0105-F08, M0107-A12, M0108-A02, M0109-G11, M0110-G05, M0129-B11, M0130-A01, M0130-C12, M0130-F06, M0130-H04, M0131-A06, M0131-D03, M0132-A04, M0133-B08, M0133-E05, M0121-E07, M0118-F11, M0125-G07, M0124-E07, M0119-D01, M0119-A02, M0122-006, M0123-G07, M0063-A02, M0063-A04, M0063-B01, M0063-B11, M0063-007, M0063-G01, M0065-E12, M0065-G03, M0065-H05, M0067-A02, M0067-B06, M0067-B09, M0067-C10, M0067-F02, M0067-F06, M0069-A04, M0069-A11, M0069-C02, M0069-D10, M0069-G07, M0071-A01, M0071-B07, M0071-D05, M0071-D09, M0071-H03, M0071-H06, M0087-F09, M0088-F07, M0088-G10, M0088-H10, M0089-001, M0089-F05, M0089-B07, M0089-H11, M0032-E01, M0034-004, M0039-F01, M0041-B05, M0041-G01, M0042-B06, M0006-B10, M0007-H06, M0008-H09, M0009-H08, M0011-H11, M0015-F02, M0016-D01, M0013-D11, M0013-G12, M0013-H06, M0014-009, M0014-G11, M0016-A11, M0016-H05, M0019-C05, M0020-B01, M0022-C07, M0025-D04, M0027-E11, 539B-X0041-D02, 539B-X0049-A01, 539B-X0049-B01, 539B-X0049-001, 539B-X0049-D01, 539B-X0049-E01, 539B-X0049-F01, 539B-X0049-G01, 539B-X0049-H01, 539B-X0049-A02, 539B-X0049-B02, 539B-X0049-D02, 539B-X0049-D02, or 539B-X0049-E02.

In some embodiments, the MMP-12 binding protein comprises the heavy chain variable region and/or the light chain variable region of DX-2712, a mutant or variant of DX-2712, 539B-X0041-D02, M0134-A02, M134-A05, M134-A07, M134-A09, M134-A10, M134-A11, M0134-B01, M134-B04, M0134-B08, M0134-B11, M0134-001, M0134-C02, M0134-006, M0134-009, M0134-C10, M0134-C11, M0134-C12, M0134-D02, M0134-D03, M0134-E04, M0134-E07, M0134-E08, M134-E11, M0134-F01, M0134-F05, M0134-G02, M0134-G04, M0134-G07, M0135-A03, M0135-A05, M0135-A06, M0135-A07, M0135-B02, M0135-B08, M0135-001, M0135-C11, M0135-E03, M0135-F03, M0135-F11, M0135-G02, M0135-G03, M0135-G07, M0135-G11, M0135-H03, M0135-H10, M0105-C05, M0105-E11, M0105-F08, M0107-A12, M0108-A02, M0109-G11, M0110-G05, M0129-B11, M0130-A01, M0130-C12, M0130-F06, M0130-H04, M0131-A06, M0131-D03, M0132-A04, M0133-B08, M0133-E05, M0121-E07, M0118-F11, M0125-G07, M0124-E07, M0119-D01, M0119-A02, M0122-006, M0123-G07, M0063-A02, M0063-A04, M0063-B01, M0063-B11, M0063-007, M0063-G01, M0065-E12, M0065-G03, M0065-H05, M0067-A02, M0067-B06, M0067-B09, M0067-C10, M0067-F02, M0067-F06, M0069-A04, M0069-A11, M0069-C02, M0069-D10, M0069-G07, M0071-A01, M0071-B07, M0071-D05, M0071-D09, M0071-H03, M0071-H06, M0087-F09, M0088-F07, M0088-G10, M0088-H10, M0089-001, M0089-F05, M0089-B07, M0089-H11, m0032-E01, M0034-004, M0039-F01, M0041-B05, M0041-G01, M0042-B06, M0006-B10, M0007-H06, M0008-H09, M0009-H08, M0011-H11, M0015-F02, M0016-D01, M0013-D11, M0013-G12, M0013-H06, M0014-009, M0014-G11, M0016-A11, M0016-H05, M0019-C05, M0020-B01, M0022-007, M0025-D04, M0027-E11, 539B-X0041-D02, 539B-X0049-A01, 539B-X0049-B01, 539B-X0049-001, 539B-X0049-D01, 539B-X0049-E01, 539B-X0049-F01, 539B-X0049-G01, 539B-X0049-H01, 539B-X0049-A02, 539B-X0049-B02, 539B-X0049-C02, 539B-X0049-D02, or 539B-X0049-E02.

In some embodiments, the MMP-12 binding protein comprises the 3 heavy chain CDRs and/or the 3 light chain CDRs of DX-2712.

In some embodiments, the MMP-12 binding protein comprises the heavy chain variable region and/or the light chain variable region of DX-2712.

In some embodiments, the second agent is an agent for the treatment of an inflammatory disorder selected from the group of inflammatory disorders consisting of: rheumatoid arthritis, psoriasis, multiple sclerosis, systemic sclerosis, asthma, chronic obstructive pulmonary disease, and inflammatory bowel disease.

In some embodiments, the second agent is an immunosuppressant agent selected from the group consisting of: gancyclovir, etanercept, cyclosporine, tacrolimus, rapamycin, cyclophosphamide, azathioprine, mycophenolate mofetil, methotrexate, cortisol, aldosterone, dexamethasone, a cyclooxygenase inhibitor, a 5-lipoxygenase inhibitor, and leukotriene receptor antagonist.

In some embodiments, the second agent is an agent for the treatment of rheumatoid arthritis and comprises a nonsteroidal anti-inflammatory drug (NSAID), a corticosteroid, a Disease-Modifying Antirheumatic Drug (DMARD), or a biological response modifier (BRM).

In some embodiments, the second agent is an agent for the treatment of rheumatoid arthritis and comprises aspirin, naproxen, ibuprofen, etodolac, gold, salsalte, methotrexate, sulfasalazine, D-penicillamine, azathioprine, cyclophosphamide, chlorambucil, cyclosporine, leflunomide, etanercept, infliximab, anakinra, adalimumab, hydroxychloroquine, chloroquine phosphate, chloroquine sulphate, minocycline, or a CTLA4-Ig.

In some embodiments, the second agent is an agent for the treatment of psoriasis and comprises a topical treatment or a systemic treatment.

In some embodiments, the second agent is an agent for the treatment of psoriasis and comprises a topical treatment selected from the group consisting of coal tar, dithranol, a corticosteroid, a vitamin D3 analogue, and a retinoid.

In some embodiments, the second agent is an agent for the treatment of psoriasis and comprises a systemic treatment selected from the group consisting of methotrexate, cyclosporine, a retinoid, tioguanine, hydroxyurea, sulfasalazine, mycophenolate mofetil, azathioprine, tacrolimus, alefacept, efalizumab, etanercept, and infliximab.

In some embodiments, the second agent is an agent for the treatment of multiple sclerosis and comprises a corticosteroid, an interferon, glatiramer acetate, an immunosuppressant or natalizumab.

In some embodiments, the second agent is an agent for the treatment of systemic sclerosis and comprises an NSAID, a calcium channel blocker, prostacyclin analogue, a dual endothelin-receptor antagonist, methotrexate, cyclosporin, an ACE inhibitor, cyclophosphamide, a steroid, or epoprostenol.

In some embodiments, the second agent is an agent for the treatment of systemic sclerosis and comprises nifedipine.

In some embodiments, the second agent is an agent for the treatment of systemic sclerosis and comprises iloprost.

In some embodiments, the second agent is an agent for the treatment of systemic sclerosis and comprises bosentan.

In some embodiments, the second agent is an agent for the treatment of asthma and comprises a glucocorticoid, a leukotriene modifier, a mast cell stabilizer, an antimuscarinic/anticholinergic, an antihistamines, an IgE blocker, or methotrexate.

In some embodiments, the second agent is an agent for the treatment of asthma and comprises a beta2-adrenoceptor agonist selected from the group consisting of: salbutamol, levalbuterol, terbutaline and bitolterol.

In some embodiments, the second agent is an agent for the treatment of asthma and comprises an adrenergic agonist selected from the group consisting of: inhaled epinephrine and ephedrine tablets.

In some embodiments, the second agent is an agent for the treatment of asthma and comprises an antimuscarinic/anticholinergic selected from the group consisting of: ipratropium, oxitropium, and tiotropium.

In some embodiments, the second agent is an agent for the treatment of asthma and comprises an inhaled glucocorticoid selected from the group consisting of: ciclesonide, beclomethasone, budesonide, flunisolide, fluticasone, mometasone, and triamcinolone.

In some embodiments, the second agent is an agent for the treatment of asthma and comprises a leukotriene modifier selected from the group consisting of: montelukast, zafirlukast, pranlukast, and zileuton.

In some embodiments, the second agent is an agent for the treatment of asthma and comprises a mast cell stabilizer selected from the group consisting of: cromoglicate and nedocromil.

In some embodiments, the second agent is an agent for the treatment of asthma and comprises a methylxanthine selected from the group consisting of: theophylline and aminophylline.

In some embodiments, the second agent is an agent for the treatment of asthma and comprises an antihistamine.

In some embodiments, the second agent is an agent for the treatment of asthma and comprises an IgE blocker selected from the group consisting of: omalizumab and methotrexate.

In some embodiments, the second agent is an agent for the treatment of asthma and comprises a long-acting beta₂-adrenoceptor agonist selected from the group consisting of: salmeterol, formoterol, bambuterol, and albuterol.

In some embodiments, the second agent is an agent for the treatment of asthma and comprises a combination of inhaled steroid and a long-acting bronchodilator selected from the group consisting of: fluticasone/salmeterol and budesonide/formoterol.

In some embodiments, the second agent is an agent for the treatment of chronic obstructive pulmonary disease (COPD)and comprises a bronchodilator.

In some embodiments, the second agent is an agent for the treatment of chronic obstructive pulmonary disease (COPD)and comprises a β₂ agonist, an M₃ antimuscarinic, a leukotriene antagonist, a cromone, a corticosteroid, or a xanthine.

In some embodiments, the second agent is an agent for the treatment of chronic obstructive pulmonary disease (COPD)and comprises a β₂ agonist selected from the group consisting of: Salbutamol, Bambuterol, Clenbuterol, Fenoterol, Formoterol, and Salmeterol.

In some embodiments, the second agent is an agent for the treatment of chronic obstructive pulmonary disease (COPD)and comprises the Ipratropium.

In some embodiments, the second agent is an agent for the treatment of chronic obstructive pulmonary disease (COPD)and comprises a cromone selected from the group consisting of: Cromoglicate and Nedocromil.

In some embodiments, the second agent is an agent for the treatment of chronic obstructive pulmonary disease (COPD)and comprises a leukotriene antagonist selected from the group consisting of: Montelukast, Pranlukast, and Zafirlukast.

In some embodiments, the second agent is an agent for the treatment of chronic obstructive pulmonary disease (COPD)and comprises a corticosteroid antagonist selected from the group consisting of: glucocorticoids, beclomethasone, mometasone, and fluticasone.

In some embodiments, the second agent is an agent for the treatment of chronic obstructive pulmonary disease (COPD)and comprises a xanthine antagonist selected from the group consisting of: theophylline, methylxanthine, and theobromine.

In some embodiments, the second agent is an agent for the treatment of chronic obstructive pulmonary disease (COPD) and comprises Ipratropium or Tiotropium.

In some embodiments, the second agent is an agent for the treatment of inflammatory bowel disease and comprises an immunosuppresant, an anti-TNF binding protein, a cytokine inhibitor, a BRM, or an anti-inflammatory.

In some embodiments, the second agent is an agent for the treatment of inflammatory bowel disease and comprises prednisone, infliximab, azathioprine, methotrexate, 6-mercaptopurine , a mesalamine, a steroid, or CDP571 antibody.

In another aspect, the disclosure features a composition (e.g., an isolated composition) that includes a protease binding protein and a second agent, wherein the second agent is an agent for the treatment of an inflammatory disorder.

In some embodiments, the protease binding protein is a protease inhibitor.

In some embodiments, the protease binding protein binds to a protease selected from the group consisting of: plasma kallikrein, plasmin, MMP-14, MMP-9, MMP-9/-2, and MMP-12.

In some embodiments, the protease binding protein is an inhibitor of plasma kallikrein.

In some embodiments, the inhibitor of plasma kallikrein is selected from the group consisting of: a Kunitz domain containing polypeptide and aplasma kallikrein binding antibody.

In some embodiments, the inhibitor of plasma kallikrein is a Kunitz domain containing polypeptide that comprises the sequence:

Xaa1 Xaa2 Xaa3 Xaa4 Cys Xaa6 Xaa7 Xaa8 Xaa9 Xaa10 Xaa11 Gly Xaa13 Cys Xaa15 Xaa16 Xaa17 Xaa18 Xaa19 Xaa20 Xaa21 Xaa22 Xaa23 Xaa24 Xaa25 Xaa26 Xaa27 Xaa28 Xaa29 Cys Xaa31 Xaa32 Phe Xaa34 Xaa35 Gly Gly Cys Xaa39 Xaa40 Xaa41 Xaa42 Xaa43 Xaa44 Xaa45 Xaa46 Xaa47 Xaa48 Xaa49 Xaa50 Cys Xaa52 Xaa53 Xaa54 Cys Xaa56 Xaa57 Xaa58 (SEQ ID NO:1), wherein

Xaa10 is Asp or Glu; Xaa11 is Asp, Gly, Ser, Val, Asn, Ile, Ala or Thr; Xaa13 is Pro, Arg, His, Asn, Ser, Thr, Ala, Gly, Lys or Gln; Xaa15 is Arg, Lys, Ala, Ser, Gly, Met, Asn or Gln; Xaa16 is Ala, Gly, Ser, Asp or Asn; Xaa17 is Ala, Asn, Ser, Ile, Gly, Val, Gln or Thr; Xaa18 is His, Leu, Gln or Ala; Xaa19 is Pro, Gln, Leu, Asn or Ile; Xaa21 is Trp, Phe, Tyr, His or Ile; Xaa31 is Glu, Asp, Gln, Asn, Ser, Ala, Val, Leu, Ile or Thr; Xaa32 is Glu, Gln, Asp Asn, Pro, Thr, Leu, Ser, Ala, Gly or Val; Xaa34 is Ile, Thr, Ser, Val, Ala, Asn, Gly or Leu; Xaa35 is Tyr, Trp or Phe; Xaa39 is Glu, Gly, Ala, Ser or Asp; amino acids Xaa6, Xaa7, Xaa8, Xaa9, Xaa20, Xaa24, Xaa25, Xaa26, Xaa27, Xaa28, Xaa29, Xaa41, Xaa42, Xaa44, Xaa46, Xaa47, Xaa48, Xaa49, Xaa50, Xaa52, Xaa53 and Xaa54 is any amino acid; each of the first four and at last three amino acids of SEQ ID NO:1 can each individually optionally be present or absent and is any non-cysteine amino acid.

In some embodiments, the inhibitor of plasma kallikrein is a Kunitz domain containing polypeptide that comprises the sequence of amino acids 3-60 of SEQ ID NO:2.

In some embodiments, the inhibitor of plasma kallikrein is a Kunitz domain containing polypeptide that consists of the sequence of amino acids 3-60 of SEQ ID NO:2.

In some embodiments, the inhibitor of plasma kallikrein is a Kunitz domain containing polypeptide that comprises the sequence of SEQ ID NO:2.

In some embodiments, the inhibitor of plasma kallikrein is a Kunitz domain containing polypeptide that consists of the sequence of SEQ ID NO:2.

In some embodiments, the protease binding protein is an inhibitor of plasmin.

In some embodiments, the inhibitor of plasmin is selected from the group consisting of: a Kunitz domain containing polypeptide and a plasmin binding antibody.

In some embodiments, the inhibitor of plasmin is a Kunitz domain containing polypeptide that comprises the sequence:

Xaa1-Xaa2-Xaa3-Xaa4-Cys-Xaa6-Xaa7-Xaa8-Xaa9-Xaa10-Xaa11-Gly-Xaa13-Cys-Xaa15-Xaa16Xaa17-Xaa18-Xaa 19-Arg-Xaa21-Xaa22-Xaa23-Xaa24-Xaa25-Xaa26-Xaa27-Xaa28-Xaa29-Cys-Xaa31-Xaa 32-Phe-Xaa34-Xaa35-Xaa36-Gly-Cys-Xaa39-Xaa40-Xaa41-Xaa42-Xaa43-Xaa44-Xaa45-Xaa46-Xaa47-Xaa48-Xaa49-Xaa50-Cys-Xaa52-Xaa53-Xaa54-Cys-Xaa56-Xaa57-Xaa58 (SEQ ID NO:300), wherein

Xaa1, Xaa2, Xaa3, Xaa4, Xaa56, Xaa57 and Xaa58 may each individually be absent; Xaa10 is Asp, Glu, Tyr, or Gln; Xaa11 is Thr, Ala, Ser, Val or Asp; Xaa13 is Pro, Leu or Ala; Xaa15 is Lys or Arg; Xaa16 is Ala or Gly; Xaa17 is Arg, Lys or Ser; Xaa18 is Phe or Ile; Xaa19 is Glu, Gln, Asp, Pro, Gly, Ser or Ile; Xaa21 is Phe, Tyr or Trp; Xaa22 is Tyr or Phe. Xaa23 is Tyr or Phe; Xaa31 is Asp, Glu, Thr, Val, Gln or Ala; Xaa32 is Thr, Ala, Glu, Pro, or Gln; Xaa34 is Val, Ile, Thr, Leu, Phe, Tyr, His, Asp, Ala, or Ser; Xaa35 is Tyr or Trp; Xaa36 is Gly or Ser; Xaa39 is Glu, Gly, Asp, Arg, Ala, Gln, Leu, Lys, or Met; Xaa40 is Gly or Ala; Xaa43 is Asn or Gly; or Xaa45 is Phe or Tyr; and where not specified, Xaa is any non-cysteine amino acid.

In some embodiments, the inhibitor of plasmin is a Kunitz domain containing polypeptide that comprises the sequence of SEQ ID NO:100.

In some embodiments, the inhibitor of plasmin is a Kunitz domain containing polypeptide that consists of the sequence of SEQ ID NO:100.

In some embodiments, the inhibitor of plasmin is a Kunitz domain containing polypeptide that comprises the sequence of SEQ ID NO:200.

In some embodiments, the inhibitor of plasmin is a Kunitz domain containing polypeptide that consists of the sequence of SEQ ID NO:200.

In some embodiments, the protease binding protein is an MMP-14 binding protein.

In some embodiments, the MMP-14 binding protein comprises the 3 heavy chain CDRs and/or the 3 light chain CDRs of DX-2400, DX-2410, M0031-C02, M0031-F01, M0033-H07, M0037-009, M0037-D01, M0038-E06, M0038-F01, M0038-F08, M0039-H08, M0040-A06, M0040-A11, or M0043-G02.

In some embodiments, the MMP-14 binding protein comprises the heavy chain variable region and/or the light chain variable region of DX-2400, DX-2410, M0031-C02, M0031-F01, M0033-H07, M0037-009, M0037-D01, M0038-E06, M0038-F01, M0038-F08, M0039-H08, M0040-A06, M0040-A11, or M0043-G02.

In some embodiments, the MMP-14 binding protein comprises the 3 heavy chain CDRs and/or the 3 light chain CDRs of DX-2400.

In some embodiments, the MMP-14 binding protein comprises the heavy chain variable region and/or the light chain variable region of DX-2400.

In some embodiments, the protease binding protein is an MMP-9 binding protein.

In some embodiments, the MMP-9 binding protein comprises the 3 heavy chain CDRs and/or the 3 light chain CDRs of DX-2802, 539A-M0240-B03, M0078-G07, M0081-D05, M0076-D03, M0072-H07, M0075-D12, M0166-F10, M0279-A03, M0279-B02, M0288-008, or M0281-F06.

In some embodiments, the MMP-9 binding protein comprises the heavy chain variable region and/or the light chain variable region of DX-2802, 539A-M0240-B03, M0078-G07, M0081-D05, M0076-D03, M0072-H07, M0075-D12, M0166-F10, M0279-A03, M0279-B02, M0288-008, or M0281-F06.

In some embodiments, the MMP-9 binding protein comprises the 3 heavy chain CDRs and/or the 3 light chain CDRs of DX-2802.

In some embodiments, the MMP-9 binding protein comprises the heavy chain variable region and/or the light chain variable region of DX-2802.

In some embodiments, the protease binding protein is an MMP-9/-2 binding protein.

In some embodiments, the MMP-9/-2 binding protein comprises the 3 heavy chain CDRs and/or the 3 light chain CDRs of M0237-D02, X0106-A01, X0106-B02, X0106-004, X0106-E4, or X0106-F05.

In some embodiments, the MMP-9/-2 binding protein comprises the heavy chain variable region and/or the light chain variable region of M0237-D02, X0106-A01, X0106-B02, X0106-004, X0106-E4, or X0106-F05.

In some embodiments, the MMP-9/-2 binding protein comprises the 3 heavy chain CDRs and/or the 3 light chain CDRs of M0237-D02.

In some embodiments, the MMP-9/-2 binding protein comprises the heavy chain variable region and/or the light chain variable region of M0237-D02.

In some embodiments, the protease binding protein is an MMP-12 binding protein.

In some embodiments, the MMP-12 binding protein comprises the 3 heavy chain CDRs and/or the 3 light chain CDRs of DX-2712, a mutant or variant of DX-2712, 539B-X0041-D02, M0134-A02, M134-A05, M134-A07, M134-A09, M134-A10, M134-A11, M0134-B01, M134-B04, M0134-B08, M0134-B11, M0134-001, M0134-C02, M0134-006, M0134-009, M0134-C10, M0134-C11, M0134-C12, M0134-D02, M0134-D03, M0134-E04, M0134-E07, M0134-E08, M134-E11, M0134-F01, M0134-F05, M0134-G02, M0134-G04, M0134-G07, M0135-A03, M0135-A05, M0135-A06, M0135-A07, M0135-B02, M0135-B08, M0135-001, M0135-C11, M0135-E03, M0135-F03, M0135-F11, M0135-G02, M0135-G03, M0135-G07, M0135-G11, M0135-H03, M0135-H10, M0105-C05, M0105-E11, M0105-F08, M0107-A12, M0108-A02, M0109-G11, M0110-G05, M0129-B11, M0130-A01, M0130-C12, M0130-F06, M0130-H04, M0131-A06, M0131-D03, M0132-A04, M0133-B08, M0133-E05, M0121-E07, M0118-F11, M0125-G07, M0124-E07, M0119-D01, M0119-A02, M0122-006, M0123-G07, M0063-A02, M0063-A04, M0063-B01, M0063-B11, M0063-007, M0063-G01, M0065-E12, M0065-G03, M0065-H05, M0067-A02, M0067-B06, M0067-B09, M0067-C10, M0067-F02, M0067-F06, M0069-A04, M0069-A11, M0069-C02, M0069-D10, M0069-G07, M0071-A01, M0071-B07, M0071-D05, M0071-D09, M0071-H03, M0071-H06, M0087-F09, M0088-F07, M0088-G10, M0088-H10, M0089-001, M0089-F05, M0089-B07, M0089-H11, M0032-E01, M0034-004, M0039-F01, M0041-B05, M0041-G01, M0042-B06, M0006-B10, M0007-H06, M0008-H09, M0009-H08, M0011-H11, M0015-F02, M0016-D01, M0013-D11, M0013-G12, M0013-H06, M0014-C09, M0014-G11, M0016-A11, M0016-H05, M0019-C05, M0020-B01, M0022-007, M0025-D04, M0027-E11, 539B-X0041-D02, 539B-X0049-A01, 539B-X0049-B01, 539B-X0049-001, 539B-X0049-D01, 539B-X0049-E01, 539B-X0049-F01, 539B-X0049-G01, 539B-X0049-H01, 539B-X0049-A02, 539B-X0049-B02, 539B-X0049-C02, 539B-X0049-D02, or 539B-X0049-E02.

In some embodiments, the MMP-12 binding protein comprises the heavy chain variable region and/or the light chain variable region of DX-2712, a mutant or variant of DX-2712, 539B-X0041-D02, M0134-A02, M134-A05, M134-A07, M134-A09, M134-A10, M134-A11, M0134-B01, M134-B04, M0134-B08, M0134-B11, M0134-001, M0134-C02, M0134-006, M0134-009, M0134-C10, M0134-C11, M0134-C12, M0134-D02, M0134-D03, M0134-E04, M0134-E07, M0134-E08, M134-E11, M0134-F01, M0134-F05, M0134-G02, M0134-G04, M0134-G07, M0135-A03, M0135-A05, M0135-A06, M0135-A07, M0135-B02, M0135-B08, M0135-001, M0135-C11, M0135-E03, M0135-F03, M0135-F11, M0135-G02, M0135-G03, M0135-G07, M0135-G11, M0135-H03, M0135-H10, M0105-C05, M0105-E11, M0105-F08, M0107-A12, M0108-A02, M0109-G11, M0110-G05, M0129-B11, M0130-A01, M0130-C12, M0130-F06, M0130-H04, M0131-A06, M0131-D03, M0132-A04, M0133-B08, M0133-E05, M0121-E07, M0118-F11, M0125-G07, M0124-E07, M0119-D01, M0119-A02, M0122-006, M0123-G07, M0063-A02, M0063-A04, M0063-B01, M0063-B11, M0063-007, M0063-G01, M0065-E12, M0065-G03, M0065-H05, M0067-A02, M0067-B06, M0067-B09, M0067-C10, M0067-F02, M0067-F06, M0069-A04, M0069-A11, M0069-C02, M0069-D10, M0069-G07, M0071-A01, M0071-B07, M0071-D05, M0071-D09, M0071-H03, M0071-H06, M0087-F09, M0088-F07, M0088-G10, M0088-H10, M0089-001, M0089-F05, M0089-B07, M0089-H11, m0032-E01, M0034-004, M0039-F01, M0041-B05, M0041-G01, M0042-B06, M0006-B10, M0007-H06, M0008-H09, M0009-H08, M0011-H11, M0015-F02, M0016-D01, M0013-D11, M0013-G12, M0013-H06, M0014-009, M0014-G11, M0016-A11, M0016-H05, M0019-C05, M0020-B01, M0022-007, M0025-D04, M0027-E11, 539B-X0041-D02, 539B-X0049-A01, 539B-X0049-B01, 539B-X0049-001, 539B-X0049-D01, 539B-X0049-E01, 539B-X0049-F01, 539B-X0049-G01, 539B-X0049-H01, 539B-X0049-A02, 539B-X0049-B02, 539B-X0049-C02, 539B-X0049-D02, or 539B-X0049-E02.

In some embodiments, the MMP-12 binding protein comprises the 3 heavy chain CDRs and/or the 3 light chain CDRs of DX-2712.

In some embodiments, the MMP-12 binding protein comprises the heavy chain variable region and/or the light chain variable region of DX-2712.

In some embodiments, the second agent is an agent for the treatment of an inflammatory disorder selected from the group of inflammatory disorders consisting of: rheumatoid arthritis, psoriasis, multiple sclerosis, systemic sclerosis, asthma, chronic obstructive pulmonary disease, and inflammatory bowel disease.

In some embodiments, the second agent is an immunosuppressant agent selected from the group consisting of: gancyclovir, etanercept, cyclosporine, tacrolimus, rapamycin, cyclophosphamide, azathioprine, mycophenolate mofetil, methotrexate, cortisol, aldosterone, dexamethasone, a cyclooxygenase inhibitor, a 5-lipoxygenase inhibitor, and leukotriene receptor antagonist.

In some embodiments, the second agent is an agent for the treatment of rheumatoid arthritis and comprises a nonsteroidal anti-inflammatory drug (NSAID), a corticosteroid, a Disease-Modifying Antirheumatic Drug (DMARD), or a biological response modifier (BRM).

In some embodiments, the second agent is an agent for the treatment of rheumatoid arthritis and comprises aspirin, naproxen, ibuprofen, etodolac, gold, salsalte, methotrexate, sulfasalazine, D-penicillamine, azathioprine, cyclophosphamide, chlorambucil, cyclosporine, leflunomide, etanercept, infliximab, anakinra, adalimumab, hydroxychloroquine, chloroquine phosphate, chloroquine sulphate, minocycline, or a CTLA4-Ig.

In some embodiments, the second agent is an agent for the treatment of psoriasis and comprises a topical treatment or a systemic treatment.

In some embodiments, the second agent is an agent for the treatment of psoriasis and comprises a topical treatment selected from the group consisting of coal tar, dithranol, a corticosteroid, a vitamin D3 analogue, and a retinoid.

In some embodiments, the second agent is an agent for the treatment of psoriasis and comprises a systemic treatment selected from the group consisting of methotrexate, cyclosporine, a retinoid, tioguanine, hydroxyurea, sulfasalazine, mycophenolate mofetil, azathioprine, tacrolimus, alefacept, efalizumab, etanercept, and infliximab.

In some embodiments, the second agent is an agent for the treatment of multiple sclerosis and comprises a corticosteroid, an interferon, glatiramer acetate, an immunosuppressant or natalizumab.

In some embodiments, the second agent is an agent for the treatment of systemic sclerosis and comprises an NSAID, a calcium channel blocker, prostacyclin analogue, a dual endothelin-receptor antagonist, methotrexate, cyclosporin, an ACE inhibitor, cyclophosphamide, a steroid, or epoprostenol.

In some embodiments, the second agent is an agent for the treatment of systemic sclerosis and comprises nifedipine.

In some embodiments, the second agent is an agent for the treatment of systemic sclerosis and comprises iloprost.

In some embodiments, the second agent is an agent for the treatment of systemic sclerosis and comprises bosentan.

In some embodiments, the second agent is an agent for the treatment of asthma and comprises a glucocorticoid, a leukotriene modifier, a mast cell stabilizer, an antimuscarinic/anticholinergic, an antihistamines, an IgE blocker, or methotrexate.

In some embodiments, the second agent is an agent for the treatment of asthma and comprises a beta2-adrenoceptor agonist selected from the group consisting of: salbutamol, levalbuterol, terbutaline and bitolterol.

In some embodiments, the second agent is an agent for the treatment of asthma and comprises an adrenergic agonist selected from the group consisting of: inhaled epinephrine and ephedrine tablets.

In some embodiments, the second agent is an agent for the treatment of asthma and comprises an antimuscarinic/anticholinergic selected from the group consisting of: ipratropium, oxitropium, and tiotropium.

In some embodiments, the second agent is an agent for the treatment of asthma and comprises an inhaled glucocorticoid selected from the group consisting of: ciclesonide, beclomethasone, budesonide, flunisolide, fluticasone, mometasone, and triamcinolone.

In some embodiments, the second agent is an agent for the treatment of asthma and comprises a leukotriene modifier selected from the group consisting of: montelukast, zafirlukast, pranlukast, and zileuton.

In some embodiments, the second agent is an agent for the treatment of asthma and comprises a mast cell stabilizer selected from the group consisting of: cromoglicate and nedocromil.

In some embodiments, the second agent is an agent for the treatment of asthma and comprises a methylxanthine selected from the group consisting of: theophylline and aminophylline.

In some embodiments, the second agent is an agent for the treatment of asthma and comprises an antihistamine.

In some embodiments, the second agent is an agent for the treatment of asthma and comprises an IgE blocker selected from the group consisting of: omalizumab and methotrexate.

In some embodiments, the second agent is an agent for the treatment of asthma and comprises a long-acting beta₂-adrenoceptor agonist selected from the group consisting of: salmeterol, formoterol, bambuterol, and albuterol.

In some embodiments, the second agent is an agent for the treatment of asthma and comprises a combination of inhaled steroid and a long-acting bronchodilator selected from the group consisting of: fluticasone/salmeterol and budesonide/formoterol.

In some embodiments, the second agent is an agent for the treatment of chronic obstructive pulmonary disease (COPD)and comprises a bronchodilator.

In some embodiments, the second agent is an agent for the treatment of chronic obstructive pulmonary disease (COPD)and comprises a β₂ agonist, an M₃ antimuscarinic, a leukotriene antagonist, a cromone, a corticosteroid, or a xanthine.

In some embodiments, the second agent is an agent for the treatment of chronic obstructive pulmonary disease (COPD)and comprises a β₂ agonist selected from the group consisting of: Salbutamol, Bambuterol, Clenbuterol, Fenoterol, Formoterol, and Salmeterol.

In some embodiments, the second agent is an agent for the treatment of chronic obstructive pulmonary disease (COPD)and comprises the Ipratropium.

In some embodiments, the second agent is an agent for the treatment of chronic obstructive pulmonary disease (COPD)and comprises a cromone selected from the group consisting of: Cromoglicate and Nedocromil.

In some embodiments, the second agent is an agent for the treatment of chronic obstructive pulmonary disease (COPD)and comprises a leukotriene antagonist selected from the group consisting of: Montelukast, Pranlukast, and Zafirlukast.

In some embodiments, the second agent is an agent for the treatment of chronic obstructive pulmonary disease (COPD)and comprises a corticosteroid antagonist selected from the group consisting of: glucocorticoids, beclomethasone, mometasone, and fluticasone.

In some embodiments, the second agent is an agent for the treatment of chronic obstructive pulmonary disease (COPD)and comprises a xanthine antagonist selected from the group consisting of: theophylline, methylxanthine, and theobromine.

In some embodiments, the second agent is an agent for the treatment of chronic obstructive pulmonary disease (COPD) and comprises Ipratropium or Tiotropium.

In some embodiments, the second agent is an agent for the treatment of inflammatory bowel disease and comprises an immunosuppresant, an anti-TNF binding protein, a cytokine inhibitor, a BRM, or an anti-inflammatory.

In some embodiments, the second agent is an agent for the treatment of inflammatory bowel disease and comprises prednisone, infliximab, azathioprine, methotrexate, 6-mercaptopurine , a mesalamine, a steroid, or CDP571 antibody.

In another aspect, the disclosure features a kit that contains a protease binding protein and a second agent, wherein the second agent is an agent for the treatment of an inflammatory disorder.

In some embodiments, the protease binding protein is a protease inhibitor.

In some embodiments, the protease binding protein binds to a protease selected from the group consisting of: plasma kallikrein, plasmin, MMP-14, MMP-9, MMP-9/-2, and MMP-12.

In some embodiments, the protease binding protein is an inhibitor of plasma kallikrein.

In some embodiments, the inhibitor of plasma kallikrein is selected from the group consisting of: a Kunitz domain containing polypeptide and aplasma kallikrein binding antibody.

In some embodiments, the inhibitor of plasma kallikrein is a Kunitz domain containing polypeptide that comprises the sequence:

Xaa1 Xaa2 Xaa3 Xaa4 Cys Xaa6 Xaa7 Xaa8 Xaa9 Xaa10 Xaa11 Gly Xaa13 Cys Xaa15 Xaa16 Xaa17 Xaa18 Xaa19 Xaa20 Xaa21 Xaa22 Xaa23 Xaa24 Xaa25 Xaa26 Xaa27 Xaa28 Xaa29 Cys Xaa31 Xaa32 Phe Xaa34 Xaa35 Gly Gly Cys Xaa39 Xaa40 Xaa41 Xaa42 Xaa43 Xaa44 Xaa45 Xaa46 Xaa47 Xaa48 Xaa49 Xaa50 Cys Xaa52 Xaa53 Xaa54 Cys Xaa56 Xaa57 Xaa58 (SEQ ID NO:1), wherein

Xaa10 is Asp or Glu; Xaa11 is Asp, Gly, Ser, Val, Asn, Ile, Ala or Thr; Xaa13 is Pro, Arg, His, Asn, Ser, Thr, Ala, Gly, Lys or Gln; Xaa15 is Arg, Lys, Ala, Ser, Gly, Met, Asn or Gln; Xaa16 is Ala, Gly, Ser, Asp or Asn; Xaa17 is Ala, Asn, Ser, Ile, Gly, Val, Gln or Thr; Xaa18 is His, Leu, Gln or Ala; Xaa19 is Pro, Gln, Leu, Asn or Ile; Xaa21 is Trp, Phe, Tyr, His or Ile; Xaa31 is Glu, Asp, Gln, Asn, Ser, Ala, Val, Leu, Ile or Thr; Xaa32 is Glu, Gln, Asp Asn, Pro, Thr, Leu, Ser, Ala, Gly or Val; Xaa34 is Ile, Thr, Ser, Val, Ala, Asn, Gly or Leu; Xaa35 is Tyr, Trp or Phe; Xaa39 is Glu, Gly, Ala, Ser or Asp; amino acids Xaa6, Xaa7, Xaa8, Xaa9, Xaa20, Xaa24, Xaa25, Xaa26, Xaa27, Xaa28, Xaa29, Xaa41, Xaa42, Xaa44, Xaa46, Xaa47, Xaa48, Xaa49, Xaa50, Xaa52, Xaa53 and Xaa54 is any amino acid; each of the first four and at last three amino acids of SEQ ID NO:1 can each individually optionally be present or absent and is any non-cysteine amino acid.

In some embodiments, the inhibitor of plasma kallikrein is a Kunitz domain containing polypeptide that comprises the sequence of amino acids 3-60 of SEQ ID NO:2.

In some embodiments, the inhibitor of plasma kallikrein is a Kunitz domain containing polypeptide that consists of the sequence of amino acids 3-60 of SEQ ID NO:2.

In some embodiments, the inhibitor of plasma kallikrein is a Kunitz domain containing polypeptide that comprises the sequence of SEQ ID NO:2.

In some embodiments, the inhibitor of plasma kallikrein is a Kunitz domain containing polypeptide that consists of the sequence of SEQ ID NO:2.

In some embodiments, the protease binding protein is an inhibitor of plasmin.

In some embodiments, the inhibitor of plasmin is selected from the group consisting of: a Kunitz domain containing polypeptide and a plasmin binding antibody.

In some embodiments, the inhibitor of plasmin is a Kunitz domain containing polypeptide that comprises the sequence:

Xaa1-Xaa2-Xaa3-Xaa4-Cys-Xaa6-Xaa7-Xaa8-Xaa9-Xaa10-Xaa11-Gly-Xaa13-Cys-Xaa15-Xaa16Xaa17-Xaa18-Xaa 19-Arg-Xaa21-Xaa22-Xaa23-Xaa24-Xaa25-Xaa26-Xaa27-Xaa28-Xaa29-Cys-Xaa31-Xaa 32-Phe-Xaa34-Xaa35-Xaa36-Gly-Cys-Xaa39-Xaa40-Xaa41-Xaa42-Xaa43-Xaa44-Xaa45-Xaa46-Xaa47-Xaa48-Xaa49-Xaa50-Cys-Xaa52-Xaa53-Xaa54-Cys-Xaa56-Xaa57-Xaa58 (SEQ ID NO:300), wherein

Xaa1, Xaa2, Xaa3, Xaa4, Xaa56, Xaa57 and Xaa58 may each individually be absent; Xaa10 is Asp, Glu, Tyr, or Gln; Xaa11 is Thr, Ala, Ser, Val or Asp; Xaa13 is Pro, Leu or Ala; Xaa15 is Lys or Arg; Xaa16 is Ala or Gly; Xaa17 is Arg, Lys or Ser; Xaa18 is Phe or Ile; Xaa19 is Glu, Gln, Asp, Pro, Gly, Ser or Ile; Xaa21 is Phe, Tyr or Trp; Xaa22 is Tyr or Phe. Xaa23 is Tyr or Phe; Xaa31 is Asp, Glu, Thr, Val, Gln or Ala; Xaa32 is Thr, Ala, Glu, Pro, or Gln; Xaa34 is Val, Ile, Thr, Leu, Phe, Tyr, His, Asp, Ala, or Ser; Xaa35 is Tyr or Trp; Xaa36 is Gly or Ser; Xaa39 is Glu, Gly, Asp, Arg, Ala, Gln, Leu, Lys, or Met; Xaa40 is Gly or Ala; Xaa43 is Asn or Gly; or Xaa45 is Phe or Tyr; and where not specified, Xaa is any non-cysteine amino acid.

In some embodiments, the inhibitor of plasmin is a Kunitz domain containing polypeptide that comprises the sequence of SEQ ID NO:100.

In some embodiments, the inhibitor of plasmin is a Kunitz domain containing polypeptide that consists of the sequence of SEQ ID NO:100.

In some embodiments, the inhibitor of plasmin is a Kunitz domain containing polypeptide that comprises the sequence of SEQ ID NO:200.

In some embodiments, the inhibitor of plasmin is a Kunitz domain containing polypeptide that consists of the sequence of SEQ ID NO:200.

In some embodiments, the protease binding protein is an MMP-14 binding protein.

In some embodiments, the MMP-14 binding protein comprises the 3 heavy chain CDRs and/or the 3 light chain CDRs of DX-2400, DX-2410, M0031-C02, M0031-F01, M0033-H07, M0037-009, M0037-D01, M0038-E06, M0038-F01, M0038-F08, M0039-H08, M0040-A06, M0040-A11, or M0043-G02.

In some embodiments, the MMP-14 binding protein comprises the heavy chain variable region and/or the light chain variable region of DX-2400, DX-2410, M0031-C02, M0031-F01, M0033-H07, M0037-009, M0037-D01, M0038-E06, M0038-F01, M0038-F08, M0039-H08, M0040-A06, M0040-A11, or M0043-G02.

In some embodiments, the MMP-14 binding protein comprises the 3 heavy chain CDRs and/or the 3 light chain CDRs of DX-2400.

In some embodiments, the MMP-14 binding protein comprises the heavy chain variable region and/or the light chain variable region of DX-2400.

In some embodiments, the protease binding protein is an MMP-9 binding protein.

In some embodiments, the MMP-9 binding protein comprises the 3 heavy chain CDRs and/or the 3 light chain CDRs of DX-2802, 539A-M0240-B03, M0078-G07, M0081-D05, M0076-D03, M0072-H07, M0075-D12, M0166-F10, M0279-A03, M0279-B02, M0288-008, or M0281-F06.

In some embodiments, the MMP-9 binding protein comprises the heavy chain variable region and/or the light chain variable region of DX-2802, 539A-M0240-B03, M0078-G07, M0081-D05, M0076-D03, M0072-H07, M0075-D12, M0166-F10, M0279-A03, M0279-B02, M0288-008, or M0281-F06.

In some embodiments, the MMP-9 binding protein comprises the 3 heavy chain CDRs and/or the 3 light chain CDRs of DX-2802.

In some embodiments, the MMP-9 binding protein comprises the heavy chain variable region and/or the light chain variable region of DX-2802.

In some embodiments, the protease binding protein is an MMP-9/-2 binding protein.

In some embodiments, the MMP-9/-2 binding protein comprises the 3 heavy chain CDRs and/or the 3 light chain CDRs of M0237-D02, X0106-A01, X0106-B02, X0106-004, X0106-E4, or X0106-F05.

In some embodiments, the MMP-9/-2 binding protein comprises the heavy chain variable region and/or the light chain variable region of M0237-D02, X0106-A01, X0106-B02, X0106-004, X0106-E4, or X0106-F05.

In some embodiments, the MMP-9/-2 binding protein comprises the 3 heavy chain CDRs and/or the 3 light chain CDRs of M0237-D02.

In some embodiments, the MMP-9/-2 binding protein comprises the heavy chain variable region and/or the light chain variable region of M0237-D02.

In some embodiments, the protease binding protein is an MMP-12 binding protein.

In some embodiments, the MMP-12 binding protein comprises the 3 heavy chain CDRs and/or the 3 light chain CDRs of DX-2712, a mutant or variant of DX-2712, 539B-X0041-D02, M0134-A02, M134-A05, M134-A07, M134-A09, M134-A10, M134-A11, M0134-B01, M134-B04, M0134-B08, M0134-B11, M0134-001, M0134-C02, M0134-006, M0134-009, M0134-C10, M0134-C11, M0134-C12, M0134-D02, M0134-D03, M0134-E04, M0134-E07, M0134-E08, M134-E11, M0134-F01, M0134-F05, M0134-G02, M0134-G04, M0134-G07, M0135-A03, M0135-A05, M0135-A06, M0135-A07, M0135-B02, M0135-B08, M0135-001, M0135-C11, M0135-E03, M0135-F03, M0135-F11, M0135-G02, M0135-G03, M0135-G07, M0135-G11, M0135-H03, M0135-H10, M0105-C05, M0105-E11, M0105-F08, M0107-A12, M0108-A02, M0109-G11, M0110-G05, M0129-B11, M0130-A01, M0130-C12, M0130-F06, M0130-H04, M0131-A06, M0131-D03, M0132-A04, M0133-B08, M0133-E05, M0121-E07, M0118-F11, M0125-G07, M0124-E07, M0119-D01, M0119-A02, M0122-006, M0123-G07, M0063-A02, M0063-A04, M0063-B01, M0063-B11, M0063-007, M0063-G01, M0065-E12, M0065-G03, M0065-H05, M0067-A02, M0067-B06, M0067-B09, M0067-C10, M0067-F02, M0067-F06, M0069-A04, M0069-A11, M0069-C02, M0069-D10, M0069-G07, M0071-A01, M0071-B07, M0071-D05, M0071-D09, M0071-H03, M0071-H06, M0087-F09, M0088-F07, M0088-G10, M0088-H10, M0089-001, M0089-F05, M0089-B07, M0089-H11, M0032-E01, M0034-004, M0039-F01, M0041-B05, M0041-G01, M0042-B06, M0006-B10, M0007-H06, M0008-H09, M0009-H08, M0011-H11, M0015-F02, M0016-D01, M0013-D11, M0013-G12, M0013-H06, M0014-009, M0014-G11, M0016-A11, M0016-H05, M0019-C05, M0020-B01, M0022-007, M0025-D04, M0027-E11, 539B-X0041-D02, 539B-X0049-A01, 539B-X0049-B01, 539B-X0049-001, 539B-X0049-D01, 539B-X0049-E01, 539B-X0049-F01, 539B-X0049-G01, 539B-X0049-H01, 539B-X0049-A02, 539B-X0049-B02, 539B-X0049-C02, 539B-X0049-D02, or 539B-X0049-E02.

In some embodiments, the MMP-12 binding protein comprises the heavy chain variable region and/or the light chain variable region of DX-2712, a mutant or variant of DX-2712, 539B-X0041-D02, M0134-A02, M134-A05, M134-A07, M134-A09, M134-A10, M134-A11, M0134-B01, M134-B04, M0134-B08, M0134-B11, M0134-001, M0134-C02, M0134-006, M0134-009, M0134-C10, M0134-C11, M0134-C12, M0134-D02, M0134-D03, M0134-E04, M0134-E07, M0134-E08, M134-E11, M0134-F01, M0134-F05, M0134-G02, M0134-G04, M0134-G07, M0135-A03, M0135-A05, M0135-A06, M0135-A07, M0135-B02, M0135-B08, M0135-001, M0135-C11, M0135-E03, M0135-F03, M0135-F11, M0135-G02, M0135-G03, M0135-G07, M0135-G11, M0135-H03, M0135-H10, M0105-C05, M0105-E11, M0105-F08, M0107-A12, M0108-A02, M0109-G11, M0110-G05, M0129-B11, M0130-A01, M0130-C12, M0130-F06, M0130-H04, M0131-A06, M0131-D03, M0132-A04, M0133-B08, M0133-E05, M0121-E07, M0118-F11, M0125-G07, M0124-E07, M0119-D01, M0119-A02, M0122-006, M0123-G07, M0063-A02, M0063-A04, M0063-B01, M0063-B11, M0063-007, M0063-G01, M0065-E12, M0065-G03, M0065-H05, M0067-A02, M0067-B06, M0067-B09, M0067-C10, M0067-F02, M0067-F06, M0069-A04, M0069-A11, M0069-C02, M0069-D10, M0069-G07, M0071-A01, M0071-B07, M0071-D05, M0071-D09, M0071-H03, M0071-H06, M0087-F09, M0088-F07, M0088-G10, M0088-H10, M0089-001, M0089-F05, M0089-B07, M0089-H11, m0032-E01, M0034-004, M0039-F01, M0041-B05, M0041-G01, M0042-B06, M0006-B10, M0007-H06, M0008-H09, M0009-H08, M0011-H11, M0015-F02, M0016-D01, M0013-D11, M0013-G12, M0013-H06, M0014-009, M0014-G11, M0016-A11, M0016-H05, M0019-C05, M0020-B01, M0022-007, M0025-D04, M0027-E11, 539B-X0041-D02, 539B-X0049-A01, 539B-X0049-B01, 539B-X0049-001, 539B-X0049-D01, 539B-X0049-E01, 539B-X0049-F01, 539B-X0049-G01, 539B-X0049-H01, 539B-X0049-A02, 539B-X0049-B02, 539B-X0049-C02, 539B-X0049-D02, or 539B-X0049-E02.

In some embodiments, the MMP-12 binding protein comprises the 3 heavy chain CDRs and/or the 3 light chain CDRs of DX-2712.

In some embodiments, the MMP-12 binding protein comprises the heavy chain variable region and/or the light chain variable region of DX-2712.

In some embodiments, the second agent is an agent for the treatment of an inflammatory disorder selected from the group of inflammatory disorders consisting of: rheumatoid arthritis, psoriasis, multiple sclerosis, systemic sclerosis, asthma, chronic obstructive pulmonary disease, and inflammatory bowel disease.

In some embodiments, the second agent is an immunosuppressant agent selected from the group consisting of: gancyclovir, etanercept, cyclosporine, tacrolimus, rapamycin, cyclophosphamide, azathioprine, mycophenolate mofetil, methotrexate, cortisol, aldosterone, dexamethasone, a cyclooxygenase inhibitor, a 5-lipoxygenase inhibitor, and leukotriene receptor antagonist.

In some embodiments, the second agent is an agent for the treatment of rheumatoid arthritis and comprises a nonsteroidal anti-inflammatory drug (NSAID), a corticosteroid, a Disease-Modifying Antirheumatic Drug (DMARD), or a biological response modifier (BRM).

In some embodiments, the second agent is an agent for the treatment of rheumatoid arthritis and comprises aspirin, naproxen, ibuprofen, etodolac, gold, salsalte, methotrexate, sulfasalazine, D-penicillamine, azathioprine, cyclophosphamide, chlorambucil, cyclosporine, leflunomide, etanercept, infliximab, anakinra, adalimumab, hydroxychloroquine, chloroquine phosphate, chloroquine sulphate, minocycline, or a CTLA4-Ig.

In some embodiments, the second agent is an agent for the treatment of psoriasis and comprises a topical treatment or a systemic treatment.

In some embodiments, the second agent is an agent for the treatment of psoriasis and comprises a topical treatment selected from the group consisting of coal tar, dithranol, a corticosteroid, a vitamin D3 analogue, and a retinoid.

In some embodiments, the second agent is an agent for the treatment of psoriasis and comprises a systemic treatment selected from the group consisting of methotrexate, cyclosporine, a retinoid, tioguanine, hydroxyurea, sulfasalazine, mycophenolate mofetil, azathioprine, tacrolimus, alefacept, efalizumab, etanercept, and infliximab.

In some embodiments, the second agent is an agent for the treatment of multiple sclerosis and comprises a corticosteroid, an interferon, glatiramer acetate, an immunosuppressant or natalizumab.

In some embodiments, the second agent is an agent for the treatment of systemic sclerosis and comprises an NSAID, a calcium channel blocker, prostacyclin analogue, a dual endothelin-receptor antagonist, methotrexate, cyclosporin, an ACE inhibitor, cyclophosphamide, a steroid, or epoprostenol.

In some embodiments, the second agent is an agent for the treatment of systemic sclerosis and comprises nifedipine.

In some embodiments, the second agent is an agent for the treatment of systemic sclerosis and comprises iloprost.

In some embodiments, the second agent is an agent for the treatment of systemic sclerosis and comprises bosentan.

In some embodiments, the second agent is an agent for the treatment of asthma and comprises a glucocorticoid, a leukotriene modifier, a mast cell stabilizer, an antimuscarinic/anticholinergic, an antihistamines, an IgE blocker, or methotrexate.

In some embodiments, the second agent is an agent for the treatment of asthma and comprises a beta2-adrenoceptor agonist selected from the group consisting of: salbutamol, levalbuterol, terbutaline and bitolterol.

In some embodiments, the second agent is an agent for the treatment of asthma and comprises an adrenergic agonist selected from the group consisting of: inhaled epinephrine and ephedrine tablets.

In some embodiments, the second agent is an agent for the treatment of asthma and comprises an antimuscarinic/anticholinergic selected from the group consisting of: ipratropium, oxitropium, and tiotropium.

In some embodiments, the second agent is an agent for the treatment of asthma and comprises an inhaled glucocorticoid selected from the group consisting of: ciclesonide, beclomethasone, budesonide, flunisolide, fluticasone, mometasone, and triamcinolone.

In some embodiments, the second agent is an agent for the treatment of asthma and comprises a leukotriene modifier selected from the group consisting of: montelukast, zafirlukast, pranlukast, and zileuton.

In some embodiments, the second agent is an agent for the treatment of asthma and comprises a mast cell stabilizer selected from the group consisting of: cromoglicate and nedocromil.

In some embodiments, the second agent is an agent for the treatment of asthma and comprises a methylxanthine selected from the group consisting of: theophylline and aminophylline.

In some embodiments, the second agent is an agent for the treatment of asthma and comprises an antihistamine.

In some embodiments, the second agent is an agent for the treatment of asthma and comprises an IgE blocker selected from the group consisting of: omalizumab and methotrexate.

In some embodiments, the second agent is an agent for the treatment of asthma and comprises a long-acting beta₂-adrenoceptor agonist selected from the group consisting of: salmeterol, formoterol, bambuterol, and albuterol.

In some embodiments, the second agent is an agent for the treatment of asthma and comprises a combination of inhaled steroid and a long-acting bronchodilator selected from the group consisting of: fluticasone/salmeterol and budesonide/formoterol.

In some embodiments, the second agent is an agent for the treatment of chronic obstructive pulmonary disease (COPD)and comprises a bronchodilator.

In some embodiments, the second agent is an agent for the treatment of chronic obstructive pulmonary disease (COPD)and comprises a β₂ agonist, an M₃ antimuscarinic, a leukotriene antagonist, a cromone, a corticosteroid, or a xanthine.

In some embodiments, the second agent is an agent for the treatment of chronic obstructive pulmonary disease (COPD)and comprises a β₂ agonist selected from the group consisting of: Salbutamol, Bambuterol, Clenbuterol, Fenoterol, Formoterol, and Salmeterol.

In some embodiments, the second agent is an agent for the treatment of chronic obstructive pulmonary disease (COPD)and comprises the Ipratropium.

In some embodiments, the second agent is an agent for the treatment of chronic obstructive pulmonary disease (COPD)and comprises a cromone selected from the group consisting of: Cromoglicate and Nedocromil.

In some embodiments, the second agent is an agent for the treatment of chronic obstructive pulmonary disease (COPD)and comprises a leukotriene antagonist selected from the group consisting of: Montelukast, Pranlukast, and Zafirlukast.

In some embodiments, the second agent is an agent for the treatment of chronic obstructive pulmonary disease (COPD)and comprises a corticosteroid antagonist selected from the group consisting of: glucocorticoids, beclomethasone, mometasone, and fluticasone.

In some embodiments, the second agent is an agent for the treatment of chronic obstructive pulmonary disease (COPD)and comprises a xanthine antagonist selected from the group consisting of: theophylline, methylxanthine, and theobromine.

In some embodiments, the second agent is an agent for the treatment of chronic obstructive pulmonary disease (COPD) and comprises Ipratropium or Tiotropium.

In some embodiments, the second agent is an agent for the treatment of inflammatory bowel disease and comprises an immunosuppresant, an anti-TNF binding protein, a cytokine inhibitor, a BRM, or an anti-inflammatory.

In some embodiments, the second agent is an agent for the treatment of inflammatory bowel disease and comprises prednisone, infliximab, azathioprine, methotrexate, 6-mercaptopurine , a mesalamine, a steroid, or CDP571 antibody.

In one aspect, this disclosure relates to a method of treating or preventing an inflammatory disorder in a subject (e.g., a subject having or suspected of having the inflammatory disorder, or at risk thereof), the method comprising:

administering an isolated protein (e.g., antibody, e.g., human antibody) (e.g., a therapeutically effective amount thereof) that binds MMP-14 to the subject, wherein the antibody binds the same epitope or competes for binding with an antibody selected from the group consisting of DX-2400, DX-2410, M0031-C02, M0031-F01, M0033-H07, M0037-009, M0037-D01, M0038-E06, M0038-F01, M0038-F08, M0039-H08, M0040-A06, M0040-A11, and M0043-G02, in combination with a second agent (e.g., a therapeutically effective amount thereof), wherein the second agent is an agent for the treatment of the inflammatory disorder. In some embodiments, the second agent is a second agent described herein.

In one aspect, this disclosure relates to a method of treating or preventing an inflammatory disorder in a subject (e.g., a subject having or suspected of having the inflammatory disorder, or at risk thereof), the method comprising:

administering an isolated protein (e.g., antibody, e.g., human antibody) (e.g., a therapeutically effective amount thereof) that binds MMP-9 to the subject, wherein the antibody binds the same epitope or competes for binding with an antibody selected from the group consisting of DX-2802, 539A-M0240-B03, M0078-G07, M0081-D05, M0076-D03, M0072-H07, M0075-D12, M0166-F10, M0279-A03, M0279-B02, M0288-008, and M0281-F06, in combination with a second agent (e.g., a therapeutically effective amount thereof), wherein the second agent is an agent for the treatment of the inflammatory disorder. In some embodiments, the second agent is a second agent described herein.

In one aspect, this disclosure relates to a method of treating or preventing an inflammatory disorder in a subject (e.g., a subject having or suspected of having the inflammatory disorder, or at risk thereof), the method comprising:

administering an isolated protein (e.g., antibody, e.g., human antibody) (e.g., a therapeutically effective amount thereof) that binds MMP-9/-2 to the subject, wherein the antibody binds the same epitope or competes for binding with an antibody selected from the group consisting of M0237-D02, X0106-A01, X0106-B02, X0106-004, X0106-E4, and X0106-F05, in combination with a second agent (e.g., a therapeutically effective amount thereof), wherein the second agent is an agent for the treatment of the inflammatory disorder. In some embodiments, the second agent is a second agent described herein.

In one aspect, this disclosure relates to a method of treating or preventing an inflammatory disorder in a subject (e.g., a subject having or suspected of having the inflammatory disorder, or at risk thereof), the method comprising:

administering an isolated protein (e.g., antibody, e.g., human antibody) (e.g., a therapeutically effective amount thereof) that binds MMP-12 to the subject, wherein the antibody binds the same epitope or competes for binding with an antibody selected from the group consisting of DX-2712, a mutant or variant of DX-2712, 539B-X0041-D02, M0134-A02, M134-A05, M134-A07, M134-A09, M134-A10, M134-A11, M0134-B01, M134-B04, M0134-B08, M0134-B11, M0134-001, M0134-C02, M0134-006, M0134-009, M0134-C10, M0134-C11, M0134-C12, M0134-D02, M0134-D03, M0134-E04, M0134-E07, M0134-E08, M134-E11, M0134-F01, M0134-F05, M0134-G02, M0134-G04, M0134-G07, M0135-A03, M0135-A05, M0135-A06, M0135-A07, M0135-B02, M0135-B08, M0135-001, M0135-C11, M0135-E03, M0135-F03, M0135-F11, M0135-G02, M0135-G03, M0135-G07, M0135-G11, M0135-H03, M0135-H10, M0105-C05, M0105-E11, M0105-F08, M0107-A12, M0108-A02, M0109-G11, M0110-G05, M0129-B11, M0130-A01, M0130-C12, M0130-F06, M0130-H04, M0131-A06, M0131-D03, M0132-A04, M0133-B08, M0133-E05, M0121-E07, M0118-F11, M0125-G07, M0124-E07, M0119-D01, M0119-A02, M0122-006, M0123-G07, M0063-A02, M0063-A04, M0063-B01, M0063-B11, M0063-007, M0063-G01, M0065-E12, M0065-G03, M0065-H05, M0067-A02, M0067-B06, M0067-B09, M0067-C10, M0067-F02, M0067-F06, M0069-A04, M0069-A11, M0069-C02, M0069-D10, M0069-G07, M0071-A01, M0071-B07, M0071-D05, M0071-D09, M0071-H03, M0071-H06, M0087-F09, M0088-F07, M0088-G10, M0088-H10, M0089-001, M0089-F05, M0089-B07, M0089-H11, M0032-E01, M0034-004, M0039-F01, M0041-B05, M0041-G01, M0042-B06, M0006-B10, M0007-H06, M0008-H09, M0009-H08, M0011-H11, M0015-F02, M0016-D01, M0013-D11, M0013-G12, M0013-H06, M0014-009, M0014-G11, M0016-A11, M0016-H05, M0019-C05, M0020-B01, M0022-007, M0025-D04, M0027-E11, 539B-X0041-D02, 539B-X0049-A01, 539B-X0049-B01, 539B-X0049-001, 539B-X0049-D01, 539B-X0049-E01, 539B-X0049-F01, 539B-X0049-G01, 539B-X0049-H01, 539B-X0049-A02, 539B-X0049-B02, 539B-X0049-C02, 539B-X0049-D02, and 539B-X0049-E02, in combination with a second agent (e.g., a therapeutically effective amount thereof), wherein the second agent is an agent for the treatment of the inflammatory disorder. In some embodiments, the second agent is a second agent described herein.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 depicts sequence information for DX-2712HEK. FIG. 1 discloses SEQ ID NOS 151, 62, 123, 118 and 120, respectively, in order of appearance.

FIG. 2 depicts the effect of treatment with a negative control, DX-2712HEK and methotrexate (MTX) on disease development in a mouse model of collagen-induced arthritis, relative to a non-diseased mouse.

FIG. 3 depicts the effect of treatment with a negative control, DX-2712HEK and methotrexate on four key joint histological parameters in a mouse model of collagen-induced arthritis, relative to a non-diseased mouse.

FIG. 4 depicts the effect of treatment with a negative control, MMP-9/2 inhibitor and methotrexate (MTX) on disease development in a mouse model of collagen-induced arthritis, relative to a non-diseased mouse.

FIG. 5 depicts the effect of treatment with a negative control, MMP-9/2 inhibitor and methotrexate on four key joint histological parameters in a mouse model of collagen-induced arthritis, relative to a non-diseased mouse.

FIG. 6 depicts the effect of the combination of DX-2712 and methotrexate (MTX) on disease progression.

FIG. 7 depicts the effect of the combination of DX-2712 and ENBREL® on disease progression.

FIG. 8 depicts the effect of the combination of DX-2712 and ORENCIA® on disease progression.

FIGS. 9(A) and 9(B): FIG. 9(A) depicts the effects of DX-2400 and DX-2410 in a collagen-induced arthritis (CIA) mouse model. FIG. 9(B) provides the histology score descriptions used for the results shown in FIG. 9(A).

### DETAILED DESCRIPTION

### Definitions

For convenience, before further description of the present invention, certain terms employed in the specification, examples and appended claims are defined here.

The singular forms “a”, “an”, and “the” include plural references unless the context clearly dictates otherwise.

The term “antibody” refers to a protein that includes at least one immunoglobulin variable domain or immunoglobulin variable domain sequence. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody” encompasses antigen-binding fragments of antibodies (e.g., single chain antibodies, Fab and sFab fragments, F(ab′)₂, Fd fragments, Fv fragments, scFv, and domain antibodies (dAb) fragments (de Wildt et al., Eur J. Immunol. 1996; 26(3):629-39.)) as well as complete antibodies. An antibody can have the structural features of IgA, IgG, IgE, IgD, IgM (as well as subtypes thereof). Antibodies may be from any source, but primate (human and non-human primate) and primatized are preferred.

The VH and VL regions can be further subdivided into regions of hypervariability, termed “complementarity determining regions” (“CDR”), interspersed with regions that are more conserved, termed “framework regions” (“FR”). The extent of the framework region and CDRs has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917, see also www.hgmp.mrc.ac.uk). Kabat definitions are used herein. Each VH and VL is typically composed of three CDRs and four FRs, arranged from amino-terminus to carboxy-terminus in the following order: FR1, CDR1, FR2, CDR2, FR3, CDR3, FR4.

The VH or VL chain of the antibody can further include all or part of a heavy or light chain constant region, to thereby form a heavy or light immunoglobulin chain, respectively. In one embodiment, the antibody is a tetramer of two heavy immunoglobulin chains and two light immunoglobulin chains, wherein the heavy and light immunoglobulin chains are inter-connected by, e.g., disulfide bonds. In IgGs, the heavy chain constant region includes three immunoglobulin domains, CH1, CH2 and CH3. The light chain constant region includes a CL domain. The variable region of the heavy and light chains contains a binding domain that interacts with an antigen. The constant regions of the antibodies typically mediate the binding of the antibody to host tissues or factors, including various cells of the immune system (e.g., effector cells) and the first component (Clq) of the classical complement system. The light chains of the immunoglobulin may be of types kappa or lambda. In one embodiment, the antibody is glycosylated. An antibody can be functional for antibody-dependent cytotoxicity and/or complement-mediated cytotoxicity.

One or more regions of an antibody can be human or effectively human. For example, one or more of the variable regions can be human or effectively human. For example, one or more of the CDRs can be human, e.g., HC CDR1, HC CDR2, HC CDR3, LC CDR1, LC CDR2, and LC CDR3. Each of the light chain CDRs can be human. HC CDR3 can be human. One or more of the framework regions can be human, e.g., FR1, FR2, FR3, and FR4 of the HC or LC. For example, the Fc region can be human. In one embodiment, all the framework regions are human, e.g., derived from a human somatic cell, e.g., a hematopoietic cell that produces immunoglobulins or a non-hematopoietic cell. In one embodiment, the human sequences are germline sequences, e.g., encoded by a germline nucleic acid. In one embodiment, the framework (FR) residues of a selected Fab can be converted to the amino-acid type of the corresponding residue in the most similar primate germline gene, especially the human germline gene. One or more of the constant regions can be human or effectively human. For example, at least 70, 75, 80, 85, 90, 92, 95, 98, or 100% of an immunoglobulin variable domain, the constant region, the constant domains (CH1, CH2, CH3, CL1), or the entire antibody can be human or effectively human.

All or part of an antibody can be encoded by an immunoglobulin gene or a segment thereof. Exemplary human immunoglobulin genes include the kappa, lambda, alpha (IgA1 and IgA2), gamma (IgG1, IgG2, IgG3, and IgG4), delta, epsilon and mu constant region genes, as well as the many immunoglobulin variable region genes. Full-length immunoglobulin “light chains” (about 25 KDa or about 214 amino acids) are encoded by a variable region gene at the NH2-terminus (about 110 amino acids) and a kappa or lambda constant region gene at the COOH-terminus. Full-length immunoglobulin “heavy chains” (about 50 KDa or about 446 amino acids), are similarly encoded by a variable region gene (about 116 amino acids) and one of the other aforementioned constant region genes, e.g., gamma (encoding about 330 amino acids). The length of human HC varies considerably because HC CDR3 varies from about 3 amino-acid residues to over 35 amino-acid residues.

The term “antigen-binding fragment” of a full length antibody refers to one or more fragments of a full-length antibody that retain the ability to specifically bind to a target of interest. Examples of binding fragments encompassed within the term “antigen-binding fragment” of a full length antibody include (i) a Fab fragment, a monovalent fragment consisting of the VL, VH, CL and CH1 domains; (ii) a F(ab′)₂ fragment, a bivalent fragment including two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR) that retains functionality. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules known as single chain Fv (scFv). See e.g., U.S. Pat. Nos. 5,260,203, 4,946,778, and 4,881,175; Bird et al. (1988) Science 242:423-426; and Huston et al. (1988) Proc. Natl. Acad. Sci. USA 85:5879-5883.

Antibody fragments can be obtained using any appropriate technique including conventional techniques known to those with skill in the art. The term “monospecific antibody” refers to an antibody that displays a single binding specificity and affinity for a particular target, e.g., epitope. This term includes a “monoclonal antibody” or “monoclonal antibody composition,” which as used herein refer to a preparation of antibodies or fragments thereof of single molecular composition, irrespective of how the antibody was generated.

The term “arthritis” refers to any one, subset or all of a set of inflammatory disorders involving inflammation and damage to the joints of the body. Arthritis thus includes, but is not limited to, osteoarthritis, rheumatoid arthritis, psoriatic arthritis, septic arthritis and gouty arthritis.

As used herein, “binding affinity” refers to the apparent association constant or Kₐ. The Kₐ is the reciprocal of the dissociation constant (K_{d}). A binding protein may, for example, have a binding affinity of at least 10⁵, 10⁶, 10⁷, 10⁸, 10⁹, 10¹⁰ and 10¹¹ M⁻¹ for a particular target molecule, e.g., MMP-9, MMP-12, MMP-2, MMP-14, etc. Higher affinity binding of a binding protein to a first target relative to a second target can be indicated by a higher Kₐ (or a smaller numerical value K_{d}) for binding the first target than the Kₐ (or numerical value K_{d}) for binding the second target. In such cases, the binding protein has specificity for the first target (e.g., a protein in a first conformation or mimic thereof) relative to the second target (e.g., the same protein in a second conformation or mimic thereof; or a second protein). Differences in binding affinity (e.g., for specificity or other comparisons) can be at least 1.5, 2, 3, 4, 5, 10, 15, 20, 37.5, 50, 70, 80, 91, 100, 500, 1000, or 10⁵ fold.

Binding affinity can be determined by a variety of methods including equilibrium dialysis, equilibrium binding, gel filtration, ELISA, surface plasmon resonance, or spectroscopy (e.g., using a fluorescence assay). Exemplary conditions for evaluating binding affinity are in TRIS-buffer (50 mM TRIS, 150 mM NaCl, 5 mM CaCl₂ at pH7.5). These techniques can be used to measure the concentration of bound and free binding protein as a function of binding protein (or target) concentration. The concentration of bound binding protein ([Bound]) is related to the concentration of free binding protein ([Free]) and the concentration of binding sites for the binding protein on the target where (N) is the number of binding sites per target molecule by the following equation:

[Bound]=N·[Free]/((1/Ka)+[Free]).

It is not always necessary to make an exact determination of Kₐ, though, since sometimes it is sufficient to obtain a quantitative measurement of affinity, e.g., determined using a method such as ELISA or FACS analysis, is proportional to Kₐ, and thus can be used for comparisons, such as determining whether a higher affinity is, e.g., 2-fold higher, to obtain a qualitative measurement of affinity, or to obtain an inference of affinity, e.g., by activity in a functional assay, e.g., an in vitro or in vivo assay.

The term “binding protein” refers to a protein or polypeptide that can interact with a target molecule. This term is used interchangeably with “ligand.” An “MMP-9 binding protein” refers to a protein that can interact with MMP-9 and includes, in particular, proteins that preferentially interact with and/or inhibit MMP-9. For example, the MMP-9 binding protein may be an antibody. Likewise, an “MMP-12 binding protein” refers to a protein that can interact with MMP-12 and includes, in particular, proteins that preferentially interact with and/or inhibit MMP-12. Further, an “MMP-9/2 binding protein” refers to a protein that can interact with both MMP-9 and MMP-2 and includes, in particular, proteins that preferentially interact with and/or inhibit MMP-9 and MMP-2.

The term “cognate ligand” refers to a naturally occurring ligand of any of the binding proteins described herein, e.g., MMP-9, MMP-12, MMP-14 binding proteins, including naturally occurring variants thereof (e.g., splice variants, naturally occurring mutants, and isoforms).

The term “combination” or “combination therapy” refers to the use of the two or more agents or therapies to treat the same patient, wherein the use or action of the agents or therapies overlap in time. The agents or therapies can be administered at the same time (e.g., as a single formulation that is administered to a patient or as two separate formulations administered concurrently) or sequentially in any order.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). It is possible for many framework and CDR amino acid residues to include one or more conservative substitutions.

Disease-modifying antirheumatic drugs (DMARDs) is a category of drugs used in many autoimmune disorders to slow down disease progression. Their use was first propagated in rheumatoid arthritis (hence their name) but has come to include many other diseases, such as Crohn's disease, systemic lupus erythematosus (SLE), idiopathic thrombocytopenic purpura (ITP), myasthenia gravis and various others.

An “effectively human” immunoglobulin variable region is an immunoglobulin variable region that includes a sufficient number of human framework amino acid positions such that the immunoglobulin variable region does not elicit an immunogenic response in a normal human. An “effectively human” antibody is an antibody that includes a sufficient number of human amino acid positions such that the antibody does not elicit an immunogenic response in a normal human.

An “epitope” refers to the site on a target compound that is bound by a binding protein (e.g., an antibody such as a Fab or full length antibody). In the case where the target compound is a protein, the site can be entirely composed of amino acid components, entirely composed of chemical modifications of amino acids of the protein (e.g., glycosyl moieties), or composed of combinations thereof. Overlapping epitopes include at least one common amino acid residue, glycosyl group, phosphate group, sulfate group, or other molecular feature.

A (first) protein, e.g., antibody “binds to the same epitope” as another (second) antibody if the antibody binds to the same site on a target compound that the second antibody binds, or binds to a site that overlaps (e.g., 50%, 60%, 70%, 80%, 90%, or 100% overlap, e.g., in terms of amino acid sequence or other molecular feature (e.g., glycosyl group, phosphate group, or sulfate group) with the site that the second antibody binds.

A (first) protein, e.g., antibody “competes for binding” with another (second) antibody if the binding of the first antibody to its epitope decreases (e.g., by 10%, 20%, 30%, 40%, 50%, 60%, 70%, 80%, 90%, 100%, or more) the amount of the second antibody that binds to its epitope. The competition can be direct (e.g., the first antibody binds to an epitope that is the same as, or overlaps with, the epitope bound by the second antibody), or indirect (e.g., the binding of the first antibody to its epitope causes a steric change in the target compound that decreases the ability of the second antibody to bind to its epitope).

Calculations of “homology” or “sequence identity” between two sequences (the terms are used interchangeably herein) are performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). The optimal alignment is determined as the best score using the GAP program in the GCG software package with a Blossum 62 scoring matrix with a gap penalty of 12, a gap extend penalty of 4, and a frameshift gap penalty of 5. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences.

In a preferred embodiment, the length of a reference sequence aligned for comparison purposes is at least 30%, preferably at least 40%, more preferably at least 50%, even more preferably at least 60%, and even more preferably at least 70%, 80%, 90%, 92%, 95%, 97%, 98%, or 100% of the length of the reference sequence. For example, the reference sequence may be the length of the immunoglobulin variable domain sequence.

A “humanized” immunoglobulin variable region is an immunoglobulin variable region that is modified to include a sufficient number of human framework amino acid positions such that the immunoglobulin variable region does not elicit an immunogenic response in a normal human. Descriptions of “humanized” immunoglobulins include, for example, U.S. Pat. No. 6,407,213 and U.S. Pat. No. 5,693,762.

As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1-6.3.6. Aqueous and nonaqueous methods are described in that reference and either can be used. Specific hybridization conditions referred to herein are as follows: (1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); (2) medium stringency hybridization conditions in 6×SSC at about 45° C., followed by one or more washes in 0.2×SSC, 0.1% SDS at 60° C.; (3) high stringency hybridization conditions in 6×SSC at about 45° C., followed by one or more washes in 0.2×SSC, 0.1% SDS at 65° C.; and (4) very high stringency hybridization conditions are 0.5M sodium phosphate, 7% SDS at 65° C., followed by one or more washes at 0.2×SSC, 1% SDS at 65° C. Very high stringency conditions (4) are the preferred conditions and the ones that should be used unless otherwise specified. The disclosure includes nucleic acids that hybridize with low, medium, high, or very high stringency to a nucleic acid described herein or to a complement thereof, e.g., nucleic acids encoding a binding protein described herein. The nucleic acids can be the same length or within 30, 20, or 10% of the length of the reference nucleic acid. The nucleic acid can correspond to a region encoding an immunoglobulin variable domain sequence described herein.

A binding protein of interest (e.g., MMP-9, MMP-12, MMP-9/2 binding protein) may have mutations (e.g., at least one, two, or four, and/or less than 15, 10, 5, or 3) relative to a binding protein described herein (e.g., a conservative or non-essential amino acid substitutions), which do not have a substantial effect on protein function. Whether or not a particular substitution will be tolerated, i.e., will not adversely affect biological properties, such as binding activity can be predicted, e.g., by evaluating whether the mutation is conservative or by the method of Bowie, et al. (1990) Science 247:1306-1310.

As used herein, an “immunoglobulin variable domain sequence” refers to an amino acid sequence which can form the structure of an immunoglobulin variable domain such that one or more CDR regions are positioned in a conformation suitable for an antigen binding site. For example, the sequence may include all or part of the amino acid sequence of a naturally-occurring variable domain. For example, the sequence may omit one, two or more N- or C-terminal amino acids, internal amino acids, may include one or more insertions or additional terminal amino acids, or may include other alterations. In one embodiment, a polypeptide that includes immunoglobulin variable domain sequence can associate with another immunoglobulin variable domain sequence to form an antigen binding site, e.g., a structure that preferentially interacts with an antigen.

The term “inflammatory disorder” or “inflammatory disease” includes both immune and autoimmune conditions as well as generalized conditions marked by systemic or localized inflammation. Thus it is understood that disclosed are methods of treating an inflammatory response or condition, wherein the inflammatory response or condition is selected from the group consisting of asthma, alopecia greata, SLE, rheumatoid arthritis, reactive arthritis, spondylarthritis, systemic vasculitis, insulin dependent diabetes mellitus, multiple sclerosis, experimental allergic encephalomyelitis, Sjogren's syndrome, graft versus host disease, inflammatory bowel disease including Crohn's disease, ulcerative colitis, ischemia reperfusion injury, myocardial infarction, Alzheimer's disease, transplant rejection (allogeneic and xenogeneic), thermal trauma, any immune complex-induced inflammation, glomerulonephritis, myasthenia gravis, cerebral lupus, Guillain-Barre syndrome, vasculitis, systemic sclerosis, anaphlaxis, catheter reactions, atheroma, infertility, thyroiditis, ARDS, post-bypass syndrome, hemodialysis, juvenile rheumatoid, Behcets syndrome, hemolytic anemia, pemphigus, bullous pemphigoid, stroke, atherosclerosis, scleroderma, psoriasis, sarcoidosis, transverse myelitis, acute disseminated encephalomyelitis, post-infectious encephalomyelitis, subacute sclerosing panencephalitis, polymyositis, dermatomyositis, incusion body myopathy, and chronic inflammatory demyelinating polyradiculopathy. Each and any of these inflammatory diseases or disorders may also be excluded. Thus, for example, in certain embodiments, the inflammatory condition is not asthma, not SLE, not rheumatoid arthritis, not myasthenia gravis, not diabetes (e.g., not insulin dependent diabetes mellitus), or not transplant rejection. Each and any inflammatory condition associated with cancer (i.e., malignancy) may be excluded, as well.

An “isolated composition” refers to a composition that is removed from at least 90% of at least one component of a natural sample from which the isolated composition can be obtained. Compositions produced artificially or naturally can be “compositions of at least” a certain degree of purity if the species or population of species of interests is at least 5, 10, 25, 50, 75, 80, 90, 92, 95, 98, or 99% pure on a weight-weight basis.

The term “Kunitz domain” refers to a molecule comprising or containing one or more folding domains of approximately 51-64 residues that form a central anti-parallel beta sheet and a short C-terminal helix, and comprise six cysteine residues that form three disulfide bonds, resulting in a double-loop structure, or parts of such domains or any fragments, variants, modifications or derivatives thereof.

A “non-essential” amino acid residue is a residue that can be altered from the wild-type sequence of the binding agent, e.g., the antibody, without abolishing or more preferably, without substantially altering a biological activity, whereas changing an “essential” amino acid residue results in a substantial loss of activity.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

The term “preventing” a disease in a subject refers to subjecting the subject to a pharmaceutical treatment, e.g., the administration of a drug, such that at least one symptom of the disease is prevented, that is, administered prior to clinical manifestation of the unwanted condition (e.g., disease or other unwanted state of the host animal) so that it protects the host against developing the unwanted condition. “Preventing” a disease may also be referred to as “prophylaxis” or “prophylactic treatment.”

A “prophylactically effective amount” refers to an amount effective, at dosages and for periods of time necessary, to achieve the desired prophylactic result. Typically, because a prophylactic dose is used in subjects prior to or at an earlier stage of disease, the prophylactically effective amount will be less than the therapeutically effective amount.

A subject is “at risk” of having an inflammatory disorder if the subject has, for example, a genetic predisposition for the disorder, a blood relative suffering from the disorder, or another risk factor thereof. For example, a subject may be at risk of having COPD if the subject is a smoker or has been exposed to airborne irritants.

As used herein, the term “substantially identical” (or “substantially homologous”) is used herein to refer to a first amino acid or nucleic acid sequence that contains a sufficient number of identical or equivalent (e.g., with a similar side chain, e.g., conserved amino acid substitutions) amino acid residues or nucleotides to a second amino acid or nucleic acid sequence such that the first and second amino acid or nucleic acid sequences have (or encode proteins having) similar activities, e.g., a binding activity, a binding preference, or a biological activity. In the case of antibodies, the second antibody has the same specificity and has at least 50%, at least 25%, or at least 10% of the affinity relative to the same antigen. Sequences similar or homologous (e.g., at least about 85% sequence identity) to the sequences disclosed herein are also part of this application. In some embodiments, the sequence identity can be about 85%, 90%, 91%, 92%, 93%, 94%, 95%, 96%, 97%, 98%, 99% or higher. In addition, substantial identity exists when the nucleic acid segments hybridize under selective hybridization conditions (e.g., highly stringent hybridization conditions), to the complement of the strand. The nucleic acids may be present in whole cells, in a cell lysate, or in a partially purified or substantially pure form.

Motif sequences for biopolymers can include positions which can be varied amino acids. For example, the symbol “X” in such a context generally refers to any amino acid (e.g., any of the twenty natural amino acids or any of the nineteen non-cysteine amino acids). Other allowed amino acids can also be indicated for example, using parentheses and slashes. For example, “(A/W/F/N/Q)” means that alanine, tryptophan, phenylalanine, asparagine, and glutamine are allowed at that particular position.

Statistical significance can be determined by any art known method. Exemplary statistical tests include: the Students T-test, Mann Whitney U non-parametric test, and Wilcoxon non-parametric statistical test. Some statistically significant relationships have a P value of less than 0.05 or 0.02. Particular binding proteins may show a difference, e.g., in specificity or binding that are statistically significant (e.g., P value <0.05 or 0.02). The terms “induce”, “inhibit”, “potentiate”, “elevate”, “increase”, “decrease” or the like, e.g., which denote distinguishable qualitative or quantitative differences between two states, and may refer to a difference, e.g., a statistically significant difference, between the two states.

A “therapeutically effective amount” refers to an amount effective, at dosages and for periods of time necessary, to achieve the desired therapeutic result. A therapeutically effective amount of the composition may vary according to factors such as the disease state, age, sex, and weight of the individual, and the ability of the protein to elicit a desired response in the individual. A therapeutically effective amount is also one in which any toxic or detrimental effects of the composition is outweighed by the therapeutically beneficial effects.

A “therapeutically effective dosage” preferably modulates a measurable parameter, e.g., levels of circulating IgG antibodies by a statistically significant degree or at least about 20%, more preferably by at least about 40%, even more preferably by at least about 60%, and still more preferably by at least about 80% relative to untreated subjects. The ability of a compound to modulate a measurable parameter, e.g., a disease-associated parameter, can be evaluated in an animal model system predictive of efficacy in human disorders and conditions, e.g., an inflammatory disease (e.g., synovitis, atherosclerosis), rheumatoid arthritis or osteoarthritis. Alternatively, this property of a composition can be evaluated by examining the ability of the compound to modulate a parameter in vitro.

“Treating” a disease in a subject or “treating” a subject having a disease refers to subjecting the subject to a pharmaceutical treatment, e.g., the administration of a drug, such that at least one symptom of the disease is cured, alleviated or decreased.

Protease Binding Proteins and/or Inhibitors—General

Exemplary proteases that may be targeted by the protease binding proteins and/or inhibitors comprising the compositions and methods described herein for use in treating inflammatory disorders include all members of the metalloprotease family, especially targets such as MMP-14, MMP-9, MMP-12, MMP-2, MMP-7, as well as other proteases such as TACE, ADAM-TS2; serine proteases, including plasmin, hepsin, matriptase, plasma kallikrein, tissue kallikrein 1, and cathepsins, including cathepsins B, S and K. The protease binding proteins and/or inhibitors may in certain embodiments be antibody, peptide or Kunitz domain-based.

Proteases are involved in a wide variety of biological processes, including inflammation and tissue injury. Serine proteases produced by inflammatory cells, including neutrophils, are implicated in various inflammatory disorders. As such, multiple different proteases can contribute to a single disorder.

Examples of particular proteases include the following: plasmin, tissue kallikrein, plasma kallikrein, Factor VIₐ, Factor XIₐ, thrombin, urokinase, trypsin 1, trypsin 2, pancreatic chymotrypsin, pancreatic elastase, tryptase, and Factor IIₐ. Classes of relevant proteases include: proteases associated with blood coagulation, proteases associated with fibrinolysis, proteases associated with complement, proteases that digest extracellular matrix components, proteases that digest basement membranes, and proteases associated with endothelial cells. In certain embodiments, the protease is a serine protease.

Putative binding proteins/inhibitors can be selected for their potency and selectivity of inhibition of the target proteases. In one example, target protease and a substrate are combined under assay conditions permitting reaction of the protease with its substrate. The assay is performed in the absence of the protein being tested, and in the presence of increasing concentrations of the protein being tested. The concentration of test protein at which the protease activity is 50% inhibited is the IC50 value (Inhibitory Concentration) or EC50 (Effective Concentration) value for that test protein. Proteins having lower IC50 or EC50 values are considered more potent inhibitors of the target protease than those proteins having higher IC50 or EC50 values. Preferred proteins according to this aspect have an IC50 value of 100 nM, 10 nM, 1 nM, or 0.1 nM or less as measured in an in vitro assay for inhibition of protease activity.

A test protein can also be evaluated for selectivity toward different protease(s). A test protein is assayed for its potency toward a panel of proteases and other enzymes and an IC50 value is determined for each. A protein that demonstrates a low IC50 value for the target protease, and a higher IC50 value for other proteases within the test panel (e.g., trypsin, plasmin, chymotrypsin), is considered to be selective toward the target protease. Generally, a protein is deemed selective if its IC50 value is at least one order of magnitude less than the next smallest IC50 value measured in the panel of enzymes.

It is also possible to evaluate binding protein/inhibitor activity in vivo or in samples (e.g., serum, joint tissue) of subjects to which a compound described herein has been administered.

Specific protease binding proteins/inhihibtors useful in the methods and compositions provided herein are described in further detail below.

Protease Inhibitors—Kallikrein Binding Proteins/Inhibitors

Kallikreins are serine proteases found in both tissues and plasma [see, for example, U.S. Pat. No. 6,333,402 to Markland]. Plasma kallikrein is involved in contact-activated (intrinsic pathway) coagulation, fibrinolysis, hypotension, and inflammation [See Bhoola, K.D., C.D. Figueroa, and K. Worthy, Pharmacological Reviews (1992) 44(1)1-80]. These effects of kallikrein are mediated through the activities of three distinct physiological substrates:

i) Factor XII (coagulation),

ii) Pro-urokinase/plasminogen (fibrinolysis), and

iii) Kininogens (hypotension and inflammation).

Kallikrein cleavage of kininogens results in the production of kinins, small highly potent bioactive peptides. The kinins act through cell surface receptors, designated BK-1 and BK-2, present on a variety of cell types including endothelia, epithelia, smooth muscle, neural, glandular and hematopoietic. Intracellular heterotrimeric G-proteins link the kinin receptors to second messenger pathways including nitric oxide, adenyl cyclase, phospholipase A₂ and phospholipase C. Among the significant physiological activities of kinins are: (i) increased vascular permeability; (ii) vasodilation; (iii) bronchospasm; and (iv) pain induction. Thus, kinins mediate the life-threatening vascular shock and edema associated with bacteremia (sepsis) or trauma, the edema and airway hyperreactivity of asthma, and both inflammatory and neurogenic pain associated with tissue injury. The consequences of inappropriate plasma kallikrein activity and resultant kinin production are dramatically illustrated in patients with hereditary angioedema (HAE).

HAE is due to a genetic deficiency of C1-inhibitor, the principal endogenous inhibitor of plasma kallikrein. Symptoms of HAE include edema of the skin, subcutaneous tissues and gastrointestinal tract, and abdominal pain and vomiting. Nearly one-third of HAE patients die by suffocation due to edema of the larynx and upper respiratory tract. Kallikrein is secreted as a zymogen (prekallikrein) that circulates as an inactive molecule until activated by a proteolytic event. [Genebank entry P03952 shows Human Plasma Prekallikrein.]

An important inhibitor of plasma kallikrein (pKA) in vivo is the Cl inhibitor; (see Schmaier, et al. in “Contact Activation and Its Abnormalities”, Chapter 2 in Hemostasis and Thrombosis, Colman, R W, J Hirsh, V J Marder, and E W Salzman, Editors, Second Edition, 1987, J. B. Lippincott Company, Philadelphia, Pa., pp. 27-28). C1 is a serpin and forms an irreversible or nearly irreversible complex with pKA. Although bovine pancreatic trypsin inhibitor (also known as BPTI, aprotinin, or TRASYLOL™) was initially thought to be a strong pKA inhibitor with Kᵢ=320 pM [Auerswald, E.-A., D. Hoerlein, G. Reinhardt, W. Schroder, and E. Schnabel, Bio. Chem. Hoppe-Seyler, (1988), 369 (Supplement):27-35], a more recent report [Berndt, et al., Biochemistry, 32:4564-70, 1993] indicates that its Kᵢ for plasma Kallikrein is 30 nM (i.e., 30,000 pM). The G36S mutant had a Kᵢ of over 500 nM.

Markland et al. [U.S. Pat. Nos. 6,333,402; 5,994,125; 6,057,287; and 5,795,865] claim a number of derivatives having high affinity and specificity in inhibiting human plasma kallikrein. One of these proteins is being tested in human patients who have HAE. Early indications are that the compound is safe and effective. Further, U.S. Pat. No. 7,064,107 by Ladner, et al., describes and claims a number of derivatives having high affinity and specificity in inhibiting human plasma kallikrein.

### Kunitz Domain Kallikrein Inhibitors

A number of useful inhibitors of kallikrein include a Kunitz domain.

As used herein, a “Kunitz domain” is a polypeptide domain having at least 51 amino acids and containing at least two, and preferably three, disulfides. The domain is folded such that the first and sixth cysteines, the second and fourth, and the third and fifth cysteines form disulfide bonds (e.g., in a Kunitz domain having 58 amino acids, cysteines can be present at positions corresponding to amino acids 5, 14, 30, 38, 51, and 55, according to the number of the BPTI sequence provided below, and disulfides can form between the cysteines at position 5 and 55, 14 and 38, and 30 and 51), or, if two disulfides are present, they can form between a corresponding subset of cysteines thereof. The spacing between respective cysteines can be within 7, 5, 4, 3 or 2 amino acids of the following spacing between positions corresponding to: 5 to 55, 14 to 38, and 30 to 51, according to the numbering of the BPTI sequence provided below. The BPTI sequence can be used a reference to refer to specific positions in any generic Kunitz domain. Comparison of a Kunitz domain of interest to BPTI can be performed by identifying the best fit alignment in which the number of aligned cysteines in maximized.

The 3D structure (at high resolution) of the Kunitz domain of BPTI is known. One of the X-ray structures is deposited in the Brookhaven Protein Data Bank as “6PTI”. The 3D structure of some BPTI homologues (Eigenbrot et al., (1990) Protein Engineering, 3(7):591-598; Hynes et al., (1990) Biochemistry, 29:10018-10022) are known. At least seventy Kunitz domain sequences are known. Known human homologues include three Kunitz domains of LACI (Wun et al., (1988) J. Biol. Chem. 263(13):6001-6004; Girard et al., (1989) Nature, 338:518-20; Novotny et al, (1989) J. Biol. Chem., 264(31):18832-18837) two Kunitz domains of Inter-a-Trypsin Inhibitor, APP-I (Kido et al., (1988) J. Biol. Chem., 263(34):18104-18107), a Kunitz domain from collagen, and three Kunitz domains of TFPI-2 (Sprecher et al., (1994) PNAS USA, 91:3353-3357). LACI is a human serum phosphoglycoprotein with a molecular weight of 39 kDa (amino acid sequence in Table 1) containing three Kunitz domains.

TABLE 1
Exemplary Natural Kunitz Domains

| | | |
|---|---|---|
| LACI: | 1 | MIYTMKKVHA LWASVCLLLN LAPAPLNAds eedeehtiit dtelpplklM |
| (SEQ ID NO. 54) | 51 | HSFCAFKADD GPCKAIMKRF FFNIFTRQCE EFIYGGCEGN QNRFESLEEC |
| | 101 | KKMCTRDnan riikttlqqe kpdfCfleed pgiCrgyitr yfynnqtkqC |
| | 151 | erfkyggClg nmnnfetlee CkniCedgpn gfqvdnygtq lnavnnsltp |
| | 201 | qstkvpslfe fhgpswCltp adrglCrane nrfyynsvig kCrpfkysgC |
| | 251 | ggnennftsk geClraCkkg fiqriskggl iktkrkrkkq rvkiayeeif |
| | 301 | vknm |

| | |
|---|---|
| | The signal sequence (1-28) is uppercase and underscored |
| | LACI-K1 is uppercase |
| | LACI-K2 is underscored |
| | LACI-K3 is bold |
| BPTI | 1 2 3 4 5 |
| (SEQ ID NO: 55) | 1234567890123456789012345678901234567890123456789012345678 |
| | RPDFCLEPPYTGPCKARIIRYFYNAKAGLCQTFVYGGCRAKRNNFKSAEDCMRTCGGA |

The Kunitz domains of LACI are referred to as LAC1-K1 (residues 50 to 107), LAC1-K2 (residues 121 to 178), and LAC1-K3 (213 to 270). The cDNA sequence of LACI is reported in Wun et al. (J. Biol. Chem., 1988, 263(13):6001-6004). Girard et al. (Nature, 1989, 338:518-20) reports mutational studies in which the P1 residues of each of the three Kunitz domains were altered. LACI-K1 inhibits Factor VIIa (F.VIIa) when F.VIIa is complexed to tissue factor and LAC1-K2 inhibits Factor Xa.

Proteins containing exemplary Kunitz domains include the following, with SWISS-PROT Accession Numbers in parentheses:

A4_HUMAN (P05067), A4_MACFA (P53601), A4_MACMU (P29216),
A4_MOUSE (P12023), A4_RAT (P08592), A4_SAISC (Q95241),
AMBP_PLEPL (P36992), APP2_HUMAN (Q06481), APP2_RAT (P15943),
AXP1_ANTAF (P81547), AXP2_ANTAF (P81548), BPT1_BOVIN (P00974),
BPT2_BOVIN (P04815), CA17_HUMAN (Q02388), CA36_CHICK (P15989),
CA36_HUMAN (P12111), CRPT_BOOMI (P81162), ELAC_MACEU (O62845),
ELAC_TRIVU (Q29143), EPPI_HUMAN (O95925), EPPI_MOUSE (Q9DA01),
HTIB_MANSE (P26227), IBP_CARCR (P00993), IBPC_BOVIN (P00976),
IBPI_TACTR (P16044), IBPS_BOVIN (P00975), ICS3_BOMMO (P07481),
IMAP_DROFU (P11424), IP52_ANESU (P10280), ISC1_BOMMO (P10831),
ISC2_BOMMO (P10832), ISH1_STOHE (P31713), ISH2_STOHE (P81129),
ISIK_HELPO (P00994), ISP2_GALME (P81906), IVB1_BUNFA (P25660),
IVB1_BUNMU (P00987), IVB1_VIPAA (P00991), IVB2_BUNMU (P00989),
IVB2_DABRU (P00990), IVB2_HEMHA (P00985), IVB2_NAJNI (P00986),
IVB3_VIPAA (P00992), IVBB_DENPO (P00983), IVBC_NAJNA (P19859),
IVBC_OPHHA (P82966), IVBE_DENPO (P00984), IVBI_DENAN (P00980),
IVBI_DENPO (P00979), IVBK_DENAN (P00982), IVBK_DENPO (P00981),
IVBT_ERIMA (P24541), IVBT_NAJNA (P20229), MCPI_MELCP (P82968),
SBPI_SARBU (P26228), SPT3_HUMAN (P49223), TKD1_BOVIN (Q28201),
TKD1_SHEEP (Q29428), TXCA_DENAN (P81658), UPTI_PIG (Q29100),
AMBP_BOVIN (P00978), AMBP_HUMAN (P02760), AMBP_MERUN (Q62577),
AMBP_MESAU (Q60559), AMBP_MOUSE (Q07456), AMBP_PIG (P04366),
AMBP_RAT (Q64240), IATR_HORSE (P04365), IATR_SHEEP (P13371),
SPT1_HUMAN (O43278), SPT1_MOUSE (Q9R097), SPT2_HUMAN (O43291),
SPT2_MOUSE (Q9WU03), TFP2_HUMAN (P48307), TFP2_MOUSE (O35536),
TFPI_HUMAN (P10646), TFPI_MACMU (Q28864), TFPI_MOUSE (O54819),
TFPI_RABIT (P19761), TFPI_RAT (Q02445), YN81_CAEEL (Q03610)

A variety of methods can be used to identify a Kunitz domain from a sequence database. For example, a known amino acid sequence of a Kunitz domain, a consensus sequence, or a motif (e.g., the ProSite Motif) can be searched against the GenBank sequence databases (National Center for Biotechnology Information, National Institutes of Health, Bethesda Md.), e.g., using BLAST; against Pfam database of HMMs (Hidden Markov Models) (e.g., using default parameters for Pfam searching); against the SMART database; or against the Propom database. For example, the Pfam Accession Number PF00014 of Pfam Release 9 provides numerous Kunitz domains and an HMM for identifying Kunitz domains. A description of the Pfam database can be found in Sonhammer et al. (1997) Proteins 28(3):405-420 and a detailed description of HMMs can be found, for example, in Gribskov et al. (1990) Meth. Enzymol. 183:146-159; Gribskov et al. (1987) Proc. Natl. Acad. Sci. USA 84:4355-4358; Krogh et al. (1994) J. Mol. Biol. 235:1501-1531; and Stultz et al. (1993) Protein Sci. 2:305-314. The SMART database (Simple Modular Architecture Research Tool, EMBL, Heidelberg, DE) of HMMs as described in Schultz et al. (1998), Proc. Natl. Acad. Sci. USA 95:5857 and Schultz et al. (2000) Nucl. Acids Res 28:231. The SMART database contains domains identified by profiling with the hidden Markov models of the HMMer2 search program (R. Durbin et al. (1998) Biological sequence analysis: probabilistic models of proteins and nucleic acids. Cambridge University Press). The database also is annotated and monitored. The Propom protein domain database consists of an automatic compilation of homologous domains (Corpet et al. (1999), Nucl. Acids Res. 27:263-267). Current versions of Propom are built using recursive PSI-BLAST searches (Altschul et al. (1997) Nucleic Acids Res. 25:3389-3402; Gouzy et al. (1999) Computers and Chemistry 23:333-340.) of the SWISS-PROT 38 and TREMBL protein databases. The database automatically generates a consensus sequence for each domain. Prosite lists the Kunitz domain as a motif and identifies proteins that include a Kunitz domain. See, e.g., Falquet et al. Nucleic Acids Res. 30:235-238 (2002).

Kunitz domains interact with target protease using, primarily, amino acids in two loop regions (“binding loops”). The first loop region is between about residues corresponding to amino acids 15-20 of BPTI. The second loop region is between about residues corresponding to amino acids 31-37 of BPTI. An exemplary library of Kunitz domains varies one or more amino acid positions in the first and/or second loop regions. Particularly useful positions to vary, when screening for Kunitz domains that interact with kallikrein or when selecting for improved affinity variants, include: positions 13, 16, 17, 18, 19, 31, 32, 34, and 39 with respect to the sequence of BPTI. At least some of these positions are expected to be in close contact with the target protease. It is also useful to vary other positions, e.g., positions that are adjacent to the aforementioned positions in the three-dimensional structure.

The “framework region” of a Kunitz domain is defined as those residues that are a part of the Kunitz domain, but specifically excluding residues in the first and second binding loop regions, i.e., about residues corresponding to amino acids 15-20 of BPTI and 31-37 of BPTI. Conversely, residues that are not in the binding loop may tolerate a wider range of amino acid substitution (e.g., conservative and/or non-conservative substitutions).

In one embodiment these Kunitz domains are variant forms of the looped structure including Kunitz domain 1 of human lipoprotein-associated coagulation inhibitor (LACI) protein. LACI contains three internal, well-defined, peptide loop structures that are paradigm Kunitz domains (Girard, T. et al., 1989. Nature, 338:518-520). Variants of Kunitz domain 1 of LACI described herein have been screened, isolated and bind kallikrein with enhanced affinity and specificity (see, for example, U.S. Pat. Nos. 5,795,865 and 6,057,287). These methods can also be applied to other Kunitz domain frameworks to obtain other Kunitz domains that interact with kallikrein, e.g., plasma kallikrein. Useful modulators of kallikrein function typically bind and/or inhibit kallikrein, as determined using kallikrein binding and inhibition assays.

An exemplary polypeptide that includes a Kunitz domain that inhibits kallikrein has the amino acid sequence defined by amino acids 3-60 of SEQ ID NO:2.

Another exemplary polypeptide that includes a Kunitz domain that inhibits kallikrein has the amino acid sequence defined by SEQ ID NO:2.

An exemplary polypeptide includes the amino acid sequence:

Xaa1 Xaa2 Xaa3 Xaa4 Cys Xaa6 Xaa7 Xaa8 Xaa9 Xaa10 Xaa11 Gly Xaa13 Cys Xaa15 Xaa16 Xaa17 Xaa18 Xaa19 Xaa20 Xaa21 Xaa22 Xaa23 Xaa24 Xaa25 Xaa26 Xaa27 Xaa28 Xaa29 Cys Xaa31 Xaa32 Phe Xaa34 Xaa35 Gly Gly Cys Xaa39 Xaa40 Xaa41 Xaa42 Xaa43 Xaa44 Xaa45 Xaa46 Xaa47 Xaa48 Xaa49 Xaa50 Cys Xaa52 Xaa53 Xaa54 Cys Xaa56 Xaa57 Xaa58 (SEQ ID NO:1)

“Xaa” refers to a position in a peptide chain that can be any of a number of different amino acids. In a first example, Xaa can by any amino acid except cysteine. In another example, one or more of the following apply: Xaa10 can be Asp or Glu; Xaa11 can be Asp, Gly, Ser, Val, Asn, Ile, Ala or Thr; Xaa13 can be Pro, Arg, His, Asn, Ser, Thr, Ala, Gly, Lys or Gln; Xaa15 can be Arg, Lys, Ala, Ser, Gly, Met, Asn or Gln; Xaa16 can be Ala, Gly, Ser, Asp or Asn; Xaa17 can be Ala, Asn, Ser, Ile, Gly, Val, Gln or Thr; Xaa18 can be His, Leu, Gln or Ala; Xaa19 can be Pro, Gln, Leu, Asn or Ile; Xaa21 can be Trp, Phe, Tyr, His or Ile; Xaa31 can be Glu, Asp, Gln, Asn, Ser, Ala, Val, Leu, Ile or Thr; Xaa32 can be Glu, Gln, Asp Asn, Pro, Thr, Leu, Ser, Ala, Gly or Val; Xaa34 can be Ile, Thr, Ser, Val, Ala, Asn, Gly or Leu; Xaa35 can be Tyr, Trp or Phe; Xaa39 can be Glu, Gly, Ala, Ser or Asp. Amino acids Xaa6, Xaa7, Xaa8, Xaa9, Xaa20, Xaa24, Xaa25, Xaa26, Xaa27, Xaa28, Xaa29, Xaa41, Xaa42, Xaa44, Xaa46, Xaa47, Xaa48, Xaa49, Xaa50, Xaa52, Xaa53 and Xaa54 can be any amino acid.

Additionally, each of the first four and at last three amino acids of SEQ ID NO:1 can optionally be present or absent and can be any amino acid, if present, e.g., any non-cysteine amino acid.

In one embodiment, the polypeptide has a sequence with one or more of the following properties: Xaa11 can be Asp, Gly, Ser or Val; Xaa13 can be Pro, Arg, His or Asn; Xaa15 can be Arg or Lys; Xaa16 can be Ala or Gly; Xaa17 can be Ala, Asn, Ser or Ile; Xaa18 can be His, Leu or Gln; Xaa19 can be Pro, Gln or Leu; Xaa21 can be Trp or Phe; Xaa31 is Glu; Xaa32 can be Glu or Gln; Xaa34 can be Ile, Thr or Ser; Xaa35 is Tyr; and Xaa39 can be Glu, Gly or Ala.

An exemplary polypeptide can include the following amino acids: Xaa10 is Asp; Xaa11 is Asp; Xaa13 can be Pro or Arg; Xaa15 is Arg; Xaa16 can be Ala or Gly; Xaa17 is Ala; Xaa18 is His; Xaa19 is Pro;Xaa21 is Trp; Xaa31 is Glu; Xaa32 is Glu; Xaa34 can be Ile or Ser; Xaa35 is Tyr; and Xaa39 is Gly.

It is also possible to use portions of the polypeptides described herein. For example, polypeptides could include binding domains for specific kallikrein epitopes. For example, the binding loops of Kunitz domains can by cyclized and used in isolation or can be grafted onto another domain, e.g., a framework of another Kunitz domain. It is also possible to remove one, two, three, or four amino acids from the N-terminus of an amino acid sequence described herein, and/or one, two, three, four, or five amino acids from the C-terminus of an amino acid sequence described herein.

Examples of sequences encompassed by SEQ ID NO:1 are described by the following (where not indicated, “Xaa” refers to any amino acid, any non-cysteine amino acid or any amino acid from the same set of amino acids that are allowed for SEQ ID NO:1):

(SEQ ID NO: 33)
Met His Ser Phe Cys Ala Phe Lys Ala Xaa10 Xaa11
Gly Xaa13 Cys Xaa15 Xaa16 Xaa17 Xaa18 Xaa19 Arg
Xaa21 Phe Phe Asn Ile Phe Thr Arg Gln Cys Xaa31
Xaa32 Phe Xaa34 Xaa35 Gly Gly Cys Xaa39 Gly
Asn Gln Asn Arg Phe Glu Ser Leu Glu Glu Cys Lys
Lys Met Cys Thr Arg Asp.
(amino acids 3-60 of SEQ ID NO: 2)
Met His Ser Phe Cys Ala Phe Lys Ala Asp Asp Gly
Pro Cys Arg Ala Ala His Pro Arg Trp Phe Phe Asn
Ile Phe Thr Arg Gln Cys Glu Glu Phe Ile Tyr Gly
Gly Cys Glu Gly Asn Gln Asn Arg Phe Glu Ser Leu
Glu Glu Cys Lys Lys Met Cys Thr Arg Asp,
(SEQ ID NO: 2)
Glu Ala Met His Ser Phe Cys Ala Phe Lys Ala Asp
Asp Gly Pro Cys Arg Ala Ala His Pro Arg Trp Phe
Phe Asn Ile Phe Thr Arg Gln Cys Glu Glu Phe Ile
Tyr Gly Gly Cys Glu Gly Asn Gln Asn Arg Phe Glu
Ser Leu Glu Glu Cys Lys Lys Met Cys Thr Arg Asp,
(SEQ ID NO: 4)
Met His Ser Phe Cys Ala Phe Lys Ala Asp Asp Gly
Pro Cys Lys Ala Asn His Leu Arg Phe Phe Phe Asn
Ile Phe Thr Arg Gln Cys Glu Glu Phe Ser Tyr Gly
Gly Cys Gly Gly Asn Gln Asn Arg Phe Glu Ser Leu
Glu Glu Cys Lys Lys Met Cys Thr Arg Asp,
(SEQ ID NO: 5)
Met His Ser Phe Cys Ala Phe Lys Ala Asp Asp Gly
His Cys Lys Ala Asn His Gln Arg Phe Phe Phe Asn
Ile Phe Thr Arg Gln Cys Glu Glu Phe Thr Tyr Gly
Gly Cys Gly Gly Asn Gln Asn Arg Phe Glu Ser Leu
Glu Glu Cys Lys Lys Met Cys Thr Arg Asp,
(SEQ ID NO: 6)
Met His Ser Phe Cys Ala Phe Lys Ala Asp Asp Gly
His Cys Lys Ala Asn His Gln Arg Phe Phe Phe Asn
Ile Phe Thr Arg Gln Cys Glu Gln Phe Thr Tyr Gly
Gly Cys Ala Gly Asn Gln Asn Arg Phe Glu Ser Leu
Glu Glu Cys Lys Lys Met Cys Thr Arg Asp,
(SEQ ID NO: 7)
Met His Ser Phe Cys Ala Phe Lys Ala Asp Asp Gly
His Cys Lys Ala Ser Leu Pro Arg Phe Phe Phe Asn
Ile Phe Thr Arg Gln Cys Glu Glu Phe Ile Tyr Gly
Gly Cys Gly Gly Asn Gln Asn Arg Phe Glu Ser Leu
Glu Glu Cys Lys Lys Met Cys Thr Arg Asp,
(SEQ ID NO: 8)
Met His Ser Phe Cys Ala Phe Lys Ala Asp Asp Gly
His Cys Lys Ala Asn His Gln Arg Phe Phe Phe Asn
Ile Phe Thr Arg Gln Cys Glu Glu Phe Ser Tyr Gly
Gly Cys Gly Gly Asn Gln Asn Arg Phe Glu Ser Leu
Glu Glu Cys Lys Lys Met Cys Thr Arg Asp,
(SEQ ID NO: 9)
Met His Ser Phe Cys Ala Phe Lys Ala Asp Asp Gly
His Cys Lys Gly Ala His Leu Arg Phe Phe Phe Asn
Ile Phe Thr Arg Gln Cys Glu Glu Phe Ile Tyr Gly
Gly Cys Glu Gly Asn Gln Asn Arg Phe Glu Ser Leu
Glu Glu Cys Lys Lys Met Cys Thr Arg Asp,
(SEQ ID NO: 10)
Met His Ser Phe Cys Ala Phe Lys Ala Asp Asp Gly
Arg Cys Lys Gly Ala His Leu Arg Phe Phe Phe Asn
Ile Phe Thr Arg Gln Cys Glu Glu Phe Ile Tyr Gly
Gly Cys Glu Gly Asn Gln Asn Arg Phe Glu Ser Leu
Glu Glu Cys Lys Lys Met Cys Thr Arg Asp,
(SEQ ID NO: 11)
Met His Ser Phe Cys Ala Phe Lys Ala Asp Gly Gly
Arg Cys Arg Gly Ala His Pro Arg Trp Phe Phe Asn
Ile Phe Thr Arg Gln Cys Glu Glu Phe Ser Tyr Gly
Gly Cys Gly Gly Asn Gln Asn Arg Phe Glu Ser Leu
Glu Glu Cys Lys Lys Met Cys Thr Arg Asp,
(SEQ ID NO: 12)
Met His Ser Phe Cys Ala Phe Lys Ala Asp Asp Gly
Pro Cys Arg Ala Ala His Pro Arg Trp Phe Phe Asn
Ile Phe Thr Arg Gln Cys Glu Glu Phe Ser Tyr Gly
Gly Cys Gly Gly Asn Gln Asn Arg Phe Glu Ser Leu
Glu Glu Cys Lys Lys Met Cys Thr Arg Asp,
(SEQ ID NO: 13)
Met His Ser Phe Cys Ala Phe Lys Ala Asp Val Gly
Arg Cys Arg Gly Ala His Pro Arg Trp Phe Phe Asn
Ile Phe Thr Arg Gln Cys Glu Glu Phe Ser Tyr Gly
Gly Cys Gly Gly Asn Gln Asn Arg Phe Glu Ser Leu
Glu Glu Cys Lys Lys Met Cys Thr Arg Asp,
(SEQ ID NO: 14)
Met His Ser Phe Cys Ala Phe Lys Ala Asp Val Gly
Arg Cys Arg Gly Ala Gln Pro Arg Phe Phe Phe Asn
Ile Phe Thr Arg Gln Cys Glu Glu Phe Ser Tyr Gly
Gly Cys Gly Gly Asn Gln Asn Arg Phe Glu Ser Leu
Glu Glu Cys Lys Lys Met Cys Thr Arg Asp,
(SEQ ID NO: 15)
Met His Ser Phe Cys Ala Phe Lys Ala Asp Asp Gly
Ser Cys Arg Ala Ala His Leu Arg Trp Phe Phe Asn
Ile Phe Thr Arg Gln Cys Glu Glu Phe Ser Tyr Gly
Gly Cys Gly Gly Asn Gln Asn Arg Phe Glu Ser Leu
Glu Glu Cys Lys Lys Met Cys Thr Arg Asp,
(SEQ ID NO: 16)
Met His Ser Phe Cys Ala Phe Lys Ala Glu Gly Gly
Ser Cys Arg Ala Ala His Gln Arg Trp Phe Phe Asn
Ile Phe Thr Arg Gln Cys Glu Glu Phe Ser Tyr Gly
Gly Cys Gly Gly Asn Gln Asn Arg Phe Glu Ser Leu
Glu Glu Cys Lys Lys Met Cys Thr Arg Asp,
(SEQ ID NO: 17)
Met His Ser Phe Cys Ala Phe Lys Ala Asp Asp Gly
Pro Cys Arg Gly Ala His Leu Arg Phe Phe Phe Asn
Ile Phe Thr Arg Gln Cys Glu Glu Phe Ser Tyr Gly
Gly Cys Gly Gly Asn Gln Asn Arg Phe Glu Ser Leu
Glu Glu Cys Lys Lys Met Cys Thr Arg Asp,
(SEQ ID NO: 18)
Met His Ser Phe Cys Ala Phe Lys Ala Asp Asp Gly
His Cys Arg Gly Ala Leu Pro Arg Trp Phe Phe Asn
Ile Phe Thr Arg Gln Cys Glu Glu Phe Ser Tyr Gly
Gly Cys Gly Gly Asn Gln Asn Arg Phe Glu Ser Leu
Glu Glu Cys Lys Lys Met Cys Thr Arg Asp,
(SEQ ID NO: 19)
Met His Ser Phe Cys Ala Phe Lys Ala Asp Ser Gly
Asn Cys Arg Gly Asn Leu Pro Arg Phe Phe Phe Asn
Ile Phe Thr Arg Gln Cys Glu Glu Phe Ser Tyr Gly
Gly Cys Gly Gly Asn Gln Asn Arg Phe Glu Ser Leu
Glu Glu Cys Lys Lys Met Cys Thr Arg Asp,
(SEQ ID NO: 20)
Met His Ser Phe Cys Ala Phe Lys Ala Asp Ser Gly
Arg Cys Arg Gly Asn His Gln Arg Phe Phe Phe Asn
Ile Phe Thr Arg Gln Cys Glu Glu Phe Ser Tyr Gly
Gly Cys Gly Gly Asn Gln Asn Arg Phe Glu Ser Leu
Glu Glu Cys Lys Lys Met Cys Thr Arg Asp,
(SEQ ID NO: 21)
Met His Ser Phe Cys Ala Phe Lys Ala Asp Gly Gly
Arg Cys Arg Ala Ile Gln Pro Arg Trp Phe Phe Asn
Ile Phe Thr Arg Gln Cys Glu Glu Phe Ser Tyr Gly
Gly Cys Gly Gly Asn Gln Asn Arg Phe Glu Ser Leu
Glu Glu Cys Lys Lys Met Cys Thr Arg Asp,
(SEQ ID NO: 22)
Met His Ser Phe Cys Ala Phe Lys Ala Asp Asp Gly
Arg Cys Arg Gly Ala His Pro Arg Trp Phe Phe Asn
Ile Phe Thr Arg Gln Cys Glu Glu Phe Ser Tyr Gly
Gly Cys Gly Gly Asn Gln Asn Arg Phe Glu Ser Leu
Glu Glu Cys Lys Lys Met Cys Thr Arg Asp.

Additional examples of sequence include those that differ by at least one amino acid, but fewer than seven, six, five, four, three, or two amino acids differences relative to an amino acid sequence described herein, e.g., an amino acid sequence provided above. In one embodiment, fewer than three, two, or one differences are in one of the binding loops. For example, the first binding loop may have no differences relative to an amino acid sequence described herein, e.g., an amino acid sequence provided above. In another example, neither the first nor the second binding loop differs from an amino acid sequence described herein, e.g., an amino acid sequence provided above.

In one embodiment, the polypeptide that inhibits kallikrein is aprotinin. In another embodiment, the polypeptide is other than aprotinin, e.g., differs from aprotinin, by at least one, two, three, five, ten, or fifteen amino acids.

Polypeptides described herein can be made synthetically using any standard polypeptide synthesis protocol and equipment. For example, the stepwise synthesis of a polypeptide can be carried out by the removal of an amino (N) terminal-protecting group from an initial (i.e., carboxy-terminal) amino acid, and coupling thereto of the carboxyl end of the next amino acid in the sequence of the polypeptide. This amino acid is also suitably protected. The carboxyl group of the incoming amino acid can be activated to react with the N-terminus of the bound amino acid by formation into a reactive group such as formation into a carbodiimide, a symmetric acid anhydride, or an “active ester” group such as hydroxybenzotriazole or pentafluorophenyl esters. Preferred solid-phase peptide synthesis methods include the BOC method, which utilizes tert-butyloxycarbonyl as the I-amino protecting group, and the FMOC method, which utilizes 9-fluorenylmethloxycarbonyl to protect the I-amino of the amino acid residues. Both methods are well known to those of skill in the art (Stewart, J. and Young, J., Solid-Phase Peptide Synthesis (W. H. Freeman Co., San Francisco 1989); Merrifield, J., 1963. Am. Chem. Soc., 85:2149-2154; Bodanszky, M. and Bodanszky, A., The Practice of Peptide Synthesis (Springer-Verlag, New York 1984)). If desired, additional amino- and/or carboxy-terminal amino acids can be designed into the amino acid sequence and added during polypeptide synthesis.

Polypeptides can also be produced using recombinant technology. Recombinant methods can employ any of a number of cells and corresponding expression vectors, including but not limited to bacterial expression vectors, yeast expression vectors, baculovirus expression vectors, mammalian viral expression vectors, and the like. A polypeptide described herein can be produced by a transgenic animal, e.g., in the mammary gland of a transgenic animal. In some cases, it could be necessary or advantageous to fuse the coding sequence for a polypeptide that inhibits kallikrein (e.g., a polypeptide that includes a Kunitz domain) to another coding sequence in an expression vector to form a fusion polypeptide that is readily expressed in a host cell. Part or all of the additional sequence can be removed, e.g., by protease digestion. An exemplary recombinant expression system for producing a polypeptide that inhibits kallikrein (e.g., a polypeptide that includes a Kunitz domain) is a yeast expression vector, which permits a nucleic acid sequence encoding the amino acid sequence for the inhibitor polypeptide to be linked in the same reading frame with a nucleotide sequence encoding the MATa prepro leader peptide sequence of Saccharomyces cerevisiae, which in turn is under the control of an operable yeast promoter. The resulting recombinant yeast expression plasmid can be transformed by standard methods into the cells of an appropriate, compatible yeast host, which cells are able to express the recombinant protein from the recombinant yeast expression vector. Preferably, a host yeast cell transformed with such a recombinant expression vector is also able to process the fusion protein to provide an active inhibitor polypeptide. Another exemplary yeast host for producing recombinant polypeptides is Pichia pastoris.

As noted above, polypeptides that inhibit kallikrein can include a Kunitz domain polypeptide described herein. Some polypeptides can include an additional flanking sequence, preferably of one to six (e.g., 1, 2, 3, 4, 5, or 6) amino acids in length, at the amino and/or carboxy-terminal end, provided such additional amino acids do not significantly diminish kallikrein binding affinity or kallikrein inhibition activity so as to preclude use in the methods and compositions described herein. Such additional amino acids can be deliberately added to express a polypeptide in a particular recombinant host cell or can be added to provide an additional function, e.g., to provide a linker to another molecule or to provide an affinity moiety that facilitates purification of the polypeptide. Preferably, the additional amino acid(s) do not include cysteine, which could interfere with the disulfide bonds of the Kunitz domain.

In one embodiment, an inhibitor of kallikrein, e.g., a polypeptide inhibitor, has a binding affinity for kallikrein that is on the order of 1000 times higher than that of aprotinin, which is currently approved for use in CABG procedures to reduce blood loss. The surprisingly high binding affinities of such kallikrein inhibitors combined with their high degree of specificity for kallikrein to the exclusion of other molecular targets provide for particularly useful inhibitors. However, inhibitors with lesser affinity or specificity also have their applications.

A typical Kunitz domain, e.g., that includes, SEQ ID NO:1, contains a number of invariant positions, e.g., positions corresponding to position 5, 14, 30, 51 and 55 in the BPTI numbering scheme are cysteine. The spacing between these positions may vary to the extent allowable within the Kunitz domain fold, e.g., such that three disulfide bonds are formed. Other positions such as, for example, positions 6, 7, 8, 9, 20, 24, 25, 26, 27, 28, 29, 41, 42, 44, 46, 47, 48, 49, 50, 52, 53 and 54, or positions corresponding to those positions, can be any amino acid (including non-naturally occurring amino acids). In a particularly preferred embodiment, one or more amino acids correspond to that of a native sequence. In another embodiment, at least one variable position is different from that of the native sequence. In yet another preferred embodiment, the amino acids can each be individually or collectively substituted by a conservative or non-conservative amino acid substitution.

Conservative amino acid substitutions replace an amino acid with another amino acid of similar chemical structure and may have no affect on protein function. Non-conservative amino acid substitutions replace an amino acid with another amino acid of dissimilar chemical structure. Examples of conserved amino acid substitutions include, for example, Asn->Asp, Arg->Lys and Ser->Thr. In a preferred embodiment, 1, 2, 3, 4, 5, 6, 7, 8, 9, 10, 11, 12, 13, 14, 15, 16, 17, 18, 19, 20 and/or 21 of these amino acids can be independently or collectively, in any combination, selected to correspond to the corresponding position of SEQ ID NO:2.

Other positions, for example, positions 10, 11, 13, 15, 16, 17, 18, 19, 21, 22, 23, 31, 32, 34, 35, 39, 40, 43 and 45, or positions corresponding to those positions can be any of a selected set of amino acids. For example, SEQ ID NO:1 defines a set of possible sequences. Each member of this set contains, for example, a cysteine at positions 5, 14, 30, 51 and 55, and any one of a specific set of amino acids at positions 10, 11, 13, 15, 16, 17, 18, 19, 221, 22, 23, 31, 32, 34, 35, 39, 40, 43 and 45, or positions corresponding to those positions. In a preferred embodiment, 1, 2, 3, 4, 5, 6, 7, 8, 9, 10, 11, 12, 13, 14, 15, 16, 17, 18 and/or 19 of these amino acids can be independently or collectively, in any combination, selected to correspond to the corresponding position of SEQ ID NO:2. The polypeptide preferably has at least 80%, 85%, 90%, 95, 97, 98, or 99% identity to SEQ ID NO:2.

Useful polypeptides can also be encoded by a nucleic acid that hybridizes to a nucleic acid that encodes a polypeptide described herein. The nucleic acids can hybridize under medium, high, or very high stringency conditions. As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1-6.3.6, which is incorporated by reference. Aqueous and nonaqueous methods are described in that reference and either can be used. Specific hybridization conditions referred to herein are as follows: (1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); (2) medium stringency hybridization conditions in 6×SSC at about 45° C., followed by one or more washes in 0.2×SSC, 0.1% SDS at 60° C.; (3) high stringency hybridization conditions in 6×SSC at about 45° C., followed by one or more washes in 0.2×SSC, 0.1% SDS at 65° C.; and (4) very high stringency hybridization conditions are 0.5M sodium phosphate, 7% SDS at 65° C., followed by one or more washes at 0.2×SSC, 1% SDS at 65° C.

### Kallikrein Inhibitors—Antibodies

One class of kallikrein inhibitors includes antibodies. Exemplary antibodies bind, e.g., specifically to kallikrein, e.g., plasma kallikrein. An antibody can inhibit kallikrein in a number of ways. For example, it can contact one or more residues of the active site, sterically hinder or obstruct access to the active site, prevent maturation of kallikrein, or destabilize a conformation required for catalytic activity.

One or more regions of an antibody can be human or effectively human. For example, one or more of the variable regions can be human or effectively human. For example, one or more of the CDRs can be human, e.g., HC CDR1, HC CDR2, HC CDR3, LC CDR1, LC CDR2, and LC CDR3. Each of the light chain CDRs can be human. HC CDR3 can be human. One or more of the framework regions can be human, e.g., FR1, FR2, FR3, and FR4 of the HC or LC. In one embodiment, all the framework regions are human, e.g., derived from a human somatic cell, e.g., a hematopoietic cell that produces immunoglobulins or a non-hematopoietic cell. In one embodiment, the human sequences are germline sequences, e.g., encoded by a germline nucleic acid. One or more of the constant regions can be human or effectively human. In another embodiment, at least 70, 75, 80, 85, 90, 92, 95, or 98% of, or the entire antibody can be human or effectively human. An “effectively human” immunoglobulin variable region is an immunoglobulin variable region that includes a sufficient number of human framework amino acid positions such that the immunoglobulin variable region does not elicit an immunogenic response in a normal human. An “effectively human” antibody is an antibody that includes a sufficient number of human amino acid positions such that the antibody does not elicit an immunogenic response in a normal human.

All or part of an antibody can be encoded by an immunoglobulin gene or a segment thereof. Exemplary human immunoglobulin genes include the kappa, lambda, alpha (IgA1 and IgA2), gamma (IgG1, IgG2, IgG3, and IgG4), delta, epsilon and mu constant region genes, as well as the myriad immunoglobulin variable region genes. Full-length immunoglobulin “light chains” (about 25 Kd or 214 amino acids) are encoded by a variable region gene at the NH2-terminus (about 110 amino acids) and a kappa or lambda constant region gene at the COOH—terminus. Full-length immunoglobulin “heavy chains” (about 50 Kd or 446 amino acids), are similarly encoded by a variable region gene (about 116 amino acids) and one of the other aforementioned constant region genes, e.g., gamma (encoding about 330 amino acids).

One exemplary method for identifying antibodies that bind to and inhibit kallikrein includes immunizing a non-human animal with kallikrein or a fragment thereof. Even small peptides can be used as immunogens. In one embodiment, a mutated kallikrein which has reduced or no catalytic activity is used as immunogen. Spleen cells can be isolated from the immunized animal and used to produce hybridoma cells using standard methods. In one embodiment, the non-human animal includes one or more human immunoglobulin genes.

Another exemplary method for identifying proteins that bind to and inhibit kallikrein includes: providing a library of proteins and selecting from the library one or more proteins that bind to a kallikrein or a fragment thereof. The selection can be performed in a number of ways. For example, the library can be provided in the format of a display library or a protein array. Prior to selecting, the library can be pre-screened (e.g., depleted) to remove members that interact with a non-target molecule, e.g., protease other than a kallikrein or a kallikrein in which the active site is inaccessible, e.g., bound by an inhibitor, e.g., aprotinin.

Antibody libraries, e.g., antibody display libraries, can be constructed by a number of processes (see, e.g., de Haard et al. (1999) J. Biol. Chem. 274:18218-30; Hoogenboom et al. (1998) Immunotechnology 4:1-20. and Hoogenboom et al. (2000) Immunol Today 21:371-8). Further, elements of each process can be combined with those of other processes. The processes can be used such that variation is introduced into a single immunoglobulin domain (e.g., VH or VL) or into multiple immunoglobulin domains (e.g., VH and VL). The variation can be introduced into an immunoglobulin variable domain, e.g., in the region of one or more of CDR1, CDR2, CDR3, FR1, FR2, FR3, and FR4, referring to such regions of either and both of heavy and light chain variable domains. In one embodiment, variation is introduced into all three CDRs of a given variable domain. In another preferred embodiment, the variation is introduced into CDR1 and CDR2, e.g., of a heavy chain variable domain. Any combination is feasible. In an exemplary system for recombinant expression of an antibody (e.g., a full length antibody or an antigen-binding portion thereof), a recombinant expression vector encoding both the antibody heavy chain and the antibody light chain is introduced into dhfr-CHO cells by calcium phosphate-mediated transfection. Within the recombinant expression vector, the antibody heavy and light chain genes are each operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, transfect the host cells, select for transformants, culture the host cells, and recover the antibody from the culture medium. For example, some antibodies can be isolated by affinity chromatography with a Protein A or Protein G. Antibodies can also be produced by a transgenic animal.

### Kallikrein Inhibitors—Peptides

A binding ligand can include a peptide of 32 amino acids or less that independently binds to a target molecule. Some such peptides can include one or more disulfide bonds. Other peptides, so-called “linear peptides,” are devoid of cysteines. In one embodiment, the peptides are artificial, i.e., not present in a nature or not present in a protein encoded by one or more genomes of interest, e.g., the human genome. Synthetic peptides may have little or no structure in solution (e.g., unstructured), heterogeneous structures (e.g., alternative conformations or “loosely” structured), or a singular native structure (e.g., cooperatively folded). Some synthetic peptides adopt a particular structure when bound to a target molecule. Some exemplary synthetic peptides are so-called “cyclic peptides” that have at least a disulfide bond and, for example, a loop of about 4 to 12 non-cysteine residues. Exemplary peptides are less than 28, 24, 20, or 18 amino acids in length.

Peptide sequences that independently bind kallkrein can be identified by any of a variety of methods. For example, they can be selected from a display library or an array of peptides. After identification, such peptides can be produced synthetically or by recombinant means. The sequences can be incorporated (e.g., inserted, appended, or attached) into longer sequences.

The following are some exemplary phage libraries that can be screened. Each library displays a short, variegated exogenous peptide on the surface of M13 phage. The peptide display of five of the libraries was based on a parental domain having a segment of 4, 5, 6, 7, 8, 10, 11, or 12 amino acids, respectively, flanked by cysteine residues. The pairs of cysteines are believed to form stable disulfide bonds, yielding a cyclic display peptide. The cyclic peptides are displayed at the amino terminus of protein III on the surface of the phage. The libraries were designated TN6/7, TN7/4, TN8/9, TN9/4, TN10/10, TN11/1, and TN12/1. A phage library with a 20-amino acid linear display was also screened; this library was designated Lin20.

The TN6/7 library was constructed to display a single cyclic peptide contained in a 12-amino acid variegated template. The TN6/6 library utilized a template sequence of Xaa₁-Xaa₂-Xaa₃-Cys₄-Xaa₅-Xaa₆-Xaa₇-Xaa₈-Cys₉-Xaa₁₀-Xaa₁₁-Xaa₁₂, where each variable amino acid position in the amino acid sequence of the template is indicated by a subscript integer. Each variable amino acid position (Xaa) in the template was varied to contain any of the common α-amino acids, except cysteine (Cys).

The TN7/4 library was constructed to display a single cyclic peptide contained in a 12-amino acid variegated template. The TN7/4 library utilized a template sequence of Xaa₁-Xaa₂-Xaa₃-Cys₄-Xaa₅-Xaa₆-Xaa₇-Xaa₈-Xaa₉-Cys₁₀-Xaa₁₁-Xaa₁₂-Xaa₁₃, where each variable amino acid position in the amino acid sequence of the template is indicated by a subscript integer. Each variable amino acid position (Xaa) in the template was varied to contain any of the common α-amino acids, except cysteine (Cys).

The TN8/9 library was constructed to display a single binding loop contained in a 14-amino acid template. The TN8/9 library utilized a template sequence of Xaa₁-Xaa₂-Xaa₃-Cys-Xaa₅-Xaa₆Xaa₇-Xaa₈-Xaa₉-Xaa₁₀-Cys-Xaa₁₂-Xaa₁₃-Xaa₁₄. Each variable amino acid position (Xaa) in the template were varied to permit any amino acid except cysteine (Cys).

The TN9/4 library was constructed to display a single binding loop contained in a 15-amino acid template. The TN9/4 library utilized a template sequence Xaa₁-Xaa₂-Xaa₃-Cys₄-Xaa₅-Xaa₆-Xaa₇-Xaa₈-Xaa₉-Xaa₁₀-Xaa₁₁ᵢ-Cysᵢ₂-Xaa₁₃-Xaa₁₄-Xaa₁₅. Each variable amino acid position (Xaa) in the template were varied to permit any amino acid except cysteine (Cys).

The TN10/10 library was constructed to display a single cyclic peptide contained in a 16-amino acid variegated template. The TN10/9 library utilized a template sequence Xaa₁-Xaa₂-Xaa₃-Cys₄-Xaa₅-Xaa₆-Xaa₇-Xaa₈-Xaa₉-Xaa₁₀-Xaa₁₁-Xaa₁₂-Cys₁₃-Xaa₁₄-Xaa₁₅-Xaa₁₆, where each variable amino acid position in the amino acid sequence of the template is indicated by a subscript integer. Each variable amino acid position (Xaa) was to permit any amino acid except cysteine (Cys).

The TN11/1 library was constructed to display a single cyclic peptide contained in a 17-amino acid variegated template. The TN11/1 library utilized a template sequence Xaa₁-Xaa₂-Xaa₃-Cys₄-Xaa₅-Xaa₆-Xaa₇-Xaa₈-Xaa₉-Xaa₁₀-Xaa₁₁-Xaa₁₂-Xaa₁₃-Cys₁₄-Xaa₁₅-Xaa₁₆-Xaa₁₇, where each variable amino acid position in the amino acid sequence of the template is indicated by a subscript integer. Each variable amino acid position (Xaa) was to permit any amino acid except cysteine (Cys).

The TN12/1 library was constructed to display a single cyclic peptide contained in an 18-amino acid template. The TN12/1 library utilized a template sequence Xaa₁-Xaa₂-Xaa₃-Cys₄-Xaa₅-Xaa₆-Xaa₇-Xaa₈-Xaa₉-Xaa₁₀-Xaa₁₁-Xaa₁₂-Xaa₁₃-Xaa₁₄-Cys₁₅-Xaa₁₆-Xaa₁₇-Xaa₁₈, where each variable amino acid position in the amino acid sequence of the template is indicated by a subscript integer. The amino acid positions Xaa₁, Xaa₂, Xaa₁₇ and Xaa₁₈ of the template were varied, independently, to permit each amino acid selected from the group of 12 amino acids consisting of Ala, Asp, Phe, Gly, His, Leu, Asn, Pro, Arg, Ser, Trp, and Tyr. The amino acid positions Xaa₃, Xaa₅, Xaa₆, Xaa₇, Xaa₈, Xaa₉, Xaa₁₀, Xaa₁₁, Xaa₁₂, Xaa₁₃, Xaa₁₄, Xaa₁₆, of the template were varied, independently, to permit any amino acid except cysteine (Cys).

The Lin20 library was constructed to display a single linear peptide in a 20-amino acid template. The amino acids at each position in the template were varied to permit any amino acid except cysteine (Cys).

The techniques discussed in Kay et al., Phage Display of Peptides and Proteins: A Laboratory Manual (Academic Press, Inc., San Diego 1996) and U.S. Pat. No. 5,223,409 are useful for preparing a library of potential binders corresponding to the selected parental template. The libraries described above can be prepared according to such techniques, and screened, e.g., as described above, for peptides that bind to and inhibit kallikrein.

In addition phage libraries or selected populations from phage libraries can be counter-selected, e.g., using kallikrein that is inactivated, e.g., by binding of aprotinin or another kallikrein inhibitor. Such procedures can be used to discard peptides that do not contact the active site.

Peptides can also be synthesized using alternative backbones, e.g., a peptoid backbone, e.g., to produce a compound which has increased protease resistance. In particular this method can be used to make a compound that binds to and inhibits kallikrein and which is not itself effectively cleaved by kallikrein.

### Protease Inhibitors—Plasmin Binding Proteins/Inhibitors

Plasmin is a serine protease derived from plasminogen. The catalytic domain of plasmin (or “CatDom”) cuts peptide bonds, particularly after arginine residues and to a lesser extent after lysines and is highly homologous to trypsin, chymotrypsin, kallikrein, and many other serine proteases. Most of the specificity of plasmin derives from the kringle domains of plasmin binding of fibrin (Lucas et al., J Biological Chem (1983) 258(7)4249-56.; Varadi & Patthy, Biochemistry (1983) 22:2440-2446.; and Varadi & Patthy, Biochemistry (1984) 23:2108-2112.). On activation, the bond between ARG₅₆₁-Val₅₆₂ is cut, allowing the newly free amino terminus to form a salt bridge. The kringles remain, nevertheless, attached to the CatDom through two disulfides (Colman, R W, J Hirsh, V J Marder, and E W Salzman, Editors, Hemostasis and Thrombosis, Second Edition, 1987, J. B. Lippincott Company, Philadelphia, Pa., Bobbins, 1987, supra.

The agent mainly responsible for fibrinolysis is plasmin, the activated form of plasminogen. Many substances can activate plasminogen, including activated Hageman factor, streptokinase, urokinase (uPA), tissue-type plasminogen activator (tPA), and plasma kallikrein (pKA). pKA is both an activator of the zymogen form of urokinase and a direct plasminogen activator.

Plasmin is undetectable in normal circulating blood, but plasminogen, the zymogen, is present at about 3 μM. An additional, unmeasured amount of plasminogen is bound to fibrin and other components of the extracellular matrix and cell surfaces. Normal blood contains the physiological inhibitor of plasmin, α₂-plasmin inhibitor (α₂-PI), at about 2 μM. Plasmin and α₂-PI form a 1:1 complex. Matrix or cell bound-plasmin is relatively inaccessible to inhibition by α₂-PI. Thus, activation of plasmin can exceed the neutralizing capacity of α₂-PI causing a profibrinolytic state.

Plasmin, once formed:
i) degrades fibrin clots, sometimes prematurely;
ii) digests fibrinogen (the building material of clots) impairing hemostasis by causing formation of friable, easily lysed clots from the degradation products, and inhibition of platelet adhesion/aggregation by the fibrinogen degradation products;
iii) interacts directly with platelets to cleave glycoproteins Ib and IIb/IIIa preventing adhesion to injured endothelium in areas of high shear blood flow and impairing the aggregation response needed for platelet plug formation (Adelman et al., Blood (1986) 68(6)1280-1284.);
iv) proteolytically inactivates enzymes in the extrinsic coagulation pathway further promoting a prolytic state. Robbins (Robbins, Chapter 21 of Hemostasis and Thrombosis, Colman, R. W., J. Hirsh, V. J. Marder, and E. W. Salzman, Editors, Second Edition, 1987, J. B. Lippincott Company, Philadelphia, Pa.) reviewed the plasminogen-plasmin system in detail.

Markland et al. [U.S. Pat. Nos. 6,010,880; 6,071,723; and 6,103,49] claim a number of derivatives having high affinity and specificity in inhibiting plasmin.

### DX-1000

DX-1000 is a Kunitz domain polypeptide that inhibits human plasmin. (Ki=88 pM). Further description of Kunitz domains is provided below. The DX-1000 protein includes the framework region of human LACI, but other frameworks can also be used. The sequence of DX-1000 can include the following amino acid sequence (SEQ ID NO:100):

```
        5 10 15 20 25 30 35 40 45
        . . . . . . . . .
MHSFCAFKAETGPCRARFDRWFFNIFTRQCEEFIYGGCEGNQNRFESL
  50 55
  . .
EECKKMCTRD
```

The sequence can also be preceded by two N-terminal amino acids (“EA”) to include the following sequence (SEQ ID NO:200):

EAMHSFCAFKAETGPCRARFDRWFFNIFTRQCEEFIYGGCEGNQNRFE
SLEECKKMCTRD

DX-1000 was tested in several functional cell-based activity assays and demonstrated potent inhibitory activity. Firstly, DX-1000 (1 nM) inhibited both DHT-stimulated invasion of LNCaPs (prostate cancer) and HT-1080 (fibrosarcoma) through Matrigel, processes known to be dependent on the plasminogen/plasmin system. Interestingly, DX-1000 down-regulated efficiently the expression and activation of gelatinases, directly involved in cancer cell invasion and ECM proteolysis. In addition, DX-1000 (1-10 nM) efficiently blocked tube formation of human and mouse endothelial cells whether plated on Matrigel or collagen type I. Concerning the haemostatic aspect, DX-1000 showed no clinically significant effects on global coagulation screening tests or a platelet function screening test.

DX-1000 can be modified, e.g., by pegylation. It has a three available lysines and an N-terminus for modification with mPEG, one, two, or more of these positions can be modified (e.g., all four of these positions can be modified). The compound 4×PEG DX-1000 is an exemplary modified DX-1000 molecule that includes four PEG moieties. DX-1000 can be combined with an mPEG succinimidyl propionic acid reagent having an average molecular weight of about 5 kDa or 7 kDa.

### DX-1000 Variants and Other Inhibitory Kunitz Domains

U.S. Pat. No. 6,103,499 describe additional plasmin inhibitors, including DX-1000 variants that inhibit plasmin. These Kunitz domains can also be used in the therapeutic methods described herein.

Exemplary plasmin-inhibitory amount of a protein comprising a Kunitz domain having the formula:

(SEQ ID NO: 300)
Xaa1-Xaa2-Xaa3-Xaa4-Cys-Xaa6-Xaa7-Xaa8-Xaa9-
Xaa10-Xaa11-Gly-Xaa13-Cys-Xaa15-Xaa16Xaa17-Xaa18-
Xaa19-Arg-Xaa21-Xaa22-Xaa23-Xaa24-Xaa25-Xaa26-
Xaa27-Xaa28-Xaa29-Cys-Xaa31-Xaa32-Phe-Xaa34-
Xaa35-Xaa36-Gly-Cys-Xaa39-Xaa40-Xaa41-Xaa42-
Xaa43-Xaa44-Xaa45-Xaa46-Xaa47-Xaa48-Xaa49-Xaa50-
Cys-Xaa52-Xaa53-Xaa54-Cys-Xaa56-Xaa57-Xaa58.

Xaa1, Xaa2, Xaa3, Xaa4, Xaa56, Xaa57 and/or Xaa58 may be absent. Xaa10 can be Asp, Glu, Tyr, or Gln. Xaa11 can be Thr, Ala, Ser, Val or Asp. Xaa13 can be Pro, Leu or Ala. Xaa15 can be Lys or Arg. Xaa16 can be Ala or Gly. Xaa17 can be Arg, Lys or Ser. Xaa18 can be Phe or Ile. Xaa19 can be Glu, Gln, Asp, Pro, Gly, Ser or Ile. Xaa21 can be Phe, Tyr or Trp. Xaa22 can be Tyr or Phe. Xaa23 can be Tyr or Phe. Xaa31 can be Asp, Glu, Thr, Val, Gln or Ala. Xaa32 can be Thr, Ala, Glu, Pro, or Gln. Xaa34 can be Val, Ile, Thr, Leu, Phe, Tyr, His, Asp, Ala, or Ser. Xaa35 can be Tyr or Trp. Xaa36 can be Gly or Ser. Xaa39 can be Glu, Gly, Asp, Arg, Ala, Gln, Leu, Lys, or Met. Xaa40 can be Gly or Ala. Xaa43 can be Asn or Gly; or Xaa45 can be Phe or Tyr. Where not specified, Xaa can be any amino acid, particularly any non-cysteine amino acid.

Further, Xaa10 can be Asp or Glu. Xaa11 can be Thr, Ala, or Ser. Xaa13 is Pro. Xaa15 is Arg. Xaa16 is Ala. Xaa17 is Arg. Xaa18 is Phe. Xaa19 can be Glu or Asp. Xaa21 can be Phe or Trp. Xaa22 can be Tyr or Phe. Xaa23 can be Tyr or Phe. Xaa31 can be Asp or Glu. Xaa32 can be Thr, Ala, or Glu. Xaa34 can be Val, Ile or Thr. Xaa35 is Tyr. Xaa36 is Gly. Xaa39 can be Glu, Gly, or Asp. Xaa40 can be Gly or Ala. Xaa43 can be Asn or Gly; or Xaa45 can be Phe or Tyr.

In one embodiment, the protein includes at least 80, 85, 90, or 95% of the amino acid of the first and/or second binding loops of DX-1000. In one embodiment, the protein includes a framework region from a human Kunitz domain (e.g., a human Kunitz domain described herein).

Exemplary DX-1000 variants include proteins that have an amino acid sequence that differs by at least one, but fewer than eight, six, five, four, three, or two amino acid differences (e.g., substitutions, insertions, or deletions) from the amino acid sequence of DX-1000 (e.g., SEQ ID NO:200) or the amino acid sequence of SEQ ID NO:100. The differences may be in regions other than the first binding loop, or in regions other than the first and second binding loops, e.g., in the framework region. Typically, the Kunitz domain does not naturally occur in humans, but may include an amino acid sequence that differs by fewer than ten, seven, or four amino acids from a human Kunitz domain (e.g., a human Kunitz domain described herein).

In one embodiment, the Kᵢ of the compound for plasmin is within a factor of 0.5 to 1.5, 0.8 to 1.2, 0.3 to 3.0, 0.1 to 10.0, or 0.02 to 50.0 of the Kᵢ of DX-1000 for plasmin.

DX-1000 includes a Kunitz domain that inhibits plasmin. DX-1000 and related Kunitz domains are described herein.

One class of plasmin inhibitors includes antibodies. Exemplary antibodies bind specifically to plasmin. An antibody can inhibit plasmin in a number of ways. For example, it can contact one or more residues of the active site, sterically hinder or obstruct access to the active site, prevent maturation of plasmin, or destabilize a conformation required for catalytic activity.

The binding ligand can include a peptide of 32 amino acids or less that independently binds to a target molecule (e.g., plasmin). Some such peptides can include one or more disulfide bonds. Other peptides, so-called “linear peptides,” are devoid of cysteines. In one embodiment, the peptides are artificial, i.e., not present in a nature or not present in a protein encoded by one or more genomes of interest, e.g., the human genome. Synthetic peptides may have little or no structure in solution (e.g., unstructured), heterogeneous structures (e.g., alternative conformations or “loosely” structured), or a singular native structure (e.g., cooperatively folded). Some synthetic peptides adopt a particular structure when bound to a target molecule. Some exemplary synthetic peptides are so-called “cyclic peptides” that have at least a disulfide bond and, for example, a loop of about 4 to 12 non-cysteine residues. Exemplary peptides are less than 28, 24, 20, or 18 amino acids in length.

### Protease Inhibitors—MMP Binding Proteins/Inhibitors

Other preferred protease inhibitor binding proteins include matrix metalloprotease binding proteins, or “MMP binding proteins”, such as those of MMP-9, MMP-12, MMP-14, and MMP-2. The binding proteins may in certain embodiments be antibody, peptide or Kunitz domain-based. Certain specific MMP binding proteins are described in further detail below.

### MMP-9 Binding Proteins

Any MMP-9 binding protein may be used in the methods and compositions for treating inflammatory disorders that are disclosed herein.

MMP-9 is encoded by a gene designated as MMP9 with full name Matrix metalloproteinase-9 precursor. Synonyms for MMP-9 include matrix metalloproteinase 9, gelatinase B (GELB), 92 kDa gelatinase (CLG4B), 92 kDa type IV collagenase (EC 3.4.24.35). The DNA sequence is known for Homo sapiens and Mus musculus. An exemplary cDNA sequence encoding human MMP9 and the amino acid sequence are shown below. Exemplary cDNA sequences encoding murine MMP9 and amino acid sequences are also shown below. An exemplary MMP-9 protein can include the human or mouse MMP-9 amino acid sequence, a sequence that is 80%, 85%, 90%, 95%, 96%, 97%, 98%, or 99% identical to one of these sequences, or a fragment thereof, e.g., a fragment without the signal sequence or prodomain.

Table 2 shows the similar genes in other organisms and the percentage of similarity with human MMP-9. No similarity-to-human data found for MMP-9 for: chimpanzee (Pan troglodytes), pig (Sus scrofa), cow (Bos taurus), fruit fly (Drosophila melanogaster), worm (Caenorhabditis elegans), baker's yeast (Saccharomyces cerevisiae), tropical clawed frog (Silurana tropicalis), African malaria mosquito (Anopheles gambiae), green algae (Chlamydomonas reinhardtii), soybean (Glycine max), barley (Hordeum vulgare), tomato (Lycopersicon esculentum), rice blast fungus (Magnaporthe grisea), sugarcane (Saccharum officinarum), loblolly pine (Pinus taeda), corn (Zea mays), wheat (Triticum aestivum), Alicante grape (Vitis vinifera), bread mold (Neurospora crassa), fission yeast (Schizosaccharomyces pombe), sea squirt (Ciona intestinalis), amoeba (Dictyostelium discoideum), A. gosspyii yeast (Ashbya gossypii), K. lactis yeast (Kluyveromyces lactis), medicago trunc (Medicago truncatula), malaria parasite (Plasmodium falciparum), schistosome parasite (Schistosoma mansoni), sorghum (Sorghum bicolor), toxoplasmosis (Toxoplasma gondii).

| cDNA and amino acid sequences of human MMP9 | |
|---|---|
| ACCESSION AK123156 | |
| VERSION AK123156.1 GI: 34528630 | |
| (SEQ ID NO: 26) | |
| translation = “MARKGARRPRQGPGSHKWLQPGSRREKERIPQPPPPARPPRDAAPRRVLVPAV | |
| RRVPESGHFAGRPWAPQCHPKGLRRPSAESHSVAQAGVQCHDLGSLQPPPPSSGDSPASASRVAGITS | |
| TVPGTLSALDDCCLITELPYKPPAVLY” | |
| (SEQ ID NO: 27) | |
| 1 | acactttgcg ttccgcggcc ccggcccctt ggtttcctag tcctggctcc attcccctct |
| 61 | caggcctagg gctgggaccc ctccccgccc ccggtcttgg ccctgccccc ttcaacagac |
| 121 | ggtccgcccc ggcccctccc cctcgtcccg cccggccctg gcaggccccg ccccctgcgg |
| 181 | cctctacctt tgacgtcttc ccccgggagg tggcgggggt ctgcgaccga atgccggcgg |
| 241 | gactctgggt cagggcttct ggcgggccct gcggggggca gcgaggtgac cgtgaacctg |
| 301 | cggctcatgg cgcggaaagg agccaggcgg ccgcggcaag gtccgggatc gcacaagtgg |
| 361 | ctgcaaccag gctctaggag ggagaaagag cggatccccc aaccccctcc gcccgcccgc |
| 421 | cccccgcgag acgcggcgcc gcgcagggtc ctagtgcccg ctgtgcgaag ggttcctgaa |
| 481 | tctggccact tcgctgggag gccctgggct ccccagtgcc acccgaaggg cctgaggagg |
| 541 | ccatctgcag aatctcactc tgtcgcccag gccggagtgc agtgtcatga tcttggctca |
| 601 | ctgcaacctc cgcctcccag ttcaggagat tctcctgcct cagcctcccg ggtggctggg |
| 661 | attacaagca cagtgcctgg cacattatcg gcacttgatg actgttgtct aataactgag |
| 721 | cttccataca aaccacctgc cgtcctgtac tgaaggagaa agagcttcca gccggggagg |
| 781 | caggaaatct gggtcctggt cttggttgca tccctgactt cctaaatgac ctggagaagg |
| 841 | cctctgcctc tgctgggatc ttgtctgtgc tggggcattt gtttccattt ccaagggctt |
| 901 | tttcttcctc gctcagaatt tgaccactca ctaagaggag cttagtgtgg tgtctcacga |
| 961 | agggatcctc ctcagccctc acctcggtac tggaagacgt cgtgcgtgtc caaaggcacc |
| 1021 | ccggggaaca tccggtccac ctcgctggcg ctccggggat ccaccatctg cgccttcacg |
| 1081 | tcgaacctgc gggcaggcgc ggaggagaca ggtgctgagc cggctagcgg acggaccgac |
| 1141 | ggcgcccggg ctccccctgc cggcggccgc ggcggcgctc acctccagag gcgccgcccg |
| 1201 | ctgaacagca gcatcttccc cctgccactc cggagggccc cggtcacctg ggccacgtcg |
| 1261 | gcgcccaggc ccagcttgtc cagacgcctc gggcccagca ccgacgcgcc tgtgtacacc |
| 1321 | cacacctggc gccctgcagg ggaggagggt cacgtcggtt tgggggcgca gagggagcac |
| 1381 | gtactcctag aacgcgagga gggagattcc ggcgaggcct ttcctagccc gcgtgcccgc |
| 1441 | agtccctgca acccaggggc agaggcgctg ggtagagcga cgcgagggcg tggagaggag |
| 1501 | ggggcagaaa ctcagccgcc cctacgtttg ctaaactgcg tccgccaggg ggcgtatttt |
| 1561 | tctaaaacgc acaagacgtt tcgtgggtta tcgatggtct cttgagcctc cttgactgat |
| 1621 | ggggattgac cgggcggggg agggaaagta ggtaactaac cagagaagaa gaaaagcttc |
| 1681 | ttggagagcg gctcctcaaa gaccgagtcc agcttgcggg gcagcgcggg ccacttgtcg |
| 1741 | gcgataagga aggggccctg cggccggctc cccctgccct cagagaatcg ccagtacttc |
| 1801 | ctgagaaagc gaggagggaa aggacgggct ctaagccttg gacacagggc cagtgggcgg |
| 1861 | gaagggacgg gcagcccctc cgcaaagccc cctcccgcat ccacacaacc ccgcctcctc |
| 1921 | acccatcctt gaacaaatac agctggttcc caatc |
| cDNA and amino acid sequences of mouse MMP9 | |
| ACCESSION NM_013599 | |
| VERSION NM_013599.2 GI: 31560795 | |
| (SEQ ID NO: 28) | |
| translation = “MSPWQPLLLALLAFGCSSAAPYQRQPTFVVFPKDLKTSNLTDTQLAEAYLYRY | |
| GYTRAAQMMGEKQSLRPALLMLQKQLSLPQTGELDSQTLKAIRTPRCGVPDVGRFQTFKGLKWDHHNI | |
| TYWIQNYSEDLPRDMIDDAFARAFAVWGEVAPLTFTRVYGPEADIVIQFGVAEHGDGYPFDGKDGLLA | |
| HAFPPGAGVQGDAHFDDDELWSLGKGVVIPTYYGNSNGAPCHFPFTFEGRSYSACTTDGRNDGTPWCS | |
| TTADYDKDGKFGFCPSERLYTEHGNGEGKPCVFPFIFEGRSYSACTTKGRSDGYRWCATTANYDQDKL | |
| YGFCPTRVDATVVGGNSAGELCVFPFVFLGKQYSSCTSDGRRDGRLWCATTSNFDTDKKWGFCPDQGY | |
| SLFLVAAHEFGHALGLDHSSVPEALMYPLYSYLEGFPLNKDDIDGIQYLYGRGSKPDPRPPATTTTEP | |
| QPTAPPTMCPTIPPTAYPTVGPTVGPTGAPSPGPTSSPSPGPTGAPSPGPTAPPTAGSSEASTESLSP | |
| ADNPCNVDVFDAIAEIQGALHFFKDGWYWKFLNHRGSPLQGPFLTARTWPALPATLDSAFEDPQTKRV | |
| FFFSGRQMWVYTGKTVLGPRSLDKLGLGPEVTHVSGLLPRRLGKALLFSKGRVWRFDLKSQKVDPQSV | |
| IRVDKEFSGVPWNSHDIFQYQDKAYFCHGKFFWRVSFQNEVNKVDHEVNQVDDVGYVTYDLLQCP” | |
| (SEQ ID NO: 29) | |
| 1 | ctcaccatga gtccctggca gcccctgctc ctggctctcc tggctttcgg ctgcagctct |
| 61 | gctgcccctt accagcgcca gccgactttt gtggtcttcc ccaaagacct gaaaacctcc |
| 121 | aacctcacgg acacccagct ggcagaggca tacttgtacc gctatggtta cacccgggcc |
| 181 | gcccagatga tgggagagaa gcagtctcta cggccggctt tgctgatgct tcagaagcag |
| 241 | ctctccctgc cccagactgg tgagctggac agccagacac taaaggccat tcgaacacca |
| 301 | cgctgtggtg tcccagacgt gggtcgattc caaaccttca aaggcctcaa gtgggaccat |
| 361 | cataacatca catactggat ccaaaactac tctgaagact tgccgcgaga catgatcgat |
| 421 | gacgccttcg cgcgcgcctt cgcggtgtgg ggcgaggtgg cacccctcac cttcacccgc |
| 481 | gtgtacggac ccgaagcgga cattgtcatc cagtttggtg tcgcggagca cggagacggg |
| 541 | tatcccttcg acggcaagga cggccttctg gcacacgcct ttccccctgg cgccggcgtt |
| 601 | cagggagatg cccatttcga cgacgacgag ttgtggtcgc tgggcaaagg cgtcgtgatc |
| 661 | cccacttact atggaaactc aaatggtgcc ccatgtcact ttcccttcac cttcgaggga |
| 721 | cgctcctatt cggcctgcac cacagacggc cgcaacgacg gcacgccttg gtgtagcaca |
| 781 | acagctgact acgataagga cggcaaattt ggtttctgcc ctagtgagag actctacacg |
| 841 | gagcacggca acggagaagg caaaccctgt gtgttcccgt tcatctttga gggccgctcc |
| 901 | tactctgcct gcaccactaa aggccgctcg gatggttacc gctggtgcgc caccacagcc |
| 961 | aactatgacc aggataaact gtatggcttc tgccctaccc gagtggacgc gaccgtagtt |
| 1021 | gggggcaact cggcaggaga gctgtgcgtc ttccccttcg tcttcctggg caagcagtac |
| 1081 | tcttcctgta ccagcgacgg ccgcagggat gggcgcctct ggtgtgcgac cacatcgaac |
| 1141 | ttcgacactg acaagaagtg gggtttctgt ccagaccaag ggtacagcct gttcctggtg |
| 1201 | gcagcgcacg agttcggcca tgcactgggc ttagatcatt ccagcgtgcc ggaagcgctc |
| 1261 | atgtacccgc tgtatagcta cctcgagggc ttccctctga ataaagacga catagacggc |
| 1321 | atccagtatc tgtatggtcg tggctctaag cctgacccaa ggcctccagc caccaccaca |
| 1381 | actgaaccac agccgacagc acctcccact atgtgtccca ctatacctcc cacggcctat |
| 1441 | cccacagtgg gccccacggt tggccctaca ggcgccccct cacctggccc cacaagcagc |
| 1501 | ccgtcacctg gccctacagg cgccccctca cctggcccta cagcgccccc tactgcgggc |
| 1561 | tcttctgagg cctctacaga gtctttgagt ccggcagaca atccttgcaa tgtggatgtt |
| 1621 | tttgatgcta ttgctgagat ccagggcgct ctgcatttct tcaaggacgg ttggtactgg |
| 1681 | aagttcctga atcatagagg aagcccatta cagggcccct tccttactgc ccgcacgtgg |
| 1741 | ccagccctgc ctgcaacgct ggactccgcc tttgaggatc cgcagaccaa gagggttttc |
| 1801 | ttcttctctg gacgtcaaat gtgggtgtac acaggcaaga ccgtgctggg ccccaggagt |
| 1861 | ctggataagt tgggtctagg cccagaggta acccacgtca gcgggcttct cccgcgtcgt |
| 1921 | ctcgggaagg ctctgctgtt cagcaagggg cgtgtctgga gattcgactt gaagtctcag |
| 1981 | aaggtggatc cccagagcgt cattcgcgtg gataaggagt tctctggtgt gccctggaac |
| 2041 | tcacacgaca tcttccagta ccaagacaaa gcctatttct gccatggcaa attcttctgg |
| 2101 | cgtgtgagtt tccaaaatga ggtgaacaag gtggaccatg aggtgaacca ggtggacgac |
| 2161 | gtgggctacg tgacctacga cctcctgcag tgcccttgaa ctagggctcc ttctttgctt |
| 2221 | caaccgtgca gtgcaagtct ctagagacca ccaccaccac caccacacac aaaccccatc |
| 2281 | cgagggaaag gtgctagctg gccaggtaca gactggtgat ctcttctaga gactgggaag |
| 2341 | gagtggaggc aggcagggct ctctctgccc accgtccttt cttgttggac tgtttctaat |
| 2401 | aaacacggat ccccaacctt ttccagctac tttagtcaat cagcttatct gtagttgcag |
| 2461 | atgcatccga gcaagaagac aactttgtag ggtggattct gaccttttat ttttgtgtgg |
| 2521 | cgtctgagaa ttgaatcagc tggcttttgt gacaggcact tcaccggcta aaccacctct |
| 2581 | cccgactcca gcccttttat ttattatgta tgaggttatg ttcacatgca tgtatttaac |
| 2641 | ccacagaatg cttactgtgt gtcgggcgcg gctccaaccg ctgcataaat attaaggtat |
| 2701 | tcagttgccc ctactggaag gtattatgta actatttctc tcttacattg gagaacacca |
| 2761 | ccgagctatc cactcatcaa acatttattg agagcatccc tagggagcca ggctctctac |
| 2821 | tgggcgttag ggacagaaat gttggttctt ccttcaagga ttgctcagag attctccgtg |
| 2881 | tcctgtaaat ctgctgaaac cagaccccag actcctctct ctcccgagag tccaactcac |
| 2941 | tcactgtggt tgctggcagc tgcagcatgc gtatacagca tgtgtgctag agaggtagag |
| 3001 | ggggtctgtg cgttatggtt caggtcagac tgtgtcctcc aggtgagatg acccctcagc |
| 3061 | tggaactgat ccaggaagga taaccaagtg tcttcctggc agtctttttt aaataaatga |
| 3121 | ataaatgaat atttacttaa aaaaaaaaaa aaaaaaaaaa aaaaaaaaaa aaaaaaaaaa |
| 3181 | aaaaa |
| ACCESSION NP_038627 | |
| VERSION NP_038627.1 GI: 7305277 | |
| (SEQ ID NO: 30) | |
| 1 | mspwqpllla llafgcssaa pyqrqptfvv fpkdlktsnl tdtqlaeayl yrygytraaq |
| 61 | mmgekqslrp allmlqkqls lpqtgeldsq tlkairtprc gvpdvgrfqt fkglkwdhhn |
| 121 | itywiqnyse dlprdmidda farafavwge vapltftrvy gpeadiviqf gvaehgdgyp |
| 181 | fdgkdgllah afppgagvqg dahfdddelw slgkgvvipt yygnsngapc hfpftfegrs |
| 241 | ysacttdgrn dgtpwcstta dydkdgkfgf cpserlyteh gngegkpcvf pfifegrsys |
| 301 | acttkgrsdg yrwcattany dqdklygfcp trvdatvvgg nsagelcvfp fvflgkqyss |
| 361 | ctsdgrrdgr lwcattsnfd tdkkwgfcpd qgyslflvaa hefghalgld hssvpealmy |
| 421 | plysylegfp lnkddidgiq ylygrgskpd prppatttte pqptapptmc ptipptaypt |
| 481 | vgptvgptga pspgptssps pgptgapspg ptapptagss easteslspa dnpcnvdvfd |
| 541 | aiaeiqgalh ffkdgwywkf lnhrgsplqg pfltartwpa lpatldsafe dpqtkrvfff |
| 601 | sgrqmwvytg ktvlgprsld klglgpevth vsgllprrlg kallfskgrv wrfdlksqkv |
| 661 | dpqsvirvdk efsgvpwnsh difqyqdkay fchgkffwrv sfqnevnkvd hevnqvddvg |
| 721 | yvtydllqcp |

TABLE 2
MMP-9 orthologs from nine species

| | | | | Human | |
|---|---|---|---|---|---|
| Organism | Gene | Locus | Description | Similarity | NCBI accessions |
| dog | MMP9¹ | — | matrix metallopeptidase 9 | 85.46(n) | 403885 NM_001003219.1 |
| (Canis | | | (gelatinase B, 92 kDa | 80.97(a) | NP_001003219.1 |
| familiaris) | | | gelatinase | | |
| rat (Rattus | Mmp9¹ | — | matrix metallopeptidase 9 | 79.15(n) | 81687 NM_031055.1 |
| norvegicus) | | | | 74.89(a) | NP_112317.1 |
| mouse | Mmp9^{1, 4} | 2 (96.00 cM)⁴ | matrix metallopeptidase 9^{1, 4} | 78.69(n)¹ | 17395¹ NM_013599.2¹ |
| (Mus | | | | 75(a)¹ | NP_038627.1¹ |
| musculus) | | | | | AK004651⁴ AK142787⁴ |
| | | | | | (see all 16) |
| chicken | LOC395387¹ | — | matrix metallopeptidase 9 | 66.96(n) | 395387 NM_204667.1 |
| (Gallus | | | (gelatinase B, 92 kDa | 62.54(a) | NP_989998.1 |
| gallus) | | | gelatinase | | |
| zebrafish | wufb02g06^{1~} | — | Danio rerio cDNA clone | 70.96(n) | BC053292.1 |
| (Danio rerio) | | | MGC64165 | | |
| | | | IMAGE6797338, | | |
| | | | complete | | |
| African | MGC69080^{1~} | — | hypothetical protein | 72.25(n) | BC057745.1 |
| clawed frog | | | MGC69080 | | |
| (Xenopus | | | | | |
| laevis) | | | | | |
| rainbow | Omy.10476^{1~} | — | Oncorhynchus mykiss | 74.67(n) | AJ320533.1 |
| trout | | | mRNA for matrix | | |
| (Oncorhynchus | | | metalloproteinase | | |
| mykiss) | | | | | |
| thale cress | MMP¹ | — | MMP (MATRIX | 53(n) | 843353 NM_105685.3 |
| (Arabidopsis | | | METALLOPROTEINASE); | 46.85(a) | NP_177174.1 |
| thaliana) | | | metalloendopeptidase/ | | |
| rice (Oryza | P0516G10.18¹ | — | putative zinc | 51.98(n) | 3063368 XM_467714.1 |
| sativa) | | | metalloproteinase | 41.81(a) | XP_467714.1 |

Domains of MMP-9. MMP-9 belongs to the peptidase M10A family. MMP-9 consists of five domains; the amino-terminal and zinc-binding domains shared by all members of the secreted metalloprotease gene family, the collagen-binding fibronectin-like domain also present in the 72-kDa type IV collagenase, a carboxyl-terminal hemopexin-like domain shared by all known enzymes of this family with the exception of PUMP-1, and a unique 54-amino-acid-long proline-rich domain homologous to the alpha 2 chain of type V collagen (Wilhelm et al. (1989) J. Biol. Chem. 264, 17213-17221) (Table 3).

TABLE 3
MMP-9 domains

| | | | | |
|---|---|---|---|---|
| FT | SIGNAL | 1 | 19 | |
| FT | PROPEP | 20 | 93 | Activation peptide. |
| FT | CHAIN | 94 | ? | 67 kDa matrix metalloproteinase-9. |
| FT | CHAIN | 107 | 707 | 82 kDa matrix metalloproteinase-9. |
| FT | PROPEP | ? | 707 | Removed in 64 kDa matrix |
| FT | | | | metalloproteinase-9 and 67 kDa |
| | | | | matrix |
| FT | | | | metalloproteinase-9. |
| FT | DOMAIN | 225 | 273 | Fibronectin type-II 1. |
| FT | DOMAIN | 283 | 331 | Fibronectin type-II 2. |
| FT | DOMAIN | 342 | 390 | Fibronectin type-II 3. |
| FT | DOMAIN | 513 | 707 | Hemopexin-like. |
| FT | ACT_SITE | 402 | 402 | |
| FT | METAL | 131 | 131 | Calcium 1. |
| FT | METAL | 165 | 165 | Calcium 2 (via carbonyl oxygen). |
| FT | METAL | 175 | 175 | Zinc 1 (structural). |
| FT | METAL | 177 | 177 | Zinc 1 (structural). |
| FT | METAL | 182 | 182 | Calcium 3. |
| FT | METAL | 183 | 183 | Calcium 3 (via carbonyl oxygen). |
| FT | METAL | 185 | 185 | Calcium 3 (via carbonyl oxygen). |
| FT | METAL | 187 | 187 | Calcium 3 (via carbonyl oxygen). |
| FT | METAL | 190 | 190 | Zinc 1 (structural). |
| FT | METAL | 197 | 197 | Calcium 2 (via carbonyl oxygen). |
| FT | METAL | 199 | 199 | Calcium 2 (via carbonyl oxygen). |
| FT | METAL | 201 | 201 | Calcium 2. |
| FT | METAL | 203 | 203 | Zinc 1 (structural). |
| FT | METAL | 205 | 205 | Calcium 3. |
| FT | METAL | 206 | 206 | Calcium 1. |
| FT | METAL | 208 | 208 | Calcium 1. |
| FT | METAL | 208 | 208 | Calcium 3. |
| FT | METAL | 401 | 401 | Zinc 2 (catalytic). |
| FT | METAL | 405 | 405 | Zinc 2 (catalytic). |
| FT | METAL | 411 | 411 | Zinc 2 (catalytic). |
| FT | SITE | 59 | 60 | Cleavage (by MMP3). |
| FT | SITE | 99 | 99 | Cysteine switch (By similarity). |
| FT | SITE | 106 | 107 | Cleavage (by MMP3). |
| FT | CARBOHYD | 38 | 38 | N-linked (GlcNAc . . .) (Potential). |
| FT | CARBOHYD | 120 | 120 | N-linked (GlcNAc . . .) (Potential). |
| FT | CARBOHYD | 127 | 127 | N-linked (GlcNAc . . .) (Potential). |
| FT | DISULFID | 230 | 256 | By similarity. |
| FT | DISULFID | 244 | 271 | By similarity. |
| FT | DISULFID | 288 | 314 | By similarity. |
| FT | DISULFID | 302 | 329 | By similarity. |
| FT | DISULFID | 347 | 373 | By similarity. |
| FT | DISULFID | 361 | 388 | By similarity. |
| FT | DISULFID | 516 | 704 | |
| FT | VARIANT | 20 | 20 | A −> V (in dbSNP: rs1805088). |
| FT | VARIANT | 82 | 82 | E −> K (in dbSNP: rs1805089). |
| FT | VARIANT | 127 | 127 | N −> K (in dbSNP: rs3918252). |
| FT | VARIANT | 239 | 239 | R −> H. |
| FT | VARIANT | 279 | 279 | R −> Q (common polymorphism; |
| FT | | | | dbSNP: rs17576). |
| FT | VARIANT | 571 | 571 | F −> V. |
| FT | VARIANT | 574 | 574 | P −> R (in dbSNP: rs2250889). |
| FT | VARIANT | 668 | 668 | R −> Q (in dbSNP: rs17577). |
| FT | TURN | 32 | 33 | |
| FT | HELIX | 41 | 51 | |
| FT | TURN | 52 | 53 | |
| FT | HELIX | 68 | 78 | |
| FT | TURN | 79 | 79 | |
| FT | HELIX | 88 | 94 | |
| FT | TURN | 95 | 95 | |
| FT | STRAND | 103 | 105 | |
| FT | STRAND | 119 | 125 | |
| FT | STRAND | 130 | 132 | |
| FT | HELIX | 134 | 149 | |
| FT | TURN | 150 | 150 | |
| FT | STRAND | 151 | 153 | |
| FT | STRAND | 155 | 158 | |
| FT | TURN | 162 | 163 | |
| FT | STRAND | 164 | 171 | |
| FT | STRAND | 176 | 178 | |
| FT | STRAND | 183 | 186 | |
| FT | STRAND | 189 | 191 | |
| FT | STRAND | 194 | 196 | |
| FT | TURN | 197 | 200 | |
| FT | STRAND | 202 | 205 | |
| FT | TURN | 206 | 207 | |
| FT | STRAND | 213 | 219 | |
| FT | HELIX | 220 | 231 | |
| FT | TURN | 232 | 233 | |
| FT | TURN | 240 | 241 | |
| FT | TURN | 243 | 244 | |
| FT | STRAND | 245 | 247 | |
| FT | STRAND | 255 | 261 | |
| FT | HELIX | 262 | 265 | |
| FT | STRAND | 268 | 270 | |
| FT | TURN | 274 | 276 | |
| FT | STRAND | 279 | 283 | |
| FT | TURN | 284 | 285 | |
| FT | STRAND | 290 | 294 | |
| FT | TURN | 295 | 296 | |
| FT | STRAND | 297 | 301 | |
| FT | TURN | 305 | 306 | |
| FT | STRAND | 313 | 319 | |
| FT | HELIX | 320 | 323 | |
| FT | STRAND | 326 | 328 | |
| FT | HELIX | 333 | 335 | |
| FT | TURN | 340 | 344 | |
| FT | STRAND | 349 | 353 | |
| FT | TURN | 354 | 355 | |
| FT | STRAND | 356 | 358 | |
| FT | TURN | 364 | 365 | |
| FT | STRAND | 372 | 378 | |
| FT | HELIX | 379 | 382 | |
| FT | STRAND | 385 | 387 | |
| FT | HELIX | 395 | 406 | |
| FT | TURN | 407 | 408 | |
| FT | TURN | 415 | 416 | |
| FT | TURN | 418 | 419 | |
| FT | HELIX | 433 | 442 | |
| FT | STRAND | 512 | 517 | |
| FT | HELIX | 515 | 517 | |
| FT | STRAND | 522 | 527 | |
| FT | TURN | 528 | 529 | |
| FT | STRAND | 530 | 535 | |
| FT | TURN | 536 | 537 | |
| FT | STRAND | 538 | 542 | |
| FT | STRAND | 545 | 547 | |
| FT | STRAND | 551 | 555 | |
| FT | HELIX | 556 | 559 | |
| FT | TURN | 561 | 562 | |
| FT | STRAND | 568 | 572 | |
| FT | TURN | 574 | 576 | |
| FT | STRAND | 579 | 583 | |
| FT | TURN | 584 | 585 | |
| FT | STRAND | 586 | 591 | |
| FT | TURN | 592 | 593 | |
| FT | STRAND | 594 | 600 | |
| FT | HELIX | 601 | 604 | |
| FT | TURN | 605 | 605 | |
| FT | TURN | 608 | 609 | |
| FT | STRAND | 615 | 618 | |
| FT | TURN | 621 | 622 | |
| FT | STRAND | 623 | 628 | |
| FT | TURN | 629 | 630 | |
| FT | STRAND | 631 | 636 | |
| FT | TURN | 637 | 640 | |
| FT | HELIX | 644 | 646 | |
| FT | HELIX | 650 | 653 | |
| FT | TURN | 655 | 656 | |
| FT | STRAND | 662 | 667 | |
| FT | TURN | 668 | 669 | |
| FT | STRAND | 670 | 675 | |
| FT | TURN | 676 | 677 | |
| FT | STRAND | 678 | 683 | |
| FT | TURN | 686 | 687 | |
| FT | STRAND | 690 | 696 | |
| FT | TURN | 697 | 700 | |
| FT | TURN | 702 | 703 | |

The catalytic activity of MMP-9 is inhibited by histatin-3 1/24 (histatin-5). MMP-9 is activated by urokinase-type plasminogen activator; plasminogen; IL-1beta, 4-aminophenylmercuric acetate and phorbol ester. MMP-9 exists as monomer, disulfide-linked homodimer, and as a heterodimer with a 25 kDa protein. Macrophages and transformed cell lines produce only the monomeric MMP-9, the hetrodimeric form is produced by normal alveolar macrophages and granulocytes. The processing of the precursor yields different active forms of 64, 67 and 82 kDa. Sequentially processing by MMP-3 yields the 82 kDa matrix metalloproteinase-9. In arthritis patients, this enzyme can contribute to the pathogenesis of joint destruction and can be a useful marker of disease status.

Endogenous inhibitors of MMP-9. MMP-9 has a number of endogenous inhibitors Like other MMPs, MMP-9 is inhibited by TIMPs (Murphy, G., and Willenbrock, F. (1995) Methods Enzymol. 248, 496-510). A characteristic of MMP-9 (and MMP-2) is the ability of their zymogens to form tight non-covalent and stable complexes with TIMPs. It has been shown that pro-MMP-2 binds TIMP-2 (Goldberg et al. (1989) Proc. Natl. Acad. Sci. U.S.A. 86, 8207-8211), whereas pro-MMP-9 binds TIMP-1 (Wilhelm et al. (1989) J. Biol. Chem. 264, 17213-17221). TIMPs typically are slow, tight binding inhibitors. A MMP-9 binding protein (e.g., antibody, peptide, Kunitz domain) selected from a library of phage-displayed proteins can be selected have more rapid kinetics. For example, recombinant TIMP-1 can be administered to inhibit MMP-9, e.g., in combination with a MMP-9 binding protein decscribed herein.

Small molecule inhibitors of MMP-9. Skiles et al. (2004, Curr Med Chem, 11:2911-77) reported that first generation small-molecule MMP inhibitors had poor bioavailability and the second generation had caused musculoskeletal pain and inflammation. Most small-molecule MMP inhibitors interact with the catalytic zinc but have fairly low affinity. Thus, a higher concentration is needed to have effect. The interaction with the catalytic zinc leads to inhibition of other MMPs and toxic side effects. A MMP-9 binding protein described herein can be used in combination with a small molecule inhibitor. For example, because the inhibitors are used in combination, the dose of the small molecule used can be decreased and therefore result in fewer side effects. Examples of small molecule MMP-9 inhibitors include small synthetic anthranilic acid-based inhibitors (see, e.g., Calbiochem Inhibitor-I, catalogue #444278 and Levin et al., 2001, Bioorg. Med. Chem. Lett. 11:2975-2978).

Small interfering RNA inhibitors of MMP-9. MMP-9 can be inhibited by small interfering RNA (siRNA). Examples of siRNA that can be used include:

MMP-9 siRNA

| | |
|---|---|
| 5′-GACUUGCCGCGAGACAUGAtt-3′ | (SEQ ID NO: 31) |
| 3′-ttCUGAACGGCGCUCUGUACU-5′ | (SEQ ID NO: 32) |

Control RNA (mismatch)

| | |
|---|---|
| 5′-GACUUCGCGGGACACAUGAtt-3′ | (SEQ ID NO: 34) |
| 3′-ttCUGAAGCGCCCUGUGUACU-5′ | (SEQ ID NO: 35) |

See also Kawasaki et al., Feb. 10, 2008, Nat. Med. advance on-line publication doi:10.1038/nm1723. The siRNA can be administered to inhibit MMP-9, e.g., in combination with a MMP-9 binding protein decscribed herein.

MMP-9 Binding Proteins

Provided are proteins that bind to MMP-9 (e.g., human MMP-9) and are either peptides, polypeptides that include at least one immunoglobin variable region, or Kunitz domains. Methods for discovering and selecting and improving such binding proteins are described further below.

In a preferred embodiment, the MMP-9 binding protein includes at least one immunoglobulin variable domain. For example, the MMP-9 binding protein includes a heavy chain (HC) immunoglobulin variable domain sequence and a light chain (LC) immunoglobulin variable domain sequence. A number of exemplary MMP-9 binding proteins are described herein. MMP-9 binding proteins may be antibodies. MMP-9 binding antibodies may have their HC and LC variable domain sequences included in a single polypeptide (e.g., scFv), or on different polypeptides (e.g., IgG or Fab).

The MMP-9 binding protein may be an isolated peptide or protein (e.g., at least 70, 80, 85, 90, 91, 92, 93, 94, 95, 96, 97, 98, or 99% free of other proteins).

The MMP-9 binding protein may additionally inhibit MMP-9, e.g., human MMP-9. The binding protein can inhibit the catalytic activity of MMP-9 (e.g., human MMP-9). In one embodiment, the protein binds the catalytic domain of human MMP-9, e.g., the protein contacts residues in or near the active site of MMP-9. In some embodiments, the protein does not contact residues in or near the active site of MMP-9 but instead binds elsewhere on MMP-9 and causes a steric change in MMP-9 that affects (e.g., inhibits) its activity.

The protein can bind to MMP-9, e.g., human MMP-9, with a binding affinity of at least 10⁵, 10⁶, 10⁷, 10⁸, 10⁹, 10¹⁰ and 10¹¹ M⁻¹. In one embodiment, the protein binds to MMP-9 with a K_{off} slower than 1×10⁻³, 5×10⁻⁴ s⁻¹, or 1×10⁻⁴ s⁻¹. In one embodiment, the protein binds to MMP-12 with a Kₒₙ faster than 1×10², 1×10³, or 5×10³ M⁻¹ s⁻¹. In one embodiment, the protein inhibits human MMP-9 activity, e.g., with a Ki of less than 10⁻⁵, 10⁻⁶, 10⁻⁷, 10⁻⁸, 10⁻⁹, and 10⁻¹⁰ M. The protein can have, for example, an IC50 of less than 100 nM, 10 nM or 1 nM. In some embodiments, the protein has an IC50 of about 1.8 nM. The affinity of the protein for MMP-9 can be characterized by a K_{D} of less than 100 nm, less than 10 nM, or about 3 nM (e.g., 3.1 nM), about 5 nM (e.g., 5 nM), about 6 nm (e.g., 5.9 nM), about 7 nM (e.g., 7.1 nM), or about 10 nM (e.g., 9.6 nM).

In some embodiments, the protein has a K_{D}<200 nM.

In some embodiments, the protein has a t½ of at least about 10 minutes (e.g., 11 minutes), at least about 20 minutes (e.g., 18 minutes), at least about 25 minutes (e.g., 25 minutes), at least about 35 minutes (e.g., 33 minutes), or at least about 60 minutes (e.g., 57 minutes).

In one embodiment, the protein binds the catalytic domain of human MMP-9, e.g., the protein contacts residues in or near the active site of MMP-9.

In some embodiments, the protein does not contact residues in or near the active site of MMP-9 but instead binds elsewhere on MMP-9 and causes a steric change in MMP-9 that affects (e.g., inhibits) its activity.

Exemplary MMP-9 binding proteins include antibodies with a heavy chain (HC) and/or light chain (LC), and in some embodiments, an HC and/or LC variable domain, that is selected from the group of antibodies consisting of: DX-2802, 539A-M0240-B03, M0078-G07, M0081-D05, M0076-D03, M0072-H07, M0075-D12, M0166-F10, M0279-A03, M0279-B02, M0288-008, and M0281-F06, or proteins that comprise the HC and/or LC CDRs of DX-2802, 539A-M0240-B03, M0078-G07, M0081-D05, M0076-D03, M0072-H07, M0075-D12, M0166-F10, M0279-A03, M0279-B02, M0288-008, or M0281-F06. These MMP-9 binding proteins are further described in U.S. Ser. No. 61/033,075, filed Mar. 3, 2008 and U.S. Ser. No. 61/054,938, filed May 21, 2008, and WO 2009/111450.

The protein, if an antibody, can include one or more of the following characteristics: (a) a human CDR or human framework region; (b) the HC immunoglobulin variable domain sequence comprises one or more CDRs that are at least 85, 88, 90, 91, 92, 93, 94, 95, 96, 97, 98, 99, or 100% identical to a CDR of a HC variable domain described above; (c) the LC immunoglobulin variable domain sequence comprises one or more CDRs that are at least 85, 88, 90, 91, 92, 93, 94, 95, 96, 97, 98, 99, or 100% identical to a CDR of a LC variable domain described above; (d) the LC immunoglobulin variable domain sequence is at least 85, 88, 90, 91, 92, 93, 94, 95, 96, 97, 98, 99, or 100% identical to a LC variable domain described above; (e) the HC immunoglobulin variable domain sequence is at least 85, 88, 90, 91, 92, 93, 94, 95, 96, 97, 98, 99, or 100% identical to a HC variable domain described above; (f) the protein binds an epitope bound by a protein described herein, or an epitope that overlaps with such epitope; and (g) a primate CDR or primate framework region.

In one embodiment, the HC and LC variable domain sequences are components of the same polypeptide chain. In another, the HC and LC variable domain sequences are components of different polypeptide chains. For example, the protein is an IgG, e.g., IgG1, IgG2, IgG3, or IgG4. The protein can be a soluble Fab (sFab). In other implementations the protein includes a Fab2′, scFv, minibody, scFv::Fc fusion, Fab::HSA fusion, HSA::Fab fusion, Fab::HSA::Fab fusion, or other molecule that comprises the antigen combining site of one of the binding proteins herein. The VH and VL regions of these Fabs can be provided as IgG, Fab, Fab2, Fab2′, scFv, PEGylated Fab, PEGylated scFv, PEGylated Fab2, VH::CH1::HSA+LC, HSA::VH::CH1+LC, LC::HSA+VH::CH1, HSA::LC+VH::CH1, or other appropriate construction.

In one embodiment, the protein is a human or humanized antibody or is non-immunogenic in a human. For example, the protein includes one or more human antibody framework regions, e.g., all human framework regions. In one embodiment, the protein includes a human Fc domain, or an Fc domain that is at least 95, 96, 97, 98, or 99% identical to a human Fc domain.

In one embodiment, the protein is a primate or primatized antibody or is non-immunogenic in a human. For example, the protein includes one or more primate antibody framework regions, e.g., all primate framework regions. In one embodiment, the protein includes a primate Fc domain, or an Fc domain that is at least 95, 96, 97, 98, or 99% identical to a primate Fc domain. “Primate” includes humans (Homo sapiens), chimpanzees (Pan troglodytes and Pan paniscus (bonobos)), gorillas (Gorilla gorilla), gibons, monkeys, lemurs, aye-ayes (Daubentonia madagascariensis), and tarsiers.

In certain embodiments, the protein includes no sequences from mice or rabbits (e.g., is not a murine or rabbit antibody).

### MMP-9/MMP-2 Binding Proteins

Any MMP-9/2 binding protein may be used in the methods and compositions for treating inflammatory disorders that are disclosed herein. MMP-9/2 binding proteins are binding proteins that bind to MMP-9 (e.g., human MMP-9) and MMP-2 (e.g., human MMP-2) and are either peptides, polypeptides that nclude at least one immunoglobin variable region, or Kunitz domains. Methods for discovering and selecting and improving such binding proteins are described further below.

In a preferred embodiment, the MMP-9/2 binding protein includes at least one immunoglobin variable region. For example, the MMP-9/MMP-2 binding protein includes a heavy chain (HC) immunoglobulin variable domain sequence and a light chain (LC) immunoglobulin variable domain sequence. MMP-9/MMP-2 binding proteins may be antibodies. MMP-9/MMP-2 binding antibodies may have their HC and LC variable domain sequences included in a single polypeptide (e.g., scFv), or on different polypeptides (e.g., IgG or Fab).

The MMP-9/MMP-2 binding protein may be an isolated protein (e.g., at least 70, 80, 85, 90, 91, 92, 93, 94, 95, 96, 97, 98, or 99% free of other proteins).

The MMP-9/MMP-2 binding protein may additionally inhibit MMP-9, e.g., human MMP-9 and/or MMP-2, e.g., human MMP-2. The binding protein can inhibit the catalytic activity of MMP-9 (e.g., human MMP-9) and/or MMP-2 (e.g., human MMP-2). In one embodiment, the protein binds the catalytic domain of human MMP-9, e.g., the protein contacts residues in or near the active site of MMP-9 and/or the protein binds the catalytic domain of human MMP-2, e.g., the protein contacts residues in or near the active site of MMP-2. In some embodiments, the protein does not contact residues in or near the active site of MMP-9 but instead binds elsewhere on MMP-9 and causes a steric change in MMP-9 that affects (e.g., inhibits) its activity. In other embodiments, the protein does not contact residues in or near the active site of MMP-2 but instead binds elsewhere on MMP-2 and causes a steric change in MMP-2 that affects (e.g., inhibits) its activity.

The protein can bind to MMP-9 and/or MMP-2 with a binding affinity of at least 10⁵, 10⁶, 10⁷, 10⁸, 10⁹, 10¹⁰ and 10¹¹ M⁻¹. In one embodiment, the protein binds to MMP-9 and/or MMP-2 with a K_{off} slower than 1×10⁻³, 5×10⁻⁴ s⁻¹, or 1×10⁻⁴ s⁻¹. In one embodiment, the protein binds to MMP-12 with a Kₒₙ faster than 1×10², 1×10³, or 5×10³ M⁻¹ s⁻¹. In one embodiment, the protein inhibits human MMP-9 and/or MMP-2 activity, e.g., with a Ki of less than 10⁻⁵, 10⁻⁶, 10⁻⁷, 10⁻⁸, 10⁻⁹, and 10⁻¹⁰ M. The protein can have, for example, an IC50 of less than 100 nM, 10 nM or 1 nM. In some embodiments, the protein has an IC50 of about 1.8 nM. The affinity of the protein for MMP-9 can be characterized by a K_{D} of less than 100 nm, less than 10 nM, or about 3 nM (e.g., 3.1 nM), about 5 nM (e.g., 5 nM), about 6 nm (e.g., 5.9 nM), about 7 nM (e.g., 7.1 nM), or about 10 nM (e.g., 9.6 nM).

In some embodiments, the protein has a K_{D}<200 nM.

In some embodiments, the protein has a t½ of at least about 10 minutes (e.g., 11 minutes), at least about 20 minutes (e.g., 18 minutes), at least about 25 minutes (e.g., 25 minutes), at least about 35 minutes (e.g., 33 minutes), or at least about 60 minutes (e.g., 57 minutes).

An exemplary MMP-9/2 binding protein includes an antibody with a heavy chain (HC) and/or light chain (LC), and in some embodiments, an HC and/or LC variable domain, that is selected from the group of antibodies consisting of: M0237-D02, X0106-A01, X0106-B02, X0106-004, X0106-E4, and X0106-F05. An exemplary MMP-14 binding protein includes an antibody with a heavy chain (HC) and/or light chain (LC), and in some embodiments, three HC and/or three LC CDRs, that are selected from the group of antibodies consisting of: M0237-D02, X0106-A01, X0106-B02, X0106-004, X0106-E4, and X0106-F05. Such MMP-9/2 binding proteins are further described in U.S. Ser. No. 61/033,075, filed Mar. 3, 2008 and U.S. Ser. No. 61/054,938, filed May 21, 2008, and WO 2009/111508.

In one embodiment, the HC and LC variable domain sequences are components of the same polypeptide chain. In another, the HC and LC variable domain sequences are components of different polypeptide chains. For example, the protein is an IgG, e.g., IgG1, IgG2, IgG3, or IgG4. The protein can be a soluble Fab (sFab). In other implementations the protein includes a Fab2′, scFv, minibody, scFv::Fc fusion, Fab::HSA fusion, HSA::Fab fusion, Fab::HSA::Fab fusion, or other molecule that comprises the antigen combining site of one of the binding proteins herein. The VH and VL regions of these Fabs can be provided as IgG, Fab, Fab2, Fab2′, scFv, PEGylated Fab, PEGylated scFv, EGylated Fab2, VH::CH₁::HSA+LC, HSA::VH::CH1+LC, LC::HSA+VH::CH1, HSA::LC+VH::CH1, or other appropriate construction.

In one embodiment, the protein is a human or humanized antibody or is non-immunogenic in a human. For example, the protein includes one or more human antibody framework regions, e.g., all human framework regions. In one embodiment, the protein includes a human Fc domain, or an Fc domain that is at least 95, 96, 97, 98, or 99% identical to a human Fc domain.

In one embodiment, the protein is a primate or primatized antibody or is non-immunogenic in a human. For example, the protein includes one or more primate antibody framework regions, e.g., all primate framework regions. In one embodiment, the protein includes a primate Fc domain, or an Fc domain that is at least 95, 96, 97, 98, or 99% identical to a primate Fc domain. “Primate” includes humans (Homo sapiens), chimpanzees (Pan troglodytes and Pan paniscus (bonobos)), gorillas (Gorilla gorilla), gibons, monkeys, lemurs, aye-ayes (Daubentonia madagascariensis), and tarsiers.

In certain embodiments, the protein includes no sequences from mice or rabbits (e.g., is not a murine or rabbit antibody).

### MMP-12 Binding Proteins

Any MMP-12 binding protein may be used in the methods and compositions for treating inflammatory disorders that are disclosed herein.

MMP-12 is encoded by a gene designated as MMP12 with full name Matrix metalloproteinase-12 precursor. Synonyms for MMP-12 include matrix metalloproteinase 12, macrophage elastase, macrophage metalloelastase. The DNA sequence is known for Homo sapiens and Mus musculus. An exemplary cDNA sequence encoding human MMP12 and the amino acid sequence are shown below. Exemplary cDNA sequences encoding murine MMP12 and amino acid sequences are also shown below. An exemplary MMP-12 protein can include the human or mouse MMP-12 amino acid sequence, a sequence that is 80%, 85%, 90%, 95%, 96%, 97%, 98%, or 99% identical to one of these sequences, or a fragment thereof, e.g., a fragment without the signal sequence or prodomain.

cDNA and amino acid sequences of human MMP12
>embl|BC112301|BC112301 Homo sapiens matrix
metallopeptidase 12 (macrophage elastase), mRNA
(cDNA clone MGC: 138506 IMAGE: 8327769),
complete cds
(SEQ ID NO: 36)
atgaagtttcttctaatactgctcctgcaggccactgcttctggagc
tcttcccctgaacagctctacaagcctggaaaaaaataatgtgctat
ttggtgaaagatacttagaaaaattttatggccttgagataaacaaa
cttccagtgacaaaaatgaaatatagtggaaacttaatgaaggaaaa
aatccaagaaatgcagcacttcttgggtctgaaagtgaccgggcaac
tggacacatctaccctggagatgatgcacgcacctcgatgtggagtc
cccgatgtccatcatttcagggaaatgccaggggggcccgtatggag
gaaacattatatcacctacagaatcaataattacacacctgacatga
accgtgaggatgttgactacgcaatccggaaagctttccaagtatgg
agtaatgttacccccttgaaattcagcaagattaacacaggcatggc
tgacattttggtggtttttgcccgtggagctcatggagacttccatg
cttttgatggcaaaggtggaatcctagcccatgcttttggacctgga
tctggcattggaggggatgcacatttcgatgaggacgaattctggac
tacacattcaggaggcacaaacttgttcctcactgctgttcacgaga
ttggccattccttaggtcttggccattctagtgatccaaaggccgta
atgttccccacctacaaatatgttgacatcaacacatttcgcctctc
tgctgatgacatacgtggcattcagtccctgtatggagacccaaaag
agaaccaacgcttgccaaatcctgacaattcagaaccagctctctgt
gaccccaatttgagttttgatgctgtcactaccgtgggaaataagat
ctttttcttcaaagacaggttcttctggctgaaggtttctgagagac
caaagaccagtgttaatttaatttcttccttatggccaaccttgcca
tctggcattgaagctgcttatgaaattgaagccagaaatcaagtttt
tctttttaaagatgacaaatactggttaattagcaatttaagaccag
agccaaattatcccaagagcatacattcttttggttttcctaacttt
gtgaaaaaaattgatgcagctgtttttaacccacgtttttataggac
ctacttctttgtagataaccagtattggaggtatgatgaaaggagac
agatgatggaccctggttatcccaaactgattaccaagaacttccaa
ggaatcgggcctaaaattgatgcagtcttctactctaaaaacaaata
ctactatttcttccaaggatctaaccaatttgaatatgacttcctac
tccaacgtatcaccaaaacactgaaaagcaatagctggtttggttgt
tagaaatggtgtaattaatggtttttgttagttcacttcagcttaat
aagtatttattgcatatttgctatgtcctcagtgtaccactacttag
agatatgtatcataaaaataaaatctgtaaaccataggtaatgatta
tataaaatacataatatttttcaattttgaaaactctaattgtccat
tcttgcttgactctactattaagtttgaaaatagttaccttcaaagg
ccaagagaattctatttgaagcatgctctgtaagttgcttcctaaca
t
>Amino acid sequence of human MMP12 (AAI12302.1)
(SEQ ID NO: 53)
MKFLLILLLQATASGALPLNSSTSLEKNNVLFGERYLEKFYGLEINK
LPVTKMKYSGNLMKEKIQEMQHFLGLKVTGQLDTSTLEMMHAPRCGV
PDVHHFREMPGGPVWRKHYITYRINNYTPDMNREDVDYAIRKAFQVW
SNVTPLKFSKINTGMADILVVFARGAHGDFHAFDGKGGILAHAFGPG
SGIGGDAHFDEDEFWTTHSGGTNLFLTAVHEIGHSLGLGHSSDPKAV
MFPTYKYVDINTFRLSADDIRGIQSLYGDPKENQRLPNPDNSEPALC
DPNLSFDAVTTVGNKIFFFKDRFFWLKVSERPKTSVNLISSLWPTLP
SGIEAAYEIEARNQVFLFKDDKYWLISNLRPEPNYPKSIHSFGFPNF
VKKIDAAVFNPRFYRTYFFVDNQYWRYDERRQMMDPGYPKLITKNFQ
GIGPKIDAVFYSKNKYYYFFQGSNQFEYDFLLQRITKTLKSNSWFGC

Polymorphisms in the MMP-12 gene are described, for example, in (2002) J Am Coll Cardiol. 3:40(1):43-8,(2002) Hum Mol. Genet. 1:11(5):569-76., (2001) Stroke 32(9):2198-202., and (2000) Circ Res. 86(9):998-1003.

| cDNA and amino acid sequences of mouse MMP12 | |
|---|---|
| >Mouse MMP-12 cDNA sequence (NM_008605) | |
| (SEQ ID NO: 56) | |
| 1 | ACTCTGCTGAAAGGAGTCTGCACAATGAAATTTCTCATGATGATTGTGTTCTTACAGGTA |
| 61 | TCTGCCTGTGGGGCTGCTCCCATGAATGACAGTGAATTTGCTGAATGGTACTTGTCAAGA |
| 121 | TTTTATGATTATGGAAAGGACAGAATTCCAATGACAAAAACAAAAACCAATAGAAACTTC |
| 181 | CTAAAAGAAAAACTCCAGGAAATGCAGCAGTTCTTTGGGCTAGAAGCAACTGGGCAACTG |
| 241 | GACAACTCAACTCTGGCAATAATGCACATCCCTCGATGTGGAGTGCCCGATGTACAGCAT |
| 301 | CTTAGAGCAGTGCCCCAGAGGTCAAGATGGATGAAGCGGTACCTCACTTACAGGATCTAT |
| 361 | AATTACACTCCGGACATGAAGCGTGAGGATGTAGACTACATATTTCAGAAAGCTTTCCAA |
| 421 | GTCTGGAGTGATGTGACTCCTCTAAGATTCAGAAAGCTTCATAAAGATGAGGCTGACATT |
| 481 | ATGATACTTTTTGCATTTGGAGCTCACGGAGACTTCAACTATTTTGATGGCAAAGGTGGT |
| 541 | ACACTAGCCCATGCTTTTTATCCTGGACCTGGTATTCAAGGAGATGCACATTTTGATGAG |
| 601 | GCAGAAACGTGGACTAAAAGTTTTCAAGGCACAAACCTCTTCCTTGTTGCTGTTCATGAA |
| 661 | CTTGGCCATTCCTTGGGGCTGCAGCATTCCAATAATCCAAAGTCAATAATGTACCCCACC |
| 721 | TACAGATACCTTAACCCCAGCACATTTCGCCTCTCTGCTGATGACATACGTAACATTCAG |
| 781 | TCCCTCTATGGAGCCCCAGTGAAACCCCCATCCTTGACAAAACCTAGCAGTCCACCATCA |
| 841 | ACTTTCTGTCACCAAAGCTTGAGTTTTGATGCTGTCACAACAGTGGGAGAGAAAATCTTT |
| 901 | TTCTTTAAAGACTGGTTCTTCTGGTGGAAGCTTCCTGGGAGTCCAGCCACCAACATTACT |
| 961 | TCTATTTCTTCCATATGGCCAAGCATCCCATCTGGTATTCAAGCTGCTTACGAAATTGAA |
| 1021 | AGCAGAAATCAACTTTTCCTTTTTAAAGATGAGAAGTACTGGTTAATAAACAACTTAGTA |
| 1081 | CCAGAGCCACACTATCCCAGGAGCATATATTCCCTGGGCTTCTCTGCATCTGTGAAGAAG |
| 1141 | GTTGATGCAGCTGTCTTTGACCCACTTCGCCAAAAGGTTTATTTCTTTGTGGATAAACAC |
| 1201 | TACTGGAGGTATGATGTGAGGCAGGAGCTCATGGACCCTGCTTACCCCAAGCTGATTTCC |
| 1261 | ACACACTTCCCAGGAATCAAGCCTAAAATTGATGCAGTCCTCTATTTCAAAAGACACTAC |
| 1321 | TACATCTTCCAAGGAGCCTATCAATTGGAATATGACCCCCTGTTCCGTCGTGTCACCAAA |
| 1381 | ACATTGAAAAGTACAAGCTGGTTTGGTTGT |

>Amino acid sequence of mouse MMP12 (based on accession
number NM_008605)
(SEQ ID NO: 57)
MKFLMMIVFLQVSACGAAPMNDSEFAEWYLSRFYDYGKDRIPMTKTKTNRNFLKEKLQEMQQFF
GLEATGQLDNSTLAIMHIPRCGVPDVQHLRAVPQRSRWMKRYLTYRIYNYTPDMKREDVDYIFQ
KAFQVWSDVTPLRFRKLHKDEADIMILFAFGAHGDFNYFDGKGGTLAHAFYPGPGIQGDAHFDE
AETWTKSFQGTNLFLVAVHELGHSLGLQHSNNPKSIMYPTYRYLNPSTFRLSADDIRNIQSLYG
APVKPPSLTKPSSPPSTFCHQSLSFDAVTTVGEKIFFFKDWFFWWKLPGSPATNITSISSIWPS
IPSGIQAAYEIESRNQLFLFKDEKYWLINNLVPEPHYPRSIYSLGFSASVKKVDAAVFDPLRQK
VYFFVDKHYWRYDVRQELMDPAYPKLISTHFPGIKPKIDAVLYFKRHYYIFQGAYQLEYDPLFR
RVTKTLKSTSWFGC

Factors that regulate MMP-12. Expression of MMP-12 is regulated by many factors. Reports of upregulation include: Oncogene. 2004 Jan. 22; 23(3):845-51. (recurrence in stage I lung cancer, 2/10 cases), Ann Neurol. 2003 June; 53(6):731-42. (collagenase-induced rat model of intracerebral hemorrhage), Cancer Res. 2005 May 15; 65(10):4261-72. (protein kinase C/p53 resistant cells), Br J. Dermatol. 2005 April; 152(4):720-6. (Samples from nine patients with squamous cell carcinoma), Cardiovasc Res. 2005 May 1; 66(2):410-9. (Aging), J. Immunol. 2005 Apr. 15; 174(8):4953-9. (Surfactant protein D −/− mice), J Biol. Chem. 2005 Jun. 3; 280(22):21653-60. (Corneal wound repair), Surgery. 2005 April; 137(4):457-62. (periaortic application of CaCl₂ in mice), J. Virol. 2005 April; 79(8):4764-73. (murine viral encephalitis), Biochem Biophys Res Commun. 2005 Apr. 29; 330(1):194-203. (cigarette smoke condensate in mice), Inflamm Res. 2005 January; 54(1):31-6. (bronchoalveolar lavage and lung tissue from COPD patients), Am J Respir Cell Mol. Biol. 2005 April; 32(4):311-8. (Induction of human IL-1beta in transgenic mice), Toxicol Pathol. 2004 May-June; 32(3):351-6. (Cigarette smoke in mice), Am J Physiol Lung Cell Mol. Physiol. 2004 April; 286(4):L801-7. (lysosomal acid lipase gene knockout mice), J. Neurosci. 2004 Feb. 11; 24(6):1521-9. (A1 adenosine receptor null mice), J. Neuroimmunol. 1998 Jul. 1; 87(1-2):62-72. (experimentally-induced delayed type hypersensitivity model of MS), Scand J. Immunol. 2005 January; 61(1):10⁻⁷. (IL-4), J Pediatr Gastroenterol Nutr. 2005 January; 40(1):60-6. (Enterocolitis), J Cell Physiol. 2005 July; 204(1):139-45. (Statins), J. Immunol. 2004 Oct. 15; 173(8):5209-18. (experimental autoimmune encephalomyelitis), Cardiovasc Pharmacol. 2004 July; 44(1):57-65. (hypercholesterolemic hamsters with endothelial injury in the carotid artery), Cancer Metastasis Rev. 2004 January-June; 23(1-2):101-17. (colorectal cancer), Free Radic Biol Med. 2004 Mar. 15; 36(6):782-801. (oxidative stress), Chest. 2004 February; 125(2):466-72. (Wood smoke, cigarette smoke, CPOD).

Down regulation or no upregulation is reported in Inflammation. 2003 April; 27(2):107-13. (Mice immunized with type II collagen), Cancer Res. 2003 Jan. 1; 63(1):256-62. (Epstein-Barr virus proteins; nasopharyngeal carcinoma), Curr Eye Res. 1998 February; 17(2):132-40. (human interphotoreceptor matrix and vitreous from postmortem human eyes), and Scand J. Immunol. 2005 January; 61(1):10-7. (dexamethasone).

Endogenous inhibitors of MMP-12. MMP-12 has a number of endogenous inhibitors Like other MMPs, MMP-12 is inhibited by TIMPs (Murphy, G., and Willenbrock, F. (1995) Methods Enzymol. 248, 496-510).

Small molecule inhibitors of MMP-12. Small molecule inhibitors of MMP-12 have been synthesized and tested. Most of these have either insufficient potency or insufficient specificity, or both. The reports include: Proc Natl Acad Sci USA. 2005 Apr. 12; 102(15):5334-9. (acetohydroxamic acid and N-isobutyl-N-[4-methoxyphenylsulfonyl]glycyl hydroxamic acid); Arthritis Rheum. 2004 October; 50(10):3275-85. (a general hydroxamate inhibitor of MMP activity); Arch Biochem Biophys. 2003 Jan. 15; 409(2):335-40. (peptide 1 in24); J Mol. Biol. 2001 Sep. 28; 312(4):743-51. (hydroxamic acid inhibitor, CGS27023A); J Mol. Biol. 2001 Sep. 28; 312(4):731-42. (batimastat (BB-94)); Anticancer Res. 2001 January-February; 21(1A):145-55. (AE-941, an orally bioavailable extract made of cartilage); J Comb Chem. 2000 November-December; 2(6):624-38. (XXX-Gpsi(PO2H—CH2)L-XXX library on beads); Biochim Biophys Acta. 2000 Mar. 16; 1478(1):51-60. (green tea polyphenols); J Leukoc Biol. 1984 May; 35(5):449-57. (peptide chloromethyl ketone); Am Rev Respir Dis. 1982 February; 125(2):203-7. (a battery of elastase inhibitors); Mem Inst Oswaldo Cruz. 2005 March; 100:167-172. (marimastat); J Mol. Biol. 2004 Aug. 20; 341(4):1063-76. (CP-271485, PF-00356231, and PD-0359601); Inflamm Res. 2003 March; 52(3):95-100.; Bioorg Med

Chem. Lett. 2004 Oct. 4; 14(19):4935-9. (inhibitors with novel oxazoline zinc binding groups); and J Cardiovasc Pharmacol. 2004 July; 44(1):57-65. (ONO-4817). Other small molecule inhibitors of MMP-12 are described, e.g., in US Patent Application No: 20050014817 (Fluorothiophene derivatives), US Patent Application No.: 20050014816 (Thiophene amino acid derivatives), U.S. Pat. No. 6,770,640 (1-Carboxylmethyl-2-oxo-azepan derivatives), PCT Publication No.: WO200040577 (1-Carboxymethyl-2-oxo-Azepan Derivatives), PCT Publication No.: WO 200532541 (Substituted Heterocyclic Mercaptosulfide Inhibitors), PCT Publication No.: WO 200183431, US Patent Application 20030158155, European Patent No.: 1288199, PCt Publication No.: WO 200040600 and U.S. Pat. No. 6,352,976, U.S. Pat. No. 6,350,907, U.S. Pat. No. 6,924,276, U.S. Pat. No. 6,916,807, U.S. Pat. No. 6,686,355, U.S. Pat. No. 6,548,477 and U.S. Pat. No. 5,506,242. The small molecule can be administered to inhibit MMP-12, e.g., in combination with a MMP-12 binding protein described herein.

Small interfering RNA inhibitors of MMP-12. MMP-12 can be inhibited by small interfering RNA (siRNA). Examples of siRNA that can be used are described in US Patent Publication No.: 20040087533 and PCt Publication No.: WO 200409098. The siRNA can be administered to inhibit MMP-12, e.g., in combination with a MMP-12 binding protein described herein.

MMP-12 Binding Proteins

Provided are proteins that bind to MMP-12 (e.g., human MMP-12) and are either peptides, polypeptides that include at least one immunoglobin variable region, or Kunitz domains. Methods for discovering and selecting and improving such binding proteins are described further below.

In one aspect, provided are proteins that bind to MMP-12 (e.g., human MMP-12 and/or murine MMP-12) and include at least one immunoglobin variable region. For example, the MMP-12 binding protein includes a heavy chain (HC) immunoglobulin variable domain sequence and a light chain (LC) immunoglobulin variable domain sequence. A number of exemplary MMP-12 binding proteins are described herein.

The MMP-12 binding protein may be an isolated protein (e.g., at least 70, 80, 85, 90, 91, 92, 93, 94, 95, 96, 97, 98, or 99% free of other proteins).

The MMP-12 binding protein may additionally inhibit MMP-12, e.g., human and/or murine MMP-12. The binding protein can inhibit the catalytic activity of MMP-12 (e.g., human MMP-12). In one embodiment, the protein binds the catalytic domain of human MMP-12, e.g., the protein contacts residues in or near the active site of MMP-12. In some embodiments, the protein does not contact residues in or near the active site of MMP-12 but instead binds elsewhere on MMP-12 and causes a steric change in MMP-12 that affects (e.g., inhibits) its activity.

The protein can bind to MMP-12, e.g., human MMP-12, with a binding affinity of at least 10⁵, 10⁶, 10⁷, 10⁸, 10⁹, 10¹⁰ and 10¹¹ M⁻¹. In one embodiment, the protein binds to MMP-12 with a K_{off} slower than 1×10⁻³, 5×10⁻⁴ s⁻¹, or 1×10⁻⁴ s⁻¹. In one embodiment, the protein binds to MMP-12 with a Kₒₙ faster than 1×10², 1×10³, or 5×10³ M⁻¹ s⁻¹. In one embodiment, the protein inhibits human MMP-12 activity, e.g., with a Ki of less than 10⁻⁵, 10⁻⁶, 1e, 10⁻⁸, 10⁻⁹, and 10⁻¹⁰ M. The protein can have, for example, an IC50 of less than 100 nM, 10 nM or 1 nM. In some embodiments, the protein has an IC50 of about 1.8 nM. The affinity of the protein for MMP-12 can be characterized by a K_{D} of less than 100 nm, less than 10 nM, or about 3 nM (e.g., 3.1 nM), about 5 nM (e.g., 5 nM), about 6 nm (e.g., 5.9 nM), about 7 nM (e.g., 7.1 nM), or about 10 nM (e.g., 9.6 nM).

In some embodiments, the protein has a K_{D}<200 nM.

In some embodiments, the protein has a t½ of at least about 10 minutes (e.g., 11 minutes), at least about 20 minutes (e.g., 18 minutes), at least about 25 minutes (e.g., 25 minutes), at least about 35 minutes (e.g., 33 minutes), or at least about 60 minutes (e.g., 57 minutes).

In one embodiment, the protein binds the catalytic domain of human MMP-12, e.g., the protein contacts residues in or near the active site of MMP-12.

In some embodiments, the protein does not contact residues in or near the active site of MMP-12 but instead binds elsewhere on MMP-12 and causes a steric change in MMP-12 that affects (e.g., inhibits) its activity.

In a preferred embodiment, the protein is a human antibody having the light and heavy chains of antibodies selected from the group consisting of DX-2712, a mutant or variant of DX-2712 (e.g., as described in WO 2009/111507), 539B-X0041-D02, M0134-A02, M134-A05, M134-A07, M134-A09, M134-A10, M134-A11, M0134-B01, M134-B04, M0134-B08, M0134-B11, M0134-001, M0134-C02, M0134-006, M0134-009, M0134-C10, M0134-C11, M0134-C12, M0134-D02, M0134-D03, M0134-E04, M0134-E07, M0134-E08, M134-E11, M0134-F01, M0134-F05, M0134-G02, M0134-G04, M0134-G07, M0135-A03, M0135-A05, M0135-A06, M0135-A07, M0135-B02, M0135-B08, M0135-001, M0135-C11, M0135-E03, M0135-F03, M0135-F11, M0135-G02, M0135-G03, M0135-G07, M0135-G11, M0135-H03, M0135-H10, M0105-C05, M0105-E11, M0105-F08, M0107-A12, M0108-A02, M0109-G11, M0110-G05, M0129-B11, M0130-A01, M0130-C12, M0130-F06, M0130-H04, M0131-A06, M0131-D03, M0132-A04, M0133-B08, M0133-E05, M0121-E07, M0118-F11, M0125-G07, M0124-E07, M0119-D01, M0119-A02, M0122-006, M0123-G07, M0063-A02, M0063-A04, M0063-B01, M0063-B11, M0063-007, M0063-G01, M0065-E12, M0065-G03, M0065-H05, M0067-A02, M0067-B06, M0067-B09, M0067-C10, M0067-F02, M0067-F06, M0069-A04, M0069-A11, M0069-C02, M0069-D10, M0069-G07, M0071-A01, M0071-B07, M0071-D05, M0071-D09, M0071-H03, M0071-H06, M0087-F09, M0088-F07, M0088-G10, M0088-H10, M0089-001, M0089-F05, M0089-B07, M0089-H11, M0032-E01, M0034-004, M0039-F01, M0041-B05, M0041-G01, M0042-B06, M0006-B10, M0007-H06, M0008-H09, M0009-H08, M0011-H11, M0015-F02, M0016-D01, M0013-D11, M0013-G12, M0013-H06, M0014-009, M0014-G11, M0016-A11, M0016-H05, M0019-C05, M0020-B01, M0022-007, M0025-D04, M0027-E11, 539B-X0041-D02, 539B-X0049-A01, 539B-X0049-B01, 539B-X0049-001, 539B-X0049-D01, 539B-X0049-E01, 539B-X0049-F01, 539B-X0049-G01, 539B-X0049-H01, 539B-X0049-A02, 539B-X0049-B02, 539B-X0049-C02, 539B-X0049-D02, and 539B-X0049-E02. In a preferred embodiment, the protein is a human antibody having its heavy chain selected from the group consisting of DX-2712, a mutant or variant of DX-2712 (e.g., as described in WO 2009/111507), 539B-X0041-D02, M0134-A02, M134-A05, M134-A07, M134-A09, M134-A10, M134-A11, M0134-B01, M134-B04, M0134-B08, M0134-B11, M0134-001, M0134-C02, M0134-006, M0134-009, M0134-C10, M0134-C11, M0134-C12, M0134-D02, M0134-D03, M0134-E04, M0134-E07, M0134-E08, M134-E11, M0134-F01, M0134-F05, M0134-G02, M0134-G04, M0134-G07, M0135-A03, M0135-A05, M0135-A06, M0135-A07, M0135-B02, M0135-B08, M0135-001, M0135-C11, M0135-E03, M0135-F03, M0135-F11, M0135-G02, M0135-G03, M0135-G07, M0135-G11, M0135-H03, M0135-H10, M0105-C05, M0105-E11, M0105-F08, M0107-A12, M0108-A02, M0109-G11, M0110-G05, M0129-B11, M0130-A01, M0130-C12, M0130-F06, M0130-H04, M0131-A06, M0131-D03, M0132-A04, M0133-B08, M0133-E05, M0121-E07, M0118-F11, M0125-G07, M0124-E07, M0119-D01, M0119-A02, M0122-006, M0123-G07, M0063-A02, M0063-A04, M0063-B01, M0063-B11, M0063-007, M0063-G01, M0065-E12, M0065-G03, M0065-H05, M0067-A02, M0067-B06, M0067-B09, M0067-C10, M0067-F02, M0067-F06, M0069-A04, M0069-A11, M0069-C02, M0069-D10, M0069-G07, M0071-A01, M0071-B07, M0071-D05, M0071-D09, M0071-H03, M0071-H06, M0087-F09, M0088-F07, M0088-G10, M0088-H10, M0089-001, M0089-F05, M0089-B07, M0089-H11, M0032-E01, M0034-004, M0039-F01, M0041-B05, M0041-G01, M0042-B06, M0006-B10, M0007-H06, M0008-H09, M0009-H08, M0011-H11, M0015-F02, M0016-D01, M0013-D11, M0013-G12, M0013-H06, M0014-009, M0014-G11, M0016-A11, M0016-H05, M0019-C05, M0020-B01, M0022-007, M0025-D04, M0027-E11, 539B-X0041-D02, 539B-X0049-A01, 539B-X0049-B01, 539B-X0049-001, 539B-X0049-D01, 539B-X0049-E01, 539B-X0049-F01, 539B-X0049-G01, 539B-X0049-H01, 539B-X0049-A02, 539B-X0049-B02, 539B-X0049-C02, 539B-X0049-D02, and 539B-X0049-E02. In a preferred embodiment, the protein is a human antibody having its light chain selected from the group consisting of DX-2712, a mutant or variant of DX-2712 (e.g., as described in WO 2009/111507), 539B-X0041-D02, M0134-A02, M134-A05, M134-A07, M134-A09, M134-A10, M134-A11, M0134-B01, M134-B04, M0134-B08, M0134-B11, M0134-001, M0134-C02, M0134-006, M0134-009, M0134-C10, M0134-C11, M0134-C12, M0134-D02, M0134-D03, M0134-E04, M0134-E07, M0134-E08, M134-E11, M0134-F01, M0134-F05, M0134-G02, M0134-G04, M0134-G07, M0135-A03, M0135-A05, M0135-A06, M0135-A07, M0135-B02, M0135-B08, M0135-001, M0135-C11, M0135-E03, M0135-F03, M0135-F11, M0135-G02, M0135-G03, M0135-G07, M0135-G11, M0135-H03, M0135-H10, M0105-C05, M0105-E11, M0105-F08, M0107-A12, M0108-A02, M0109-G11, M0110-G05, M0129-B11, M0130-A01, M0130-C12, M0130-F06, M0130-H04, M0131-A06, M0131-D03, M0132-A04, M0133-B08, M0133-E05, M0121-E07, M0118-F11, M0125-G07, M0124-E07, M0119-D01, M0119-A02, M0122-006, M0123-G07, M0063-A02, M0063-A04, M0063-B01, M0063-B11, M0063-007, M0063-G01, M0065-E12, M0065-G03, M0065-H05, M0067-A02, M0067-B06, M0067-B09, M0067-C10, M0067-F02, M0067-F06, M0069-A04, M0069-A11, M0069-C02, M0069-D10, M0069-G07, M0071-A01, M0071-B07, M0071-D05, M0071-D09, M0071-H03, M0071-H06, M0087-F09, M0088-F07, M0088-G10, M0088-H10, M0089-001, M0089-F05, M0089-B07, M0089-H11, M0032-E01, M0034-004, M0039-F01, M0041-B05, M0041-G01, M0042-B06, M0006-B10, M0007-H06, M0008-H09, M0009-H08, M0011-H11, M0015-F02, M0016-D01, M0013-D11, M0013-G12, M0013-H06, M0014-009, M0014-G11, M0016-A11, M0016-H05, M0019-035, M0020-B01, M0022-007, M0025-D04, M0027-E11, 539B-X0041-D02, 539B-X0049-A01, 539B-X0049-B01, 539B-X0049-001, 539B-X0049-D01, 539B-X0049-E01, 539B-X0049-F01, 539B-X0049-G01, 539B-X0049-H01, 539B-X0049-A02, 539B-X0049-B02, 539B-X0049-C02, 539B-X0049-D02, and 539B-X0049-E02. In a preferred embodiment, the protein is a human antibody having one or more heavy chain CDRs picked from the corresponding CDRs of the heavy chains selected from the group consisting of DX-2712, a mutant or variant of DX-2712 (e.g., as described in WO 2009/111507), 539B-X0041-D02, M0134-A02, M134-A05, M134-A07, M134-A09, M134-A10, M134-A11, M0134-B01, M134-B04, M0134-B08, M0134-B11, M0134-001, M0134-C02, M0134-006, M0134-009, M0134-C10, M0134-C11, M0134-C12, M0134-D02, M0134-D03, M0134-E04, M0134-E07, M0134-E08, M134-E11, M0134-F01, M0134-F05, M0134-G02, M0134-G04, M0134-G07, M0135-A03, M0135-A05, M0135-A06, M0135-A07, M0135-B02, M0135-B08, M0135-001, M0135-C11, M0135-E03, M0135-F03, M0135-F11, M0135-G02, M0135-G03, M0135-G07, M0135-G11, M0135-H03, M0135-H10, M0105-C05, M0105-E11, M0105-F08, M0107-A12, M0108-A02, M0109-G11, M0110-G05, M0129-B11, M0130-A01, M0130-C12, M0130-F06, M0130-H04, M0131-A06, M0131-D03, M0132-A04, M0133-B08, M0133-E05, M0121-E07, M0118-F11, M0125-G07, M0124-E07, M0119-D01, M0119-A02, M0122-006, M0123-G07, M0063-A02, M0063-A04, M0063-B01, M0063-B11, M0063-007, M0063-G01, M0065-E12, M0065-G03, M0065-H05, M0067-A02, M0067-B06, M0067-B09, M0067-C10, M0067-F02, M0067-F06, M0069-A04, M0069-A11, M0069-C02, M0069-D10, M0069-G07, M0071-A01, M0071-B07, M0071-D05, M0071-D09, M0071-H03, M0071-H06, M0087-F09, M0088-F07, M0088-G10, M0088-H10, M0089-001, M0089-F05, M0089-B07, M0089-H11, M0032-E01, M0034-C04, M0039-F01, M0041-B05, M0041-G01, M0042-B06, M0006-B10, M0007-H06, M0008-H09, M0009-H08, M0011-H11, M0015-F02, M0016-D01, M0013-D11, M0013-G12, M0013-H06, M0014-009, M0014-G11, M0016-A11, M0016-H05, M0019-035, M0020-B01, M0022-007, M0025-D04, M0027-E11, 539B-X0041-D02, 539B-X0049-A01, 539B-X0049-B01, 539B-X0049-001, 539B-X0049-D01, 539B-X0049-E01, 539B-X0049-F01, 539B-X0049-G01, 539B-X0049-H01, 539B-X0049-A02, 539B-X0049-B02, 539B-X0049-C02, 539B-X0049-D02, and 539B-X0049-E02. In a preferred embodiment, the protein is a human antibody having one or more light chain CDRs picked from the corresponding CDRs of the light chains selected from the group consisting of DX-2712, a mutant or variant of DX-2712 (e.g., as described in WO 2009/111507), 539B-X0041-D02, M0134-A02, M134-A05, M134-A07, M134-A09, M134-A10, M134-A11, M0134-B01, M134-B04, M0134-B08, M0134-B11, M0134-001, M0134-C02, M0134-006, M0134-009, M0134-C10, M0134-C11, M0134-C12, M0134-D02, M0134-DO3, M0134-E04, M0134-E07, M0134-E08, M134-E11, M0134-F01, M0134-F05, M0134-G02, M0134-G04, M0134-G07, M0135-A03, M0135-A05, M0135-A06, M0135-A07, M0135-B02, M0135-B08, M0135-001, M0135-C11, M0135-E03, M0135-F03, M0135-F11, M0135-G02, M0135-G03, M0135-G07, M0135-G11, M0135-H03, M0135-H10, M0105-C05, M0105-E11, M0105-F08, M0107-A12, M0108-A02, M0109-G11, M0110-G05, M0129-B11, M0130-A01, M0130-C12, M0130-F06, M0130-H04, M0131-A06, M0131-D03, M0132-A04, M0133-B08, M0133-E05, M0121-E07, M0118-F11, M0125-G07, M0124-E07, M0119-D01, M0119-A02, M0122-006, M0123-G07, M0063-A02, M0063-A04, M0063-B01, M0063-B11, M0063-007, M0063-G01, M0065-E12, M0065-G03, M0065-H05, M0067-A02, M0067-B06, M0067-B09, M0067-C10, M0067-F02, M0067-F06, M0069-A04, M0069-A11, M0069-C02, M0069-D10, M0069-G07, M0071-A01, M0071-B07, M0071-D05, M0071-D09, M0071-H03, M0071-H06, M0087-F09, M0088-F07, M0088-G10, M0088-H10, M0089-001, M0089-F05, M0089-B07, M0089-H11, M0032-E01, M0034-004, M0039-F01, M0041-B05, M0041-G01, M0042-B06, M0006-B10, M0007-H06, M0008-H09, M0009-H08, M0011-H11, M0015-F02, M0016-D01, M0013-D11, M0013-G12, M0013-H06, M0014-009, M0014-G11, M0016-A11, M0016-H05, M0019-C05, M0020-B01, M0022-007, M0025-D04, M0027-E11, 539B-X0041-D02, 539B-X0049-A01, 539B-X0049-B01, 539B-X0049-001, 539B-X0049-D01, 539B-X0049-E01, 539B-X0049-F01, 539B-X0049-G01, 539B-X0049-H01, 539B-X0049-A02, 539B-X0049-B02, 539B-X0049-C02, 539B-X0049-D02, and 539B-X0049-E02. In a preferred embodiment, the protein is a human antibody having one or more light chain CDRs and one or more heavy chain CDRs picked from the corresponding CDRs of the light chains selected from the group consisting of DX-2712, a mutant or variant of DX-2712 (e.g., as described in WO 2009/111507), 539B-X0041-D02, M0134-A02, M134-A05, M134-A07, M134-A09, M134-A10, M134-A11, M0134-B01, M134-B04, M0134-B08, M0134-B11, M0134-001, M0134-C02, M0134-006, M0134-009, M0134-C10, M0134-C11, M0134-C12, M0134-D02, M0134-D03, M0134-E04, M0134-E07, M0134-E08, M134-E11, M0134-F01, M0134-F05, M0134-G02, M0134-G04, M0134-G07, M0135-A03, M0135-A05, M0135-A06, M0135-A07, M0135-B02, M0135-B08, M0135-001, M0135-C11, M0135-E03, M0135-F03, M0135-F11, M0135-G02, M0135-G03, M0135-G07, M0135-G11, M0135-H03, M0135-H10, M0105-C05, M0105-E11, M0105-F08, M0107-A12, M0108-A02, M0109-G11, M0110-G05, M0129-B11, M0130-A01, M0130-C12, M0130-F06, M0130-H04, M0131-A06, M0131-D03, M0132-A04, M0133-B08, M0133-E05, M0121-E07, M0118-F11, M0125-G07, M0124-E07, M0119-D01, M0119-A02, M0122-006, M0123-G07, M0063-A02, M0063-A04, M0063-B01, M0063-B11, M0063-007, M0063-G01, M0065-E12, M0065-G03, M0065-H05, M0067-A02, M0067-B06, M0067-B09, M0067-C10, M0067-F02, M0067-F06, M0069-A04, M0069-A11, M0069-C02, M0069-D10, M0069-G07, M0071-A01, M0071-B07, M0071-D05, M0071-D09, M0071-H03, M0071-H06, M0087-F09, M0088-F07, M0088-G10, M0088-H10, M0089-001, M0089-F05, M0089-B07, M0089-H11, M0032-E01, M0034-004, M0039-F01, M0041-B05, M0041-G01, M0042-B06, M0006-B10, M0007-H06, M0008-H09, M0009-H08, M0011-H11, M0015-F02, M0016-D01, M0013-D11, M0013-G12, M0013-H06, M0014-009, M0014-G11, M0016-A11, M0016-H05, M0019-C05, M0020-B01, M0022-007, M0025-D04, M0027-E11, 539B-X0041-D02, 539B-X0049-A01, 539B-X0049-B01, 539B-X0049-001, 539B-X0049-D01, 539B-X0049-E01, 539B-X0049-F01, 539B-X0049-G01, 539B-X0049-H01, 539B-X0049-A02, 539B-X0049-B02, 539B-X0049-C02, 539B-X0049-D02, and 539B-X0049-E02.

These MMP-12 binding proteins are further described in U.S. Ser. No. 61/033,348, filed Mar. 3, 2008 and U.S. Ser. No. 61/127,830, filed May 14, 2008, and WO 2009/111507.

In a more preferred embodiment, the protein is a human antibody having the light and heavy chains of antibodies DX-2712, M0008-H09, M0131-A06 or M0121-E07. In another preferred embodiment, the protein is a human antibody having its heavy chain from DX-2712, M0008-H09, M0131-A06 and M0121-E07. In yet another preferred embodiment, the protein is a human antibody having its light chain from DX-2712, M0008-H09, M0131-A06 or M0121-E07. In an even more preferred embodiment, the protein is a human antibody having one or more heavy chain CDRs from the corresponding CDRs of the heavy chain of DX-2712, M0008-H09, M0131-A06 or M0121-E07. In another even more preferred embodiment, the protein is a human antibody having one or more light chain CDRs from the corresponding CDRs of the light chain of DX-2712, M0008-H09, M0131-A06 or M0121-E07.

In one embodiment, the HC and LC variable domain sequences are components of the same polypeptide chain. In another, the HC and LC variable domain sequences are components of different polypeptide chains. For example, the protein is an IgG, e.g., IgG1, IgG2, IgG3, or IgG4. The protein can be a soluble Fab (sFab). In other implementations the protein includes a Fab2′, scFv, minibody, scFv::Fc fusion, Fab::HSA fusion, HSA::Fab fusion, Fab::HSA::Fab fusion, or other molecule that comprises the antigen combining site of one of the binding proteins herein. The VH and VL regions of these Fabs can be provided as IgG, Fab, Fab2, Fab2′, scFv, PEGylated Fab, PEGylated scFv, PEGylated Fab2, VH::CH1::HSA+LC, HSA::VH::CH1+LC, LC::HSA+VH::CH1, HSA::LC+VH::CH1, or other appropriate construction.

In one embodiment, the protein is a human or humanized antibody or is non-immunogenic in a human. For example, the protein includes one or more human antibody framework regions, e.g., all human framework regions. In one embodiment, the protein includes a human Fc domain, or an Fc domain that is at least 95, 96, 97, 98, or 99% identical to a human Fc domain.

In one embodiment, the protein is a primate or primatized antibody or is non-immunogenic in a human. For example, the protein includes one or more primate antibody framework regions, e.g., all primate framework regions. In one embodiment, the protein includes a primate Fc domain, or an Fc domain that is at least 95, 96, 97, 98, or 99% identical to a primate Fc domain. “Primate” includes humans (Homo sapiens), chimpanzees (Pan troglodytes and Pan paniscus (bonobos)), gorillas (Gorilla gorilla), gibons, monkeys, lemurs, aye-ayes (Daubentonia madagascariensis), and tarsiers.

In certain embodiments, the protein includes no sequences from mice or rabbits (e.g., is not a murine or rabbit antibody).

### MMP-14 Binding Proteins

Any MMP-14 binding protein may be used in the methods and compositions for treating inflammatory disorders that are disclosed herein.

MMP-14 is encoded by a gene designated as MMP14, matrix metalloproteinase-14 precursor. Synonyms for MMP-14 include matrix metalloproteinase 14 (membrane-inserted), membrane-type-1 matrix metalloproteinase, membrane-type matrix metalloproteinase 1, MMP-14, MMP-X1, MT1MMP, MT1-MMP, MTMMP1, MT-MMP 1. MT-MMPs have similar structures, including a signal peptide, a prodomain, a catalytic domain, a hinge region, and a hemopexin domain (Wang, et al., 2004, J Biol Chem, 279:51148-55). According to SwissProt entry P50281, the signal sequence of MMP-14 precursor includes amino acid residues 1-20. The pro-peptide includes residues 21-111. Cys93 is annotated as a possible cysteine switch. Residues 112 through 582 make up the mature, active protein. The catalytic domain includes residues 112-317. The hemopexin domains includes residues 318-523. The transmembrane segment comprises residues 542 through 562.

MMP-14 can be shed from cells or found on the surface of cells, tethered by a single transmembrane amino-acid sequence. See, e.g., Osnkowski et al. (2004, J Cell Physiol, 200:2-10).

An exemplary amino acid sequence of human MMP14 is shown in Table 4:

TABLE 4
Amino-acid sequence of human MMP14

(SEQ ID NO: 60)
MSPAPRPPRCLLLPLLTLGTALASLGSAQSSSFSPEAWLQQYGYLPPG
DLRTHTQRSPQSLSAAIAAMQKFYGLQVTGKADADTMKAMRRPRCGVP
DKFGAEIKANVRRKRYAIQGLKWQHNEITFCIQNYTPKVGEYATYEAI
RKAFRVWESATPLRFREVPYAYIREGHEKQADIMIFFAEGFHGDSTPF
DGEGGFLAHAYFPGPNIGGDTHFDSAEPWTVRNEDLNGNDIFLVAVHE
LGHALGLEHSSDPSAIMAPFYQWMDTENFVLPDDDRRGIQQLYGGESG
FPTKMPPQPRTTSRPSVPDKPKNPTYGPNICDGNFDTVAMLRGEMFVF
KERWFWRVRNNQVMDGYPMPIGQFWRGLPASINTAYERKDGKFVFFKG
DKHWVFDEASLEPGYPKHIKELGRGLPTDKIDAALFWMPNGKTYFFRG
NKYYRFNEELRAVDSEYPKNIKVWEGIPESPRGSFMGSDEVFTYFYKG
NKYWKFNNQKLKVEPGYPKSALRDWMGCPSGGRPDEGTEEETEVIIIE
VDEEGGGAVSAAAVVLPVLLLLLVLAVGLAVFFFRRHGTPRRLLYCQR
SLLDKV
Genbank Accession No. CAA88372.1).

An exemplary amino acid sequence of mouse MMP14 is shown in Table 5.

TABLE 5
Amino-acid sequence of mouse MMP14

(SEQ ID NO: 61)
MSPAPRPSRSLLLPLLTLGTALASLGWAQGSNFSPEAWLQQYGYLPP
GDLRTHTQRSPQSLSAAIAAMQKFYGLQVTGKADLATMMAMRRPRCG
VPDKFGTEIKANVRRKRYAIQGLKWQHNEITFCIQNYTPKVGEYATF
EAIRKAFRVWESATPLRFREVPYAYIREGHEKQADIMILFAEGFHGD
STPFDGEGGFLAHAYFPGPNIGGDTHFDSAEPWTVQNEDLNGNDIFL
VAVHELGHALGLEHSNDPSAIMSPFYQWMDTENFVLPDDDRRGIQQL
YGSKSGSPTKMPPQPRTTSRPSVPDKPKNPAYGPNICDGNFDTVAML
RGEMFVFKERWFWRVRNNQVMDGYPMPIGQFWRGLPASINTAYERKD
GKFVFFKGDKHWVFDEASLEPGYPKHIKELGRGLPTDKIDAALFWMP
NGKTYFFRGNKYYRFNEEFRAVDSEYPKNIKVWEGIPESPRGSFMGS
DEVFTYFYKGNKYWKFNNQKLKVEPGYPKSALRDWMGCPSGRRPDEG
TEEETEVIIIEVDEEGSGAVSAAAVVLPVLLLLLVLAVGLAVFFFRR
HGTPKRLLYCQRSLLDKV
GenBank Accession No. NP_032634.2.

An exemplary MMP-14 protein against which MMP14 binding proteins may be developed can include the human or mouse MMP-14 amino acid sequence, a sequence that is 80%, 85%, 90%, 95%, 96%, 97%, 98%, or 99% identical to one of these sequences, or a fragment thereof, e.g., a fragment without the signal sequence or prodomain.

Provided are proteins that bind to MMP-14 (e.g., human MMP-14) and are either peptides, polypeptides that include at least one immunoglobin variable region, or Kunitz domains. Methods for discovering and selecting and improving such binding proteins are described further below.

The MMP-14 binding protein may be an isolated protein (e.g., at least 70, 80, 85, 90, 91, 92, 93, 94, 95, 96, 97, 98, or 99% free of other proteins).

The MMP-14 binding protein may additionally inhibit MMP-14, e.g., human and/or murine MMP-14. The binding protein can inhibit the catalytic activity of MMP-14 (e.g., human MMP-14). In one embodiment, the protein binds the catalytic domain of human MMP-14, e.g., the protein contacts residues in or near the active site of MMP-14. In some embodiments, the protein does not contact residues in or near the active site of MMP-14 but instead binds elsewhere on MMP-14 and causes a steric change in MMP-14 that affects (e.g., inhibits) its activity.

In certain embodiments, proteins that bind to MMP-14 (e.g., human MMP-14) and include at least one immunoglobulin variable region are used in the methods and compositions. For example, the MMP-14 binding protein includes a heavy chain (HC) immunoglobulin variable domain sequence and a light chain (LC) immunoglobulin variable domain sequence. A number of exemplary MMP-14 binding proteins are described herein.

The protein can bind to MMP-14 with a binding affinity of at least 10⁵, 10⁶, 10⁷, 10⁸, 10⁹, 10¹⁰ and 10¹¹ M⁻¹. In one embodiment, the protein binds to MMP-14 with a K_{off} slower than 1×10⁻³, 5×10⁻⁴ S⁻¹, or 1×10⁻⁴ S⁻¹. In one embodiment, the protein binds to MMP-14 with a Kₒₙ faster than 1×10², 1×10³, or 5×10³ M⁻¹s⁻¹. In one embodiment, the protein inhibits human MMP-14 activity, e.g., with a Ki of less than 10⁻⁵, 10⁻⁶, 10⁻⁷, 10⁻⁸, 10⁻⁹, and 10⁻¹⁰ M. The protein can have, for example, an IC50 of less than 100 nM, 10 nM or 1 nM. In some embodiments, the protein has an IC50 of about 1.8 nM. The affinity of the protein for MMP-14 can be characterized by a K_{D} of less than 100 nm, less than 10 nM, or about 3 nM (e.g., 3.1 nM), about 5 nM (e.g., 5 nM), about 6 nm (e.g., 5.9 nM), about 7 nM (e.g., 7.1 nM), or about 10 nM (e.g., 9.6 nM).

In some embodiments, the protein has a K_{D}<200 nM.

In some embodiments, the protein has a t½ of at least about 10 minutes (e.g., 11 minutes), at least about 20 minutes (e.g., 18 minutes), at least about 25 minutes (e.g., 25 minutes), at least about 35 minutes (e.g., 33 minutes), or at least about 60 minutes (e.g., 57 minutes).

MMP-14 binding proteins may also be antibodies. MMP-14 binding antibodies may have their HC and LC variable domain sequences included in a single polypeptide (e.g., scFv), or on different polypeptides (e.g., IgG or Fab).

An exemplary MMP-14 binding protein includes an antibody with a heavy chain (HC) and/or light chain (LC), and in some embodiments, an HC and/or LC variable domain, that is selected from the group of antibodies consisting of: DX-2400, DX-2410, M0031-C02, M0031-F01, M0033-H07, M0037-009, M0037-D01, M0038-E06, M0038-F01, M0038-F08, M0039-H08, M0040-A06, M0040-A11, and M0043-G02. An exemplary MMP-14 binding protein includes an antibody with a heavy chain (HC) and/or light chain (LC), and in some embodiments, three HC and/or three LC CDRs, that are selected from the group of antibodies consisting of: DX-2400, DX-2410, M0031-C02, M0031-F01, M0033-H07, M0037-009, M0037-D01, M0038-E06, M0038-F01, M0038-F08, M0039-H08, M0040-A06, M0040-A11, and M0043-G02. Further description of them and their discovery and production is provided in pending application U.S. Ser. No. 11/648,423 (US 2007-0217997) and WO 2007/079218.

In some embodiments, the MMP-14 binding protein comprises one or moe (e.g., 1, 2, or 3) HC CDRs and/or one or more (e.g., 1, 2, or 3) LC CDRs of DX-2400.

In some embodiments, the MMP-14 binding protein comprises the HC variable region and/or the LC variable region of DX-2400.

In some embodiments, the MMP-14 binding protein comprises one or moe (e.g., 1, 2, or 3) HC CDRs and/or one or more (e.g., 1, 2, or 3) LC CDRs of DX-2410.

In some embodiments, the MMP-14 binding protein comprises the HC variable region and/or the LC variable region of DX-2410.

In one embodiment, the HC and LC variable domain sequences are components of the same polypeptide chain. In another, the HC and LC variable domain sequences are components of different polypeptide chains. For example, the protein is an IgG, e.g., IgG1, IgG2, IgG3, or IgG4. The protein can be a soluble Fab (sFab). In other implementations the protein includes a Fab2′, scFv, minibody, scFv::Fc fusion, Fab::HSA fusion, HSA::Fab fusion, Fab::HSA::Fab fusion, or other molecule that comprises the antigen combining site of one of the binding proteins herein. The VH and VL regions of these Fabs can be provided as IgG, Fab, Fab2, Fab2′, scFv, PEGylated Fab, PEGylated scFv, PEGylated Fab2, VH::CH1::HSA+LC, HSA::VH::CH1+LC, LC::HSA+VH::CH1, HSA::LC+VH::CH1, or other appropriate construction.

In one embodiment, the protein is a human or humanized antibody or is non-immunogenic in a human. For example, the protein includes one or more human antibody framework regions, e.g., all human framework regions. In one embodiment, the protein includes a human Fc domain, or an Fc domain that is at least 95, 96, 97, 98, or 99% identical to a human Fc domain.

In one embodiment, the protein is a primate or primatized antibody or is non-immunogenic in a human. For example, the protein includes one or more primate antibody framework regions, e.g., all primate framework regions. In one embodiment, the protein includes a primate Fc domain, or an Fc domain that is at least 95, 96, 97, 98, or 99% identical to a primate Fc domain. “Primate” includes humans (Homo sapiens), chimpanzees (Pan troglodytes and Pan paniscus (bonobos)), gorillas (Gorilla gorilla), gibons, monkeys, lemurs, aye-ayes (Daubentonia madagascariensis), and tarsiers.

In certain embodiments, the protein includes no sequences from mice or rabbits (e.g., is not a murine or rabbit antibody).

Other MMP-14 inhibitors known in the art include, but are not limited to, those disclosed in the following patents and patent applications: U.S. Pat. No. 6,114,159; U.S. Pat. No. 6,399,348; JP 3802560 and EP 0750672 (all in the name of Max Delbrueck Center for Molecular Medicine); U.S. Pat. No. 6,184,022; U.S. Pat. No. 6,825,024; EP 0685557; JP 2694604 (all in the name of Daiichi Fine Chemicals); and U.S. Ser. No. 11/648,423 (US 2007-0217997) and WO 2007/079218.

### Discovery and Optimization of Protease Inhibitor Binding Proteins

Protease binding proteins may be discovered by any method of ligand discovery known in the art. In certain embodiments, protease binding proteins may be discovered by screening a library. In certain embodiments, the library is a display library. A display library is a collection of entities; each entity includes an accessible polypeptide component and a recoverable component that encodes or identifies the polypeptide component. The polypeptide component is varied so that different amino acid sequences are represented. The polypeptide component can be of any length, e.g. from three amino acids to over 300 amino acids. A display library entity can include more than one polypeptide component, for example, the two polypeptide chains of a sFab. In one exemplary implementation, a display library can be used to identify proteins that bind to an protease of interest. In a selection, the polypeptide component of each member of the library is probed with the protease of interest (e.g., a catalytic domain of an MMP or other fragment) and if the polypeptide component binds to the protease, the display library member is identified, typically by retention on a support. In certain embodiments, the display library comprises antibodies. In other embodiments, the display library comprises Kunitz domains. In still other embodiments, the display library comprises peptides.

After selecting candidate library members that bind to a target, each candidate library member can be further analyzed, e.g., to further characterize its binding properties for the target, e.g., the protease of interest, or for binding to other protein, e.g., another metalloproteinase. Each candidate library member can be subjected to one or more secondary screening assays. The assay can be for a binding property, a catalytic property, an inhibitory property, a physiological property (e.g., cytotoxicity, renal clearance, immunogenicity), a structural property (e.g., stability, conformation, oligomerization state) or another functional property. The same assay can be used repeatedly, but with varying conditions, e.g., to determine pH, ionic, or thermal sensitivities.

As appropriate, the assays can use a display library member directly, a recombinant polypeptide produced from the nucleic acid encoding the selected polypeptide, or a synthetic peptide synthesized based on the sequence of the selected polypeptide. In the case of selected Fabs, the Fabs can be evaluated or can be modified and produced as intact IgG proteins. Exemplary assays for binding properties include ELISAs, homogenous binding assays, surface plasmon resonance (SPR) and cellular assays, the practice of which are well-known to those of skill in the art.

In addition to the use of display libraries, other methods can be used to obtain an protease binding antibody. For example, a protease or a region thereof can be used as an antigen in a non-human animal, e.g., a rodent. Humanized antibodies can be generated by replacing sequences of the Fv variable region that are not directly involved in antigen binding with equivalent sequences from human Fv variable regions. General methods for generating humanized antibodies are provided by Morrison, S. L., 1985, Science 229:1202-1207, by Oi et al., 1986, BioTechniques 4:214, and by Queen et al. U.S. Pat. Nos. 5,585,089, U.S. Pat. No. 5,693,761 and U.S. Pat. No. 5,693,762. Those methods include isolating, manipulating, and expressing the nucleic acid sequences that encode all or part of immunoglobulin Fv variable regions from at least one of a heavy or light chain. Numerous sources of such nucleic acid are available. For example, nucleic acids may be obtained from a hybridoma producing an antibody against a predetermined target, as described above. The recombinant DNA encoding the humanized antibody, or fragment thereof, can then be cloned into an appropriate expression vector.

Immunoglobin protease binding proteins (e.g., IgG or Fab MMP binding proteins) may be modified to reduce immunogenicity. Reduced immunogenicity is desirable in protease binding proteins intended for use as therapeutics, as it reduces the chance that the subject will develop an immune response against the therapeutic molecule. Techniques useful for reducing immunogenicity of protease binding proteins include deletion/modification of potential human T cell epitopes and ‘germlining’ of sequences outside of the CDRs (e.g., framework and Fc).

A protease -binding antibody may be modified by specific deletion of human T cell epitopes or “deimmunization” by the methods disclosed in WO 98/52976 and WO 00/34317. Briefly, the heavy and light chain variable regions of an antibody are analyzed for peptides that bind to MHC Class II; these peptides represent potential T-cell epitopes (as defined in WO 98/52976 and WO 00/34317). For detection of potential T-cell epitopes, a computer modeling approach termed “peptide threading” can be applied, and in addition a database of human MHC class II binding peptides can be searched for motifs present in the VH and VL sequences, as described in WO 98/52976 and WO 00/34317. These motifs bind to any of the 18 major MHC class II DR allotypes, and thus constitute potential T cell epitopes. Potential T-cell epitopes detected can be eliminated by substituting small numbers of amino acid residues in the variable regions, or preferably, by single amino acid substitutions. As far as possible conservative substitutions are made, often but not exclusively, an amino acid common at this position in human germline antibody sequences may be used. Human germline sequences are disclosed in Tomlinson, I. A. et al., 1992, J. Mol. Biol. 227:776-798; Cook, G. P. et al., 1995, Immunol. Today Vol. 16 (5): 237-242; Chothia, D. et al., 1992, J. Mol. Bio. 227:799-817. The V BASE directory provides a comprehensive directory of human immunoglobulin variable region sequences (compiled by Tomlinson, I. A. et al. MRC Centre for Protein Engineering, Cambridge, UK). After the deimmunizing changes are identified, nucleic acids encoding V_{H} and V_{L} can be constructed by mutagenesis or other synthetic methods (e.g., de novo synthesis, cassette replacement, and so forth). Mutagenized variable sequence can, optionally, be fused to a human constant region, e.g., human IgG1 or K constant regions.

In some cases a potential T cell epitope will include residues which are known or predicted to be important for antibody function. For example, potential T cell epitopes are usually biased towards the CDRs. In addition, potential T cell epitopes can occur in framework residues important for antibody structure and binding. Changes to eliminate these potential epitopes will in some cases require more scrutiny, e.g., by making and testing chains with and without the change. Where possible, potential T cell epitopes that overlap the CDRs were eliminated by substitutions outside the CDRs. In some cases, an alteration within a CDR is the only option, and thus variants with and without this substitution should be tested. In other cases, the substitution required to remove a potential T cell epitope is at a residue position within the framework that might be critical for antibody binding. In these cases, variants with and without this substitution should be tested. Thus, in some cases several variant deimmunized heavy and light chain variable regions were designed and various heavy/light chain combinations tested in order to identify the optimal deimmunized antibody. The choice of the final deimmunized antibody can then be made by considering the binding affinity of the different variants in conjunction with the extent of deimmunization, i.e., the number of potential T cell epitopes remaining in the variable region. Deimmunization can be used to modify any antibody, e.g., an antibody that includes a non-human sequence, e.g., a synthetic antibody, a murine antibody other non-human monoclonal antibody, or an antibody isolated from a display library.

Protease binding antibodies are “germlined” by reverting one or more non-germline amino acids in framework regions to corresponding germline amino acids of the antibody, so long as binding properties are substantially retained. Similar methods can also be used in the constant region, e.g., in constant immunoglobulin domains.

Antibodies that bind to a protease, such as MMP, e.g., an antibody described above that binds to, for example, MMP-9, MMP-9/2, MMP-12 or MMP-14, may be modified in order to make the variable regions of the antibody more similar to one or more germline sequences. For example, an antibody can include one, two, three, or more amino acid substitutions, e.g., in a framework, CDR, or constant region, to make it more similar to a reference germline sequence. One exemplary germlining method can include identifying one or more germline sequences that are similar (e.g., most similar in a particular database) to the sequence of the isolated antibody. Mutations (at the amino acid level) are then made in the isolated antibody, either incrementally or in combination with other mutations. For example, a nucleic acid library that includes sequences encoding some or all possible germline mutations is made. The mutated antibodies are then evaluated, e.g., to identify an antibody that has one or more additional germline residues relative to the isolated antibody and that is still useful (e.g., has a functional activity). In one embodiment, as many germline residues are introduced into an isolated antibody as possible.

In one embodiment, mutagenesis is used to substitute or insert one or more germline residues into a framework and/or constant region. For example, a germline framework and/or constant region residue can be from a germline sequence that is similar (e.g., most similar) to the non-variable region being modified. After mutagenesis, activity (e.g., binding or other functional activity) of the antibody can be evaluated to determine if the germline residue or residues are tolerated (i.e., do not abrogate activity). Similar mutagenesis can be performed in the framework regions.

Selecting a germline sequence can be performed in different ways. For example, a germline sequence can be selected if it meets a predetermined criteria for selectivity or similarity, e.g., at least a certain percentage identity, e.g., at least 75, 80, 85, 90, 91, 92, 93, 94, 95, 96, 97, 98, 99, or 99.5% identity. The selection can be performed using at least 2, 3, 5, or 10 germline sequences. In the case of CDR1 and CDR2, identifying a similar germline sequence can include selecting one such sequence. In the case of CDR3, identifying a similar germline sequence can include selecting one such sequence, but may including using two germline sequences that separately contribute to the amino-terminal portion and the carboxy-terminal portion. In other implementations more than one or two germline sequences are used, e.g., to form a consensus sequence.

In one embodiment, with respect to a particular reference variable domain sequence, e.g., a sequence described herein, a related variable domain sequence has at least 30, 40, 50, 60, 70, 80, 90, 95 or 100% of the CDR amino acid positions that are not identical to residues in the reference CDR sequences, residues that are identical to residues at corresponding positions in a human germline sequence (i.e., an amino acid sequence encoded by a human germline nucleic acid).

In one embodiment, with respect to a particular reference variable domain sequence, e.g., a sequence described herein, a related variable domain sequence has at least 30, 50, 60, 70, 80, 90 or 100% of the FR regions identical to FR sequence from a human germline sequence, e.g., a germline sequence related to the reference variable domain sequence.

Accordingly, it is possible to isolate an antibody which has similar activity to a given antibody of interest, but is more similar to one or more germline sequences, particularly one or more human germline sequences. For example, an antibody can be at least 90, 91, 92, 93, 94, 95, 96, 97, 98, 99, or 99.5% identical to a germline sequence in a region outside the CDRs (e.g., framework regions). Further, an antibody can include at least 1, 2, 3, 4, or 5 germline residues in a CDR region, the germline residue being from a germline sequence of similar (e.g., most similar) to the variable region being modified. Germline sequences of primary interest are human germline sequences. The activity of the antibody (e.g., the binding activity as measured by K_{A}) can be within a factor or 100, 10, 5, 2, 0.5, 0.1, and 0.001 of the original antibody.

Germline sequences of human immunoglobin genes have been determined and are available from a number of sources, including the international ImMunoGeneTics information System® (IMGT), available via the world wide web at imgt.cines.fr, and the V BASE directory (compiled by Tomlinson, I. A. et al. MRC Centre for Protein Engineering, Cambridge, UK, available via the world wide web at vbase.mrc-cpe.cam.ac.uk).

Exemplary germline reference sequences for Vₖₐₚₚₐ include: O12/O2, O18/O8, A20, A30, L14, L1, L15, L4/18a, L5/L19, L8, L23, L9, L24, L11, L12, O11/O1, A17, A1, A18, A2, A19/A3, A23, A27, A11, L2/L16, L6, L20, L25, B3, B2, A26/A10, and A14. See, e.g., Tomlinson et al., 1995, EMBO J. 14(18):4628-3.

A germline reference sequence for the HC variable domain can be based on a sequence that has particular canonical structures, e.g., 1-3 structures in the H1 and H2 hypervariable loops. The canonical structures of hypervariable loops of an immunoglobulin variable domain can be inferred from its sequence, as described in Chothia et al., 1992, J. Mol. Biol. 227:799-817; Tomlinson et al., 1992, J. Mol. Biol. 227:776-798); and Tomlinson et al., 1995, EMBO J. 14(18):4628-38. Exemplary sequences with a 1-3 structure include: DP-1, DP-8, DP-12, DP-2, DP-25, DP-15, DP-7, DP-4, DP-31, DP-32, DP-33, DP-35, DP-40, 7-2, hv3005, hv3005f3, DP-46, DP-47, DP-58, DP-49, DP-50, DP-51, DP-53, and DP-54.

In one embodiment, a protease binding protein is physically associated with a moiety that improves its stabilization and/or retention in circulation, e.g., in blood, serum, lymph, or other tissues, e.g., by at least 1.5, 2, 5, 10, or 50 fold. For example, a protease binding protein can be associated with a polymer, e.g., a substantially non-antigenic polymers, such as polyalkylene oxides or polyethylene oxides. Suitable polymers will vary substantially by weight. Polymers having molecular number average weights ranging from about 200 to about 35,000 (or about 1,000 to about 15,000, and 2,000 to about 12,500) can be used. For example, a protease binding protein can be conjugated to a water soluble polymer, e.g., hydrophilic polyvinyl polymers, e.g. polyvinylalcohol and polyvinylpyrrolidone. A non-limiting list of such polymers include polyalkylene oxide homopolymers such as polyethylene glycol (PEG) or polypropylene glycols, polyoxyethylenated polyols, copolymers thereof and block copolymers thereof, provided that the water solubility of the block copolymers is maintained.

A protease binding protein can also be associated with a carrier protein, e.g., a serum albumin, such as a human serum albumin. For example, a translational fusion can be used to associate the carrier protein with the protease binding protein.

### Drug Conjugates

The protease binding proteins described herein can be conjugated to a drug (e.g., a DMARD, BRM or other immunosuppressive agent). The conjugates can be used therapeutically or prophylactically, e.g., the binding protein can target the drug, e.g., in vivo, e.g., to a site of disease (e.g., a site of inflammation), e.g., such that the drug affects the site of disease.

In some embodiments, the binding protein itself has therapeutic or prophylactic efficacy (e.g., the protein can modulate (e.g., antagonize) protease activity, or cause an effect on a cell that expresses a protease). The binding protein-drug conjugate can be used such that the binding protein and drug both contribute (e.g., additively or synergistically) to an effect on protease activity (e.g., a therapeutic effect, e.g., in vivo, e.g., to a site of disease (e.g., a site of undesired inflammation)). The drug and/or binding protein can be, for example, anti-inflammatory, immunosuppressive, etc. For example, if the targeted cell is a cancer cell, the drug and/or binding protein can prevent or reduce the ability of a tissue to become inflamed.

In certain embodiments wherein the inflammatory disorder being treated is rheumatoid arthtiris, classes of drugs that can be used as the “other member” in addition to the binding protein in the binding protein-drug conjugates described herein are as follows:

In some embodiments, the drug is an immunosuppressive agent such as gancyclovir, etanercept, cyclosporine, tacrolimus, rapamycin, cyclophosphamide, azathioprine, mycophenolate mofetil, methotrexate, cortisol, aldosterone, dexamethasone, a cyclooxygenase inhibitor, a 5-lipoxygenase inhibitor, or a leukotriene receptor antagonist. See generally US 2007-0292441; US 2007-0292422; US 2007-0264266; and US 2006-0233794. Also note that in some cases an “immunosuppressive agent” may be considered as a member of one of the other classes of drugs below, if that drug or agent has immunosuppressive activity.

In other embodiments, the drug is a DMARD such as methotrexate, Leflunomide, Sulphasalazine, Gold—parenteral (Myocrisin), Gold—oral (auranofin), antimalarials such as Hydroxychloroquine, Chloroquine phosphate, Chloroquine sulphate, antibiotics such as Minocycline, D-Penicillamine, Azathioprine, Cyclophosphamide, Cyclosporine, Leflunomide/Arava, TNF-α inhibitors/anti-TNF agents such as Etanercept (ENBREL®), Adalimumab (HUMIRA®), and Infliximab (REMICADE®). Note that in some cases a “DMARD” may be considered as a member of one of the other classes of drugs above or below, if that drug or agent has immunosuppressive activity or is a BRM.

In still other embodiments, the drug is a BRM such as a TNF-α inhibitor (Etanercept (ENBREL®), Adalimumab (HUMIRA®), and Infliximab (REMICADE®)), a CTLA4-Ig (Abatacept (ORENCIA®)) or anti-CD20 (RITUXAN®), any monoclonal antibody, interferon, interleukin-2, various types of colony-stimulating factors (CSF, GM-CSF, G-CSF), and Kineret (anakinra, IL-1 antagonist).

In other embodiments, the drug is an NSAID such as aspirin, naproxen, ibuprofen, etodolac, or salsalte.

In other embodiments, the drug is a corticosteroid.

In certain embodiments, a protease binding protein is physically linked to an immunosuppressive drug, DMARD or BRM. For example, by gentic fusion, a Kunitz domain inhibitor may be added to the C-terminus of a TNF-receptor-Fc fusion (e.g., ENBREL®) or anti-TNF IgG (e.g., HUMIRA® or REMICADE®). An anti-TNF Fab could be linked to a second Fab protease inhibitor at opposite termini of human serum albumin by genetic fusion. The Kunitz domain or Fab inhibitor could be linked to a Fab-PEG (e.g., CIMZIA®) conjugate by chemical modification.

Treatments for other inflammatory disorders such as COPD, asthma, MS, systemic sclerosis, inflammatory bowel disease and psoriasis that also may be incorporated into drug conjugates are described further below.

### Linkers

The binding proteins described herein can be associated with a drug to form a binding protein-drug conjugate by being linked to the drug directly. In some embodiments, the binding protein is directly conjugated to the drug. Alternatively, the binding proteins described herein can be associated with a drug to form a binding protein-drug conjugate by use of a linker region between the drug and the binding protein. In some embodiments, the binding protein is conjugated to the drug via a linker. The linker can be cleavable under intracellular conditions, e.g., such that cleavage of the linker releases the drug from the binding protein in the intracellular environment. In some embodiments, the cleavable linker is a peptide linker cleavable by an intracellular protease. In some embodiments, the peptide linker is a dipeptide linker.

In some embodiments, the dipeptide linker is a val-cit (vc) linker or a phe-lys (fk) linker. In some embodiments, the cleavable linker is hydrolyzable at a pH of less than 5.5. In some embodiments, the hydrolyzable linker is a hydrazone linker. In some embodiments, the cleavable linker is a disulfide linker.

For example, in some embodiments, the linker is cleavable by a cleaving agent that is present in the intracellular environment (e.g., within a lysosome or endosome or caveolea). The linker can be, e.g., a peptidyl linker that is cleaved by an intracellular peptidase or protease enzyme, including, but not limited to, a lysosomal or endosomal protease. In some embodiments, the peptidyl linker is at least two amino acids long or at least three amino acids long. Cleaving agents can include cathepsins B and D and plasmin, which are known to hydrolyze dipeptide drug derivatives resulting in the release of active drug inside target cells (see, e.g., Dubowchik and Walker Pharm. Therapeutics 83:67-123 (1999)). In some embodiments, peptidyl linkers are cleavable by enzymes that are present in targeted cells (e.g., cancer cells). For example, a peptidyl linker that is cleavable by the thiol-dependent protease cathepsin-B, which is highly expressed in cancerous tissue, can be used (e.g., a Phe-Leu or a Gly-Phe-Leu-Gly linker (SEQ ID NO: 58)). Other such linkers are described, e.g., in U.S. Pat. No. 6,214,345. In some embodiments, the peptidyl linker cleavable by an intracellular protease is a Val-Cit (vc) linker or a Phe-Lys linker (fk) (see, e.g., U.S. Pat. No. 6,214,345, which describes the synthesis of doxorubicin with the val-cit linker). One advantage of using intracellular proteolytic release of the drug is that the drug can be attenuated when conjugated and the serum stabilities of the conjugates are typically high.

In some preferred embodiments, a vc linker is used in the binding protein-drug conjugates described herein. For example, a binding protein-vcAFP or a binding protein-vcMMAF conjugate (e.g., a MMP binding protein-vcAFP or a MMP binding protein-vcMMAF conjugate) is prepared.

In other embodiments, the cleavable linker is pH-sensitive, i.e., sensitive to hydrolysis at certain pH values. For example, the pH-senstive linker is hydrolyzable under acidic conditions. For example, an acid-labile linker that is hydrolyzable in the lysosome (e.g., a hydrazone, semicarbazone, thiosemicarbazone, cis-aconitic amide, orthoester, acetal., ketal., or the like) can be used. See, e.g., U.S. Pat. Nos. 5,122,368; 5,824,805; 5,622,929; Dubowchik and Walker Pharm. Therapeutics 83:67-123 (1999); Neville et al. Biol. Chem. 264:14653-14661 (1989). Such linkers are relatively stable under neutral pH conditions, such as those in the blood, but are unstable at below pH 5.5 or 5.0, the approximate pH of the lysosome. In certain embodiments, the hydrolyzable linker is a thioether linker (such as, e.g., a thioether attached to the therapeutic agent via an acylhydrazone bond (see, e.g., U.S. Pat. No. 5,622,929)).

In yet other embodiments, the linker is cleavable under reducing conditions (e.g., a disulfide linker). A variety of disulfide linkers are known in the art, including, for example, those that can be formed using SATA (N-succinimidyl-5-acetylthioacetate), SPDP(N-succinimidyl-3-(2-pyridyldithio)propionate), SPDB (N-succinimidyl-3-(2-pyridyldithio)butyrate) and SMPT (N-succinimidyl-oxycarbonyl-alpha-methyl-alpha-(2-pyridyl-dithio)toluene)-, SPDB and SMPT (See, e.g., Thorpe et al. Cancer Res. 47:5924-5931 (1987); Wawrzynczak et al., In Immunoconjugates: Antibody Conjugates in Radioimagery and Therapy of Cancer (C. W. Vogel ed., Oxford U. Press, 1987). See also U.S. Pat. No. 4,880,935.

In yet other embodiments, the linker is a malonate linker (Johnson et al. Anticancer Res. 15:1387-93 (1995)), a maleimidobenzoyl linker (Lau et al. Bioorg-Med-Chem. 3(10):1299-1304 (1995), or a 3′-N-amide analog (Lau et al. Bioorg-Med-Chem. 3(10):1305-12 (1995)).

In some embodiments, the linker is not substantially sensitive to the extracellular environment. As used herein, “not substantially sensitive to the extracellular environment,” in the context of a linker, means that no more than about 20%, typically no more than about 15%, more typically no more than about 10%, and even more typically no more than about 5%, no more than about 3%, or no more than about 1% of the linkers, in a sample of a binding protein-drug conjugate, are cleaved when the binding protein-drug conjugate is present in an extracellular environment (e.g., in plasma). Whether a linker is not substantially sensitive to the extracellular environment can be determined, for example, by incubating independently with plasma both (a) the binding protein-drug conjugate (the “conjugate sample”) and (b) an equal molar amount of unconjugated binding protein or drug (the “control sample”) for a predetermined time period (e.g., 2, 4, 8, 16, or 24 hours) and then comparing the amount of unconjugated binding protein or drug present in the conjugate sample with that present in control sample, as measured, for example, by high performance liquid chromatography.

In other, non-mutually exclusive embodiments, the linker promotes cellular internalization. In certain embodiments, the linker promotes cellular internalization when conjugated to the drug (i.e., in the milieu of the linker-drug moiety of the binding protein-drug conjugate described herein). In yet other embodiments, the linker promotes cellular internalization when conjugated to both the drug and the binding protein.

A variety of linkers that can be used with the present compositions and methods are described in WO 2004/010957.

In some embodiments, the binding protein-drug conjugates described herein are used therapeutically in the treatment of an inflammatory disorder. In certain embodiments, it is desirable to only target a binding protein-drug conjugate to a cell that expresses the target to which the protease binding protein binds (e.g., to only target a MMP expressing cell to which a MMP binding protein binds, and not target a nearby “bystander” cell), e.g., to minimize toxicity. In other embodiments, it is desirable to target a binding protein-drug conjugate to a cell expressing the target to which the binding protein binds and also to bystander cells (e.g., to elicit a “bystander effect”).

Techniques for conjugating therapeutic agents to proteins (such as protease binding proteins, e.g., MMP binding proteins) are known. See, e.g., Amon et al., “Monoclonal Antibodies For Immunotargeting Of Drugs In Cancer Therapy,” in Monoclonal Antibodies And Cancer Therapy (Reisfeld et al eds., Alan R. Liss, Inc., 1985); Hellstrom et al., “Antibodies For Drug Delivery,” in Controlled Drug Delivery (Robinson et al. eds., Marcel Deiker, Inc., 2nd ed. 1987); Thorpe, “Antibody Carriers Of Cytotoxic Agents In Cancer Therapy: A Review,” in Monoclonal Antibodies '84: Biological And Clinical Applications (Pinchera et al. eds., 1985); “Analysis, Results, and Future Prospective of the Therapeutic Use of Radiolabeled Antibody In Cancer Therapy,” in Monoclonal Antibodies For Cancer Detection And Therapy (Baldwin et al. eds., Academic Press, 1985); and Thorpe et al. Immunol. Rev. 62:119-58 (1982). See also, e.g., US 2006-0233794 and PCT publication WO 89/12624.

In one embodiment, a protease binding protein (e.g., an MMP binding protein, an antibody) is conjugated to the cytotoxic agent via a linker. In one embodiment, the linker is cleavable under intracellular conditions, e.g., the cleavable linker is a peptide linker cleavable by an intracellular protease. In one embodiment, the linker is a peptide linker, e.g., a dipeptide linker, e.g., a val-cit linker or a phe-lys linker. In one embodiment, the cleavable linker is hydrolyzable at a pH of less than 5.5, e.g., the hydrolyzable linker is a hydrazone linker. In another embodiment, the cleavable linker is a disulfide linker.

In certain embodiments, a desired protease inhibitor drug, e.g., an MMP binding protein, may also be physically linked to an immunosuppressive drug, DMARD or BRM. For example, by gentic fusion, a Kunitz domain inhibitor could be added to the C-terminus of a TNF-receptor-Fc fusion (eg Enbrel) or anti-TNF IgG (e.g., HUMIRA® or REMICADE®). An anti-TNF Fab could be linked to a second Fab protease inhibitor at opposite termini of human serum albumin by genetic fusion. The Kunitz domain or Fab inhibitor could be linked to a Fab-PEG (e.g., CIMZIA®) conjugate by chemical modification.

### Pharmaceutical Compositions

In another aspect, the disclosure provides compositions, e.g., pharmaceutically acceptable compositions or pharmaceutical compositions, which include a protease binding protein, particularly a MMP binding protein, e.g., an antibody molecule, other polypeptide or peptide identified as binding to an MMP described herein. The protease binding protein can be formulated together with a pharmaceutically acceptable carrier. Pharmaceutical compositions include therapeutic compositions and diagnostic compositions, e.g., compositions that include labeled protease binding proteins for in vivo imaging.

A pharmaceutically acceptable carrier includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal, or epidermal administration (e.g., by injection or infusion), although carriers suitable for inhalation and intranasal administration are also contemplated. Depending on the route of administration, the protease binding protein may be coated in a material to protect the compound from the action of acids and other natural conditions that may inactivate the compound.

A pharmaceutically acceptable salt is a salt that retains the desired biological activity of the parent compound and does not impart any undesired toxicological effects (see e.g., Berge, S. M., et al., 1977, J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous, and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids, and the like. Base addition salts include those derived from alkaline earth metals, such as sodium, potassium, magnesium, calcium, and the like, as well as from nontoxic organic amines, such as N,N′-dibenzylethylenediamine, N-methylglucamine, chloroprocaine, choline, diethanolamine, ethylenediamine, procaine, and the like.

The compositions may be in a variety of forms. These include, for example, liquid, semi-solid and solid dosage forms, such as liquid solutions (e.g., injectable and infusible solutions), dispersions or suspensions, tablets, pills, powders, liposomes and suppositories. The form can depend on the intended mode of administration and therapeutic application. Many compositions are in the form of injectable or infusible solutions, such as compositions similar to those used for administration of humans with antibodies. An exemplary mode of administration is parenteral (e.g., intravenous, subcutaneous, intraperitoneal, intramuscular). In one embodiment, the protease binding protein is administered by intravenous infusion or injection. In another preferred embodiment, the protease binding protein is administered by intramuscular or subcutaneous injection.

The composition can be formulated as a solution, microemulsion, dispersion, liposome, or other ordered structure suitable to high drug concentration. Sterile injectable solutions can be prepared by incorporating the binding protein in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

A protease binding protein can be administered by a variety of methods, although for many applications, the preferred route/mode of administration is intravenous injection or infusion. For example, for therapeutic applications, the protease binding protein can be administered by intravenous infusion at a rate of less than 30, 20, 10, 5, or 1 mg/min to reach a dose of about 1 to 100 mg/m² or 7 to 25 mg/m². The route and/or mode of administration will vary depending upon the desired results. In certain embodiments, the active compound may be prepared with a carrier that will protect the compound against rapid release, such as a controlled release formulation, including implants, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are available. See, e.g., Sustained and Controlled Release Drug Delivery Systems, J. R. Robinson, ed., 1978, Marcel Dekker, Inc., New York.

Pharmaceutical compositions can be administered with medical devices. For example, in one embodiment, a pharmaceutical composition disclosed herein can be administered with a device, e.g., a needleless hypodermic injection device, a pump, or implant.

In certain embodiments, a protease binding protein can be formulated to ensure proper distribution in vivo. For example, the blood-brain barrier (BBB) excludes many highly hydrophilic compounds. To ensure that the therapeutic compounds disclosed herein cross the BBB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties that are selectively transported into specific cells or organs, thus enhance targeted drug delivery (see, e.g., V.V. Ranade, 1989, J. Clin. Pharmacol. 29:685).

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms can be dictated by and directly dependent on (a) the unique characteristics of the active compound and the particular therapeutic effect to be achieved, and (b) the limitations inherent in the art of compounding such an active compound for the treatment of sensitivity in individuals.

An exemplary, non-limiting range for a therapeutically or prophylactically effective amount of a protease binding protein disclosed herein is 0.1-20 mg/kg, more preferably 1-10 mg/kg. A protease binding protein can be administered, e.g., by intravenous infusion, e.g., at a rate of less than 30, 20, 10, 5, or 1 mg/min to reach a dose of about 1 to 100 mg/m² or about 5 to 30 mg/m². For protease binding proteins smaller in molecular weight than an antibody, appropriate amounts can be proportionally less. Dosage values may vary with the type and severity of the condition to be alleviated. For a particular subject, specific dosage regimens can be adjusted over time according to the individual need and the professional judgment of the person administering or supervising the administration of the compositions.

The pharmaceutical compositions disclosed herein may include a “therapeutically effective amount” or a “prophylactically effective amount” of a protease inhibitor such as a MMP binding protein disclosed herein.

A protease binding protein described herein can be provided as a pharmaceutical composition, e.g., including a pharmaceutically acceptable carrier. The composition can be at least 10, 20, 30, 50, 75, 85, 90, 95, 98, 99, or 99.9% free of other protein species. In some embodiments, the binding protein can be produced under GMP (good manufacturing practices). In some embodiments, the protease binding protein is provided in pharmaceutically acceptable carriers, e.g., suitable buffers or excipients.

The dose of a protease binding protein (e.g., a pharmaceutical composition containing a binding protein described herein) is sufficient to block about 20%, about 30%, about 40%, about 50%, about 60%, about 70%, about 80%, about 85%, about 90%, about 95%, about 99%, or about 100% of the activity of MMP in the patient, e.g., at the site of disease. Depending on the disease, this may require a dose, e.g., of between about 0.01 mg/Kg to about 100 mg/Kg, e.g., between about 0.1 and about 10 mg/Kg. For example, the dose can be a dose of about 0.1, about 1, about 3, about 6, or about 10 mg/Kg. For example, for an IgG having a molecular mass of 150,000g/mole (2 binding sites), these doses correspond to approximately 18 nM, 180 nM, 540 nM, 1.08 microM, and 1.8 microM, respectively, of binding sites for a 5 L blood volume. Medicine being partly an art, the optimal dose will be established by clinical trials, but will most likely lie in this range.

### Treatments

Protease binding proteins especially MMP binding proteins described and identified by the methods described herein and/or detailed herein (or protein-drug conjugates thereof) have therapeutic and prophylactic utilities, particularly in human subjects. These protease binding proteins are administered to a subject to treat, prevent, and/or diagnose inflammatory disorders. Treating includes administering an amount effective to alleviate, relieve, alter, remedy, ameliorate, improve or affect the disorder, the symptoms of the disorder or the predisposition toward the disorder. The treatment may also delay onset, e.g., prevent onset, or prevent deterioration of a disease or condition.

Exemplary inflammatory disorders include, but are not limited to, asthma, alopecia greata, systemic lupus erythematosus, rheumatoid arthritis, reactive arthritis, spondylarthritis, systemic vasculitis, insulin dependent diabetes mellitus, multiple sclerosis, experimental allergic encephalomyelitis, Sjogren's syndrome, graft versus host disease, inflammatory bowel disease including Crohn's disease, ulcerative colitis, ischemia reperfusion injury, myocardial infarction, Alzheimer's disease, transplant rejection (allogeneic and xenogeneic), thermal trauma, any immune complex-induced inflammation, glomerulonephritis, myasthenia gravis, cerebral lupus, Guillain-Barre syndrome, vasculitis, systemic sclerosis, anaphlaxis, catheter reactions, atheroma, infertility, thyroiditis, ARDS, post-bypass syndrome, hemodialysis, juvenile rheumatoid, Behcets syndrome, hemolytic anemia, pemphigus, bullous pemphigoid, stroke, atherosclerosis, scleroderma, psoriasis, sarcoidosis, transverse myelitis, acute disseminated encephalomyelitis, post-infectious encephalomyelitis, subacute sclerosing panencephalitis, polymyositis, dermatomyositis, incusion body myopathy, and chronic inflammatory demyelinating polyradiculopathy.

Certain inflammatory disorders of particular interest are described in more detail below. As used herein, an amount of a protease binding protein effective to prevent an inflammatory disorder, or a prophylactically effective amount of the binding agent refers to an amount of a target binding agent, e.g., a kallikrein inhibitor, a plasmin inhibitor, cathepsin B inhibitor, an MMP (e.g., MMP-14, -12, -9, -9/2) binding protein (or MMP binding protein-drug conjugate), e.g., an anti-MT-MMP (e.g., anti-MMP-14) antibody (or anti-MT-MMP antibody-drug conjugate)described herein, which is effective, upon single- or multiple-dose administration to the subject, for preventing or delaying the occurrence of the onset or recurrence of a disorder, e.g., a disorder described herein.

A protease binding protein described herein can be used to reduce an inflammatory disorder in a subject, e.g., to treat rheumatoid arthritis. The method includes administering the binding agent to the subject, e.g., in an amount effective to modulate inflammation, a symptom of the disorder, or progression of the disorder. The protease binding protein (e.g., a kallikrein inhibitor, a plasmin inhibitor, cathepsin B inhibitor, an MMP (e.g., MMP-14, -12, -9, -9/2) binding protein (or MMP binding protein-drug conjugate), e.g., an anti-MMP (e.g., anti-MMP-14, -12, -9, -9/2) antibody (or anti-MMP antibody-drug conjugate)) may be administered multiple times (e.g., at least two, three, five, or ten times) before a therapeutically effective amount is attained.

Methods of administering protease binding proteins, or protease binding protein-drug conjugates, and other agents are also described in “Pharmaceutical Compositions.” Suitable dosages of the molecules used can depend on the age and weight of the subject and the particular drug used. The protease binding proteins (or protein-drug conjugates thereof) can be used as competitive agents to inhibit, reduce an undesirable interaction, e.g., between a natural or pathological agent and the protease (e.g., MMP-14). The dose of the protease (e.g., MMP-14) binding protein, or protease binding protein-drug conjugate, can be the amount sufficient to block 90%, 95%, 99%, or 99.9% of the activity of protease (e.g., MMP-14) in the patient, especially at the site of disease. Depending on the disease, this may require 0.1, 1.0, 3.0, 6.0, or 10.0 mg/Kg. For an IgG having a molecular mass of 150,000g/mole (two binding sites), these doses correspond to approximately 18 nM, 180 nM, 540 nM, 1.08 μM, and 1.8 μM of binding sites for a 5 L blood volume.

In one embodiment, the protease (e.g., plasmin, kallikrein, cathepsin B, MMP-14, -12, -9, -9/2) binding proteins, or protease binding protein-drug conjugates, are used to inhibit an activity (e.g., inhibit at least one activity of, reduce proliferation, migration, growth or viability) of a cell, e.g., a cancer cell in vivo. The protease binding proteins can be used by themselves or conjugated to an agent, e.g., a cytotoxic drug, cytotoxin enzyme, or radioisotope. This method includes: administering the protease binding protein alone or attached to an agent (e.g., a cytotoxic drug), to a subject requiring such treatment. For example, MMP (e.g., MMP-14, -12, -9, -9/2) binding proteins that do not substantially inhibit MMP (e.g., MMP-14, -12, -9, -9/2) may be used to deliver nanoparticles containing agents to MMP (e.g., MMP-14, -12, -9, -9/2) associated cells or tissues, e.g., joint tissue. Such MMP binding proteins can be conjugated to a drug (to form a MMP binding protein-drug conjugate) and deliver the drug to the MMP associated cells or tissues.

Alternatively, the protease binding proteins, or protease binding protein-drug conjugates, bind to cells in the vicinity of the inflamed tissue, but are sufficiently close to the tissue to directly or indirectly inhibit (e.g., inhibit at least one activity, reduce growth and proliferation, or kill) the inflammation.

The protease binding proteins may be used to deliver an agent (e.g., any of a variety of therapeutic drugs) to cells and tissues where the MMP (e.g., MMP-14, -12, -9, -9/2) is present. Exemplary agents include DMARDs, BRMs, immunosuppressive agents, etc.

Methods of administering protease (e.g., kallikrein, plasmin, cathepsin B, MMP-14, -12, -9, -9/2) binding proteins, or MMP binding protein-drug conjugates, are described in “Pharmaceutical Compositions.” Suitable dosages of the molecules used will depend on the age and weight of the subject and the particular drug used. The binding proteins can be used as competitive agents to inhibit or reduce an undesirable interaction, e.g., between a natural or pathological agent and the protease.

### Exemplary Diseases and Conditions

Diseases to be treated include rheumatoid arthritis, psoriasis, multiple sclerosis, systemic sclerosis, asthma, chronic obstructive pulmonary disease, inflammatory bowel disease (e.g., Crohn's and Ulcerative Colitis).

### Rheumatoid Arthritis and Associated Conditions

Rheumatoid arthritis (RA) is an autoimmune, chronic inflammatory disease that causes joint swelling and pain and normally results in joint destruction. RA generally follows a relapsing/remitting course, with “flares” of disease activity interspersed with remissions of disease symptoms. RA is associated with a number of additional inflammatory disorders, including Sjogren's syndrome (dry eyes and mouth caused by inflammation of tear and saliva glands), pleuritis (inflammation of the pleura that causes pain upon deep breath and coughing), rheumatoid nodules (nodular sites of inflammation that develop within the lungs), pericarditis (inflammation of the pericardium that causes pain when lying down or leaning forward), Felty syndrome (splenomegaly and leucopenia observed in conjunction with RA, making the subject prone to infection), and vasculitis (an inflammation of the blood vessels which can block blood flow). MMP-14 and MMP-16 have been implicated in rheumatoid arthritis.

Symptoms of active RA include fatigue, lack of appetite, low grade fever, muscle and joint aches, and stiffness. Muscle and joint stiffness are usually most notable in the morning and after periods of inactivity. During flares, joints frequently become red, swollen, painful, and tender, generally as a consequence of synovitis.

Treatment for rheumatoid arthritis involves a combination of medications, rest, joint strengthening exercises, and joint protection. Two classes of medications are used in treating rheumatoid arthritis: anti-inflammatory “first-line drugs,” and “Disease-Modifying Antirheumatic Drugs” (DMARDs). The first-line drugs, include NSAIDS (e.g., aspirin, naproxen, ibuprofen, and etodolac) and cortisone (corticosteroids). DMARDS, such as gold (e.g., gold salts, gold thioglucose , gold thiomalate, oral gold), methotrexate, sulfasalazine, D-penicillamine, azathioprine, cyclophosphamide, chlorambucil, and cyclosporine, leflunomide, etanercept, infliximab, anakinra, and adalimumab, and hydroxychloroquine, promote disease remission and prevent progressive joint destruction, but they are not anti-inflammatory agents.

The disclosure provides methods of treating (e.g., ameliorating, stabilizing, or eliminating one or more symptoms or ameliorating or stabilizing the subject's score on a RA scale) rheumatoid arthritis by administering a therapeutically effective amount of a protease binding protein such as a kallikrein-, plasmin-, cathepsin B-, or MMP-binding protein, particularly an MMP (e.g., MMP-14, -12, -9, -9/2) binding protein in combination with another therapeutic agent such as a DMARD, BRM, corticosteroid, or NSAID, or protease binding protein-drug conjugate (e.g., with one of the aforementioned therapeutic agents), to a subject having or suspected of having RA. The disclosure provides methods preventing rheumatoid arthritis by administering a therapeutically effective amount of a protease binding proteins such as a kallikrein-, plasmin-, cathepsin B-, or MMP-binding protein, particularly an MMP (e.g., MMP-14, -12, -9, -9/2) binding protein in combination with another therapeutic agent such as a DMARD, BRM, corticosteroid, or NSAID, or protease binding protein-drug conjugate (e.g., with one of the aforementioned therapeutic agents), to a subject at risk of having RA.

Further provided are methods of treating (e.g., ameliorating, stabilizing, or eliminating one or more symptoms) rheumatoid arthritis associated disorder (e.g., Sjogren's syndrome, pleuritis, pulmonary rheumatoid nodules, pericarditis, Felty syndrome, or vasculitis) by administering a therapeutically effective amount of a protease binding protein such as a MMP binding protein, particularly a MMP binding protein in combination with another therapeutic agent such as a DMARD, BRM, corticosteroid, or NSAID, or MMP binding protein-drug conjugate (such as with one of the aforementioned therapeutic agents). Further provided are methods of preventing a rheumatoid arthritis associated disorder (e.g., Sjogren's syndrome, pleuritis, pulmonary rheumatoid nodules, pericarditis, Felty syndrome, or vasculitis) by administering a therapeutically effective amount of a protease binding protein such as a MMP binding protein, particularly a MMP binding protein in combination with another therapeutic agent such as a DMARD, BRM, corticosteroid, or NSAID, or MMP binding protein-drug conjugate (such as with one of the aforementioned therapeutic agents).

Scales useful for assessing RA and symptoms of RA include the Rheumatoid Arthritis Severity Scale (RASS; Bardwell et al., (2002) Rheumatology 41(1):38-45), SF-36 Arthritis Specific Health Index (ASHI; Ware et al., (1999) Med. Care. 37(5 Suppl):MS40-50), Arthritis Impact Measurement Scales or Arthritis Impact Measurement Scales 2 (AIMS or AIMS2; Meenan et al. (1992) Arthritis Rheum. 35(1):1-10); the Stanford Health Assessment Questionnaire (HAQ), HAQII, or modified HAQ (see, e.g., Pincus et al. (1983) Arthritis Rheum. 26(11):1346-53).

Guidance for the determination of the dosage that delivers a therapeutically effective amount of an agent or combination of agents may be obtained from animal models of rheumatoid arthritis, such as collagen-induced arthritis (CIA), which is induced, typically in rodents, by immunization with autologous or heterologous type II collagen in adjuvant (Williams et al. Methods Mol. Med. 98:207-16 (2004)).

### COPD

Chronic Obstructive Pulmonary Disease (COPD), also known as chronic obstructive airway disease (COAD), is a group of diseases characterized by the pathological limitation of airflow in the airway that is not fully reversible. COPD is the umbrella term for chronic bronchitis, emphysema and a range of other lung disorders. It is most often due to tobacco smoking, but can be due to other airborne irritants such as coal dust, asbestos or solvents, as well as congenital conditions such as alpha-1-antitrypsin deficiency.

The main symptoms of COPD include dyspnea (shortness of breath) lasting for months or perhaps years, possibly accompanied by wheezing, and a persistent cough with sputum production. It is possible the sputum may contain blood (hemoptysis) and become thicker, usually due to damage of the blood vessels of the airways. Severe COPD could lead to cyanosis caused by a lack of oxygen in the blood. In extreme cases it could lead to cor pulmonale due to the extra work required by the heart to get blood to flow through the lungs.

COPD is particularly characterised by the spirometric measurement of a ratio of forced expiratory volume over 1 second (FEV₁) to forced vital capacity (FVC) being <0.7 and the FEV₁<80% of the predicted value as measured by a plethysmograph. Other signs include a rapid breathing rate (tachypnea) and a wheezing sound heard through a stethoscope. Pulmonary emphysema is NOT the same as subcutaneous emphysema, which is a collection of air under the skin that may be detected by the crepitus sounds produced on palpation.

Treatment for COPD includes inhalers that dilate the airways (bronchodilators) and sometimes theophylline. The COPD patient must stop smoking. In some cases inhaled steroids are used to suppress lung inflammation, and, in severe cases or flare-ups, intravenous or oral steroids are given. Antibiotics are used during flare-ups of symptoms as infections can worsen COPD. Chronic, low-flow oxygen, non-invasive ventilation, or intubation may be needed in some cases. Surgery to remove parts of the disease lung has been shown to be helpful for some patients with COPD. Lung rehabilitation programs may help some patients. Lung transplant is sometimes performed for severe cases. There are several types of bronchodilators used clinically with varying efficacy: for example, β₂ agonists, M₃ antimuscarinics, leukotriene antagonists, cromones, corticosteroids, and xanthines. These drugs relax the smooth muscles of the airway allowing for improved airflow. β₂ agonists include: Salbutamol (Ventolin), Bambuterol, Clenbuterol, Fenoterol, and Formoterol, and long acting β₂ agonists (LABAs) such as Salmeterol. M₃ muscarinic antagonists (anticholinergics) include the quaternary M₃ muscarinic antagonist Ipratropium, which is widely prescribed with the β₂ agonist salbutamol, Ipratropium, and Tiotropium, which can be combined with a LABA and inhaled steroid. Cromones include Cromoglicate and Nedocromil. Leukotriene antagonists can be used and include Montelukast, Pranlukast, and Zafirlukast. Xanthines include theophylline, methylxanthines, theobromine. More aggressive EMR interventions include IV H₁ antihistamines and IV dexamethasone. Phosphodiesterase-4 antagonists inlcude roflumilast and cilomilast. Corticosteroids can be used and include glucocorticoids, beclomethasone, mometasone, and fluticasone. Corticosteroids are often combined with bronchodilators in a single inhaler. Salmeterol and fluticasone can be combined (Advair). TNF antagonists include cachexin, cachectin infliximab, adalimumab and etanercept.

The disclosure provides methods of treating (e.g., ameliorating, stabilizing, or eliminating one or more symptoms of or ameliorating COPD by administering a therapeutically effective amount of a protease binding protein such as a kallikrein, plasmin, cathepsin B, or MMP binding protein, particularly a MMP binding protein in combination with COPD treatment (e.g., β₂ agonists, M₃ antimuscarinics, leukotriene antagonists, cromones, corticosteroids, and xanthines) or protease binding protein-drug conjugate (such as with one of the aforementioned COPD treatments), to a subject having or suspected of having COPD. The disclosure provides methods preventing COPD by administering therapeutically effective amount of a protease binding protein such as a kallikrein-, plasmin-, cathepsin B-, or MMP-binding protein, particularly a MMP (e.g., MMP-14, -12, -9, -9/2) binding protein in combination with COPD treatment (e.g., β₂ agonists, M₃ antimuscarinics, leukotriene antagonists, cromones, corticosteroids, and xanthines) or protease binding protein-drug conjugate (such as with one of the aforementioned COPD treatments), to a subject at risk of having COPD.

Guidance regarding the efficacy and dosage a therapeutic agent which will deliver a therapeutically effective amount of the protein can be obtained from an animal model of COPD, see e.g., PCT publication WO 2007/084486 and references cited therein.

### Asthma

Asthma is a chronic condition involving the respiratory system in which the airway occasionally constricts, becomes inflamed, and is lined with excessive amounts of mucus, often in response to one or more triggers. These episodes may be triggered by such things as exposure to an environmental stimulant (or allergen) such as cold air, warm air, moist air, exercise or exertion, or emotional stress. In children, the most common triggers are viral illnesses such as those that cause the common cold. This airway narrowing causes symptoms such as wheezing, shortness of breath, chest tightness, and coughing. The airway constriction responds to bronchodilators.

In some individuals asthma is characterized by chronic respiratory impairment. In others it is an intermittent illness marked by episodic symptoms that may result from a number of triggering events, including upper respiratory infection, stress, airborne allergens, air pollutants (such as smoke or traffic fumes), or exercise. Some or all of the following symptoms may be present in those with asthma: dyspnea, wheezing, stridor, coughing, an inability for physical exertion. Some asthmatics who have severe shortness of breath and tightening of the lungs never wheeze or have stridor and their symptoms may be confused with a COPD-type disease.

An acute exacerbation of asthma is commonly referred to as an asthma attack. The clinical hallmarks of an attack are shortness of breath (dyspnea) and either wheezing or stridor.

During an asthma episode, inflamed airways react to environmental triggers such as smoke, dust, or pollen. The airways narrow and produce excess mucus, making it difficult to breathe. In essence, asthma is the result of an immune response in the bronchial airways.

The airways of asthmatics are “hypersensitive” to certain triggers/stimuli. In response to exposure to these triggers, the bronchi (large airways) contract into spasm (an “asthma attack”). Inflammation soon follows, leading to a further narrowing of the airways and excessive mucus production, which leads to coughing and other breathing difficulties.

The most effective treatment for asthma is identifying triggers, such as pets or aspirin, and limiting or eliminating exposure to them. Desensitization is currently the only known “cure” to the disease.

Symptomatic control of episodes of wheezing and shortness of breath is generally achieved with fast-acting bronchodilators.

Relief medication: Short-acting, selective beta₂-adrenoceptor agonists, such as salbutamol (albuterol USAN), levalbuterol, terbutaline and bitolterol, can be used. Older, less selective adrenergic agonists, such as inhaled epinephrine and ephedrine tablets, can be used. Anticholinergic medications, such as ipratropium bromide may be used.

Preventative medication: Current treatment protocols recommend prevention medications such as an inhaled corticosteroid, which helps to suppress inflammation and reduces the swelling of the lining of the airways, in anyone who has frequent (greater than twice a week) need of relievers or who has severe symptoms. If symptoms persist, additional preventive drugs are added until the asthma is controlled. With the proper use of prevention drugs, asthmatics can avoid the complications that result from overuse of relief medications. Preventive agents include: inhaled glucocorticoids (e.g., ciclesonide, beclomethasone, budesonide, flunisolide, fluticasone, mometasone, and triamcinolone), leukotriene modifiers (e.g., montelukast, zafirlukast, pranlukast, and zileuton), mast cell stabilizers (e.g., cromoglicate (cromolyn), and nedocromil), antimuscarinics/anticholinergics (e.g., ipratropium, oxitropium, and tiotropium), methylxanthines (e.g., theophylline and aminophylline), antihistamines, an IgE blocker such as omalizumab, methotrexate).

Long-acting beta₂-adrenoceptor agonists can be used and include salmeterol, formoterol, bambuterol, and sustained-release oral albuterol. Combinations of inhaled steroids and long-acting bronchodilators are becoming more widespread; the most common combination currently in use is fluticasone/salmeterol (Advair in the United States, and Seretide in the United Kingdom). Another combination is budesonide/formoterol which is commercially known as Symbicort.

The disclosure provides methods of treating (e.g., ameliorating, stabilizing, or eliminating one or more symptoms of or ameliorating asthma by administering a therapeutically effective amount of a protease binding protein combination with asthma treatment (e.g., glucocorticoids, leukotriene modifiers, mast cell stabilizers, antimuscarinics/anticholinergics, antihistamines, an IgE blocker, methotrexate) or protease binding protein-drug conjugate (such as with one of the aforementioned asthma treatments), to a subject having or suspected of having asthma. The disclosure provides methods preventing asthma by administering a therapeutically effective amount of a protease binding protein combination with asthma treatment (e.g., glucocorticoids, leukotriene modifiers, mast cell stabilizers, antimuscarinics/anticholinergics, antihistamines, an IgE blocker, methotrexate) or protease binding protein-drug conjugate (such as with one of the aforementioned asthma treatments), to a subject at risk of having asthma.

Guidance regarding the efficacy and dosage for a therapeutic agent can be obtained from an animal model of asthma, see e.g., U.S. Pat. No. 5,602,302, or European Pat. No. EP1192944 B1, and references cited therein.

### Multiple Sclerosis

Multiple sclerosis (MS) is an autoimmune condition in which the immune system attacks the central nervous system (CNS), leading to demyelination. It may cause numerous physical and mental symptoms, and often progresses to physical and cognitive disability. Disease onset usually occurs in young adults, and is more common in women.

MS presents with a variety of symptoms, including changes in sensation (hypoesthesia); muscle weakness, abnormal muscle spasms, or difficulty in moving; difficulties with coordination and balance (ataxia); problems in speech (dysarthria) or swallowing (dysphagia); visual problems (nystagmus, optic neuritis, or diplopia); fatigue and acute or chronic pain syndromes; and bladder and bowel difficulties. Cognitive impairment of varying degrees, or emotional symptomatology in the form of depression or pseudobulbar affect are also common. Neuropathic pain is usual, and this can be in the form of Lhermitte's sign. Paraesthesias can be present and include pins and needles; tingling; shivering; burning pains; feelings of pressure; and areas of skin with heightened sensitivity to touch. The pains associated with these can be aching, throbbing, stabbing, shooting, gnawing, tingling, tightness and numbness. The main clinical measure of disability progression and severity of the symptoms is the Expanded Disability Status Scale or EDSS.

The initial attacks (also known as exacerbations or relapses) are often transient, mild (or asymptomatic), and self-limited. The common initial symptoms reported are: changes in sensation in the arms, legs or face (33%), complete or partial vision loss (optic neuritis) (16%), weakness (13%), double vision (7%), unsteadiness when walking (5%), and balance problems (3%); but many rare initial symptoms have been reported such as aphasia or psychosis. Optic neuritis or focal leg weakness may lead to falls and other serious accidents.

Several therapies have proven helpful for treatment of multiple sclerosis. The aims of treatment include returning function after an attack, preventing new attacks, and preventing disability. During symptomatic attacks administration of high doses of intravenous corticosteroids, such as methylprednisolone, is the routine therapy for acute relapses. Disease-modifying treatments including interferons (e.g., AVONEX®, REBIF®, BETAFERON®), glatiramer acetate (e.g., COPAXONE®), immunosuppressant (e.g., mitoxantrone) and natalizumab (e.g., TYSABRI®) are used for relapsing-remitting MS.

In human active demyelinating lesions, phagocytic macrophages are MMP-12 positive, suggesting a role for MMP-12 during demyelination in MS (Vos et al. J Neuroimmunol 2003: 138(1-2): 106).

The disclosure provides methods of treating (e.g., ameliorating, stabilizing, or eliminating one or more symptoms of or ameliorating MS by administering a therapeutically effective amount of a protease binding protein in combination with MS treatment (e.g., interferons (e.g., AVONEX®, REBIF®, BETAFERON®), glatiramer acetate (e.g., COPAXONE®), immunosuppressant (e.g., mitoxantrone) and natalizumab (e.g., TYSABRI®)) or protease binding protein-drug conjugate (such as with one of the aforementioned MS treatments), to a subject having or suspected of having MS. The disclosure provides methods of preventing MS by administering a therapeutically effective amount of a protease binding protein in combination with MS treatment (e.g., interferons (e.g., AVONEX®, REBIF®, BETAFERON®), glatiramer acetate (e.g., COPAXONE®), immunosuppressant (e.g., mitoxantrone) and natalizumab (e.g., TYSABRI®)) or protease binding protein-drug conjugate (such as with one of the aforementioned MS treatments), to a subject at risk of having MS.

Guidance regarding the efficacy and dosage for a therapeutic agent vcan be obtained from animal models of MS, see e.g., those described in Toft-Hansen et al. J. Immunol. 2004: 173(8): 5209, Tsutsui et al. J Neurosci 2004:24:1521, Anthony et al. J Neuroimmunol 1998: 87(1-2): 62 and references cited therein.

### Sclerosis

Systemic sclerosis (SSc) is the generalized type of scleroderma, which is a chronic disease characterized by excessive deposits of collagen in the skin or other organs. SSc can be fatal as a result of heart, kidney, lung or intestinal damage autoimmune disease.

Scleroderma affects the skin, and in more serious cases it can affect the blood vessels and internal organs. The more evident symptom is usually the hardening of the skin and associated scarring. Blood vessels may also be more visible. Many SSc patients (over 80%) have vascular symptoms and Raynaud's phenomenon. During an attack, there is discoloration of the hands and feet in response to cold. Raynaud's normally affects the fingers and toes. SSc and Raynaud's can cause painful ulcers on the fingers or toes which are known as digital ulcers. Calcinosis is also common in SSc, and is often seen near the elbows, knees or other joints. Diffuse scleroderma can cause musculoskeletal, pulmonary, gastrointestinal, renal and other complications. Patients with larger amounts of cutaneous involvement are more likely to have involvement of the internal tissues and organs.

Overproduction of MMP-12 by SSc microvascular endothelial cells (MVECs) has been shown to account for the cleavage of urokinase-type plasminogen activator receptor (uPAR) and the impairment of angiogenesis in vitro and may contribute to reduced angiogenesis in SSc patients (D'Alessio et al. Arthritis Rheum. 2004: 50(10): 3275).

Treatment for some of the symptoms of scleroderma includes drugs that soften the skin and reduce inflammation. Topical treatment for the skin changes of scleroderma do not alter the disease course, but may improve pain and ulceration. A range of NSAIDs (nonsteroidal anti-inflammatory drugs) can be used to ease painful symptoms, such as naproxen. Episodes of Raynaud's phenomenon sometimes respond to nifedipine or other calcium channel blockers; severe digital ulceration may respond to prostacyclin analogue iloprost, and the dual endothelin-receptor antagonist bosentan may be beneficial for Raynaud's phenomenon. The skin tightness may be treated systemically with methotrexate and cyclosporin. Scleroderma renal crisis, the occurrence of acute renal failure and malignant hypertension (very high blood pressure with evidence of organ damage) in people with scleroderma, is effectively treated with drugs from the class of the ACE inhibitors. Active alveolitis is often treated with pulses of cyclophosphamide, often together with a small dose of steroids. Pulmonary hypertension may be treated with epoprostenol, bosentan and possibly aerolized iloprost.

The disclosure provides methods of treating (e.g., ameliorating, stabilizing, or eliminating one or more symptoms of or ameliorating SSc by administering a therapeutically effective amount of a protease binding protein in combination with SSc treatment (e.g., NSAIDs, calcium channel blockers, prostacyclin analogue, the dual endothelin-receptor antagonist, methotrexate, cyclosporin, ACE inhibitors, cyclophosphamide, epoprostenol, and bosentan) or protease binding protein-drug conjugate (such as with one of the aforementioned SSc treatments), to a subject having or suspected of having SSc. The disclosure provides methods of preventing SSc by administering a therapeutically effective amount of a protease binding protein in combination with SSc treatment (e.g., NSAIDs, calcium channel blockers, prostacyclin analogue, the dual endothelin-receptor antagonist, methotrexate, cyclosporin, ACE inhibitors, cyclophosphamide, epoprostenol, and bosentan) or protease binding protein-drug conjugate (such as with one of the aforementioned SSc treatments), to a subject at risk of having SSc.

### Inflammatory Bowel Disease

Inflammatory bowel disease (IBD) is a group of serious, chronic relapsing inflammatory diseases affecting both the small and large intestine, which remains relatively resistant to current treatments. IBD is characterized by spontaneously occurring, chronic relapsing inflammation of unknown origin, in which current treatment options are inadequate. Despite extensive research into the disease in both humans and experimental animals, the precise mechanisms of pathology remain to be elucidated. A host of immune and inflammatory mediators are thought to be involved, including biogenic amines, kinins, arachidonic acid metabolites, free radicals, nitric oxide, various proinflammatory cytokines, and complement proteins.

By IBD is meant Crohn's Disease and ulcerative colitis including ulcerative proctitis, ulcerative proctosigmoiditis, lymphocytic colitis, intractable distal colitis, ileocolitis, collagenous colitis, microscopic colitis, pouchitis, radiation colitis, and antibiotic-associated colitis.

Depending on the level of severity, IBD may require immunosuppression to control the symptom, such as prednisone, infliximab (REMICADE®), azathioprine (IMURAN®), methotrexate, or 6-mercaptopurine. More commonly, treatment of IBD requires a form of mesalamine. Often, steroids are used to control disease flares and were once acceptable as a maintenance drug. In use for several years in Crohn's disease patients and recently in patients with ulcerative colitis, biologicals have been used such as the intravenously administered REMICADE®. Severe cases may require surgery, such as bowel resection, strictureplasty or a temporary or permanent colostomy or ileostomy.

Alternative medicine treatments for bowel disease exist in various forms, however such methods concentrate on controlling underlying pathology in order to avoid prolonged steroidal exposure or surgical excisement

Usually the treatment is started by administering drugs with high anti-inflammatory affects, such as prednisone. Once the inflammation is successfully controlled, the patient is usually switched to a lighter drug to keep the disease in remission, such as Asacol, a mesalamine. If unsuccessful, a combination of the aforementioned immunosuppression drugs with a mesalamine (which may also have an anti-inflammatory effect) may or may not be administered, depending on the patient.

Recent advances in drug development for IBD have involved the use of monoclonal antibodies to inhibit pro-inflammatory cytokines, such as interleukins, interferons and tumour necrosis factor alpha (TNF-α). In particular, the anti-TNF-α antibodies CDP571 and infliximab have been used clinically to treat Crohn's disease with some success.

The disclosure provides methods of treating (e.g., ameliorating, stabilizing, or eliminating one or more symptoms of or ameliorating IBD by administering a therapeutically effective amount of a protease binding protein in combination with IBD treatment (e.g., immunosuppresants, anti-TNF binding proteins, cytokine inhibitors, BRMs, anti-inflammatories, etc.) or protease binding protein-drug conjugate (such as with one of the aforementioned IBD treatments), to a subject having or suspected of having IBD. The disclosure provides methods of preventing IBD by administering a therapeutically effective amount of a protease binding protein in combination with IBD treatment (e.g., immunosuppresants, anti-TNF binding proteins, cytokine inhibitors, BRMs, anti-inflammatories, etc.) or protease binding protein-drug conjugate (such as with one of the aforementioned IBD treatments), to a subject at risk of having IBD.

### Psoriasis

Psoriasis is a well known condition which affects the skin of affected patients. The disease manifests itself as chronic, recurring silvery papules, scaling papules and plaques of various sizes. The condition may consist of one or two lesions or may be a widespread dermatosis with disabling arthritis or exfoliation. Psoriasis presents as elevated lesions that vary in size between one to several centimeters.

Psoriasis is one of the most common dermatologic diseases, affecting about 2 percent of the population to some degree. There are actually several types of psoriasis, with plaque psoriasis being the most common, but the underlying problem in all of them is overproduction of epidermal cells. Instead of adhering to the 21 to 28 day cycle of cell turnover, those afflicted with psoriasis race through the cycle in 3 or 4 days. The epidermis may grow to 5 to 10 times its normal thickness. The thickened epidermis, overgrowth of blood vessels, and infiltrate of neutrophils and lymphocytes account for the psoriatic lesions being raised and easily palpable. The lesions may range from a few to many at any given time.

The etiology of psoriasis is still poorly understood, however evidence has accumulated clearly indicating a role for T cells. Hordes of activated T cells are found in psoriatic skin and almost none in healthy skin. It has also been discovered that these activated T cells secrete interleukin-6, which has as one of its effects the ability to stimulate skin cell growth. The infiltration of activated white blood cells suggest that an immune response has been mustered against something.

There can be substantial variation between individuals in the effectiveness of specific psoriasis treatments. Because of this, dermatologists often use a trial-and-error approach to finding the most appropriate treatment for their patient. The decision to employ a particular treatment is based on the type of psoriasis, its location, extent and severity. The patient's age, sex, quality of life, comorbidities, and attitude toward risks associated with the treatment are also taken into consideration.

Medications with the least potential for adverse reactions are preferentially employed. If the treatment goal is not achieved then therapies with greater potential toxicity may be used. Medications with significant toxicity are reserved for severe unresponsive psoriasis. As a first step, medicated ointments or creams, called topical treatments, are applied to the skin. If topical treatment fails to achieve the desired goal then the next step would be to expose the skin to ultraviolet (UV) radiation. This type of treatment is called phototherapy. The third step involves the use of medications which are taken internally by pill or injection. This approach is called systemic treatment.

Over time, psoriasis can become resistant to a specific therapy. Treatments may be periodically changed to prevent resistance developing (tachyphylaxis) and to reduce the chance of adverse reactions occurring. This is called treatment rotation.

Topical Treatment

Bath solutions and moisturizers help soothe affected skin and reduce the dryness which accompanies the build-up of skin on psoriatic plaques. Medicated creams and ointments applied directly to psoriatic plaques can help reduce inflammation, remove built-up scale, reduce skin turn over, and clear affected skin of plaques. Ointment and creams containing coal tar, dithranol (anthralin), corticosteroids like desoximetasone (Topicort), vitamin D3 analogues (for example, calcipotriol), and retinoids are routinely used. Argan oil has also been used with some promising results. The mechanism of action of each is probably different but they all help to normalise skin cell production and reduce inflammation. Activated vitamin D and its analogues are highly effective inhibitors of skin cell proliferation.

Some topical agents are used in conjunction with other therapies, especially phototherapy.

Phototherapy

It has long been recognized that daily, short, non-burning exposure to sunlight helped to clear or improve psoriasis. Sunlight contains many different wavelengths of light. It was during the early part of the 20th century that it was recognised that for psoriasis the therapeutic property of sunlight was due to the wavelengths classified as ultraviolet (UV) light. Ultraviolet wavelengths are subdivided into UVA (380-315 nm) UVB (315-280 nm), and UVC (<280 nm). Ultraviolet B (UVB) (315-280 nm) is absorbed by the epidermis and has a beneficial effect on psoriasis. Narrowband UVB (311 to 312 nm), is that part of the UVB spectrum that is most helpful for psoriasis. Exposure to UVB several times per week, over several weeks can help people attain a remission from psoriasis.

Ultraviolet light treatment is frequently combined with topical (coal tar, calcipotriol) or systemic treatment (retinoids) as there is a synergy in their combination. The Ingram regime, involves UVB and the application of anthralin paste. The Goeckerman regime combines coal tar ointment with UVB.

Photochemotherapy

Psoralen and ultraviolet A phototherapy (PUVA) combines the oral or topical administration of psoralen with exposure to ultraviolet A (UVA) light. Precisely how PUVA works is not known. The mechanism of action probably involves activation of psoralen by UVA light which inhibits the abnormally rapid production of the cells in psoriatic skin. There are multiple mechanisms of action associated with PUVA, including effects on the skin immune system.

Systemic Treatment

Psoriasis which is resistant to topical treatment and phototherapy is treated by medications that are taken internally by pill or injection. This is called systemic treatment. Patients undergoing systemic treatment are required to have regular blood and liver function tests because of the toxicity of the medication. Pregnancy must be avoided for the majority of these treatments. Most people experience a recurrence of psoriasis after systemic treatment is discontinued.

The three main traditional systemic treatments are methotrexate, cyclosporine and retinoids. Methotrexate and cyclosporine are immunosupressant drugs; retinoids are synthetic forms of vitamin A. Other additional drugs, not specifically licensed for psoriasis, have been found to be effective. These include the antimetabolite tioguanine, the cytotoxic agent hydroxyurea, sulfasalazine, the immunosupressants mycophenolate mofetil, azathioprine and oral tacrolimus. These have all been used effectively to treat psoriasis when other treatments have failed. Although not licensed in many other countries fumaric acid esters have also been used to treat severe psoriasis in Germany for over 20 years.

Several immunomodulator drugs are approved for the treatment of moderate to severe cases of psoriasis. They include alefacept (AMEVIVE®), efalizumab (RAPTIVA®), etanercept (ENBREL®) and infliximab (REMICADE®). These drugs are given by intravenous infusion, intramuscular injection or subcutaneous injection and are usually used for people who have failed to respond to traditional therapy or for people with associated psoriatic arthritis. Biologics work by blocking interactions between certain immune system cells. They have strong effects on the immune system and likely pose many of the same risks as other immunosuppressant drugs.

The disclosure provides methods of treating (e.g., ameliorating, stabilizing, or eliminating one or more symptoms of or ameliorating psoriasis by administering a therapeutically effective amount of a protease binding protein in combination with psoriasis treatment (e.g., immunosuppresants, retinoids, immunomodulator (biologics) drugs, etc.) or protease binding protein-drug conjugate (such as with one of the aforementioned psoriasis treatments), to a subject having or suspected of having psoriasis. The disclosure provides methods of preventing psoriasis by administering a therapeutically effective amount of a protease binding protein in combination with psoriasis treatment (e.g., immunosuppresants, retinoids, immunomodulator (biologics) drugs, etc.) or protease binding protein-drug conjugate (such as with one of the aforementioned psoriasis treatments), to a subject at risk of having psoriasis.

### Combination Therapies

The protease binding proteins, e.g., plasmin-, kallikrein-, cathepsin B-, or MMP-binding proteins described herein, e.g., anti-MMP-9, MMP-9/2, MMP-12 or MMP-14 binding proteins, can be administered in combination with one or more of the above-described therapies for treating the particular inflammatory disorder of interest. The combination can result in a lower dose of the protease binding protein or other therapy being needed, such that side effects are reduced. The combination may result in enhanced delivery and efficacy of one or both agents. The agents or therapies can be administered at the same time (e.g., as a single formulation that is administered to a patient or as two separate formulations administered concurrently) or sequentially in any order. Sequential administrations are administrations that are given at different times. The time between administration of the one agent and another agent can be minutes, hours, days, or weeks. The use of a protease binding protein described herein can also be used to reduce the dosage of another therapy, e.g., to reduce the side-effects associated with another agent that is being administered, e.g., to reduce the side-effects of an immunosuppressive agent. Accordingly, a combination can include administering a second agent at a dosage at least 10, 20, 30, or 50% lower than would be used in the absence of the protease binding protein.

Second agents that can be used in combination with the protease binding proteins for treating RA are as follows.

In some embodiments for RA, the second agent is an immunosuppressive agent such as gancyclovir, etanercept, cyclosporine, tacrolimus, rapamycin, cyclophosphamide, azathioprine, mycophenolate mofetil, methotrexate, cortisol, aldosterone, dexamethasone, a cyclooxygenase inhibitor, a 5-lipoxygenase inhibitor, or a leukotriene receptor antagonist. See generally US 2007-0292441; US 2007-0292422; US 2007-0264266; and US 2006-0233794. Also note that in some cases an “immunosuppressive agent” may be considered as a member of one of the other classes of drugs below, if that drug or agent has immunosuppressive activity.

In other embodiments, the second agent is a DMARD such as methotrexate, Leflunomide, Sulphasalazine, Gold—parenteral (Myocrisin), Gold—oral (auranofin), antimalarials such as Hydroxychloroquine, Chloroquine phosphate, Chloroquine sulphate, antibiotics such as Minocycline, D-Penicillamine, Azathioprine, Cyclophosphamide, Cyclosporine, Leflunomide/Arava, TNF-α inhibitors/anti-TNF agents such as Etanercept (Enbrel®), Adalimumab (Humira®), and Infliximab (Remicade®). Note that in some cases a “DMARD” may be considered as a member of one of the other classes of drugs above or below, if that drug or agent has immunosuppressive activity or is a BRM.

In still other embodiments, the second agent is a BRM such as a TNF-α inhibitor (Etanercept (ENBREL®), Adalimumab (HUMIRA®), and Infliximab (REMICADE®)), a CTLA4-Ig (Abatacept (ORENCIA®)) or anti-CD20 (RITUXAN®), any monoclonal antibody, interferon, interleukin-2, various types of colony-stimulating factors (CSF, GM-CSF, G-CSF), and Kineret (anakinra, IL-1 antagonist).

In other embodiments, the second agent is an NSAID such as aspirin, naproxen, ibuprofen, etodolac, or salsalte.

In other embodiments, the second agent is a corticosteroid.

Second agents for use in treating other inflammatory disorders such as COPD, asthma, MS, systemic sclerosis, inflammatory bowel disease and psoriasis in such combination treatments are described above.

### Kits

A protease binding protein described herein can be provided in a kit, e.g., as a component of a kit. For example, such a kit may include (a) a protease binding protein, e.g., a composition that includes a protease binding protein, and, optionally (b) informational material. The informational material can be descriptive, instructional, marketing or other material that relates to the methods described herein and/or the use of a protease binding protein for the methods described herein. Optionally, the kit can include a second therapy, e.g., e.g., a therapy for an inflammatory disorder, e.g., for use in a combination therapy, e.g., as described herein.

The informational material of the kits is not limited in its form. In one embodiment, the informational material can include information about production of the compound, molecular weight of the compound, concentration, date of expiration, batch or production site information, and so forth. In one embodiment, the informational material relates to using the binding protein in combination with another agent to treat, prevent, or diagnose an inflammatory disorder.

In one embodiment, the informational material can include instructions to administer a protease binding protein in a suitable manner to perform the combination therapeutic methods described herein, e.g., in a suitable dose, dosage form, or mode of administration (e.g., a dose, dosage form, or mode of administration described herein). In another embodiment, the informational material can include instructions to administer a protease binding protein to a suitable subject, e.g., a human, e.g., a human having, or at risk for, an inflammatory disorder. For example, the material can include instructions to administer an MMP-9, -9/2, -12 or -14 binding protein to a patient with rheumatoid arthritis. The informational material of the kits is not limited in its form. In many cases, the informational material, e.g., instructions, is provided in print but may also be in other formats, such as computer readable material.

A protease binding protein can be provided in any form, e.g., liquid, dried or lyophilized form. It is preferred that a protease binding protein be substantially pure and/or sterile. When a protease binding protein is provided in a liquid solution, the liquid solution preferably is an aqueous solution, with a sterile aqueous solution being preferred. When a protease binding protein is provided as a dried form, reconstitution generally is by the addition of a suitable solvent. The solvent, e.g., sterile water or buffer, can optionally be provided in the kit.

The kit can include one or more containers for the composition containing a protease binding protein. In some embodiments, the kit contains separate containers, dividers or compartments for the composition and informational material. For example, the composition can be contained in a bottle, vial, or syringe, and the informational material can be contained association with the container. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of an binding protein. For example, the kit includes a plurality of syringes, ampules, foil packets, or blister packs, each containing a single unit dose of an binding protein. The containers of the kits can be air tight, waterproof (e.g., impermeable to changes in moisture or evaporation), and/or light-tight.

The kit optionally includes a device suitable for administration of the composition, e.g., a syringe, inhalant, dropper (e.g., eye dropper), swab (e.g., a cotton swab or wooden swab), or any such delivery device. In one embodiment, the device is an implantable device that dispenses metered doses of the protease binding protein. The disclosure also features a method of providing a kit, e.g., by combining components described herein.

### EXEMPLIFICATION

The following examples provide further illustration and are not limiting.

The contents of all references, pending patent applications and published patents, cited throughout this application are hereby expressly incorporated by reference.

### Example 1

### Evaluation of MMP-12 Inhibition in Mouse Collagen-Induced Arthritis

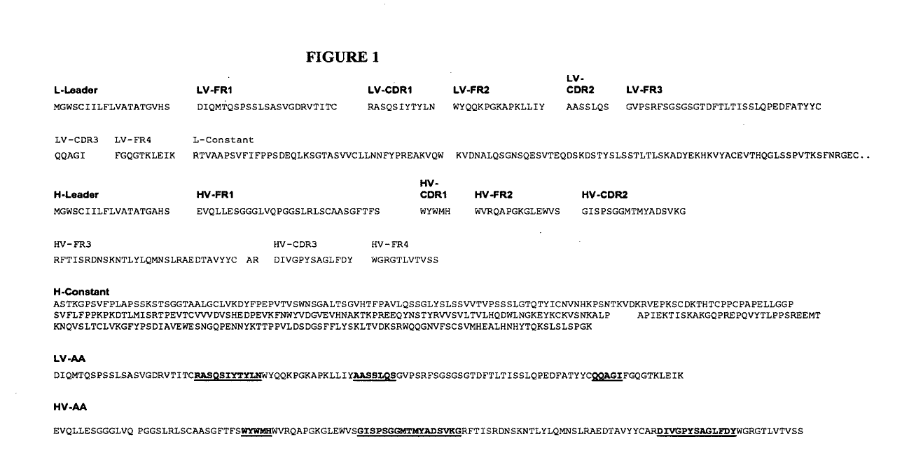

Inhibition of disease severity in a model of collagen-induced arthritis (CIA) was demonstrated using a Dyax inhibitor of MMP-12, DX-2712HEK (also referred to DX-2712 or M131A06-GA-S)) (FIG. 1).

The study design was as depicted in Table 6 below.

TABLE 6
MMP-12 Inhibition in CIA Study Design:

| | # of | | Dose | | Dose |
|---|---|---|---|---|---|
| Group # | Mice | Test Material | (mg/kg) | ROA | Regimen |
| 1 | 10 | Vehicle | N/A | IP | Q2D** |
| 2 | 10 | 539B-M131A06-GA-S | 20 | IP | Q2D** |
| 3 | 10 | Methotrexate | 3 | IP | QD |
| 4 | 10 | Non-sensitized | N/A | N/A | N/A |

Note:
539B-M131A06-GA-S = DX-2712HEK = MMP-12 antibody

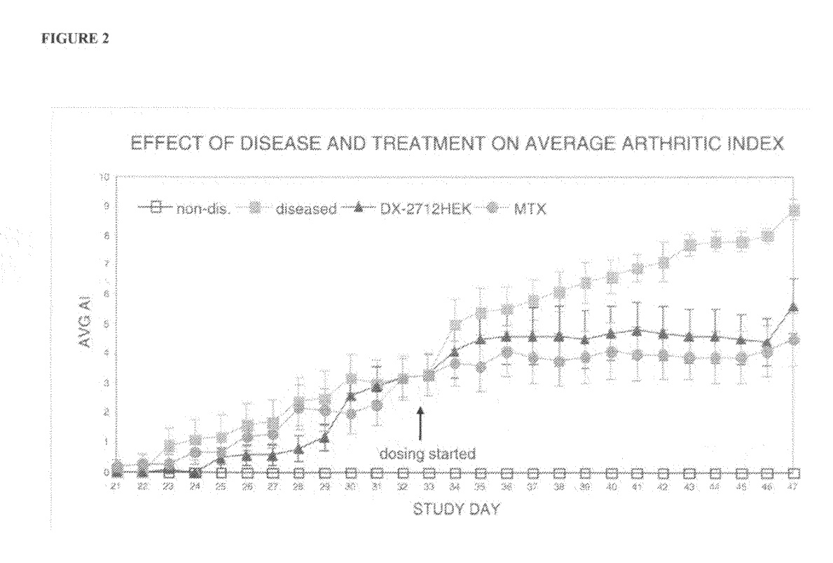

As shown in FIG. 2, treatment with DX-2712 resulted in delayed disease development through day 35. Disease development was arrested from days 36-46, but a sharp increase in arthritic score was recorded on the last day of the study.

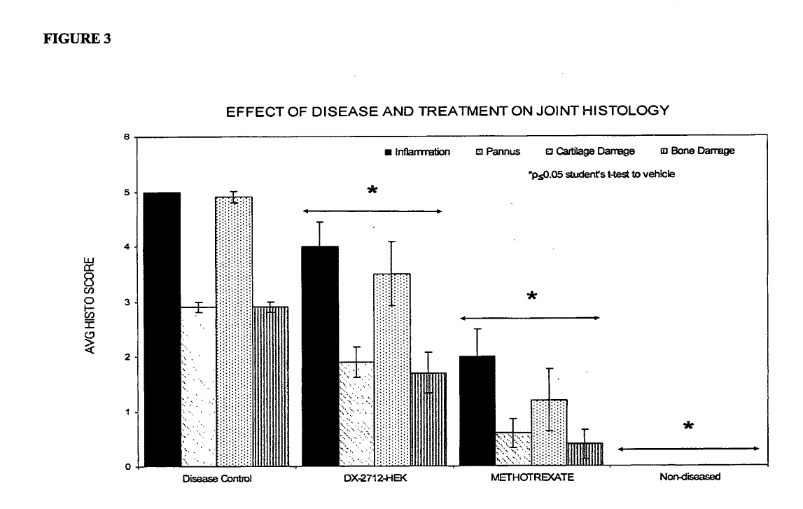

As shown in FIG. 3, DX-2712 treatment resulted in significant (p<0.05) overall 29% inhibition of the arthritic histological indices as compared to untreated mice.

### Example 2

### Evaluation of MMP-9/2 Inhibition in Mouse Collagen-Induced Arthritis

Inhibition of disease severity in a model of collagen-induced arthritis (CIA) was demonstrated using a Dyax inhibitor of MMP-9/2.

The study design was as depicted in Table 7 below.

TABLE 7
MMP-9/2 Inhibition in CIA Study Design:

| Group | Arthritic | Test Material | Dose |
|---|---|---|---|
| 1 | YES | PBS | N/A |
| 3 | YES | MMP-9/2 | 20 mg/kg |
| 4 | YES | Methotrexate | 3 mg/kg |
| 5 | NO | PBS | N/A |

Note:
Animals were dosed IP, Q2D

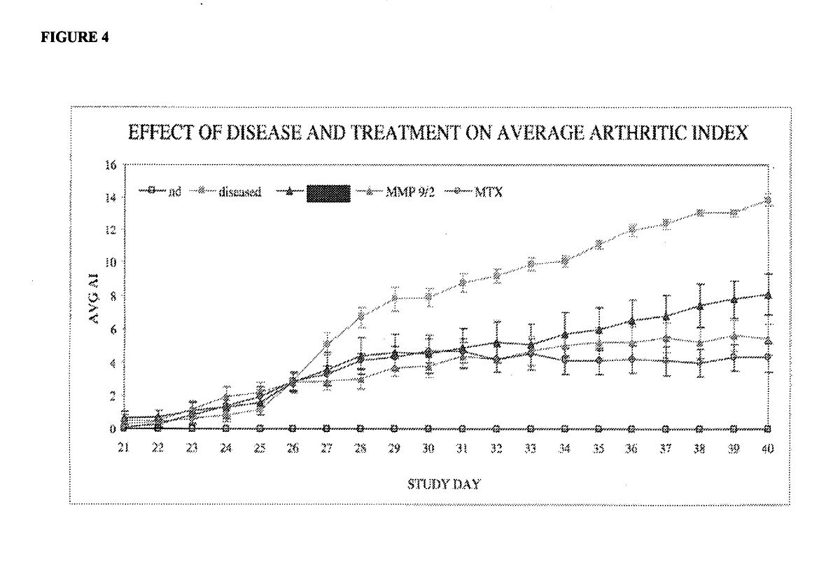

As shown in FIG. 4, therapeutic administration of 20 mg/kg of Dyax MMP-9/2 inhibitor, every other day by intraperitoneal route starting on day 26, not only had no affect on mouse weight gain, but also appeared to inhibit any further disease development as expressed by the Arthritic Index.

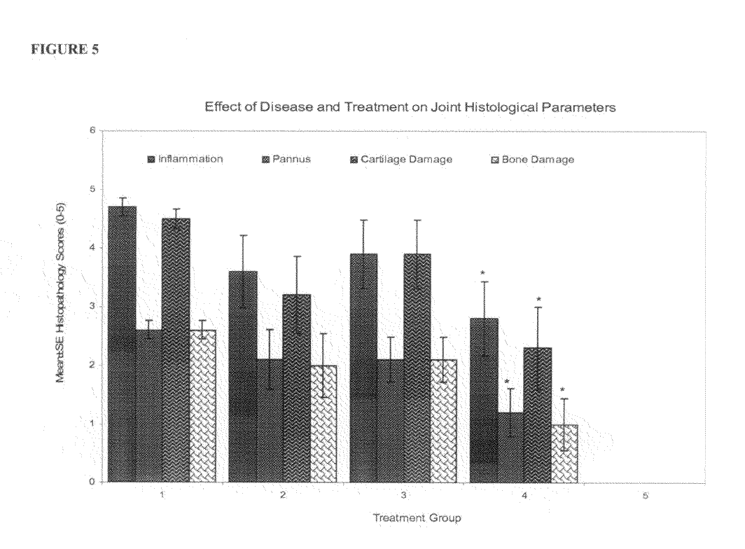

As shown in FIG. 5, animals in Group 3 were similar to the disease controls (Group 1) histologically, with no more than a 20% decrease for any parameter. Inflammation was chronic whenever noted. There was one animal with no arthritis in the submitted joint.

### Example 3

### Exemplary MMP-14 Binding Proteins

Examples of exemplary MMP-14 binding proteins include DX-2400 and DX-2410.

DX-2400. An exemplary MMP-14 antibody is DX-2400. The variable domain sequences for DX-2400 are:

### VH:

| (SEQ ID NO: 59) | |
|---|---|
| | FR1--------------------------- CDR1- FR2----------- CDR2------- |
| DX-2400 | EVQLLESGGGLVQPGGSLRLSCAASGFTFS LYSMN WVRQAPGKGLEWVS SIYSSGGSTLY |
| | CDR2-- FR3----------------------------- CDR3-- FR4--------- |
| DX-2400 | ADSVKG RFTISRDNSKNTLYLQMNSLRAEDTAVYYCAR GRAFDI WGQGTMVTVSS |

CDR regions are in bold.

### VL:

| (SEQ ID NO: 23) | |
|---|---|
| | FR1-------------------- CDR1------- FR2------------ CDR2--- |
| DX-2400 | DIQMTQSPSSLSASVGDRVTITC RASQSVGTYLN WYQQKPGKAPKLLIY ATSNLRS GVPS |
| | FR3------------------------- CDR3------ FR4------- |
| DX-2400 | RFSGSGSGTDFTLTISSLQPEDFATYYC QQSYSIPRFT FGPGTKVDIK |

CDR regions are in bold.

DX-2410. Another exemplary MMP-14 antibody is DX-2410. The variable domain sequences for DX-2410 are:

### VH:

| (SEQ ID NO: 24) | |
|---|---|
| | FR1--------------------------- CDR1- FR2----------- CDR2------- |
| DX2410 | EVQLLESGGGLVQPGGSLRLSCAASGFTFS VYGMV WVRQAPGKGLEWVS VISSSGGSTWY |
| | CDR2-- FR3----------------------------- CDR3------- FR4-------- |
| DX2410 | ADSVKG RFTISRDNSKNTLYLQMNSLRAEDTAVYYCAR PFSRRYGVFDY WGQGTLVTVSS |

CDR regions are in bold.

### VL:

| (SEQ ID NO: 25) | |
|---|---|
| | FR1-------------------- CDR1------- FR2------------ CDR2--- |
| DX2410 | DIQMTQSPSSLSASVGDRVTITC RASQGIRNFLA WYQQKPGKVPKLLIY GASALQS |
| | FR3----------------------------- CDR3----- FR4------- |
| DX2410 | GVPSRFSGSGSGTDFTLTISSLQPEDVATYYC QKYNGVPLT FGGGTKVEIK |

CDR regions are in bold.

See also WO 2009/079585.

### Example 4

### Exemplary MMP-9 Binding Proteins

Examples of exemplary MMP-9 binding proteins are provided below.

The DNA and amino acid sequences of variable regions of 539A-M0166-F10 sFAB are as follows:

539A-M0166-F10 (phage/SFAB) VL leader+VL

SEQ ID NO: [symbol] 63:
TTCTATTCTCACAGTGCACAGAGCGAATTGACTCAGCCACCGTCAGCG
TCTGCGGCCCCCGGGCAGAGGGTCACCATCTCTTGTTCTGGAAGCAGC
TCCAACATCGGAAGTAACACTGTAACCTGGTACCAGAAGCTCCCAGGA
ACGGCCCCCAAGCTCCTCATTTACAATAATTATGAGCGGCCCTCAGGG
GTCCCTGCCCGATTCTCTGGCTCCAAGTCTGGCACCTCAGCCTCCCTG
GCCATCAGTGGGCTCCAGTCTGAGGATGAGGCTGATTATTACTGTGCA
ACATGGGATGACAGCCTGATTGCCAATTACGTCTTCGGAAGTGGGACC
AAGGTCACCGTCCTAGGTCAGCCCAAGGCCAACCCC
SEQ ID NO: 3:
FYSHSAQSELTQPPSASAAPGQRVTISCSGSSSNIGSNTVTWYQKLPG
TAPKLLIYNNYERPSGVPARFSGSKSGTSASLAISGLQSEDEADYYCA
TWDDSLIANYVFGSGTKVTVLGQPKANP

539A-M0166-F10 (phage/SFAB) VH leader+VH

SEQ ID NO: [symbol] 64:
ATGAAGAAGCTCCTCTTTGCTATCCCGCTCGTCGTTCCTTTTGTGGCC
CAGCCGGCCATGGCCGAAGTTCAATTGTTAGAGTCTGGTGGCGGTCTT
GTTCAGCCTGGTGGTTCTTTACGTCTTTCTTGCGCTGCTTCCGGATTC
ACTTTCTCTCCTTACCTTATGAATTGGGTTCGCCAAGCTCCTGGTAAA
GGTTTGGAGTGGGTTTCTTCTATCTATTCTTCTGGTGGCGGTACTGGT
TATGCTGACTCCGTTAAAGGTCGCTTCACTATCTCTAGAGACAACTCT
AAGAATACTCTCTACTTGCAGATGAACAGCTTAAGGGCTGAGGACACG
GCCGTGTATTACTGTGCGAGAATATACCATAGCAGCAGTGGACCTTTC
TACGGTATGGACGTCTGGGGCCAAGGGACCACGGTCACCGTCTCAAGC
GCCTCCACCAAGGGCCCATCGGTCTTCCCGCTAGCACCCTCCTCCAAG
AGC
SEQ ID NO: [symbol] 65:
MKKLLFAIPLVVPFVAQPAMAEVQLLESGGGLVQPGGSLRLSCAASGF
TFSPYLMNWVRQAPGKGLEWVSSIYSSGGGTGYADSVKGRFTISRDNS
KNTLYLQMNSLRAEDTAVYYCARIYHSSSGPFYGMDVWGQGTTVTVSS
ASTKGPSVFPLAPSSKS

539A-M0240-B03 is a selective inhibitor of MMP-9. 539A-M0240-B03 can decrease or inhibit the activity of human and mouse MMP-9.

The sequences of the complememtarity determining regions (CDRs) of 539A-M0240-B03 light chain (LC) and heavy chain (HC) are as follows:

| LC CDR1: | |
|---|---|
| TGTSSDVGGYNYVS | (SEQ ID NO: 66) |
| LC CDR2: | |
| DVSKRPS | (SEQ ID NO: 67) |
| LC CDR3: | |
| CSYAGSYTLV | (SEQ ID NO: 68) |
| HC CDR1: | |
| TYQMV | (SEQ ID NO: 69) |
| HC CDR2: | |
| VIYPSGGPTVYADSVKG | (SEQ ID NO: 70) |
| HC CDR3: | |
| GEDYYDSSGPGAFDI | (SEQ ID NO: 71) |

A protein containing the HC CDR sequences of 539A-M0240-B03 (M0240-B03) and the light chain sequence shown below can be used in the methods described herein. A protein containing the LC CDRs shown below and the HC CDRs of 539A-M0240-B03, or a protein containing the LC variable region (light V gene) shown below and the 539A-M0240-B03 HC CDRs can also be used in the methods described herein. The protein can include a constant region sequence, such as the constant region (LC-lambda1) shown below.

```
Light V gene = VL2_2e; J gene = JL3
(SEQ ID NO: 72)
        FR1-L CDR1-L
QSALTQPRSVSGSPGQSVTISC TGTSSDVGGYNYVS
    FR2-L CDR2-L
WYQQHPGKAPKLMIY DVSKRPS GVPD
            FR3-L CDR3-L
RFSGSKSGNTASLTISGLQAEDEADYYC CSYAGSYTLV
FR4-L
FGGGTKLTVL
LC-lambdal
(SEQ ID NO: 73)
GQPKAAPSVTLFPPSSEELQANKATLVCLISDFYPGAVTVAWKADSSP
VKAGVETTTPSKQSNNKYAASSYLSLTPEQWKSHRSYSCQVTHEGSTV
EKTVAPTECS
```

CDR regions are in bold.

The amino acid and nucleic acid sequences for another exemplary protein that can be used in the methods described herein are provided below. A protein containing the LC and HC CDRs shown below, or a protein containing the light chain and heavy chain variable regions (LV and HV, respectively) shown below can also be used in the methods described herein.

539A-M0240-B03: Parental isolate (sFab; IgG-pBh1(f)).

539A-X0034-C02 (GS clone) (X0034-C02): DX-2802: Germlined, sequence optimized. The entire antibody fragment, containing the signal sequence, variable region and constant region of both the light and heavy chains were sequenced. The sequence data is available in 539A-R0108-A01 (539A-X0034-C02).

Light Chain

Light V gene=VL2_{—}2e 2e.2.2/V1-3/DPL12
Light J gene=JL3

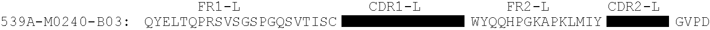

(SEQ ID NO: 74)

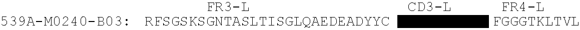

Heavy Chain

Heavy V gene: VH3_{—}3-23 DP-47/V3-23
Heavy J gene: JH3

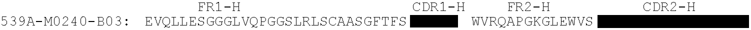

(SEQ ID NO: 75)

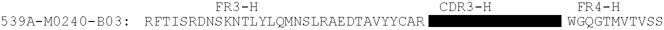

Light Variable

539A-M0240-B03-Light: Parental clone (sFab; IgG in pBhl(f)) light variable

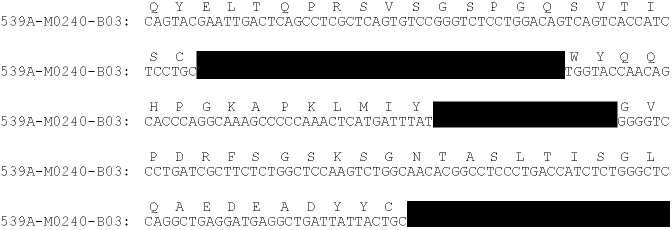

(SEQ ID NO: 74)

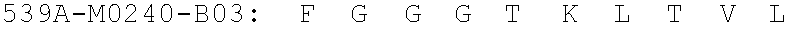

(SEQ ID NO: 76)

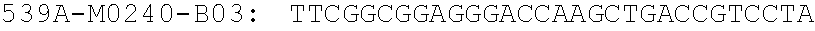

### Heavy Variable

539A-M0240-B03-Heavy: Parental clone (sFab; IgG in pBhl(f)) Heavy variable

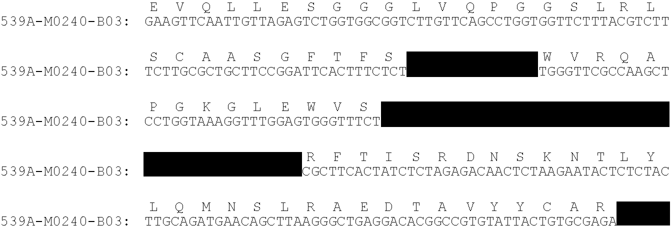

(SEQ ID NO: 75)

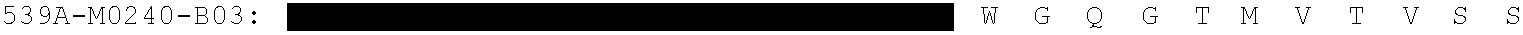

(SEQ ID NO: 77)

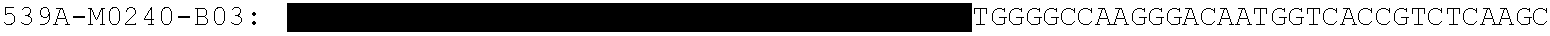

The amino acid and nucleic acid sequences for another exemplary protein that can be used in the methods described herein are provided below. A protein containing the LC and HC CDRs shown below, or a protein containing the light chain and heavy chain variable regions (LV and HV, respectively) shown below can also be used in the methods described herein. A protein containing the light chain and heavy chain (designated as LV+LC and HV+HC, respectively, below) sequences can also be used.

Light Chain

Light V gene=VL2_{—}2e 2e.2.2/V1-3/DPL12
Light J gene=JL3

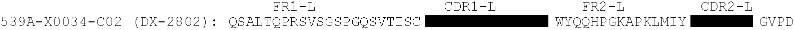

(SEQ ID NO: 72)

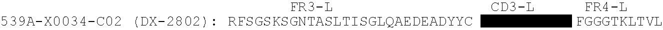

Heavy Chain

Heavy V gene: VH3_{—}3-23 DP-47/V3-23
Heavy J gene: JH3

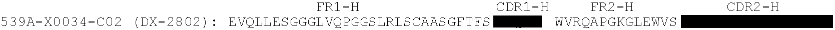

(SEQ ID NO: 75)

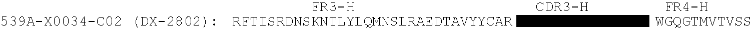

### Light Variable

539A-X0034-C02 (DX-2802)-Light: Germlined, codon optimized in GS vector

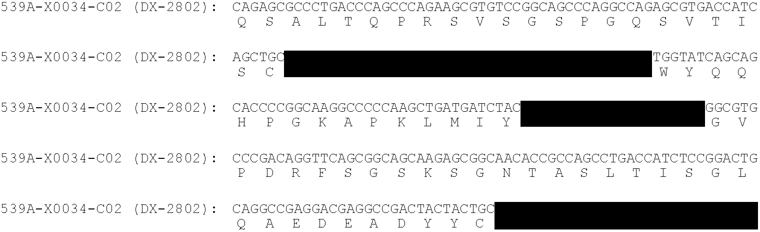

(SEQ ID NO: 78)

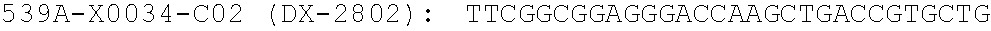

(SEQ ID NO: 72)

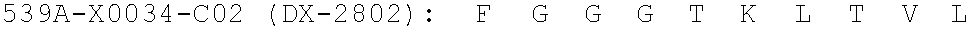

Heavy Variable

539A-X0034-C02 (DX-2802)-Heavy: Germlined, codon optimized in GS vector

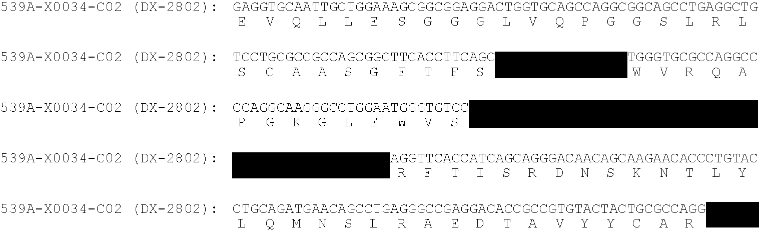

(SEQ ID NO: 79)

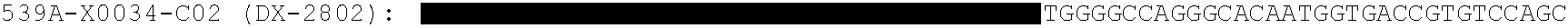

(SEQ ID NO: 75)

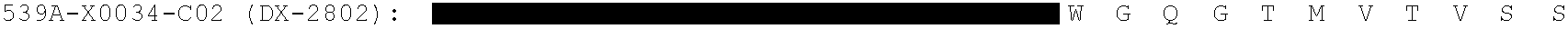

>539A-X0034-C02 (DX-2802): LV + LC dna
(SEQ ID NO: 80)
CAGAGCGCCCTGACCCAGCCCAGAAGCGTGTCCGGCAGCCCAGGCCAG
AGCGTGACCATCAGCTGCACCGGCACCAGCAGCGACGTGGGCGGCTAC
AACTACGTGTCCTGGTATCAGCAGCACCCCGGCAAGGCCCCCAAGCTG
ATGATCTACGACGTGTCCAAGAGGCCCAGCGGCGTGCCCGACAGGTTC
AGCGGCAGCAAGAGCGGCAACACCGCCAGCCTGACCATCTCCGGACTG
CAGGCCGAGGACGAGGCCGACTACTACTGCTGCAGCTACGCCGGCAGC
TACACCCTGGTGTTCGGCGGAGGGACCAAGCTGACCGTGCTGGGCCAG
CCCAAGGCTGCCCCCAGCGTGACCCTGTTCCCCCCCAGCAGCGAGGAA
CTGCAGGCCAACAAGGCCACACTGGTGTGCCTGATCAGCGACTTCTAC
CCAGGCGCCGTGACCGTGGCCTGGAAGGCCGACAGCAGCCCCGTGAAG
GCCGGCGTGGAGACAACCACCCCCAGCAAGCAGAGCAACAACAAGTAC
GCCGCCAGCAGCTACCTGAGCCTGACCCCCGAGCAGTGGAAGTCCCAC
AGGTCCTACAGCTGCCAGGTGACCCACGAGGGCAGCACCGTGGAGAAA
ACCGTGGCCCCCACCGAGTGTAGCTGATGA
> 539A-X0034-C02 (DX-2802): HV + HC dna
(SEQ ID NO: 81)
GAGGTGCAATTGCTGGAAAGCGGCGGAGGACTGGTGCAGCCAGGCGGC
AGCCTGAGGCTGTCCTGCGCCGCCAGCGGCTTCACCTTCAGCACCTAC
CAGATGGTGTGGGTGCGCCAGGCCCCAGGCAAGGGCCTGGAATGGGTG
TCCGTGATCTACCCCAGCGGCGGACCCACCGTGTACGCCGACAGCGTG
AAGGGCAGGTTCACCATCAGCAGGGACAACAGCAAGAACACCCTGTAC
CTGCAGATGAACAGCCTGAGGGCCGAGGACACCGCCGTGTACTACTGC
GCCAGGGGCGAGGACTACTACGACAGCAGCGGCCCAGGCGCCTTCGAC
ATCTGGGGCCAGGGCACAATGGTGACCGTGTCCAGCGCCAGCACCAAG
GGCCCCAGCGTGTTCCCGCTAGCACCTTCCTCCAAGTCCACCTCTGGC
GGCACCGCCGCTCTGGGCTGCCTGGTGAAGGACTACTTCCCTGAGCCT
GTGACCGTGAGCTGGAACTCTGGCGCCCTGACCTCCGGCGTGCATACC
TTCCCTGCCGTGCTGCAGTCCTCCGGCCTGTACTCCCTGTCCTCCGTG
GTGACAGTGCCTTCCTCCTCCCTGGGCACCCAGACCTACATCTGCAAC
GTGAACCACAAGCCTTCCAACACCAAGGTGGACAAGCGGGTGGAGCCT
AAGTCCTGCGACAAGACCCACACCTGCCCTCCCTGCCCTGCCCCTGAG
CTGCTGGGCGGACCCTCCGTGTTCCTGTTCCCTCCTAAGCCTAAGGAC
ACCCTGATGATCTCCCGGACCCCTGAGGTGACCTGCGTGGTGGTGGAC
GTGTCCCACGAGGACCCAGAGGTGAAGTTTAATTGGTATGTGGACGGC
GTGGAGGTCCACAACGCCAAGACCAAGCCTCGGGAGGAACAGTACAAC
TCCACCTACCGGGTGGTGTCCGTGCTGACCGTGCTGCACCAGGACTGG
CTGAACGGCAAGGAATACAAGTGCAAAGTCTCCAACAAGGCCCTGCCT
GCCCCCATCGAGAAAACCATCTCCAAGGCCAAGGGCCAGCCTCGCGAG
CCTCAGGTGTACACCCTGCCTCCTAGCCGGGAGGAAATGACCAAGAAC
CAGGTGTCCCTGACCTGTCTGGTGAAGGGCTTCTACCCTTCCGATATC
GCCGTGGAGTGGGAGTCCAACGGCCAGCCTGAGAACAACTACAAGACC
ACCCCTCCTGTGCTGGACTCCGACGGCTCCTTCTTCCTGTACTCCAAG
CTGACCGTGGACAAGTCCCGGTGGCAGCAGGGCAACGTGTTCTCCTGC
TCCGTGATGCACGAGGCCCTGCACAACCACTACACCCAGAAGTCCCTG
TCCCTGAGCCCTGGCAAGTGA
> 539A-X0034-C02 (DX-2802): LV + LC aa
(SEQ ID NO: 82)
QSALTQPRSVSGSPGQSVTISCTGTSSDVGGYNYVSWYQQHPGKAPKL
MIYDVSKRPSGVPDRFSGSKSGNTASLTISGLQAEDEADYYCCSYAGS
YTLVFGGGTKLTVLGQPKAAPSVTLFPPSSEELQANKATLVCLISDFY
PGAVTVAWKADSSPVKAGVETTTPSKQSNNKYAASSYLSLTPEQWKSH
RSYSCQVTHEGSTVEKTVAPTECSss
> 539A-X0034-C02 (DX-2802): HV + HC aa
(SEQ ID NO: 83)
EVQLLESGGGLVQPGGSLRLSCAASGFTFSTYQMVWVRQAPGKGLEWV
SVIYPSGGPTVYADSVKGRFTISRDNSKNTLYLQMNSLRAEDTAVYYC
ARGEDYYDSSGPGAFDIWGQGTMVTVSSASTKGPSVFPLAPSSKSTSG
GTAALGCLVKDYFPEPVTVSWNSGALTSGVHTFPAVLQSSGLYSLSSV
VTVPSSSLGTQTYICNVNHKPSNTKVDKRVEPKSCDKTHTCPPCPAPE
LLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALP
APIEKTISKAKGQPREPQVYTLPPSREEMTKNQVSLTCLVKGFYPSDI
AVEWESNGQPENNYKTTPPVLDSDGSFFLYSKLTVDKSRWQQGNVFSC
SVMHEALHNHYTQKSLSLSPGKs

Table 8 gives the LV and HV CDR sequences of M0279-A03, M0279-B02, M0288-008, and M0281-F06.

TABLE 8

| Initial Name | LV-CDR1 | LV-CDR2 | LV-CDR3 | HV-CDR1 | HV-CDR2 | HV-CDR3 |
|---|---|---|---|---|---|---|
| M0279-A03 | KASHSISRNLA | GASTRAT | QQRRNWPVT | QYPMW | YIVPSGGRTYYADSVKG | DRAYGDYVGWNGFDY |
| | (SEQ ID NO: | (SEQ ID | (SEQ ID | (SEQ ID | (SEQ ID NO: 94) | (SEQ ID NO: 95) |
| | 84) | NO: 88) | NO: 90) | NO: 93) | | |
| M0279-B02 | RASQSVSSNLA | GASTRAT | QQRRNWPVT | QYPMW | YIVPSGGRTYYADSVKG | DRAYGDYVGWNGFDY |
| | (SEQ ID NO: | (SEQ ID | (SEQ ID | (SEQ ID | (SEQ ID NO: 94) | (SEQ ID NO: 95) |
| | 85) | NO: 88) | NO: 90) | NO: 93) | | |
| M0288-C08 | RASQSVSSYLA | GASTRVT | QQRSSWPIT | QYPMW | YIVPSGGRTYYADSVKG | DRAYGDYVGWNGFDY |
| | (SEQ ID NO: | (SEQ ID | (SEQ ID | (SEQ ID | (SEQ ID NO: 94) | (SEQ ID NO: 95) |
| | 86) | NO: 89) | NO: 91) | NO: 93) | | |
| M0281-F06 | RASQSVSSDLA | GASTRAT | QQRSNWPVT | QYPMW | YIVPSGGRTYYADSVKG | DRAYGDYVGWNGFDY |
| | (SEQ ID NO: | (SEQ ID | (SEQ ID | (SEQ ID | (SEQ ID NO: 94) | (SEQ ID NO: 95) |
| | 87) | NO: 88) | NO: 92) | NO: 93) | | |

VL and VH for Fabs affinity matured for binding to hMMP9 and mMMP9

| M0279-A03-LC | |
|---|---|
| SEQ ID NO: 37 | |
| QDIQMTQSPATLSVSPGERVTLSC KASHSISRNLA WYQQKPGQAPRLLIF GASTRAT | |
| GIPARFSGSGSGTEFTLTISSLEAEDFAVYYC QQRRNWPVT FGPGTKLDFK | |
| M0279-A03-HC | |
| SEQ ID NO: 38 | |
| EVQLLESGGGLVQPGGSLRLSCAASGFTFS QYPMW WVRQAPGKGLEWVS YIVPSGGRTYYADSVKG | |
| RFTISRDNSKNTLYLQMNSLRAEDTAVYYCAK DRAYGDYVGWNGFDY WGQGTLVTVSS | |
| M0279-B02-LC | |
| SEQ ID NO: 39 | |
| QDIQMTQSPATLSVSPGERATLSC RASQSVSSNLA WYQQKPGQAPRLLIY GASTRAT | |
| GIPARFSGSGSGTEFTLTISSLQSEDFAVYYC QQRRNWPVT FGQGTRLEII | |
| M0279-B02-HC | |
| SEQ ID NO: 40 | |
| EVQLLESGGGLVQPGGSLRLSCAASGFTFS QYPMW WVRQAPGKGLEWVS YIVPSGGRTYYADSVKG | |
| RFTISRDNSKNTLYLQMNSLRAEDTAVYYCAK DRAYGDYVGWNGFDY WGQGTLVTVSS | |
| M0288-C08-LC | |
| SEQ ID NO: 41 | |
| QDIQMTQSPGTLSVSPGERVTLSC RASQSVSSYLA WYQQKPGQAPRLLIY GASTRVT | |
| GIPARFSGSGSGTEFTLTISSLQSEDFAVYYC QQRSSWPIT FGQGTRLEIK | |
| M0288-C08-HC | |
| SEQ ID NO: 42 | |
| EVQLLESGGGLVQPGGSLRLSCAASGFTFS QYPMW WVRQAPGKGLEWVS YIVPSGGRTYYADSVKG | |
| RFTISRDNSKNTLYLQMNSLRAEDTAVYYCAK DRAYGDYVGWNGFDY WGQGTLVTVSS | |
| LC for Fab M281-F06 | |
| (SEQ ID NO: 44) | |
| QDIQMTQSPA ALSLSPGERA TLSCRASQSV SSDLAWYQQK PGQAPRLLIY GASTRATGIP | 60 |
| ARFSGSRSGT AFTLTISSLE PEDFAVYYCQ QRSNWPVTFG QGTKLEIK | 108 |
| HC for Fab M281-F06 | |
| (SEQ ID NO: 45) | |
| EVQLLESGGG LVQPGGSLRL SCAASGFTFS QYPMWWVRQA PGKGLEWVSY IVPSGGRTYY | 60 |
| ADSVKGRFTI SRDNSKNTLY LQMNSLRAED TAVYYCAKDR AYGDYVGWNG FDYWGQGTLV | 120 |
| TVSS | 124 |

TABLE 9
Abs that bind MMP-9 but do not inhibit

M0076-D03-LC
SEQ ID NO [symbol] 96
QDIQMTQSPSSLSASVGDRVTITC RASQGIRNDLD
WYQQKPGTAPKRLIY SASNLQS
GVPSRFSGSGSGTEFTLTISNLQPEDLATYFC LQHNSFPLT
FGQGTKVEIK
M0076-D03-HC
SEQ ID NO [symbol] 97
EVQLLESGGGLVQPGGSLRLSCAASGFTFS LYRMN
WVRQAPGKGLEWVS YIGSSGGATAYADSVKG
RFTISRDNSKNTLYLQMNSLRAEDTAVYYCAR GAWYLDS
WGQGTLVTVSS
M0078-G07-LC
SEQ ID NO [symbol] 98
QDIQMTQSPGTLSVSPGERATLSC RASQSVSSDLA
WYQHKPGQAPRLLIY GVSTKAT
GVPARFSGSGSGTEFTLTISSLQSEDLAVYYC QQYHNWPPLT
FGGGTKVEIK
M0078-G07-HC
SEQ ID NO [symbol] 99
EVQLLESGGGLVQPGGSLRLSCAASGFTFS SYTME
WVRQAPGKGLEWVS WISPSGGYTFYADSVKG
RFTISRDNSKNTLYLQMNSLRAEDTALYYCAR GYSYGSIDL
WGRGTLVTVSS

See also WO 2009/111450 and WO 2009/111508.

### Example 5

### Exemplary MMP-9/-2 Binding Proteins

Examples of exemplary MMP-9/-2 binding proteins include M0237-D02, X0106-A01, X0106-B02, X0106-004, X0106-E4, and X0106-F05.

The DNA and amino acid sequences of variable regions of 539A-M0237-D02 sFAB are as follows:

539A-M0237-D02 (phage/SFAB) VL leader+VL

SEQ ID NO: [symbol] 101
TTCTATTCTCACAGTGCACAAGACATCCAGATGACCCAGTCTCCAGCC
ACCCTGTCTTTGTCTCCAGGGGAAAGAGCCACCCTCTCCTGCAGGGCC
AGTCAGAGTATTAGCAGCTTCTTAGCCTGGTACCAACAGAAACCTGGC
CAGGCTCCCAGGCTCCTCATCTATGATGCATCGTATAGGGCCACTGGC
ATCCCAGCCAGGTTCAGTGGCAGTGGGTCTGGGACAGACTTCACTCTC
ACCATCAGCAGCCTGGAGCCTGAAGATTATGCAGTTTATTACTGTCAG
CAGCGTGGCAACTGGCCTATCACCTTCGGCCAAGGGACGCGGCTGGAG
ATTAAACGAACTGTGGCTGCACCATCT
SEQ ID NO: [symbol] 102
FYSHSAQDIQMTQSPATLSLSPGERATLSCRASQSISSFLAWYQQKPG
QAPRLLIYDASYRATGIPARFSGSGSGTDFTLTISSLEPEDYAVYYCQ
QRGNWPITFGQGTRLEIKRTVAAPS

539A-M0237-D02 (phage/SFAB) VH leader+VH

SEQ ID NO: [symbol] 103
ATGAAGAAGCTCCTCTTTGCTATCCCGCTCGTCGTTCCTTTTGTGGCC
CAGCCGGCCATGGCCGAAGTTCAATTGTTAGAGTCTGGTGGCGGTCTT
GTTCAGCCTGGTGGTTCTTTACGTCTTTCTTGCGCTGCTTCCGGATTC
ACTTTCTCTCAGTACCCTATGTGGTGGGTTCGCCAAGCTCCTGGTAAA
GGTTTGGAGTGGGTTTCTTATATCGTTCCTTCTGGTGGCCGTACTTAT
TATGCTGACTCCGTTAAAGGTCGCTTCACTATCTCTAGAGACAACTCT
AAGAATACTCTCTACTTGCAGATGAACAGCTTAAGGGCTGAGGACACG
GCCGTGTATTACTGTGCGAAAGATCGGGCCTACGGTGATTACGTGGGC
TGGAACGGTTTTGACTACTGGGGCCAGGGCACCCTGGTCACCGTCTCA
AGCGCCTCCACCAAGGGCCCATCGGTCTTCCCGCTAGCACCCTCCTCC
AAGAGC
SEQ ID NO: [symbol] 104:
MKKLLFAIPLVVPFVAQPAMAEVQLLESGGGLVQPGGSLRLSCAASGF
TFSQYPMWWVRQAPGKGLEWVSYIVPSGGRTYYADSVKGRFTISRDNS
KNTLYLQMNSLRAEDTAVYYCAKDRAYGDYVGWNGFDYWGQGTLVTVS
SASTKGPSVFPLAPSSKS

Antibodies X0106-A01, X0106-B02, X0106-004, X0106-E4, and X0106-F05 were produced as IgG1s and tested for inhibition of human and mouse MMP-9 and MMP-2. X0106-A01, X0106-B02, X0106-C03, X0106-E4, and X0106-F05 are the germlined, optimized version of M0256-D11, M0276-F11, M0274-G08, M0275-D03, and M0307-F04, respectively.

Table 10 gives the LV and HV CDR sequences of the germlined, optimized MMP-9/MMP-2 specific antibodis (X0106-A01, X0106-B02, X0106-004, X0106-E4, and X0106-F05). Following Table 10 is a listing of the sequences of VL and VH plus part of the constant regions for these antibodies.

See also WO 2009/111508.

TABLE 10
CDRs of germlined, optimized hMMP2/hMMP9/mMMP2/mMMP9-inhibiting Abs

| Initial Name | LV-CDR1 | LV-CDR2 | LV-CDR3 | HV-CDR1 | HV-CDR2 | HV-CDR3 |
|---|---|---|---|---|---|---|
| X0106-A01 | RASQSISSFLA | DASYRAT | QQRGNWPIT | HYDMW | VIYSSGGPTFYADSVKG | DRAYGDYVGWNGFDY |
| | (SEQ ID NO: | (SEQ ID | (SEQ ID | (SEQ ID | (SEQ ID NO: 113) | (SEQ ID NO: 95) |
| | 105) | NO: 106) | NO: 107) | NO: 108) | | |
| X0106-B02 | RASQSISSFLA | DASYRAT | QQRGNWPIT | WYDMW | VIYSSGGPTFYADSVKG | DRAYGDYVGWNGFDY |
| | (SEQ ID NO: | (SEQ ID | (SEQ ID | (SEQ ID | (SEQ ID NO: 113) | (SEQ ID NO: 95) |
| | 105) | NO: 106) | NO: 107) | NO: 109) | | |
| X0106-C03 | RASQSISSFLA | DASYRAT | QQRGNWPIT | VYDMW | VIYPSGGATYYADSVKG | DRAYGDYVGWNGFDY |
| | (SEQ ID NO: | (SEQ ID | (SEQ ID | (SEQ ID | (SEQ ID NO: 114) | (SEQ ID NO: 95) |
| | 105) | NO: 106) | NO: 107) | NO: 110) | | |
| X0106-E04 | RASQSISSFLA | DASYRAT | QQRGNWPIT | QYDMW | VIYPSGTTFYADSVKG | DRAYGDYVGWNGFDY |
| | (SEQ ID NO: | (SEQ ID | (SEQ ID | (SEQ ID | (SEQ ID NO: 115) | (SEQ ID NO: 95) |
| | 105) | NO: 106) | NO: 107) | NO: 111) | | |
| X0106-F05 | RASQSISSFLA | DASYRAT | QQRGNWPIT | LYDMW | VIYSSGGYTGYADSVKG | DRAYGDYVGWNGFDY |
| | (SEQ ID NO: | (SEQ ID | (SEQ ID | SEQ ID | (SEQ ID NO: 116) | (SEQ ID NO: 95) |
| | 105) | NO: 106) | NO: 107) | NO: 112) | | |

VL and VH and part of constant region for Fabs germlined, optimized for binding to hMMP9, hMMP2, mMMP9, and mMMP2

X0106-A01-LC
SEQ ID NO: 43
EIVLTQSPATLSLSPGERATLSC RASQSISSFLA
WYQQKPGQAPRLLIY DASYRAT
GIPARFSGSGSGTDFTLTISSLEPEDFAVYYC QQRGNWPIT
FGQGTRLEIK
X0106-A01-HC
SEQ ID NO: [symbol] 117
EVQLLESGGGLVQPGGSLRLSCAASGFTFS HYDMW
WVRQAPGKGLEWVS VIYSSGGPTFYADSVKG
RFTISRDNSKNTLYLQMNSLRAEDTAVYYCAK DRAYGDYVGWNGFDY
WGQGTLVTVSS
X0106-B02-LC
SEQ ID NO: [symbol] 43
EIVLTQSPATLSLSPGERATLSC RASQSISSFLA
WYQQKPGQAPRLLIY DASYRAT
GIPARFSGSGSGTDFTLTISSLEPEDFAVYYC QQRGNWPIT
FGQGTRLEIK
X0106-B02-HC
SEQ ID NO: 46
EVQLLESGGGLVQPGGSLRLSCAASGFTFS WYDMW
WVRQAPGKGLEWVS VIYSSGGPTFYADSVKG
RFTISRDNSKNTLYLQMNSLRAEDTAVYYCAK DRAYGDYVGWNGFDY
WGQGTLVTVSS
X0106-C03-LC
SEQ ID NO: 47
EIVLTQSPATLSLSPGERATLSC RASQSISSFLA
WYQQKPGQAPRLLIY DASYRAT
GIPARFSGSGSGTDFTLTISSLEPEDFAVYYC QQRGNWPIT
FGQGTRLEIK
X0106-C03-HC
SEQ ID NO: 48
EVQLLESGGGLVQPGGSLRLSCAASGFTFS VYDMW
WVRQAPGKGLEWVS VIYPSGGATYYADSVKG
RFTISRDNSKNTLYLQMNSLRAEDTAVYYCAK DRAYGDYVGWNGFDY
WGQGTLVTVSS
X0106-E04-LC
SEQ ID NO: 49
EIVLTQSPATLSLSPGERATLSC RASQSISSFLA
WYQQKPGQAPRLLIY DASYRAT
GIPARFSGSGSGTDFTLTISSLEPEDFAVYYC QQRGNWPIT
FGQGTRLEIK
X0106-E04-HC
SEQ ID NO: 50
EVQLLESGGGLVQPGGSLRLSCAASGFTFS QYDMW
WVRQAPGKGLEWVS VIYPSGTTFYADSVKG
RFTISRDNSKNTLYLQMNSLRAEDTAVYYCAK DRAYGDYVGWNGFDY
WGQGTLVTVSS
X0106-F05-LC
SEQ ID NO: 51
EIVLTQSPATLSLSPGERATLSC RASQSISSFLA
WYQQKPGQAPRLLIY DASYRAT
GIPARFSGSGSGTDFTLTISSLEPEDFAVYYC QQRGNWPIT
FGQGTRLEIK
X0106-F05-HC
SEQ ID NO: 52
EVQLLESGGGLVQPGGSLRLSCAASGFTFS LYDMW
WVRQAPGKGLEWVS VIYSSGGYTGYADSVKG
RFTISRDNSKNTLYLQMNSLRAEDTAVYYCAK DRAYGDYVGWNGFDY
WGQGTLVTVSS

### Example 6

### Exemplary MMP-12 Binding Proteins

Examples of exemplary MMP-12 binding proteins include DX-2712, 539B-M0008-H09-GA-S, and 539B-M0121-E07-GA-S.

DX-2712 is also o referred to as M0131-A06-GA-S.

See also WO 2009/111507.

TABLE 11

| DX | ID | |
|---|---|---|
| number | NO. | Clone origin |
| DX-2712 | 1 | 539B-M0131-A06-GA-S |
| | 2 | 539E-M0008-H09-GA-S |
| | 3 | 539E-M0121-E07-GA-S |

| ID NO. | |
|---|---|
| (Contd) | VL |
| 1 | DIQMTQSPSSLSASVGDRVTITCRASQSIYTYLNWYQQKPG |
| | KAPKLLIYAASSLQSGVPSRFSGSGSGTDFTLTISSLQPED |
| | FATYYCQQAGIFGQGTKLEIK |
| | (SEQ ID NO: 118) |
| 2 | DIQMTQSPSSLSASVGDRVTITCRASQSIYTYLNWYQQKPG |
| | KAPKLLIYAASSLQSGVPSRFSGSGSGTDFTLTISSLQPED |
| | FATYYCQQAGIFGQGTKLEIK |
| | (SEQ ID NO: 118) |
| 3 | DIQMTQSPSSLSASVGDRVTITCRASQSIYTYLNWYQQKPG |
| | KAPKLLIYAASSLQSGVPSRFSGSGSGTDFTLTISSLQPED |
| | FATYYCQQAGIFGQGTKLEIK |
| | (SEQ ID NO: 118) |
| ID NO. | |
| (Contd) | CL |
| 1 | RTVAAPSVFIFPPSDEQLKSGTASVVCLLNNFYPREAKVQW |
| | KVDNALQSGNSQESVTEQDSKDSTYSLSSTLTLSKADYEKH |
| | KVYACEVTHQGLSSPVTKSFNRGEC |
| | (SEQ ID NO: 119) |
| 2 | RTVAAPSVFIFPPSDEQLKSGTASVVCLLNNFYPREAKVQW |
| | KVDNALQSGNSQESVTEQDSKDSTYSLSSTLTLSKADYEKH |
| | KVYACEVTHQGLSSPVTKSFNRGEC |
| | (SEQ ID NO: 119) |
| 3 | RTVAAPSVFIFPPSDEQLKSGTASVVCLLNNFYPREAKVQW |
| | KVDNALQSGNSQESVTEQDSKDSTYSLSSTLTLSKADYEKH |
| | KVYACEVTHQGLSSPVTKSFNRGEC |
| | (SEQ ID NO: 119) |
| ID NO. | |
| (Contd) | VH |
| 1 | EVQLLESGGGLVQPGGSLRLSCAASGFTFSWYWMHWVRQAP |
| | GKGLEWVSGISPSGGMTMYADSVKGRFTISRDNSKNTLYLQ |
| | MNSLRAEDTAVYYCARDIVGPYSAGLFDYWGRGTLVTVSS |
| | (SEQ ID NO: 120) |
| 2 | EVQLLESGGGLVQPGGSLRLSCAASGFTFSDYNMHWVRQAP |
| | GKGLEWVSYIGPSGGYTHYADSVKGRFTISRDNSKNTLYLQ |
| | MNSLRAEDTAVYYCAKDIRGAYSSGLFDYWGRGTLVTVSS |
| | (SEQ ID NO: 121) |
| 3 | EVQLLESGGGLVQPGGSLRLSCAASGFTFSWYGMHWVRQAP |
| | GKGLEWVSGIVSSGGETFYADSVKGRFTISRDNSKNTLYLQ |
| | MNSLRAEDTAVYYCAKDIRGVFLSGLFDHWGRGTLVTVSS |
| | (SEQ ID NO: 122) |
| ID | |
| NO. | CH |
| 123 | ASTKGPSVFPLAPSSKSTSGGTAALGCLVKDYFPEPVTVSW |
| | NSGALTSGVHTFPAVLQSSGLYSLSSVVTVPSSSLGTQTYI |
| | CNVNHKPSNTKVDKRVEPKSCDKTHTCPPCPAPELLGGPSV |
| | FLFTPKPKDTLMISRTPEVTCVVVDVSHEDPEVKFNWYVDG |
| | VEVHNAKTKPREEQYNSTYRVVSVLTVLHQDWLNGKEYKCK |
| | VSNKALPAPIEKTISKAKGQPREPQVYTLPPSREEMTKNQV |
| | SLTCLVKGFYPSDIAVEWESNGQPENNYKTTPPVLDSDGSF |
| | FLYSKLTVDKSRWQQGNVFSCSVMHEALHNHYTQKSLSLSP |
| | GK |
| 123 | ASTKGPSVFPLAPSSKSTSGGTAALGCLVKDYFPEPVTVSW |
| | NSGALTSGVHTFPAVLQSSGLYSLSSVVTVPSSSLGTQTYI |
| | CNVNHKPSNTKVDKRVEPKSCDKTHTCPPCPAPELLGGPSV |
| | FLFTPKPKDTLMISRTPEVTCVVVDVSHEDPEVKFNWYVDG |
| | VEVHNAKTKPREEQYNSTYRVVSVLTVLHQDWLNGKEYKCK |
| | VSNKALPAPIEKTISKAKGQPREPQVYTLPPSREEMTKNQV |
| | SLTCLVKGFYPSDIAVEWESNGQPENNYKTTPPVLDSDGSF |
| | FLYSKLTVDKSRWQQGNVFSCSVMHEALHNHYTQKSLSLSP |
| | GK |
| 123 | ASTKGPSVFPLAPSSKSTSGGTAALGCLVKDYFPEPVTVSW |
| | NSGALTSGVHTFPAVLQSSGLYSLSSVVTVPSSSLGTQTYI |
| | CNVNHKPSNTKVDKRVEPKSCDKTHTCPPCPAPELLGGPSV |
| | FLFTPKPKDTLMISRTPEVTCVVVDVSHEDPEVKFNWYVDG |
| | VEVHNAKTKPREEQYNSTYRVVSVLTVLHQDWLNGKEYKCK |
| | VSNKALPAPIEKTISKAKGQPREPQVYTLPPSREEMTKNQV |
| | SLTCLVKGFYPSDIAVEWESNGQPENNYKTTPPVLDSDGSF |
| | FLYSKLTVDKSRWQQGNVFSCSVMHEALHNHYTQKSLSLSP |
| | GK |

539B-X0041-D02 plus 13 mutants of 539B-X0041-D02:

| | Initial | L- | | | | | | | | |
|---|---|---|---|---|---|---|---|---|---|---|
| Isolate | Name | Leader | LV-FR1 | LV-CDR1 | LV-FR2 | LV-CDR2 | LV-FR3 | LV-CDR3 | LV-FR4 | L-Constant |
| 539B- | 539B- | GVHS | DIQMTQSPS | RASQSIY | WYQQKP | AASSLQS | GVPSRFSGSG | QQAGI | FGQGTKL | RTVAAPSVFIFPPS |
| X0041- | X0041-D02 | (SEQ ID | SLSASVGDR | TYLN | GKAPKL | (SEQ ID | SGTDFTLTIS | (SEQ ID | EIK | DEQLKSGTASVVCL |
| D02 | | NO: 124) | VTITC | (SEQ ID | LIY | NO: 128) | SLQPEDFATY | NO: 130) | (SEQ ID | LNNFYPREAKVQWK |
| | | | (SEQ NO: | NO: 126) | (SEQ ID | | YC | | NO: 131) | VDNALQSGNSQESV |
| | | | NO: 125) | | NO: 127) | | (SEQ ID | | | TEQDSKDSTYSLSS |
| | | | | | | | NO: 129) | | | TLTLSKADYEKHKV |
| | | | | | | | | | | YACEVTHQGLSSPV |
| | | | | | | | | | | TKSFNRGEC.. |
| | | | | | | | | | | (SEQ ID NO: |
| | | | | | | | | | | 119) |
| 539B- | X49-A01- | GVHS | DIQMTQSPS | RASQSIY | WYQQKP | AASSLQS | GVPSRFSGSG | QQAGI | FGQGTKL | RTVAAPSVFIFPPS |
| X0049- | M at | (SEQ ID | SLSASVGDR | TYLN | GKAPKL | (SEQ ID | SGTDFTLTIS | (SEQ ID | EIK | DEQLKSGTASVVCL |
| A01 | H34(germ- | NO: 124) | VTITC | (SEQ ID | LIY | NO: 128) | SLQPEDFATY | NO: 130) | (SEQ ID | LNNFYPREAKVQWK |
| | line)-LEU | | (SEQ ID | NO:126) | (SEQ ID | | YC | | NO: 131) | VDNALQSGNSQESV |
| | (L) | | NO: 125) | | NO: 127) | | (SEQ ID | | | TEQDSKDSTYSLSS |
| | | | | | | | NO: 129) | | | TLTLSKADYEKHKV |
| | | | | | | | | | | YACEVTHQGLSSPV |
| | | | | | | | | | | TKSFNRGEC.. |
| | | | | | | | | | | (SEQ ID NO: |
| | | | | | | | | | | 119) |
| 539B- | X49-B01- | GVHS | DIQMTQSPS | RASQSIY | WYQQKP | AASSLQS | GVPSRFSGSG | QQAGI | FGQGTKL | RTVAAPSVFIFPPS |
| X0049- | M at | (SEQ ID | SLSASVGDR | TYLN | GKAPKL | (SEQ ID | SGTDFTLTIS | (SEQ ID | EIK | DEQLKSGTASVVCL |
| B01 | H34(germ- | NO: 124) | VTITC | (SEQ ID | LIY | NO: 128) | SLQPEDFATY | NO: 130 | (SEQ ID | LNNFYPREAKVQWK |
| | line)-THR | | (SEQ ID | NO: 126) | (SEQ ID | | YC | | NO: 131) | VDNALQSGNSQESV |
| | (T) | | NO: 125) | | NO: 127) | | (SEQ ID | | | TEQDSKDSTYSLSS |
| | | | | | | | NO: 129) | | | TLTLSKADYEKHKV |
| | | | | | | | | | | YACEVTHQGLSSPV |
| | | | | | | | | | | TKSFNRGEC.. |
| | | | | | | | | | | (SEQ ID NO: |
| | | | | | | | | | | 119) |
| 539B- | X49-C01- | GVHS | DIQMTQSPS | RASQSIY | WYQQKP | AASSLQS | GVPSRFSGSG | QQAGI | FGQGTKL | RTVAAPSVFIFPPS |
| X0049- | M at H56- | (SEQ ID | SLSASVGDR | TYLN | GKAPKL | (SEQ ID | SGTDFTLTIS | (SEQ ID | EIK | DEQLKSGTASVVCL |
| C01 | SER (S) | NO: 124) | VTITC | (SEQ ID | LIY | NO: 128) | SLQPEDFATY | NO: 130) | (SEQ ID | LNNFYPREAKVQWK |
| | | | (SEQ ID | NO: 126) | (SEQ ID | | YC | | NO: 131) | VDNALQSGNSQESV |
| | | | NO: 125) | | NO: 127) | | (SEQ ID | | | TEQDSKDSTYSLSS |
| | | | | | | | NO: 129) | | | TLTLSKADYEKHKV |
| | | | | | | | | | | YACEVTHQGLSSPV |
| | | | | | | | | | | TKSFNRGEC.. |
| | | | | | | | | | | (SEQ ID NO: |
| | | | | | | | | | | 119) |
| 539B- | X49-D01- | GVHS | DIQMTQSPS | RASQSIY | WYQQKP | AASSLQS | GVPSRFSGSG | QQAGI | FGQGTKL | RTVAAPSVFIFPPS |
| X0049- | M at H58- | (SEQ ID | SLSASVGDR | TYLN | GKAPKL | (SEQ ID | SGTDFTLTIS | (SEQ ID | EIK | DEQLKSGTASVVCL |
| D01 | TYR (Y) | NO: 124) | VTITC | (SEQ ID | LIY | NO: 128) | SLQPEDFATY | NO: 130) | (SEQ ID | LNNFYPREAKVQWK |
| | | | (SEQ ID | NO: 126) | (SEQ ID | | YC | | NO: 131) | VDNALQSGNSQESV |
| | | | NO: 125) | | NO: 127) | | (SEQ ID | | | TEQDSKDSTYSLSS |
| | | | | | | | NO: 129) | | | TLTLSKADYEKHKV |
| | | | | | | | | | | YACEVTHQGLSSPV |
| | | | | | | | | | | TKSFNRGEC.. |
| | | | | | | | | | | (SEQ ID NO: |
| | | | | | | | | | | 119) |
| 539B- | X49-E01- | GVHS | DIQMTQSPS | RASQSIY | WYQQKP | AASSLQS | GVPSRFSGSG | QQAGI | FGQGTKL | RTVAAPSVFIFPPS |
| X0049- | M at H56, | (SEQ ID | SLSASVGDR | TYLN | GKAPKL | (SEQ ID | SGTDFTLTIS | (SEQ ID | EIK | DEQLKSGTASVVCL |
| E01 | H58- | NO: 124) | VTITC | (SEQ ID | LIY | NO: 128) | SLQPEDFATY | NO: 130) | (SEQ ID | LNNFYPREAKVQWK |
| | SER, TYR | | (SEQ ID | NO: 126) | (SEQ ID | | YC | | NO: 131) | VDNALQSGNSQESV |
| | (S, Y) | | NO: 125) | | NO: 127) | | (SEQ ID | | | TEQDSKDSTYSLSS |
| | | | | | | | NO: 129) | | | TLTLSKADYEKHKV |
| | | | | | | | | | | YACEVTHQGLSSPV |
| | | | | | | | | | | TKSFNRGEC.. |
| | | | | | | | | | | (SEQ ID NO: |
| | | | | | | | | | | 119) |
| 539B- | X49-F01- | GVHS | DIQMTQSPS | RASQSIY | WYQQKP | AASSLQS | GVPSRFSGSG | QQAGI | FGQGTKL | RTVAAPSVFIFPPS |
| X0049- | M at H34, | (SEQ ID | SLSASVGDR | TYLN | GKAPKL | (SEQ ID | SGTDFTLTIS | (SEQ ID | EIK | DEQLKSGTASVVCL |
| F01 | H56, H58- | NO: 124) | VTITC | (SEQ ID | LIY | NO: 128) | SLQPEDFATY | NO: 130) | (SEQ ID | LNNFYPREAKVQWK |
| | LEU, SER | | (SEQ ID | NO: 126) | (SEQ ID | | YC | | NO: 131) | VDNALQSGNSQESV |
| | TYR | | NO: 125) | | NO: 127) | | (SEQ ID | | | TEQDSKDSTYSLSS |
| | (L, S, Y) | | | | | | NO: 129) | | | TLTLSKADYEKHKV |
| | | | | | | | | | | YACEVTHQGLSSPV |
| | | | | | | | | | | TKSFNRGEC.. |
| | | | | | | | | | | (SEQ ID NO: |
| | | | | | | | | | | 119) |
| 539B- | X49-G01- | GVHS | DIQMTQSPS | RASQSIY | WYQQKP | AASSLQS | GVPSRFSGSG | QQAGI | FGQGTKL | RTVAAPSVFIFPPS |
| X0049- | P at H52a- | (SEQ ID | SLSASVGDR | TYLN | GKAPKL | (SEQ ID | SGTDFTLTIS | (SEQ ID | EIK | DEQLKSGTASVVCL |
| G01 | GLY (G) | NO: 124) | VTITC | (SEQ ID | LIY | NO: 128) | SLQPEDFATY | NO: 130) | (SEQ ID | LNNFYPREAKVQWK |
| | | | (SEQ ID | NO: 126) | (SEQ ID | | YC | | NO: 131) | VDNALQSGNSQESV |
| | | | NO: 125) | | NO: 127) | | (SEQ ID | | | TEQDSKDSTYSLSS |
| | | | | | | | NO: 129) | | | TLTLSKADYEKHKV |
| | | | | | | | | | | YACEVTHQGLSSPV |
| | | | | | | | | | | TKSFNRGEC.. |
| | | | | | | | | | | (SEQ ID NO: |
| | | | | | | | | | | 119) |
| 539B- | X49-H01- | GVHS | DIQMTQSPS | RASQSIY | WYQQKP | AASSLQS | GVPSRFSGSG | QQAGI | FGQGTKL | RTVAAPSVFIFPPS |
| X0049- | M at H52a | (SEQ ID | SLSASVGDR | TYLN | GKAPKL | (SEQ ID | SGTDFTLTIS | (SEQ ID | EIK | DEQLKSGTASVVCL |
| H01 | M at | NO: 124) | VTITC | (SEQ ID | LIY | NO: 128) | SLQPEDFATY | NO: 130) | (SEQ ID | LNNFYPREAKVQWK |
| | H34(germ- | | (SEQ ID | NO: 126) | (SEQ ID | | YC | | NO: 131) | VDNALQSGNSQESV |
| | line)-LEU | | NO: 125) | | NO: 127) | | (SEQ ID | | | TEQDSKDSTYSLSS |
| | (L) | | | | | | NO: 129) | | | TLTLSKADYEKHKV |
| | | | | | | | | | | YACEVTHQGLSSPV |
| | | | | | | | | | | TKSFNRGEC.. |
| | | | | | | | | | | (SEQ ID NO: |
| | | | | | | | | | | 119) |
| 539B- | X49-A02- | GVHS | DIQMTQSPS | RASQSIYT | WYQQKP | AASSLQS | GVPSRFSGSG | QQAGI | FGQGTKL | RTVAAPSVFIFPPS |
| X0049- | P at H52a | (SEQ ID | SLSASVGDR | YLN | GKAPKL | (SEQ ID | SGTDFTLTIS | (SEQ ID | EIK | DEQLKSGTASVVCL |
| A02 | M at | NO: 124) | VTITC | (SEQ ID | LIY | NO: 128) | SLQPEDFATY | NO: 130) | (SEQ ID | LNNFYPREAKVQWK |
| | H34(germ- | | (SEQ ID | NO: 126) | (SEQ ID | | YC | | NO: 131) | VDNALQSGNSQESV |
| | line)-THR | | NO: 125) | | NO: 127) | | (SEQ ID | | | TEQDSKDSTYSLSS |
| | (T) | | | | | | NO: 129) | | | TLTLSKADYEKHKV |
| | | | | | | | | | | YACEVTHQGLSSPV |
| | | | | | | | | | | TKSFNRGEC.. |
| | | | | | | | | | | (SEQ ID NO: |
| | | | | | | | | | | 119) |
| 539B- | X49-B02- | GVHS | DIQMTQSPS | RASQSIYT | WYQQKP | AASSLQS | GVPSRFSGSG | QQAGI | FGQGTKL | RTVAAPSVFIFPPS |
| X0049- | P at H52a | (SEQ ID | SLSASVGDR | YLN | GKAPKL | (SEQ ID | SGTDFTLTIS | (SEQ ID | EIK | DEQLKSGTASVVCL |
| B02 | M at H56- | NO: 124) | VTITC | (SEQ ID | LIY | NO: 128) | SLQPEDFATY | NO: 130) | (SEQ ID | LNNFYPREAKVQWK |
| | SER (S) | | (SEQ ID | NO: 126) | (SEQ ID | | YC | | NO: 131) | VDNALQSGNSQESV |
| | | | NO: 125) | | NO: 127) | | (SEQ ID | | | TEQDSKDSTYSLSS |
| | | | | | | | NO: 129) | | | TLTLSKADYEKHKV |
| | | | | | | | | | | YACEVTHQGLSSPV |
| | | | | | | | | | | TKSFNRGEC.. |
| | | | | | | | | | | (SEQ ID NO: |
| | | | | | | | | | | 119) |
| 539B- | X49-C02- | GVHS | DIQMTQSPS | RASQSIYT | WYQQKP | AASSLQS | GVPSRFSGSG | QQAGI | FGQGTKL | RTVAAPSVFIFPPS |
| X0049- | P at H52a | (SEQ ID | SLSASVGDR | YLN | GKAPKL | (SEQ ID | SGTDFTLTIS | (SEQ ID | EIK | DEQLKSGTASVVCL |
| C02 | M at H58- | NO: 124) | VTITC | (SEQ ID | LIY | NO: 128) | SLQPEDFATY | NO: 130) | (SEQ ID | LNNFYPREAKVQWK |
| | TYR (Y) | | (SEQ ID | NO: 126) | (SEQ ID | | YC | | NO: 131) | VDNALQSGNSQESV |
| | | | NO: 125) | | NO: 127) | | (SEQ ID | | | TEQDSKDSTYSLSS |
| | | | | | | | NO: 129) | | | TLTLSKADYEKHKV |
| | | | | | | | | | | YACEVTHQGLSSPV |
| | | | | | | | | | | TKSFNRGEC.. |
| | | | | | | | | | | (SEQ ID NO: |
| | | | | | | | | | | 119) |
| 539B- | X49-D02- | GVHS | DIQMTQSPS | RASQSIYT | WYQQKP | AASSLQS | GVPSRFSGSG | QQAGI | FGQGTKL | RTVAAPSVFIFPPS |
| X0049- | P at H52a | (SEQ ID | SLSASVGDR | YLN | GKAPKL | (SEQ ID | SGTDFTLTIS | (SEQ ID | EIK | DEQLKSGTASVVCL |
| D02 | M at H56, | NO: 124) | VTITC | (SEQ ID | LIY | NO: 128) | SLQPEDFATY | NO: 130) | (SEQ ID | LNNFYPREAKVQWK |
| | H58- | | (SEQ ID | NO: 126) | (SEQ ID | | YC | | NO: 131) | VDNALQSGNSQESV |
| | SER, TYR | | NO: 125) | | NO: 127) | | (SEQ ID | | | TEQDSKDSTYSLSS |
| | (S, Y) | | | | | | NO: 129) | | | TLTLSKADYEKHKV |
| | | | | | | | | | | YACEVTHQGLSSPV |
| | | | | | | | | | | TKSFNRGEC.. |
| | | | | | | | | | | (SEQ ID NO: |
| | | | | | | | | | | 119) |
| 539B- | X49-E02- | GVHS | DIQMTQSPS | RASQSIYT | WYQQKP | AASSLQS | GVPSRFSGSG | QQAGI | FGQGTKL | RTVAAPSVFIFPPS |
| X0049- | P at H52a | (SEQ ID | SLSASVGDR | YLN | GKAPKL | (SEQ ID | SGTDFTLTIS | (SEQ ID | EIK | DEQLKSGTASVVCL |
| E02 | M at H34, | NO: 124) | VTITC | (SEQ ID | LIY | NO: 128) | SLQPEDFATY | NO: 130) | (SEQ ID | LNNFYPREAKVQWK |
| | H56, H58- | | (SEQ ID | NO: 126) | (SEQ ID | | YC | | NO: 131) | VDNALQSGNSQESV |
| | LEU, SER, | | NO: 125) | | NO: 127) | | (SEQ ID | | | TEQDSKDSTYSLSS |
| | TYR | | | | | | NO: 129) | | | TLTLSKADYEKHKV |
| | (L, S, Y) | | | | | | | | | YACEVTHQGLSSPV |
| | | | | | | | | | | TKSFNRGEC.. |
| | | | | | | | | | | (SEQ ID NO: |
| | | | | | | | | | | 119) |
| | Initial | | | HV- | | | | | | H- |
| Isolate | Name | H-leader | HV-FR1 | CDR1 | HV-FR2 | HV-CDR2 | HV-FR3 | HV-CDR4 | HV-FR4 | Constant |
| 539B- | 539B- | MGWSCIIL | EVQLLESG | DYWMH | WVRQAPG | GISPSGGM | RFTISRDNSKN | DIVGPYS | WGRGTLV | ASTKGPSV |
| X0041- | X0041-D02 | FLVATATG | GGLVQPGG | (SEQ ID | KGLEWVS | TMYADSVK | TLYLQMMSLRA | AGLFDY | TVSS | FPLAPSSK |
| D02 | | AHS | SLRLSCAA | NO: 134 | (SEQ ID | G | EDTAVYYCAR | (SEQ ID | (SEQ ID | S |
| | | (SEQ ID | SGFTFS | | NO: 137) | (SEQ ID | (SEQ ID | NO: 147) | NO: 148) | (SEQ ID |
| | | NO: 132) | (SEQ ID | | | NO: 138) | NO: 146) | | | NO: 149) |
| | | | NO: 133) | | | | | | | |
| 539B- | X49-A01- | MGWSCIIL | EVQLLESG | DYWMH | WVRQAPG | GISPSGGM | RFTISRDNSKN | DIVGPYS | WGRGTLV | ASTKGPSV |
| X0049- | M at | FLVATATG | GGLVQPGG | (SEQ ID | KGLEWVS | TMYADSVK | TLYLQMMSLRA | AGLFDY | TVSS | FPLAPSSK |
| A01 | H34(germ- | AHS | SLRLSCAA | NO: 134 | (SEQ ID | G | EDTAVYYCAR | (SEQ ID | (SEQ ID | S |
| | line)-LEU | (SEQ ID | SGFTFS | | NO: 137) | (SEQ ID | (SEQ ID | NO: 147) | NO: 148) | (SEQ ID |
| | (L) | NO: 132) | (SEQ ID | | | NO: 138) | NO: 146) | | | NO: 149) |
| | | | NO: 133) | | | | | | | |
| 539B- | X49-B01- | MGWSCIIL | EVQLLESG | DYWMH | WVRQAPG | GISPSGGM | RFTISRDNSKN | DIVGPYS | WGRGTLV | ASTKGPSV |
| X0049- | M at | FLVATATG | GGLVQPGG | (SEQ ID | KGLEWVS | TMYADSVK | TLYLQMMSLRA | AGLFDY | TVSS | FPLAPSSK |
| B01 | H34(germ- | AHS | SLRLSCAA | NO: 134 | (SEQ ID | G | EDTAVYYCAR | (SEQ ID | (SEQ ID | S |
| | line)-THR | (SEQ ID | SGFTFS | | NO: 137) | (SEQ ID | (SEQ ID | NO: 147) | NO: 148) | (SEQ ID |
| | (T) | NO: 132) | (SEQ ID | | | NO: 138) | NO: 146) | | | NO: 149) |
| | | | NO: 133) | | | | | | | |
| 539B- | X49-C01- | MGWSCIIL | EVQLLESG | DYWMH | WVRQAPG | GISPSGGM | RFTISRDNSKN | DIVGPYS | WGRGTLV | ASTKGPSV |
| X0049- | M at H56- | FLVATATG | GGLVQPGG | (SEQ ID | KGLEWVS | TMYADSVK | TLYLQMMSLRA | AGLFDY | TVSS | FPLAPSSK |
| C01 | SER (S) | AHS | SLRLSCAA | NO: 134 | (SEQ ID | G | EDTAVYYCAR | (SEQ ID | (SEQ ID | S |
| | | (SEQ ID | SGFTFS | | NO: 137) | (SEQ ID | (SEQ ID | NO: 147) | NO: 148) | (SEQ ID |
| | | NO: 132) | (SEQ ID | | | NO: 138) | NO: 146) | | | NO: 149) |
| | | | NO: 133) | | | | | | | |
| 539B- | X49-D01- | MGWSCIIL | EVQLLESG | DYWMH | WVRQAPG | GISPSGGM | RFTISRDNSKN | DIVGPY | WGRGTLV | ASTKGPSV |
| X0049- | M at H58- | FLVATATG | GGLVQPGG | (SEQ ID | KGLEWVS | TMYADSVK | TLYLQMMSLRA | SAGLFDY | TVSS | FPLAPSSK |
| D01 | TYR (Y) | AHS | SLRLSCAA | NO: 134 | (SEQ ID | G | EDTAVYYCAR | (SEQ ID | (SEQ ID | S |
| | | (SEQ ID | SGFTFS | | NO: 137) | (SEQ ID | (SEQ ID | NO: 147) | NO: 148) | (SEQ ID |
| | | NO: 132) | (SEQ ID | | | NO: 138) | NO: 146) | | | NO: 149) |
| | | | NO: 133) | | | | | | | |
| 539B- | X49-E01- | MGWSCIIL | EVQLLESG | DYWMH | WVRQAPG | GISPSGGM | RFTISRDNSKN | DIVGPY | WGRGTLV | ASTKGPSV |
| X0049- | M AT H56, | FLVATATG | GGLVQPGG | (SEQ ID | KGLEWVS | TMYADSVK | TLYLQMMSLRA | SAGLFDY | TVSS | FPLAPSSK |
| E01 | H58- | AHS | SLRLSCAA | NO: 134 | (SEQ ID | G | EDTAVYYCAR | (SEQ ID | (SEQ ID | S |
| | SER, TYR | (SEQ ID | SGFTFS | | NO: 137) | (SEQ ID | (SEQ ID | NO: 147) | NO: 148) | (SEQ ID |
| | (S, Y) | NO: 132) | (SEQ ID | | | NO: 138) | NO: 146) | | | NO: 149) |
| | | | NO: 133) | | | | | | | |
| 539B- | X49-F01- | MGWSCIIL | EVQLLESG | DYWMH | WVRQAPG | GISPSGGM | RFTISRDNSKN | DIVGPY | WGRGTLV | ASTKGPSV |
| X0049- | M at H34, | FLVATATG | GGLVQPGG | (SEQ ID | KGLEWVS | TMYADSVK | TLYLQMMSLRA | SAGLFDY | TVSS | FPLAPSSK |
| F01 | H56, H58- | AHS | SLRLSCAA | NO: 134 | (SEQ ID | G | EDTAVYYCAR | (SEQ ID | (SEQ ID | S |
| | LEU, SER, | (SEQ ID | SGFTFS | | NO: 137) | (SEQ ID | (SEQ ID | NO: 147) | NO: 148) | (SEQ ID |
| | TYR | NO: 132) | (SEQ ID | | | NO: 138) | NO: 146) | | | NO: 149) |
| | (L, S, Y) | | NO: 133) | | | | | | | |
| 539B- | X49-G01- | MGWSCIIL | EVQLLESG | DYWMH | WVRQAPG | GISPSGGM | RFTISRDNSKN | DIVGPY | WGRGTLV | ASTKGPSV |
| X0049- | P at H52a- | FLVATATG | GGLVQPGG | (SEQ ID | KGLEWVS | TMYADSVK | TLYLQMMSLRA | SAGLFDY | TVSS | FPLAPSSK |
| G01 | GLY (G) | AHS | SLRLSCAA | NO: 134 | (SEQ ID | G | EDTAVYYCAR | (SEQ ID | (SEQ ID | S |
| | | (SEQ ID | SGFTFS | | NO: 137) | (SEQ ID | (SEQ ID | NO: 147) | NO: 148) | (SEQ ID |
| | | NO: 132) | (SEQ ID | | | NO: 138) | NO: 146) | | | NO: 149) |
| | | | NO: 133) | | | | | | | |
| 539B- | X49-H01- | MGWSCIIL | EVQLLESG | DYWMH | WVRQAPG | GISPSGGM | RFTISRDNSKN | DIVGPY | WGRGTLV | ASTKGPSV |
| X0049- | P at H52a | FLVATATG | GGLVQPGG | (SEQ ID | KGLEWVS | TMYADSVK | TLYLQMMSLRA | SAGLFDY | TVSS | FPLAPSSK |
| H01 | M at | AHS | SLRLSCAA | NO: 134 | (SEQ ID | G | EDTAVYYCAR | (SEQ ID | (SEQ ID | S |
| | H34(germ- | (SEQ ID | SGFTFS | | NO: 137) | (SEQ ID | (SEQ ID | NO: 147) | NO: 148) | (SEQ ID |
| | line)-LEU | NO: 132) | (SEQ ID | | | NO: 138) | NO: 146) | | | NO: 149) |
| | (L) | | NO: 133) | | | | | | | |
| 539B- | X49-A02- | MGWSCIIL | EVQLLESG | DYWMH | WVRQAPG | GISPSGGM | RFTISRDNSKN | DIVGPY | WGRGTLV | ASTKGPSV |
| X0049- | P at H52a | FLVATATG | GGLVQPGG | (SEQ ID | KGLEWVS | TMYADSVK | TLYLQMMSLRA | SAGLFDY | TVSS | FPLAPSSK |
| A02 | M at | AHS | SLRLSCAA | NO: 134 | (SEQ ID | G | EDTAVYYCAR | (SEQ ID | (SEQ ID | S |
| | H34(germ- | (SEQ ID | SGFTFS | | NO: 137) | (SEQ ID | (SEQ ID | NO: 147) | NO: 148) | (SEQ ID |
| | line)-THR | NO: 132) | (SEQ ID | | | NO: 138) | NO: 146) | | | NO: 149) |
| | (T) | | NO: 133) | | | | | | | |
| 539B- | X49-B02- | MGWSCIIL | EVQLLESG | DYWMH | WVRQAPG | GISPSGGM | RFTISRDNSKN | DIVGPY | WGRGTLV | ASTKGPSV |
| X0049- | P at H52a | FLVATATG | GGLVQPGG | (SEQ ID | KGLEWVS | TMYADSVK | TLYLQMMSLRA | SAGLFDY | TVSS | FPLAPSSK |
| B02 | M at H56- | AHS | SLRLSCAA | NO: 134 | (SEQ ID | G | EDTAVYYCAR | (SEQ ID | (SEQ ID | S |
| | SER (S) | (SEQ ID | SGFTFS | | NO: 137) | (SEQ ID | (SEQ ID | NO: 147) | NO: 148) | (SEQ ID |
| | | NO: 132) | (SEQ ID | | | NO: 138) | NO: 146) | | | NO: 149) |
| | | | NO: 133) | | | | | | | |
| 539B- | X49-C02- | MGWSCIIL | EVQLLESG | DYWMH | WVRQAPG | GISPSGGM | RFTISRDNSKN | DIVGPY | WGRGTLV | ASTKGPSV |
| X0049- | P at H52a | FLVATATG | GGLVQPGG | (SEQ ID | KGLEWVS | TMYADSVK | TLYLQMMSLRA | SAGLFDY | TVSS | FPLAPSSK |
| C02 | M at H58- | AHS | SLRLSCAA | NO: 134 | (SEQ ID | G | EDTAVYYCAR | (SEQ ID | (SEQ ID | S |
| | TYR (Y) | (SEQ ID | SGFTFS | | NO: 137) | (SEQ ID | (SEQ ID | NO: 147) | NO: 148) | (SEQ ID |
| | | NO: 132) | (SEQ ID | | | NO: 138) | NO: 146) | | | NO: 149) |
| | | | NO: 133) | | | | | | | |
| 539B- | X49-D02- | MGWSCIIL | EVQLLESG | DYWMH | WVRQAPG | GISPSGGM | RFTISRDNSKN | DIVGPY | WGRGTLV | ASTKGPSV |
| X0049- | P at H52a | FLVATATG | GGLVQPGG | (SEQ ID | KGLEWVS | TMYADSVK | TLYLQMMSLRA | SAGLFDY | TVSS | FPLAPSSK |
| D02 | M at H56, | AHS | SLRLSCAA | NO: 134 | (SEQ ID | G | EDTAVYYCAR | (SEQ ID | (SEQ ID | S |
| | H58- | (SEQ ID | SGFTFS | | NO: 137) | (SEQ ID | (SEQ ID | NO: 147) | NO: 148) | (SEQ ID |
| | SER, TYR | NO: 132) | (SEQ ID | | | NO: 138) | NO: 146) | | | NO: 149) |
| | (S, Y) | | NO: 133) | | | | | | | |
| 539B- | X49-E02- | MGWSCIIL | EVQLLESG | DYWMH | WVRQAPG | GISPSGGM | RFTISRDNSKN | DIVGPY | WGRGTLV | ASTKGPSV |
| X0049- | P at H52a | FLVATATG | GGLVQPGG | (SEQ ID | KGLEWVS | TMYADSVK | TLYLQMMSLRA | SAGLFDY | TVSS | FPLAPSSK |
| E02 | M at H34, | AHS | SLRLSCAA | NO: 134 | (SEQ ID | G | EDTAVYYCAR | (SEQ ID | (SEQ ID | S |
| | H56, H58- | (SEQ ID | SGFTFS | | NO: 137) | (SEQ ID | (SEQ ID | NO: 147) | NO: 148) | (SEQ ID |
| | LEU, SER, | NO: 132) | (SEQ ID | | | NO: 138) | NO: 146) | | | NO: 149) |
| | TYR | | NO: 133) | | | | | | | |
| | (L, S, Y) | | | | | | | | | |

| Isolate | Initial Name | LV-AA | HV-AA |
|---|---|---|---|
| 539B- | 539B-X0041-D02 | DIQMTQSPSSLSASVGDRVTITCRASQSIYTYLNW | EVQLLESGGGLVQPGGSLRLSCAASGFTFSDY |
| X0041- | | YQQKPGKAPKLLIYAASSLQSGVPSRFSGSGSGTD | WMHWVRQAPGKGLEWVSGISPSGGMTMYADSV |
| D02 | | FTLTISSLQPEDFATYYCQQAGIFGQGTKLEIK | KGRFTISRDNSKNTLYLQMNSLRAEDTAVYYC |
| | | (SEQ ID NO: 118) | ARDIVGPYSAGLFDYWGRGTLVTVSS |
| | | | (SEQ ID NO: 150) |
| 539B- | X49-A01-M | DIQMTQSPSSLSASVGDRVTITCRASQSIYTYLNW | EVQLLESGGGLVQPGGSLRLSCAASGFTFSDY |
| X0049- | at | YQQKPGKAPKLLIYAASSLQSGVPSRFSGSGSGTD | WMHWVRQAPGKGLEWVSGISPSGGMTMYADSV |
| A01 | H34(germline)- | FTLTISSLQPEDFATYYCQQAGIFGQGTKLEIK | KGRFTISRDNSKNTLYLQMNSLRAEDTAVYYC |
| | LEU (L) | (SEQ ID NO: 118) | ARDIVGPYSAGLFDYWGRGTLVTVSS |
| | | | (SEQ ID NO: 150) |
| 539B- | X49-B01-M | DIQMTQSPSSLSASVGDRVTITCRASQSIYTYLNW | EVQLLESGGGLVQPGGSLRLSCAASGFTFSDY |
| X0049- | at | YQQKPGKAPKLLIYAASSLQSGVPSRFSGSGSGTD | WMHWVRQAPGKGLEWVSGISPSGGMTMYADSV |
| E01 | H34(germline)- | FTLTISSLQPEDFATYYCQQAGIFGQGTKLEIK | KGRFTISRDNSKNTLYLQMNSLRAEDTAVYYC |
| | THR (T) | (SEQ ID NO: 118) | ARDIVGPYSAGLFDYWGRGTLVTVSS |
| | | | (SEQ ID NO: 150) |
| 539B- | X49-C01-M | DIQMTQSPSSLSASVGDRVTITCRASQSIYTYLNW | EVQLLESGGGLVQPGGSLRLSCAASGFTFSDY |
| X0049- | at H56- | YQQKPGKAPKLLIYAASSLQSGVPSRFSGSGSGTD | WMHWVRQAPGKGLEWVSGISPSGGMTMYADSV |
| C01 | SER (S) | FTLTISSLQPEDFATYYCQQAGIFGQGTKLEIK | KGRFTISRDNSKNTLYLQMNSLRAEDTAVYYC |
| | | (SEQ ID NO: 118) | ARDIVGPYSAGLFDYWGRGTLVTVSS |
| | | | (SEQ ID NO: 150) |
| 539B- | X49-D01-M | DIQMTQSPSSLSASVGDRVTITCRASQSIYTYLNW | EVQLLESGGGLVQPGGSLRLSCAASGFTFSDY |
| X0049- | at H58- | YQQKPGKAPKLLIYAASSLQSGVPSRFSGSGSGTD | WMHWVRQAPGKGLEWVSGISPSGGMTMYADSV |
| D01 | TYR (Y) | FTLTISSLQPEDFATYYCQQAGIFGQGTKLEIK | KGRFTISRDNSKNTLYLQMNSLRAEDTAVYYC |
| | | (SEQ ID NO: 118) | ARDIVGPYSAGLFDYWGRGTLVTVSS |
| | | | (SEQ ID NO: 150) |
| 539B- | X49-E01-M | DIQMTQSPSSLSASVGDRVTITCRASQSIYTYLNW | EVQLLESGGGLVQPGGSLRLSCAASGFTFSDY |
| X0049- | at H56, H58- | YQQKPGKAPKLLIYAASSLQSGVPSRFSGSGSGTD | WMHWVRQAPGKGLEWVSGISPSGGMTMYADSV |
| E01 | SER, TYR | FTLTISSLQPEDFATYYCQQAGIFGQGTKLEIK | KGRFTISRDNSKNTLYLQMNSLRAEDTAVYYC |
| | (S, Y) | (SEQ ID NO: 118) | ARDIVGPYSAGLFDYWGRGTLVTVSS |
| | | | (SEQ ID NO: 150) |
| 539B- | X49-F01-M | DIQMTQSPSSLSASVGDRVTITCRASQSIYTYLNW | EVQLLESGGGLVQPGGSLRLSCAASGFTFSDY |
| X0049- | at H34, H56, | YQQKPGKAPKLLIYAASSLQSGVPSRFSGSGSGTD | WMHWVRQAPGKGLEWVSGISPSGGMTMYADSV |
| F01 | H58-LEU, | FTLTISSLQPEDFATYYCQQAGIFGQGTKLEIK | KGRFTISRDNSKNTLYLQMNSLRAEDTAVYYC |
| | SER, TYR | (SEQ ID NO: 118) | ARDIVGPYSAGLFDYWGRGTLVTVSS |
| | (L, S, Y) | | (SEQ ID NO: 150) |
| 539B- | X49-G01-P | DIQMTQSPSSLSASVGDRVTITCRASQSIYTYLNW | EVQLLESGGGLVQPGGSLRLSCAASGFTFSDY |
| X0049- | at H52a- | YQQKPGKAPKLLIYAASSLQSGVPSRFSGSGSGTD | WMHWVRQAPGKGLEWVSGISPSGGMTMYADSV |
| G01 | GLY (G) | FTLTISSLQPEDFATYYCQQAGIFGQGTKLEIK | KGRFTISRDNSKNTLYLQMNSLRAEDTAVYYC |
| | | (SEQ ID NO: 118) | ARDIVGPYSAGLFDYWGRGTLVTVSS |
| | | | (SEQ ID NO: 150) |
| 539B- | X49-H01-P | DIQMTQSPSSLSASVGDRVTITCRASQSIYTYLNW | EVQLLESGGGLVQPGGSLRLSCAASGFTFSDY |
| X0049- | at H52a M at | YQQKPGKAPKLLIYAASSLQSGVPSRFSGSGSGTD | WMHWVRQAPGKGLEWVSGISPSGGMTMYADSV |
| H01 | H34(germline)- | FTLTISSLQPEDFATYYCQQAGIFGQGTKLEIK | KGRFTISRDNSKNTLYLQMNSLRAEDTAVYYC |
| | LEU (L) | (SEQ ID NO: 118) | ARDIVGPYSAGLFDYWGRGTLVTVSS |
| | | | (SEQ ID NO: 150) |
| 539B- | X49-A02-P | DIQMTQSPSSLSASVGDRVTITCRASQSIYTYLNW | EVQLLESGGGLVQPGGSLRLSCAASGFTFSDY |
| X0049- | at H52a M at | YQQKPGKAPKLLIYAASSLQSGVPSRFSGSGSGTD | WMHWVRQAPGKGLEWVSGISPSGGMTMYADSV |
| A02 | H34(germline)- | FTLTISSLQPEDFATYYCQQAGIFGQGTKLEIK | KGRFTISRDNSKNTLYLQMNSLRAEDTAVYYC |
| | THR (T) | (SEQ ID NO: 118) | ARDIVGPYSAGLFDYWGRGTLVTVSS |
| | | | (SEQ ID NO: 150) |
| 539B- | X49-B02-P | DIQMTQSPSSLSASVGDRVTITCRASQSIYTYLNW | EVQLLESGGGLVQPGGSLRLSCAASGFTFSDY |
| X0049- | at H52a M at | YQQKPGKAPKLLIYAASSLQSGVPSRFSGSGSGTD | WMHWVRQAPGKGLEWVSGISPSGGMTMYADSV |
| B02 | H56- | FTLTISSLQPEDFATYYCQQAGIFGQGTKLEIK | KGRFTISRDNSKNTLYLQMNSLRAEDTAVYYC |
| | SER (S) | (SEQ ID NO: 118) | ARDIVGPYSAGLFDYWGRGTLVTVSS |
| | | | (SEQ ID NO: 150) |
| 539B- | X49-C02-P | DIQMTQSPSSLSASVGDRVTITCRASQSIYTYLNW | EVQLLESGGGLVQPGGSLRLSCAASGFTFSDY |
| X0049- | at H52a M at | YQQKPGKAPKLLIYAASSLQSGVPSRFSGSGSGTD | WMHWVRQAPGKGLEWVSGISPSGGMTMYADSV |
| C02 | H58- | FTLTISSLQPEDFATYYCQQAGIFGQGTKLEIK | KGRFTISRDNSKNTLYLQMNSLRAEDTAVYYC |
| | TYR (Y) | (SEQ ID NO: 118) | ARDIVGPYSAGLFDYWGRGTLVTVSS |
| | | | (SEQ ID NO: 150) |
| 539E- | X49-D02-P | DIQMTQSPSSLSASVGDRVTITCRASQSIYTYLNW | EVQLLESGGGLVQPGGSLRLSCAASGFTFSDY |
| X0049- | at H52a M at | YQQKPGKAPKLLIYAASSLQSGVPSRFSGSGSGTD | WMHWVRQAPGKGLEWVSGISPSGGMTMYADSV |
| D02 | H56, H58- | FTLTISSLQPEDFATYYCQQAGIFGQGTKLEIK | KGRFTISRDNSKNTLYLQMNSLRAEDTAVYYC |
| | SER, TYR (S, Y) | (SEQ ID NO: 118) | ARDIVGPYSAGLFDYWGRGTLVTVSS |
| | | | (SEQ ID NO: 150) |
| 539B- | X49-E02-P | DIQMTQSPSSLSASVGDRVTITCRASQSIYTYLNW | EVQLLESGGGLVQPGGSLRLSCAASGFTFSDY |
| X0049- | at H52a M at | YQQKPGKAPKLLIYAASSLQSGVPSRFSGSGSGTD | WMHWVRQAPGKGLEWVSGISPSGGMTMYADSV |
| E02 | H34, H56, H58- | FTLTISSLQPEDFATYYCQQAGIFGQGTKLEIK | KGRFTISRDNSKNTLYLQMNSLRAEDTAVYYC |
| | LEU, SER, TYR | (SEQ ID NO: 118) | ARDIVGPYSAGLFDYWGRGTLVTVSS |
| | (L, S, Y) | | (SEQ ID NO: 150) |

### Example 7

### Evaluation of DX-2712 in a Collagen-Induced Arthritis (CIA) Mouse Model as a Monotherpy or in Combination With Other Disease-Modifying Antirheumatic Drugs (DMARDs) or Biologic Response Modifiers (BRMs)

Previous CIA study with DX-2712, a specific MMP-12 antibody inhibitor, demonstrated that DX-2712 was effective in delaying arthritic disease development and reducing arthritic severity. To further evaluate if DX-2712 can lower the optimal therapeutic doses of DMARDs or BRMs, DX-2712 was tested in a CIA model as a monotherapy or in combination with other DMARDs (methotrexate) or BRMs (ENBREL® or ORENCIA®). The study design is shown in Table 12. In combination groups, DX-2712 was combined with the suboptimal dose of methotrexate, ENBREL® or ORENCIA®, which was determined based on literature reports (Neurath et al. 1999. Clin. Exp. Immunol. 115:42-55; Newton et al. 2001. Ann. Rheum. Dis. 60:iii25-iii32; Zalevsky et al. 2007. J. Immunol. 179:1872-1883).

TABLE 12
CIA combination study design

| | | | Dose | | |
|---|---|---|---|---|---|
| Group | Animals | Test Article | (mg/kg) | ROA | Regimen |
| 1 | 10 DBA/1 | Vehicle | N/A | IP | Q2D |
| 2 | 10 DBA/1 | DX-2712 | 20 | IP | Q2D |
| 3 | 10 DBA/1 | methotrexate | 3 | IP | QD |
| 4 | 10 DBA/1 | ENBREL ® | 30 | SC | QD |
| 5 | 10 DBA/1 | ORENCIA ® | 2 | IP | Q2D |
| 6 | 10 DBA/1 | methotrexate | 1 | IP | QD |
| | | DX-2712 | 20 | IP | Q2D |
| 7 | 10 DBA/1 | ENBREL ® | 10 | SC | QD |
| | | DX-2712 | 20 | IP | Q2D |
| 8 | 10 DBA/1 | methotrexate | 1 | IP | QD |
| | | ENBREL ® | 10 | SC | QD |
| 9 | 10 DBA/1 | ORENCIA ® | 1 | IP | Q2D |
| | | DX-2712 | 20 | IP | Q2D |
| 10 | NON-DISEASED | Vehicle | N/A | IP | Q2D |
| | 10 DBA/1 | | | | |

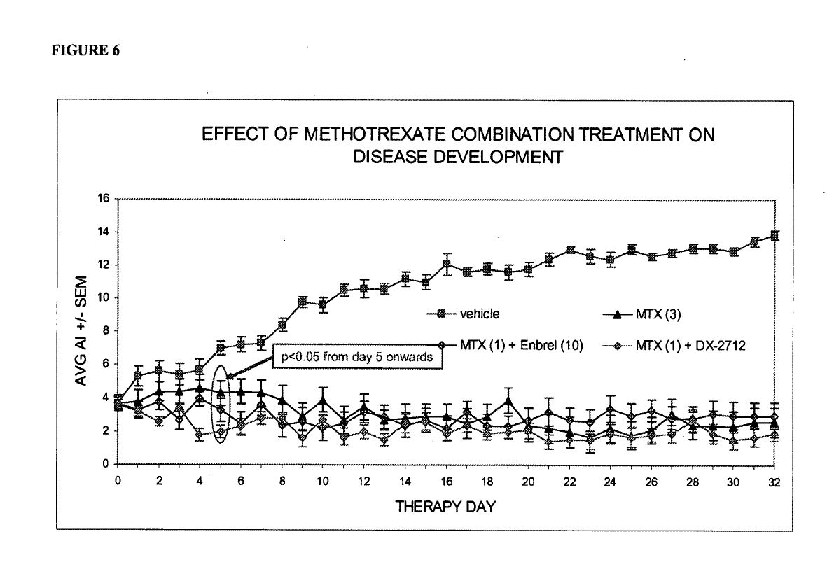

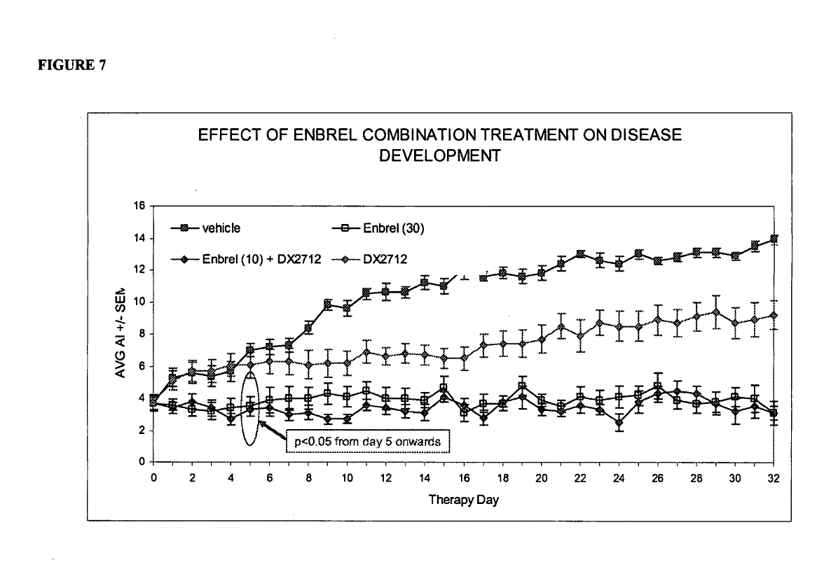

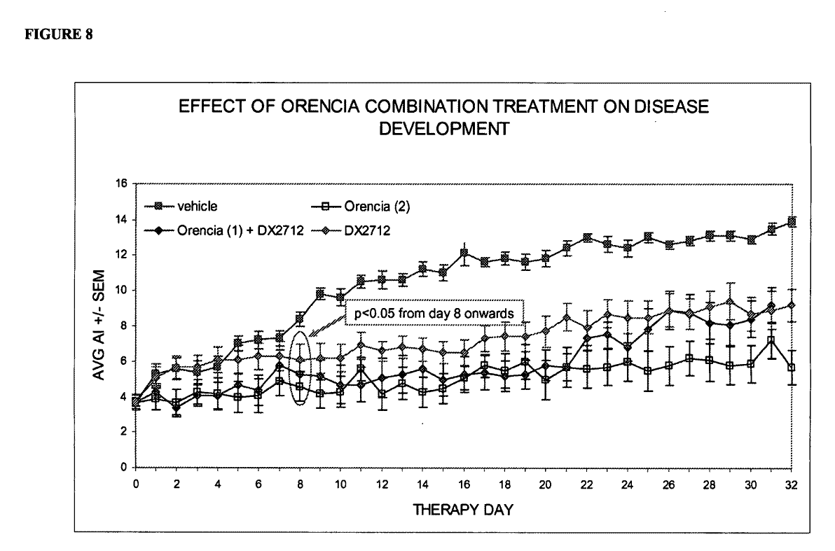

Results are shown in FIGS. 6, 7, and 8, and summarized in Table 13. Combination of suboptimal dose of methotrexate or ENBREL® with DX-2712 brought down the arthritic score to the level of optimally dosed DMARD or BRM monotherapy. Combination with ORENCIA® appeared to lower the arthritic score up to therapy day 22 after which scores increased to the level of DX-2712. However, due to the lack of monotherapy with suboptimal dose of DMARD or BRM (1 mg/kg for methotrexate, 10 mg/kg for ENBREL®, and 1 mg/kg for ORENCIA®), a further in-depth study is carried out to determine the best suboptimal doses of the DMARD or BRMs to use in combination with DX-2712 and to confirm these preliminary findings. The study design is shown in Table 14.

TABLE 13
Result summary of combination study
with DX-2712 and DMARD or BRM

| Group | % change in AI Score* |
|---|---|
| Vehicle | 0.00 |
| DX2712 | −34.07 |
| MTX (3) | −80.74 |
| MTX (1) + DX-2712 | −87.41 |
| Enbrel (30) | −70.37 |
| Enbrel (10) + DX2712 | −74.07 |
| Orencia (2) | −46.67 |
| Orencia (1) + DX2712 | −31.85 |

*End of study 31 days of therapy

TABLE 14
Study design of the further combination CIA study

| | | | Dose | | |
|---|---|---|---|---|---|
| Group | Animals | Test Article | (mg/kg) | ROA | Regimen |
| 1 | 10 DBA/1 | Vehicle | N/A | IP | Q2D |
| 2 | 10 DBA/1 | DX-2712 | 20 | IP | Q2D |
| 3 | 10 DBA/1 | ENBREL ® | TBD | IP | Q2D |
| 4 | 10 DBA/1 | ENBREL ® | TBD | IP | Q2D |
| 5 | 10 DBA/1 | ENBREL ® | TBD | IP | Q2D |
| 6 | 10 DBA/1 | Human IgG control | 20 | IP | Q2D |
| 7 | 10 DBA/1 | ENBREL ® | TBD | IP | Q2D |
| | | DX-2712 | 20 | IP | Q2D |
| 8 | 10 DBA/1 | ENBREL ® | TBD | IP | Q2D |
| | | DX-2712 | 20 | IP | Q2D |
| 9 | 10 DBA/1 | ENBREL ® | TBD | IP | Q2D |
| | | DX-2712 | 20 | IP | Q2D |
| 10 | 10 DBA/1 | ENBREL ® | TBD | IP | Q2D |
| | | Human IgG control | 20 | IP | Q2D |

### Example 8

### Effect of DX-2400 and DX-2410 on Collagen Film Degradation Activity by Human Synovial Cells

Human rheumatoid synovial fibroblasts spontaneously express high levels of MMP-14 (Miller et al, 2009 Arthritis Rheum. 2009 March; 60(3):686-97). When they are cultured on collagen film, MMP-14 expressed in synovial cells effectively degrades the collagen film. Human fibroblasts were cultured on bovine type I collagen film at 37° C. for 4 days in the presence or absence of a broad spectrum MMP-inhibitor GM6001, DX-2400 or DX-2410. Cells were then removed from collagen surface by trypsin/EDTA to reveal collagen film. Films were then fixed with 3% PFA for 30 min. and stained with Coomassie blue. In the presence of GM6001, this degradation was completely abolished (data not shown). Addition of either DX-2400 or DX-2410 also inhibited collagen degradation in a dose-dependent manner (data not shown). At 500 nM, almost complete inhibition was achieved with DX-2400. Inhibitory effect of DX-2410 was slightly lower than DX-2400, showing degraded area even at 500 nM (data not shown).

### Example 10

### Evaluation of DX-2400 and DX-2410 in a Collagen-Induced Arthritis (CIA) Mouse Model

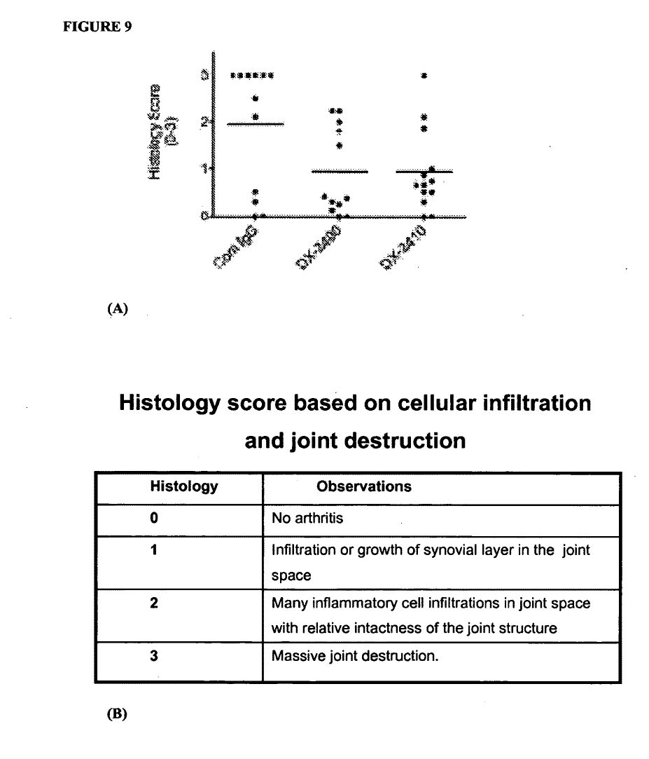

200 μg of chicken type II collagen was injected subcutaneously with complete Freunds adjuvant in DBA mice (male, 3-4 months old). After 21 days, test compounds were administered i.p. at 20 mg/kg, 3×/week. The first mouse which developed clinical symptom of arthritis was detected 26 days after collagen injection. DX-2400 or DX-2410 did not significantly improve redness and swelling of paws but significantly attenuated development of arthritis as assessed by histological analysis of the joints (FIG. 9(A)). FIG. 9(B) provides a description of the histology scoring system used.

### REFERENCES

The contents of all cited references including literature references, issued patents, published or non-published patent applications cited throughout this application are hereby expressly incorporated by reference in their entireties. In case of conflict, the present application, including any definitions herein, will control.

### EQUIVALENTS

A number of embodiments of the invention have been described. Nevertheless, it will be understood that various modifications may be made without departing from the spirit and scope of the invention. Accordingly, other embodiments are within the scope of the following claims.

What is claimed is:

1. A method of treating an inflammatory disorder, the method comprising:
administering a therapeutically effective amount of a protease binding protein in combination with a therapeutically effective amount of a second agent, wherein the second agent is an agent for the treatment of the inflammatory disorder,
to a subject having or suspected of having the inflammatory disorder.

2. The method of claim 1, wherein the inflammatory disorder is selected from the group consisting of: rheumatoid arthritis, psoriasis, multiple sclerosis, systemic sclerosis, asthma, chronic obstructive pulmonary disease, and inflammatory bowel disease.

3. The method of claim 1, wherein the protease binding protein is a protease inhibitor.

4. The method of claim 1, wherein the protease binding protein binds to a protease selected from the group consisting of: MMP-14, plasma kallikrein, plasmin, MMP-9, MMP-9/-2, and MMP-12.

5. The method of claim 1, wherein the protease binding protein is an MMP-14 binding protein.

6. The method of claim 5, wherein the MMP-14 binding protein comprises the 3 heavy chain CDRs and/or the 3 light chain CDRs of DX-2400, DX-2410, M0031-C02, M0031-F01, M0033-H07, M0037-009, M0037-D01, M0038-E06, M0038-F01, M0038-F08, M0039-H08, M0040-A06, M0040-A11, or M0043-G02.

7. The method of claim 5, wherein the MMP-14 binding protein comprises the heavy chain variable region and/or the light chain variable region of DX-2400, DX-2410, M0031-C02, M0031-F01, M0033-H07, M0037-009, M0037-D01, M0038-E06, M0038-F01, M0038-F08, M0039-H08, M0040-A06, M0040-A11, or M0043-G02.

8. The method of claim 5, wherein the MMP-14 binding protein comprises the 3 heavy chain CDRs and/or the 3 light chain CDRs of DX-2400.

9. The method of claim 5, wherein the MMP-14 binding protein comprises the heavy chain variable region and/or the light chain variable region of DX-2400.

10. The method of claim 1, wherein the protease binding protein is an inhibitor of plasma kallikrein.

11. The method of claim 10, wherein the inhibitor of plasma kallikrein is selected from the group consisting of: a Kunitz domain containing polypeptide and a plasma kallikrein binding antibody.

12. The method of claim 10, wherein the inhibitor of plasma kallikrein is a Kunitz domain containing polypeptide that comprises the sequence:
Xaa1 Xaa2 Xaa3 Xaa4 Cys Xaa6 Xaa7 Xaa8 Xaa9 Xaa10 Xaa11 Gly Xaa13 Cys Xaa15 Xaa16 Xaa17 Xaa18 Xaa19 Xaa20 Xaa21 Xaa72 Xaa23 Xaa24 Xaa25 Xaa26 Xaa27 Xaa28 Xaa29 Cys Xaa31 Xaa32 Phe Xaa34 Xaa35 Gly Gly Cys Xaa39 Xaa40 Xaa41 Xaa42 Xaa43 Xaa44 Xaa45 Xaa46 Xaa47 Xaa48 Xaa49 Xaa50 Cys Xaa52 Xaa53 Xaa54 Cys Xaa56 Xaa57 Xaa58 (SEQ ID NO:1), wherein
Xaa10 is Asp or Glu; Xaa11 is Asp, Gly, Ser, Val, Asn, Ile, Ala or Thr; Xaa13 is Pro, Arg, His, Asn, Ser, Thr, Ala, Gly, Lys or Gln; Xaa15 is Arg, Lys, Ala, Ser, Gly, Met, Asn or Gln; Xaa16 is Ala, Gly, Ser, Asp or Asn; Xaa17 is Ala, Asn, Ser, Ile, Gly, Val, Gln or Thr; Xaa18 is His, Leu, Gln or Ala; Xaa19 is Pro, Gln, Leu, Asn or Ile; Xaa21 is Trp, Phe, Tyr, His or Be; Xaa31 is Glu, Asp, Gln, Asn, Ser, Ala, Val, Leu, Be or Thr; Xaa32 is Glu, Gln, Asp Asn, Pro, Thr, Leu, Ser, Ala, Gly or Val; Xaa34 is Be, Thr, Ser, Val, Ala, Asn, Gly or Leu; Xaa35 is Tyr, Trp or Phe; Xaa39 is Glu, Gly, Ala, Ser or Asp; amino acids Xaa6, Xaa7, Xaa8, Xaa9, Xaa20, Xaa24, Xaa25, Xaa26, Xaa27, Xaa28, Xaa29, Xaa41, Xaa42, Xaa44, Xaa46, Xaa47, Xaa48, Xaa49, Xaa50, Xaa52, Xaa53 and Xaa54 is any amino acid; each of the first four and at last three amino acids of SEQ ID NO:1 can each individually optionally be present or absent and is any non-cysteine amino acid.

13. The method of claim 10, wherein the inhibitor of plasma kallikrein is a Kunitz domain containing polypeptide that comprises the sequence of amino acids 3-60 of SEQ ID NO:2.

14. The method of claim 10, wherein the inhibitor of plasma kallikrein is a Kunitz domain containing polypeptide that consists of the sequence of amino acids 3-60 of SEQ ID NO:2.

15. The method of claim 10, wherein the inhibitor of plasma kallikrein is a Kunitz domain containing polypeptide that comprises the sequence of SEQ ID NO:2.

16. The method of claim 10, wherein the inhibitor of plasma kallikrein is a Kunitz domain containing polypeptide that consists of the sequence of SEQ ID NO:2.

17. The method of claim 1, wherein the protease binding protein is an inhibitor of plasmin.

18. The method of claim 17, wherein the inhibitor of plasmin is selected from the group consisting of: a Kunitz domain containing polypeptide and a plasmin binding antibody.

19. The method of claim 17, wherein the inhibitor of plasmin is a Kunitz domain containing polypeptide that comprises the sequence:
Xaa1-Xaa2-Xaa3-Xaa4-Cys-Xaa6-Xaa7-Xaa8-Xaa9-Xaa10-Xaa11-Gly-Xaa13-Cys-Xaa15-Xaa16Xaa17-Xaa18-Xaa 19-Arg-Xaa21-Xaa72-Xaa23-Xaa24-Xaa25-Xaa26-Xaa27-Xaa28-Xaa29-Cys-Xaa31-Xaa 32-Phe-Xaa34-Xaa35-Xaa36-Gly-Cys-Xaa39-Xaa40-Xaa41-Xaa42-Xaa43-Xaa44-Xaa45-Xaa46-Xaa47-Xaa48-Xaa49-Xaa50-Cys-Xaa52-Xaa53-Xaa54-Cys-Xaa56-Xaa57-Xaa58 (SEQ ID NO:300), wherein
Xaa1, Xaa2, Xaa3, Xaa4, Xaa56, Xaa57 and Xaa58 may each individually be absent; Xaa10 is Asp, Glu, Tyr, or Gln; Xaal 1 is Thr, Ala, Ser, Val or Asp; Xaa13 is Pro, Leu or Ala; Xaa15 is Lys or Arg; Xaa16 is Ala or Gly; Xaa17 is Arg, Lys or Ser; Xaa18 is Phe or Ile; Xaa19 is Glu, Gln, Asp, Pro, Gly, Ser or Ile; Xaa21 is Phe, Tyr or Trp; Xaa22 is Tyr or Phe. Xaa23 is Tyr or Phe; Xaa31 is Asp, Glu, Thr, Val, Gln or Ala; Xaa32 is Thr, Ala, Glu, Pro, or Gln; Xaa34 is Val, Ile, Thr, Leu, Phe, Tyr, His, Asp, Ala, or Ser; Xaa35 is Tyr or Tip; Xaa36 is Gly or Ser; Xaa39 is Glu, Gly, Asp, Arg, Ala, Gln, Leu, Lys, or Met; Xaa40 is Gly or Ala; Xaa43 is Asn or Gly; or Xaa45 is Phe or Tyr; and where not specified, Xaa is any non-cysteine amino acid.

20. The method of claim 17, wherein the inhibitor of plasmin is a Kunitz domain containing polypeptide that comprises the sequence of SEQ ID NO:100.

21. The method of claim 17, wherein the inhibitor of plasmin is a Kunitz domain containing polypeptide that consists of the sequence of SEQ ID NO:100.

22. The method of claim 17, wherein the inhibitor of plasmin is a Kunitz domain containing polypeptide that comprises the sequence of SEQ ID NO:200.

23. The method of claim 17, wherein the inhibitor of plasmin is a Kunitz domain containing polypeptide that consists of the sequence of SEQ ID NO:200.

24. The method of claim 1, wherein the protease binding protein is an MMP-9 binding protein.

25. The method of claim 24, wherein the MMP-9 binding protein comprises the 3 heavy chain CDRs and/or the 3 light chain CDRs of DX-2802, 539A-M0240-B03, M0078-G07, M0081-D05, M0076-D03, M0072-H07, M0075-D12, M0166-F10, M0279-A03, M0279-B02, M0288-008, or M0281-F06.

26. The method of claim 24, wherein the MMP-9 binding protein comprises the heavy chain variable region and/or the light chain variable region of DX-2802, 539A-M0240-B03, M0078-G07, M0081-D05, M0076-D03, M0072-H07, M0075-D12, M0166-F10, M0279-A03, M0279-B02, M0288-008, or M0281-F06.

27. The method of claim 24, wherein the MMP-9 binding protein comprises the 3 heavy chain CDRs and/or the 3 light chain CDRs of DX-2802.

28. The method of claim 24, wherein the MMP-9 binding protein comprises the heavy chain variable region and/or the light chain variable region of DX-2802.

29. The method of claim 1, wherein the protease binding protein is an MMP-9/-2 binding protein.

30. The method of claim 29, wherein the MMP-9/-2 binding protein comprises the 3 heavy chain CDRs and/or the 3 light chain CDRs of M0237-D02, X0106-A01, X0106-B02, X0106-004, X0106-E4, or X0106-F05.

31. The method of claim 29, wherein the MMP-9/-2 binding protein comprises the heavy chain variable region and/or the light chain variable region of M0237-D02, X0106-A01, X0106-B02, X0106-004, X0106-E4, or X0106-F05.

32. The method of claim 29, wherein the MMP-9/-2 binding protein comprises the 3 heavy chain CDRs and/or the 3 light chain CDRs of M0237-D02.

33. The method of claim 29, wherein the MMP-9/-2 binding protein comprises the heavy chain variable region and/or the light chain variable region of M0237-D02.

34. The method of claim 1, wherein the protease binding protein is an MMP-12 binding protein.

35. The method of claim 34, wherein the MMP-12 binding protein comprises the 3 heavy chain CDRs and/or the 3 light chain CDRs of DX-2712, a mutant or variant of DX-2712, 539B-X0041-D02, M0134-A02, M134-A05, M134-A07, M134-A09, M134-A10, M134-A11, M0134-B01, M134-B04, M0134-B08, M0134-B11, M0134-001, M0134-C02, M0134-006, M0134-009, M0134-C10, M0134-C11, M0134-C12, M0134-D02, M0134-D03, M0134-E04, M0134-E07, M0134-E08, M134-E11, M0134-F01, M0134-F05, M0134-G02, M0134-004, M0134-G07, M0135-A03, M0135-A05, M0135-A06, M0135-A07, M0135-B02, M0135-B08, M0135-001, M0135-C11, M0135-E03, M0135-F03, M0135-F11, M0135-G02, M0135-G03, M0135-G07, M0135-G11, M0135-H03, M0135-H10, M0105-C05, M0105-E11, M0105-F08, M0107-A12, M0108-A02, M0109-G11, M0110-G05, M0129-B11, M0130-A01, M0130-C12, M0130-F06, M0130-H04, M0131-A06, M0131-D03, M0132-A04, M0133-B08, M0133-E05, M0121-E07, M0118-F11, M0125-G07, M0124-E07, M0119-D01, M0119-A02, M0122-006, M0123-G07, M0063-A02, M0063-A04, M0063-B01, M0063-B11, M0063-007, M0063-G01, M0065-E12, M0065-003, M0065-H05, M0067-A02, M0067-B06, M0067-B09, M0067-C10, M0067-F02, M0067-F06, M0069-A04, M0069-A11, M0069-C02, M0069-D10, M0069-007, M0071-A01, M0071-B07, M0071-D05, M0071-D09, M0071-H03, M0071-H06, M0087-F09, M0088-F07, M0088-G10, M0088-H10, M0089-001, M0089-F05, M0089-B07, M0089-H11, M0032-E01, M0034-004, M0039-F01, M0041-B05, M0041-G01, M0042-B06, M0006-B10, M0007-H06, M0008-H09, M00094108, M0011-H11, M0015-F02, M0016-D01, M0013-D11, M0013-G12, M0013-H06, M0014-009, M0014-G11, M0016-A11, M0016-H05, M0019-C05, M0020-B01, M0022-007, M0025-D04, M0027-E11, 539B-X0041-D02, 539B-X0049-A01, 539B-X0049-B01, 539B-X0049-C01, 539B-X0049-D01, 539B-X0049-E01, 539B-X0049-F01, 539B-X0049-G01, 539B-X0049-H01, 539B-X0049-A02, 539B-X0049-B02, 539B-X0049-C02, 539B-X0049-D02, or 539B-X0049-E02.

36. The method of claim 34, wherein the MMP-12 binding protein comprises the heavy chain variable region and/or the light chain variable region of DX-2712, a mutant or variant of DX-2712, 539B-X0041-D02, M0134-A02, M134-A05, M134-A07, M134-A09, M134-A10, M134-A11, M0134-B01, M134-B04, M0134-B08, M0134-B11, M0134-001, M0134-C02, M0134-006, M0134-009, M0134-C10, M0134-C11, M0134-C12, M0134-D02, M0134-D03, M0134-E04, M0134-E07, M0134-E08, M134-E11, M0134-F01, M0134-F05, M0134-G02, M0134-G04, M0134-G07, M0135-A03, M0135-A05, M0135-A06, M0135-A07, M0135-B02, M0135-B08, M0135-001, M0135-C11, M0135-E03, M0135-F03, M0135-F11, M0135-G02, M0135-G03, M0135-G07, M0135-G11, M0135-H03, M0135-H10, M0105-C05, M0105-E11, M0105-F08, M0107-A12, M0108-A02, M0109-G11, M0110-G05, M0129-B11, M0130-A01, M0130-C12, M0130-F06, M0130-H04, M0131-A06, M0131-D03, M0132-A04, M0133-B08, M0133-E05, M0121-E07, M0118-F11, M0125-G07, M0124-E07, M0119-D01, M0119-A02, M0122-C06, M0123-G07, M0063-A02, M0063-A04, M0063-B01, M0063-B11, M0063-007, M0063-G01, M0065-E12, M0065-G03, M0065-H05, M0067-A02, M0067-B06, M0067-B09, M0067-C10, M0067-F02, M0067-F06, M0069-A04, M0069-A11, M0069-C02, M0069-D10, M0069-G07, M0071-A01, M0071-B07, M007′-D05, M0071-D09, M0071-H03, M0071-H06, M0087-F09, M0088-F07, M0088-G10, M0088-H10, M0089-001, M0089-F05, M0089-B07, M0089-H11, m0032-E01, M0034-004, M0039-F01, M0041-B05, M0041-601, M0042-B06, M0006-B10, M0007-H06, M0008-H09, M0009-H08, M0011-H11, M0015-F02, M0016-D01, M0013-D11, M0013-G12, M0013-H06, M0014-009, M0014-G11, M0016-A11, M0016-H05, M0019-C05, M0020-B01, M0022-007, M0025-D04, M0027-E11, 539B-X0041-D02, 539B-X0049-A01, 539B-X0049-B01, 539B-X0049-001, 539B-X0049-D01, 539B-X0049-E01, 539B-X0049-F01, 539B-X0049-G01, 539B-X0049-H01, 539B-X0049-A02, 539B-X0049-B02, 539B-X0049-C02, 539B-X0049-D02, or 539B-X0049-E02.

37. The method of claim 34, wherein the MMP-12 binding protein comprises the 3 heavy chain CDRs and/or the 3 light chain CDRs of DX-2712.

38. The method of claim 34, wherein the MMP-12 binding protein comprises the heavy chain variable region and/or the light chain variable region of DX-2712.

39. The method of claim 1, wherein the second agent is an immunosuppressant agent selected from the group consisting of: gancyclovir, etanercept, cyclosporine, tacrolimus, rapamycin, cyclophosphamide, azathioprine, mycophenolate mofetil, methotrexate, cortisol, aldosterone, dexamethasone, a cyclooxygenase inhibitor, a 5-lipoxygenase inhibitor, and leukotriene receptor antagonist.

40. The method of claim 1, wherein the second agent is an agent for the treatment of an inflammatory disorder selected from the group of inflammatory disorders consisting of: rheumatoid arthritis, psoriasis, multiple sclerosis, systemic sclerosis, asthma, chronic obstructive pulmonary disease, and inflammatory bowel disease.

41. The method of claim 40, wherein the second agent is an agent for the treatment of rheumatoid arthritis and comprises a nonsteroidal anti-inflammatory drug (NSAID), a corticosteroid, a Disease-Modifying Antirheumatic Drug (DMARD), or a biological response modifier (BRM).

42. The method of claim 40, wherein the second agent is an agent for the treatment of rheumatoid arthritis and comprises aspirin, naproxen, ibuprofen, etodolac, gold, salsalte, methotrexate, sulfasalazine, D-penicillamine, azathioprine, cyclophosphamide, chlorambucil, cyclosporine, leflunomide, etanercept, infliximab, anakinra, adalimumab, hydroxychloroquine, chloroquine phosphate, chloroquine sulphate, minocycline, or a CTLA4-Ig.

43. The method of claim 40, wherein the second agent is an agent for the treatment of psoriasis and comprises a topical treatment or a systemic treatment.

44. The method of claim 40, wherein the second agent is an agent for the treatment of psoriasis and comprises a topical treatment selected from the group consisting of coal tar, dithranol, a corticosteroid, a vitamin D3 analogue, and a retinoid.

45. The method of claim 40, wherein the second agent is an agent for the treatment of psoriasis and comprises a systemic treatment selected from the group consisting of methotrexate, cyclosporine, a retinoid, tioguanine, hydroxyurea, sulfasalazine, mycophenolate mofetil, azathioprine, tacrolimus, alefacept, efalizumab, etanercept, and infliximab.

46. The method of claim 40, wherein the second agent is an agent for the treatment of multiple sclerosis and comprises a corticosteroid, an interferon, glatiramer acetate, an immunosuppressant or natalizumab.

47. The method of claim 40, wherein the second agent is an agent for the treatment of systemic sclerosis and comprises an NSAID, a calcium channel blocker, prostacyclin analogue, a dual endothelin-receptor antagonist, methotrexate, cyclosporin, an ACE inhibitor, cyclophosphamide, a steroid, or epoprostenol.

48. The method of claim 40, wherein the second agent is an agent for the treatment of systemic sclerosis and comprises nifedipine.

49. The method of claim 40, wherein the second agent is an agent for the treatment of systemic sclerosis and comprises iloprost.

50. The method of claim 40, wherein the second agent is an agent for the treatment of systemic sclerosis and comprises bosentan.

51. The method of claim 40, wherein the second agent is an agent for the treatment of asthma and comprises a glucocorticoid, a leukotriene modifier, a mast cell stabilizer, an antimuscarinic/anticholinergic, an antihistamines, an IgE blocker, or methotrexate.

52. The method of claim 40, wherein the second agent is an agent for the treatment of asthma and comprises a beta₂-adrenoceptor agonist selected from the group consisting of: salbutamol, levalbuterol, terbutaline and bitolterol.

53. The method of claim 40, wherein the second agent is an agent for the treatment of asthma and comprises an adrenergic agonist selected from the group consisting of: inhaled epinephrine and ephedrine tablets.

54. The method of claim 40, wherein the second agent is an agent for the treatment of asthma and comprises an antimuscarinic/anticholinergic selected from the group consisting of: ipratropium, oxitropium, and tiotropium.

55. The method of claim 40, wherein the second agent is an agent for the treatment of asthma and comprises an inhaled glucocorticoid selected from the group consisting of: ciclesonide, beclomethasone, budesonide, flunisolide, fluticasone, mometasone, and triamcinolone.

56. The method of claim 40, wherein the second agent is an agent for the treatment of asthma and comprises a leukotriene modifier selected from the group consisting of: montelukast, zafirlukast, pranlukast, and zileuton.

57. The method of claim 40, wherein the second agent is an agent for the treatment of asthma and comprises a mast cell stabilizer selected from the group consisting of: cromoglicate and nedocromil.

58. The method of claim 40, wherein the second agent is an agent for the treatment of asthma and comprises a methylxanthine selected from the group consisting of: theophylline and aminophylline.

59. The method of claim 40, wherein the second agent is an agent for the treatment of asthma and comprises an antihistamine.

60. The method of claim 40, wherein the second agent is an agent for the treatment of asthma and comprises an IgE blocker selected from the group consisting of: omalizumab and methotrexate.

61. The method of claim 40, wherein the second agent is an agent for the treatment of asthma and comprises a long-acting beta₂-adrenoceptor agonist selected from the group consisting of: salmeterol, formoterol, bambuterol, and albuterol.

62. The method of claim 40, wherein the second agent is an agent for the treatment of asthma and comprises a combination of inhaled steroid and a long-acting bronchodilator selected from the group consisting of: fluticasone/salmeterol and budesonide/formoterol.

63. The method of claim 40, wherein the second agent is an agent for the treatment of chronic obstructive pulmonary disease (COPD) and comprises a bronchodilator.

64. The method of claim 40, wherein the second agent is an agent for the treatment of chronic obstructive pulmonary disease (COPD) and comprises a β₂ agonist, an M₃ antimuscarinic, a leukotriene antagonist, a cromone, a corticosteroid, or a xanthine.

65. The method of claim 40, wherein the second agent is an agent for the treatment of chronic obstructive pulmonary disease (COPD) and comprises a β₂ agonist selected from the group consisting of: Salbutamol, Bambuterol, Clenbuterol, Fenoterol, Formoterol, and Salmeterol.

66. The method of claim 40, wherein the second agent is an agent for the treatment of chronic obstructive pulmonary disease (COPD) and comprises the Ipratropium.

67. The method of claim 40, wherein the second agent is an agent for the treatment of chronic obstructive pulmonary disease (COPD) and comprises a cromone selected from the group consisting of: Cromoglicate and Nedocromil.

68. The method of claim 40, wherein the second agent is an agent for the treatment of chronic obstructive pulmonary disease (COPD) and comprises a leukotriene antagonist selected from the group consisting of: Montelukast, Pranlukast, and Zafirlukast.

69. The method of claim 40, wherein the second agent is an agent for the treatment of chronic obstructive pulmonary disease (COPD) and comprises a corticosteroid antagonist selected from the group consisting of: glucocorticoids, beclomethasone, mometasone, and fluticasone.

70. The method of claim 40, wherein the second agent is an agent for the treatment of chronic obstructive pulmonary disease (COPD) and comprises a xanthine antagonist selected from the group consisting of: theophylline, methylxanthine, and theobromine.

71. The method of claim 40, wherein the second agent is an agent for the treatment of chronic obstructive pulmonary disease (COPD) and comprises Ipratropium or Tiotropium.

72. The method of claim 40, wherein the second agent is an agent for the treatment of inflammatory bowel disease and comprises an immunosuppresant, an anti-TNF binding protein, a cytokine inhibitor, a BRM, or an anti-inflammatory.

73. The method of claim 40, wherein the second agent is an agent for the treatment of inflammatory bowel disease and comprises prednisone, infliximab, azathioprine, methotrexate, 6-mercaptopurine , a mesalamine, a steroid, or CDP571 antibody.

74. A method of preventing an inflammatory disorder, the method comprising:
administering a therapeutically effective amount of a protease binding protein in combination with a therapeutically effective amount of a second agent, wherein the second agent is an agent for the treatment of the inflammatory disorder,
to a subject at risk of having the inflammatory disorder.

75. A composition comprising a protease binding protein and a second agent, wherein the second agent is an agent for the treatment of an inflammatory disorder.

76. A kit comprising a protease binding protein and a second agent, wherein the second agent is an agent for the treatment of an inflammatory disorder.
